(19) United States
(12) Patent Application Publication
(10) Pub. No.: US 2026/0116874 A1
(43) Pub. Date: Apr. 30, 2026

(54) HETEROAROMATIC INDOLESULFONAMIDES

(71) Applicant: SEED Therapeutics, Inc., King of Prussia, PA (US)

(72) Inventors: Haihong Jin, King of Prussia, PA (US); Dong Liu, King of Prussia, PA (US); Xing Liu, King of Prussia, PA (US); James Finn, King of Prussia, PA (US); James Tonra, Doylestown, PA (US); Lan Huang, New York, NY (US)

(21) Appl. No.: 19/480,279

(22) PCT Filed: Apr. 30, 2024
(86) PCT No.: PCT/US2024/027089

§ 371 Date: Oct. 30, 2025

Related U.S. Application Data
(60) Provisional application No. 63/499,427, filed on May 1, 2023.

(51) Int. Cl. C07D 417/12; A61K 31/404; A61K 31/4162; A61K 31/422; A61K 31/4245; A61K 31/427; A61K 31/433; A61K 31/437; A61K 31/454; A61K 31/5377; A61K 31/541; A61K 45/06; A61P 35/00; C07D 209/42; C07D 413/12; C07D 413/14; C07D 417/14; C07D 471/04; C07D 487/04

(52) U.S. Cl. CPC C07D 417/12; A61K 31/404; A61K 31/4162; A61K 31/422; A61K 31/4245; A61K 31/427; A61K 31/433; A61K 31/437; A61K 31/454; A61K 31/5377; A61K 31/541; A61K 45/06; A61P 35/00; C07D 209/42; C07D 413/12; C07D 413/14; C07D 417/14; C07D 471/04; C07D 487/04

(57) ABSTRACT

The present disclosures relate to compounds that can be useful as modulators of splicing factor RBM39. Also disclosed herein are pharmaceutical compositions that can include a compound of Formula (I), the use and preparation thereof.

### INCORPORATION BY REFERENCE TO ANY PRIORITY APPLICATIONS

This application claims the benefit of priority to U.S. Provisional Application Nos. 63/499,427, filed May 1, 2023.

### BACKGROUND

### Field

The present disclosure relates generally to the fields of chemistry and medicine. More specifically, the present disclosure relates to the field of small molecule drugs for the treatment of cancer.

### Description of the Related Art

Aryl sulfonamides can act as molecular glues to induce aggregation between two or more proteins to modulate biological functions. More particularly, Indisulam and E7820 are both associated with the degradation of splicing factor RBM39 to achieve anticancer effects. In the presence of indisulam or E7820, RBM39 associates with the E3 ligase CUL4-DDB1-DDA1-DCAF15, leading to RBM39 polyubiquitination and proteasomal degradation. Indisulam and E7820 have been investigated in multiple phase I and II clinical trials involving advanced cancers with modest clinical responses. Therefore, a need exists for new sulfonamide compounds with more potent anticancer activity.

### SUMMARY

Some embodiments disclosed herein include a compound of Formula (I):

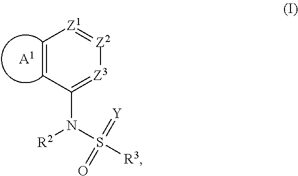

or a pharmaceutically acceptable salt thereof, wherein:
A¹ is selected from

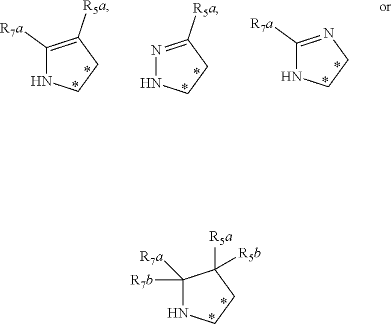

and * represents points of attachment to form a fused bicyclic ring;
Y is O or NH;
Z¹, Z² and Z³ are each independently C(R^{1a}) or N;
each R^{1a} is independently selected from the group consisting of H, halogen, —(C₁-C₆)alkyl and —(C₁-C₆)haloalkyl;
R² is H, —(C₁-C₆)alkyl or —C(O)R⁶;
R³ is a —(C₁-C₆)alkyl, furan, thiophene, a 5-membered monocyclic nitrogen-containing heteroaryl, or a 6-12 membered nitrogen-containing bicyclic heterocyclyl; wherein the —(C₁-C₆)alkyl, furan, thiophene, 5-membered monocyclic nitrogen-containing heteroaryl and the 6-12 membered nitrogen-containing bicyclic heterocyclyl can be optionally substituted with one or two or three substituents selected from R⁴;
each R⁴ is independently selected independently selected from —R^{x1}, —R^{x2}, —(C₁-C₆)alkyl, —(C₁-C₆)haloalkyl, —(C₁-C₆)alkoxy, —CN, halogen, —NH₂, —N((C₁-C₆)alkyl)₂, —NHC(O)(C₁-C₆)alkyl, —NHBoc, —(CH₂)ₙS(O)₂(C₁-C₆)alkyl and —C(O)R^{z1};
R^{5a} is selected from the group consisting of —H, —CN, halogen, —(C₁-C₆)alkyl, —(C₁-C₆)haloalkyl, —(C₂-C₆)alkenyl and —(C₂-C₆)alkynyl;
R^{5b} is —(C₁-C₆)alkyl; or
R^{5a} is taken together with R^{5b} and the atom to which R^{5a} and R^{5b} are attached to form an optionally substituted 3-7 membered monocyclic cycloalkyl;
R⁶ is H or —(C₁-C₆)alkyl;
R^{7a} and R^{7b} are each independently selected from the group consisting of H, halogen, —CN, —(C₁-C₆)alkyl, —(C₁-C₆)alkoxy, 3-7 membered monocyclic cycloalkyl and —(C₁-C₆)haloalkyl; or R^{7a} is taken together with R^{7b} and the atom to which R^{7a} and R^{7b} are attached to be —C(═O);
R^{x1} is selected from the group consisting of cycloalkyl, heterocyclyl, and heterocyclyl(alkyl), wherein the cycloalkyl, heterocyclyl and heterocyclyl(alkyl) are each optionally substituted with R^{y1};
R^{x2} is selected from the group consisting of —(C₁-C₆)alkyl, —(C₁-C₆)alkoxy, alkylamino, and amino; wherein the —(C₁-C₆)alkyl, —(C₁-C₆)alkoxy, alkylamino, and amino are optionally substituted with one or two R^{y2};
R^{y1} is selected from the group consisting of H, —CN, —OH, —C(O)O(C₁-C₆)alkyl, —(C₁-C₆)alkyl, —(C₁-C₅)haloalkyl, —(C₁-C₆)alkoxy, heterocyclyl, BOC, —C(O)(C₁-C₆)alkyl, —S(O)₂(C₁-C₆)alkyl, —CH₂S(O)₂(C₁-C₆)alkyl and —CH₂CN;
each R^{y2} is independently selected from the group consisting of —CN, —OH, —(C₁-C₆)alkyl, —(C₁-C₆)haloalkyl, —(C₁-C₆)alkoxy, —N((C₁-C₆)alkyl)₂, —CH₂CN, —C(O)CH₂CH₂N((C₁-C₆)alkyl)₂, —C(O)(heterocyclyl) and —(CH₂)ₙS(O)₂(C₁-C₆)alkyl;
n is 0, 1, 2, 3 or 4; and
R^{z1} is

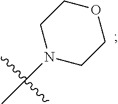 [SMILES: *N1CCOCC1]

with the proviso that the compound is not

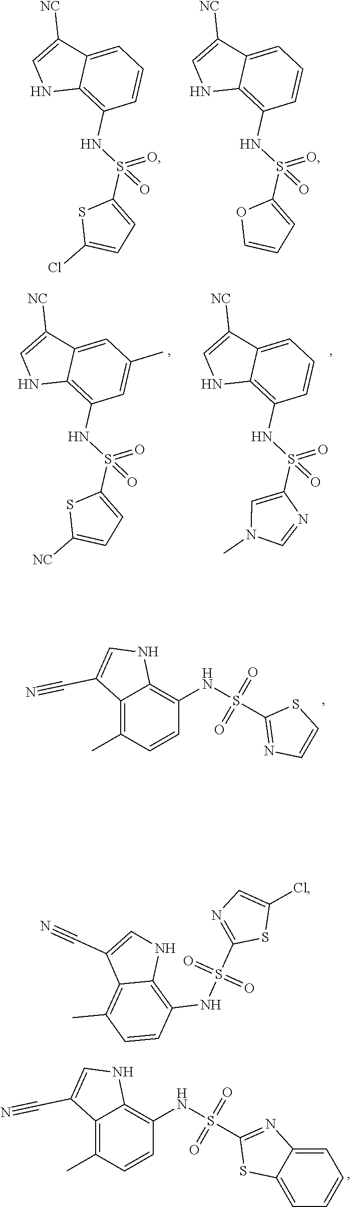

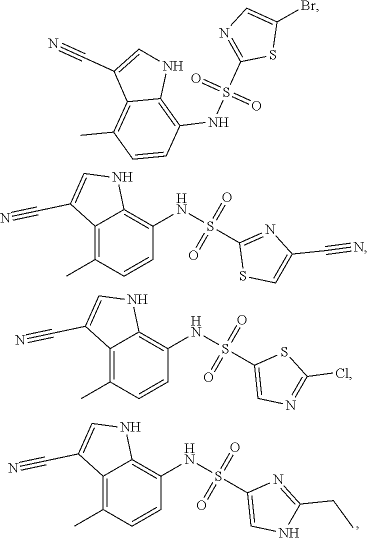

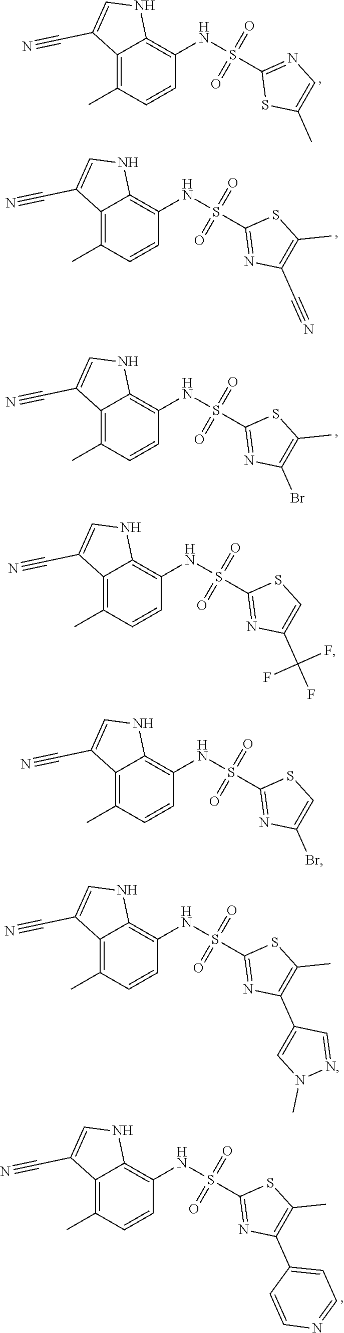

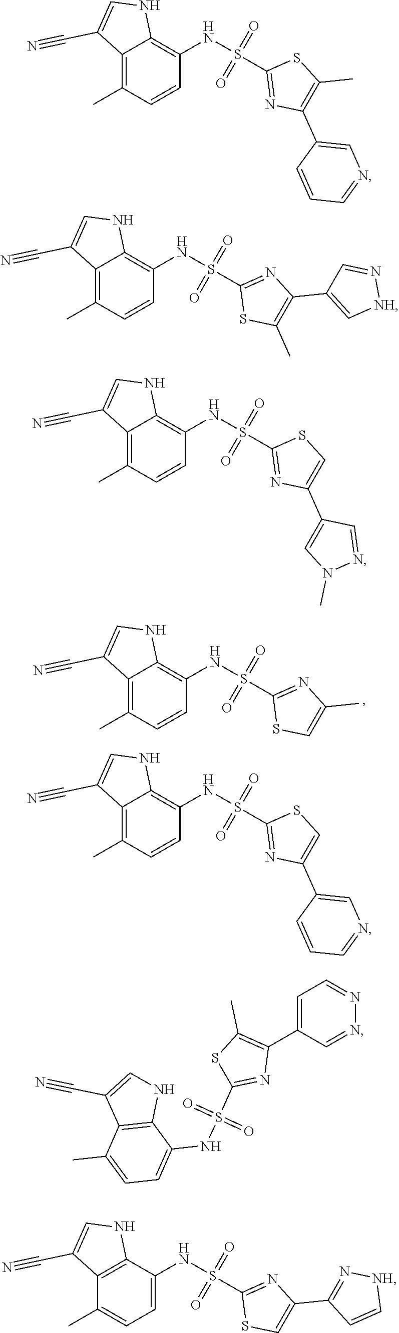

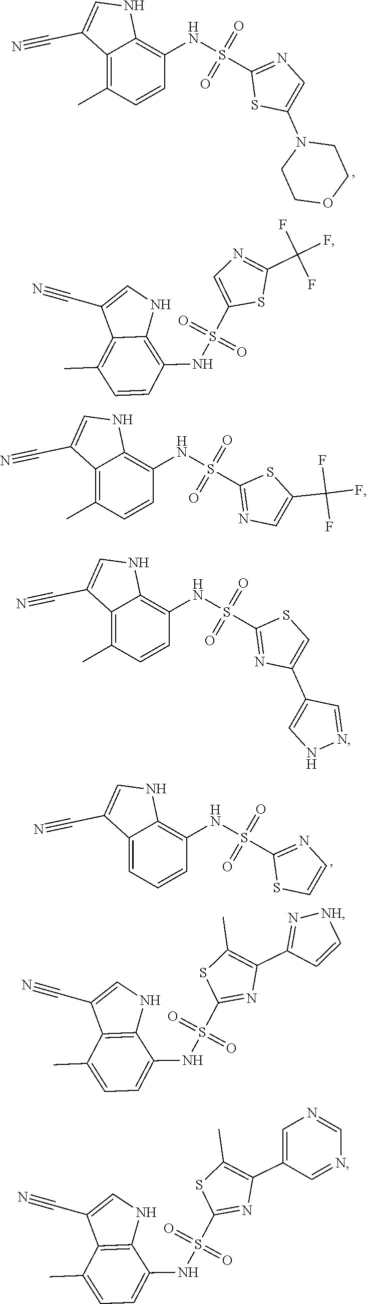

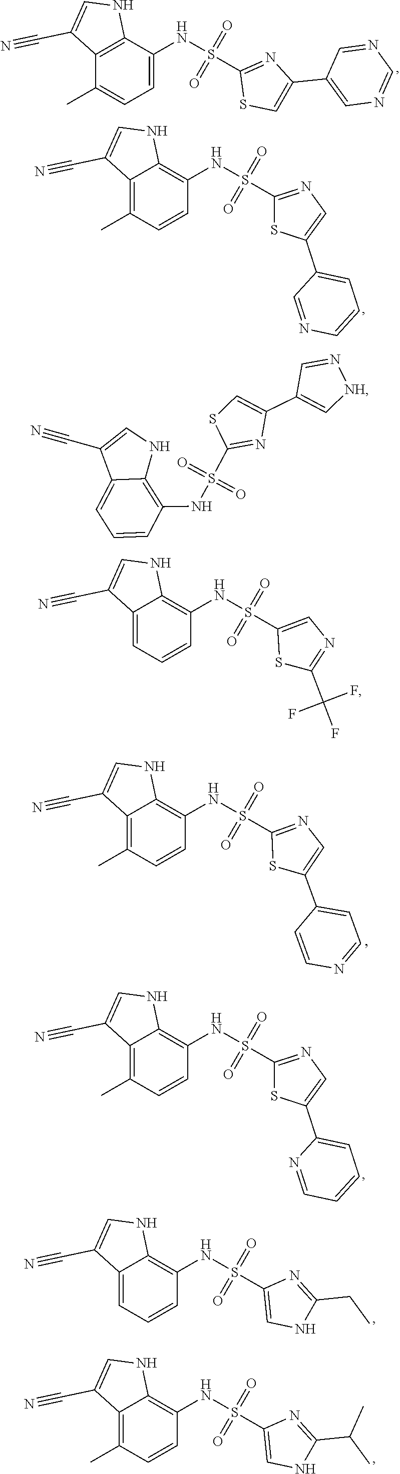

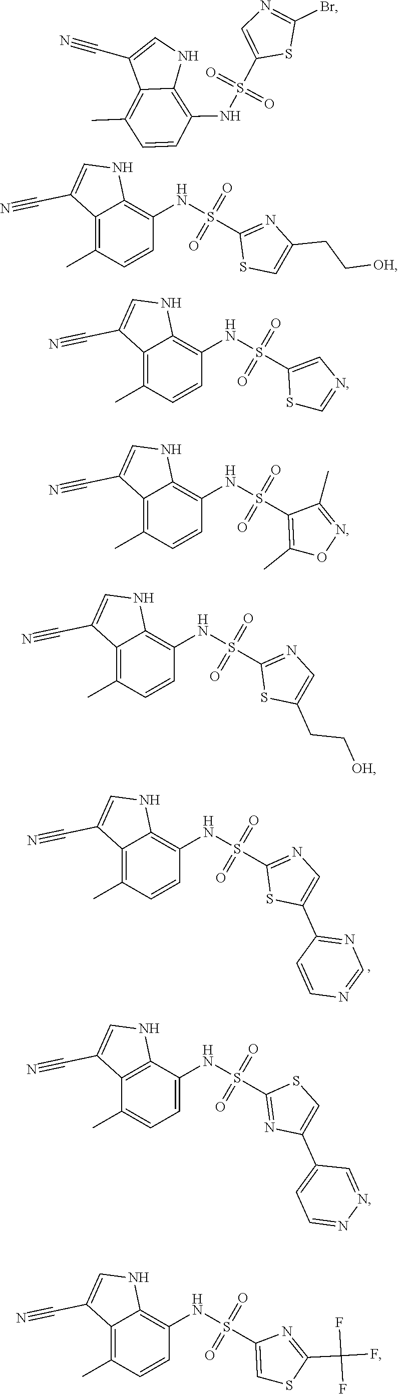

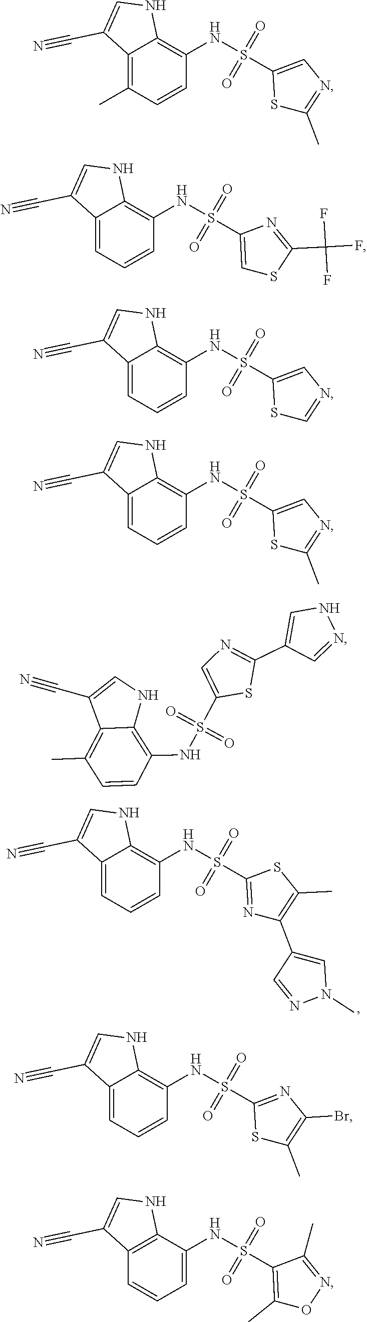

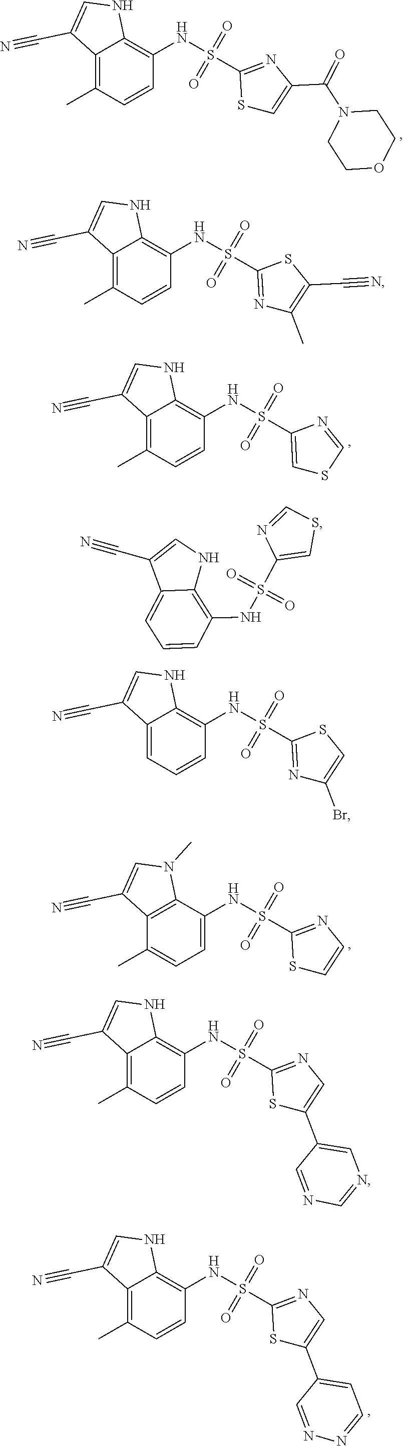

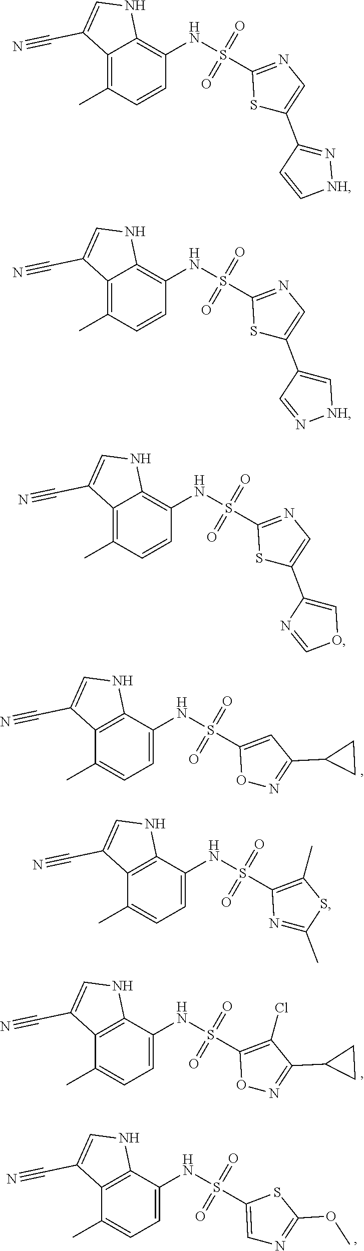

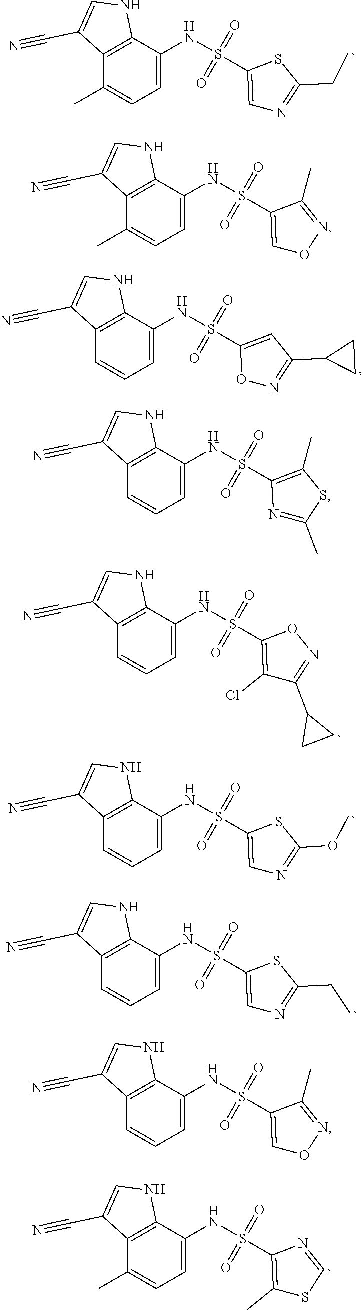

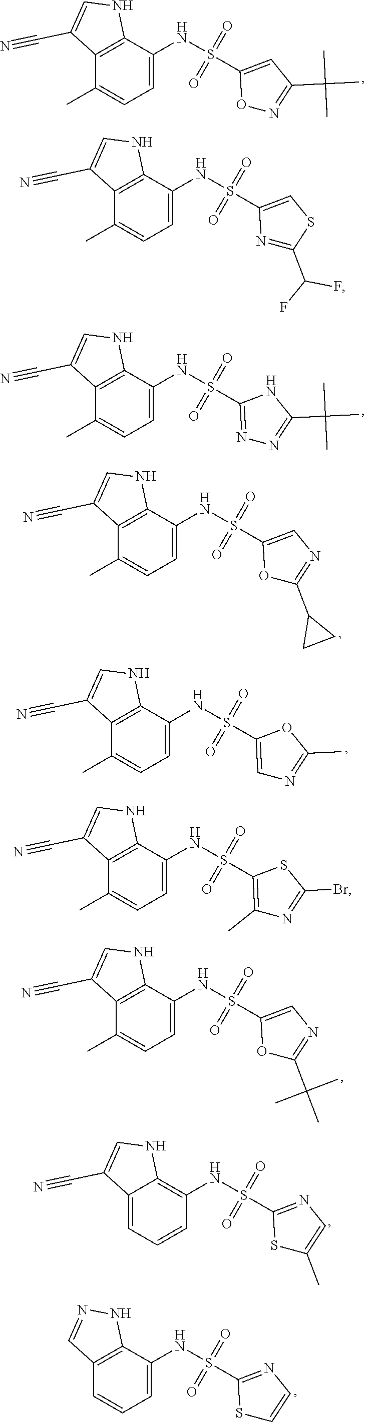

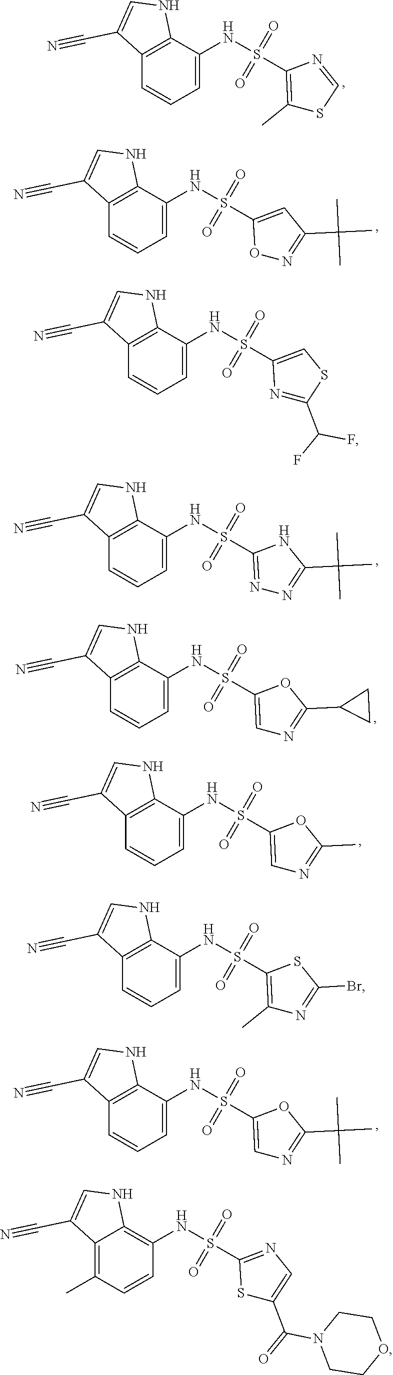

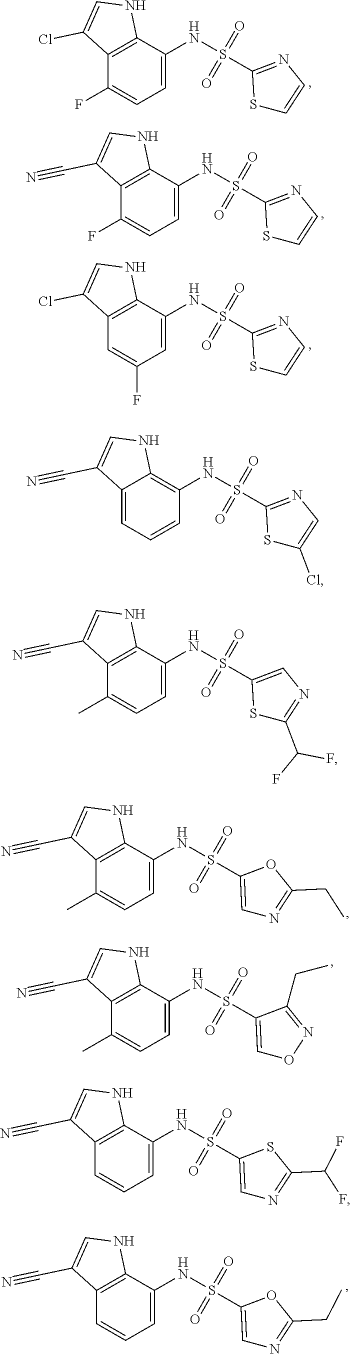

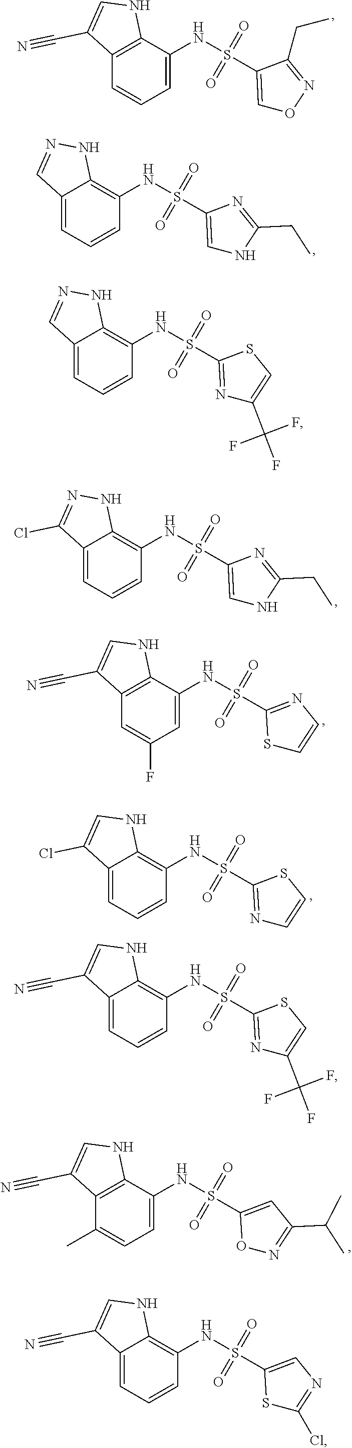

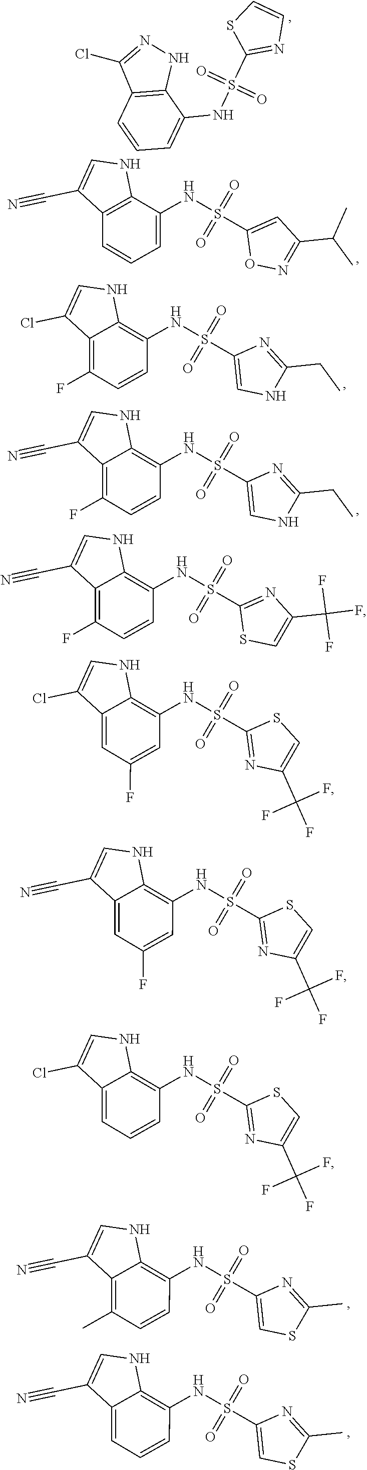

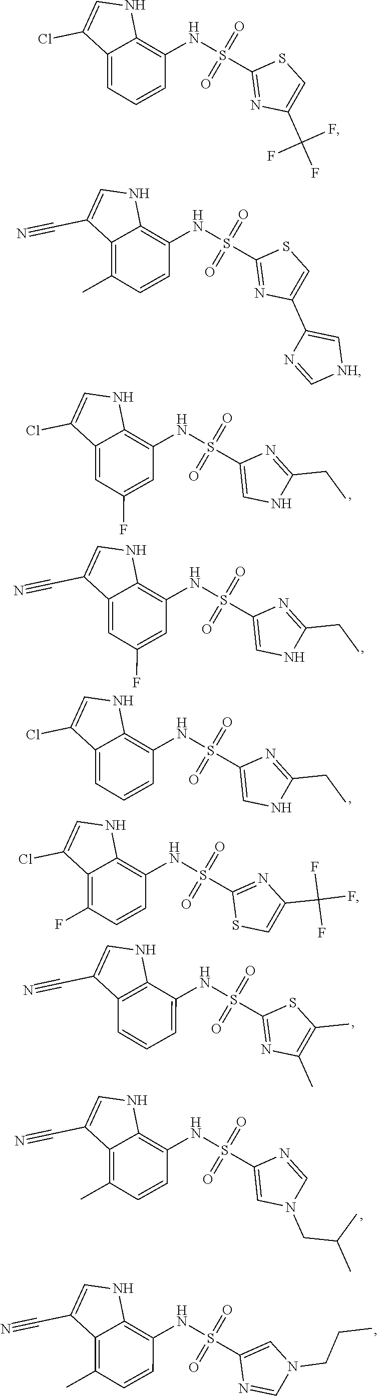

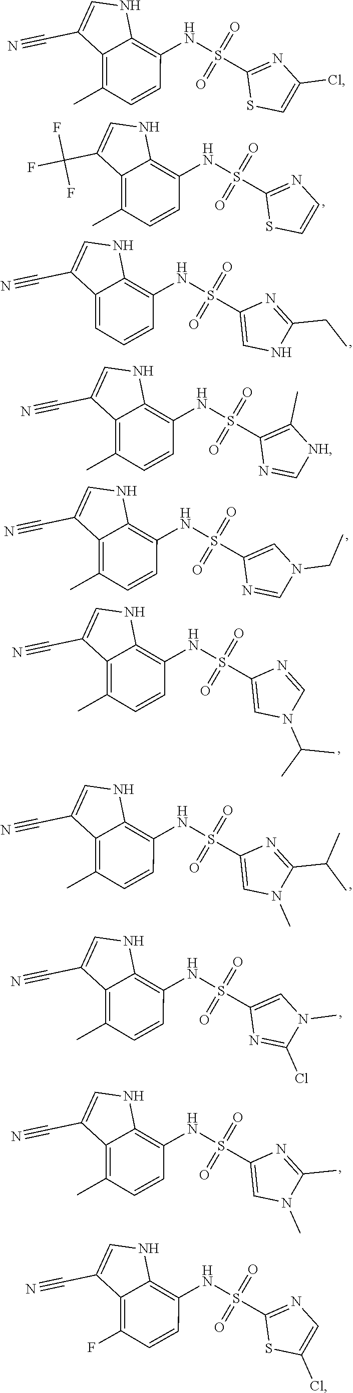

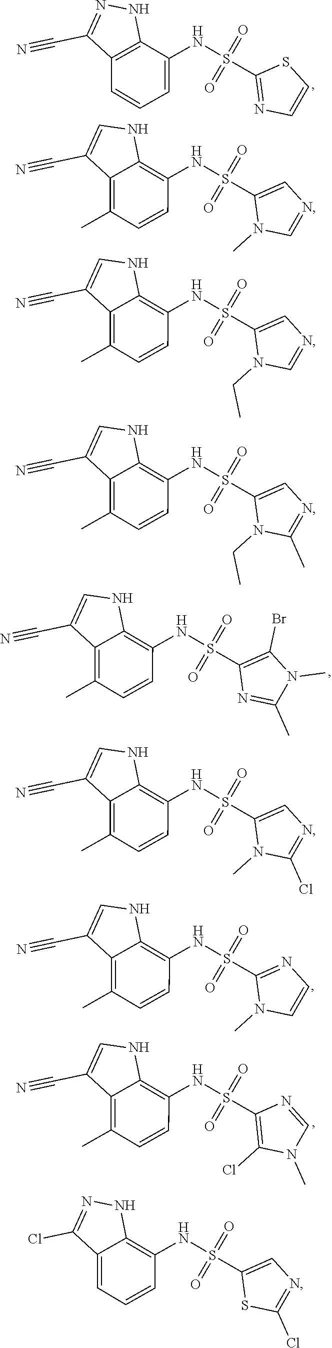

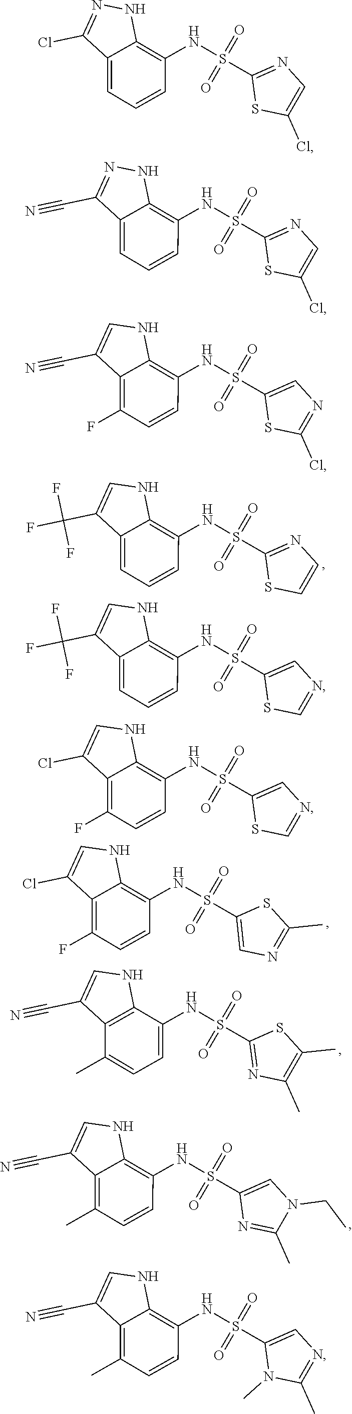

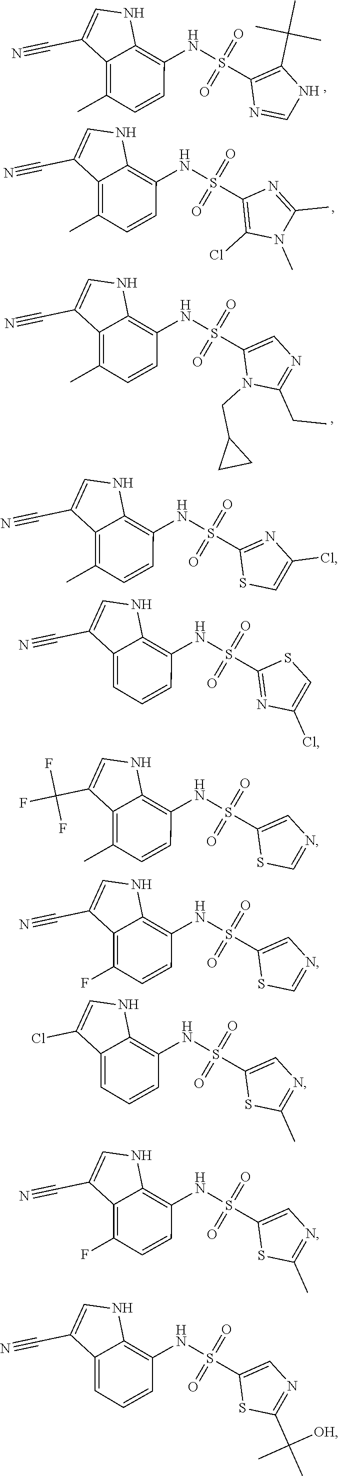

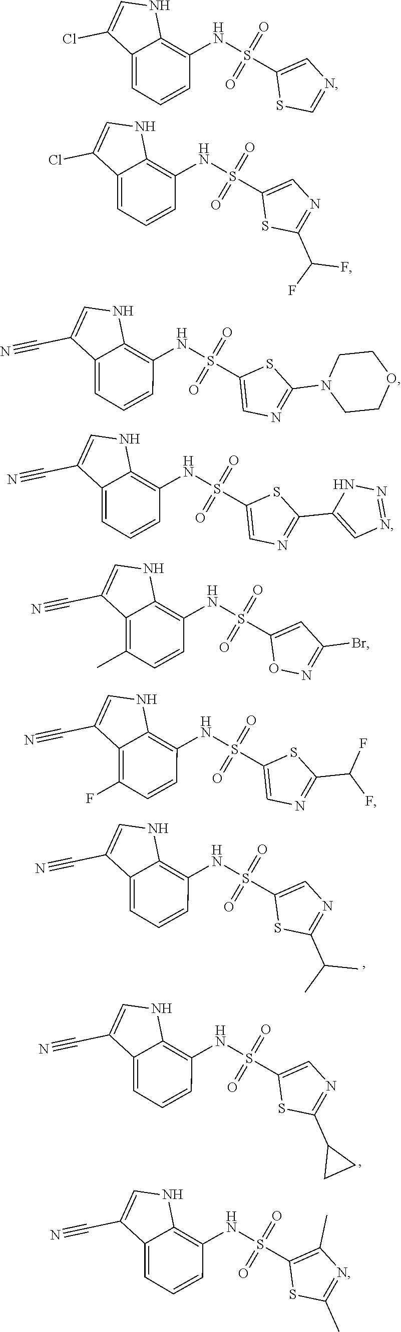

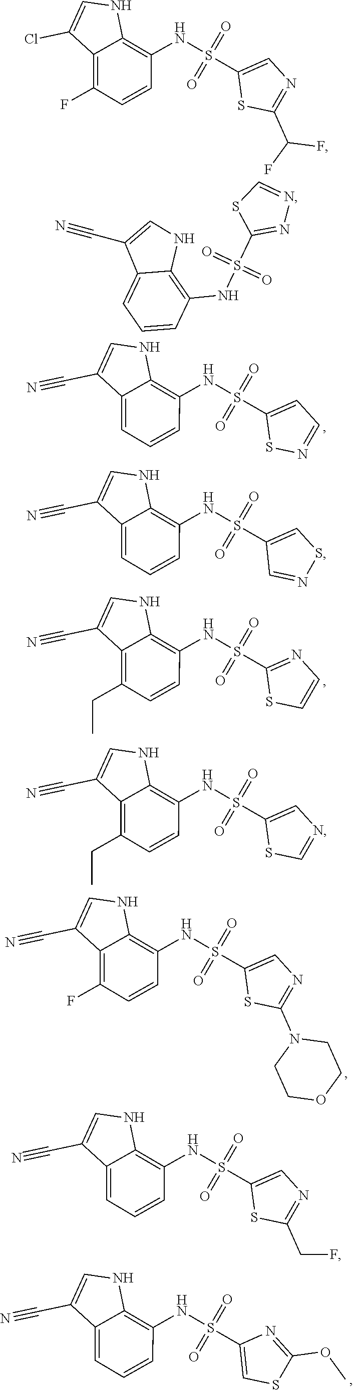

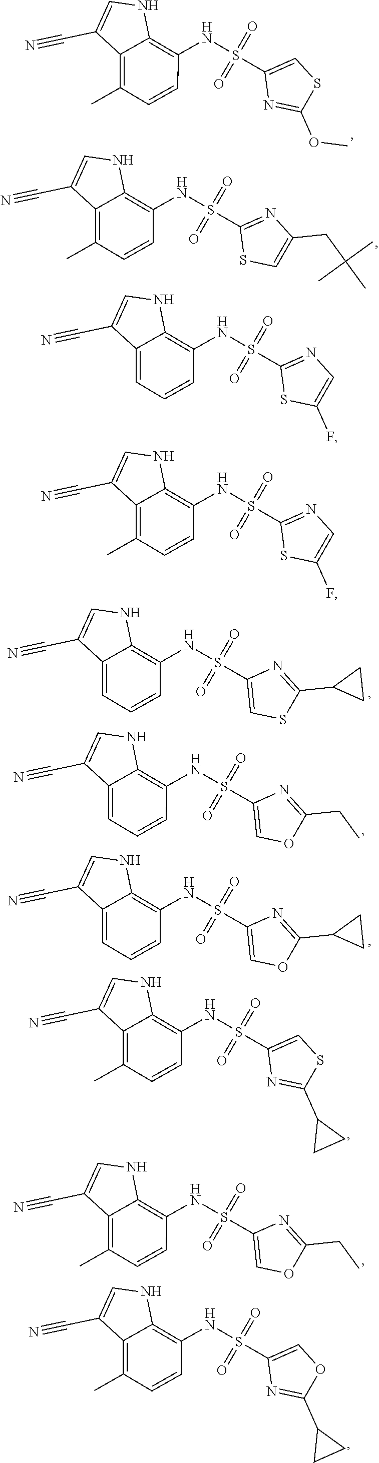

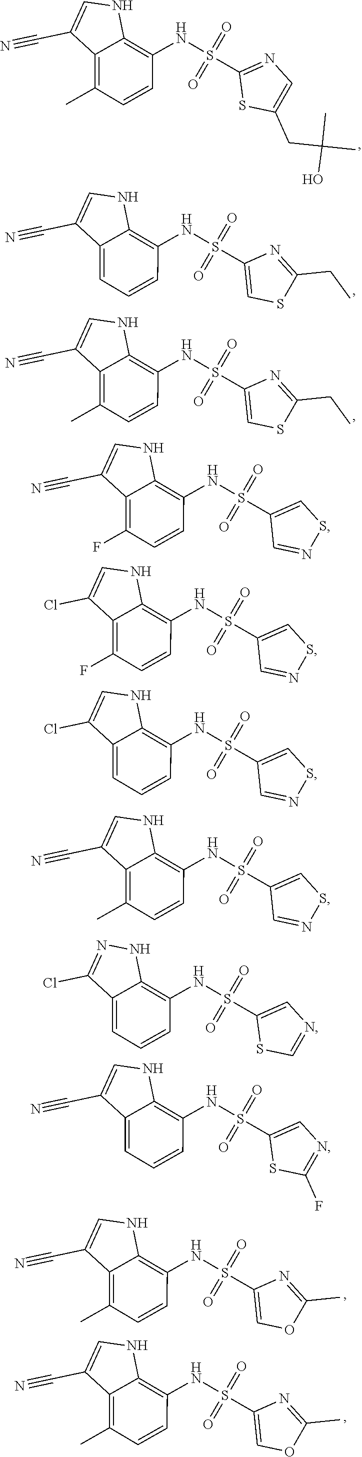

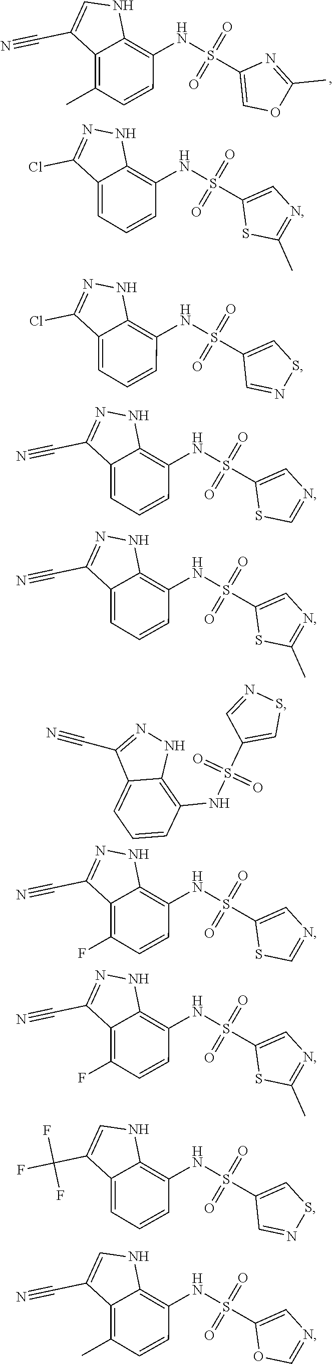

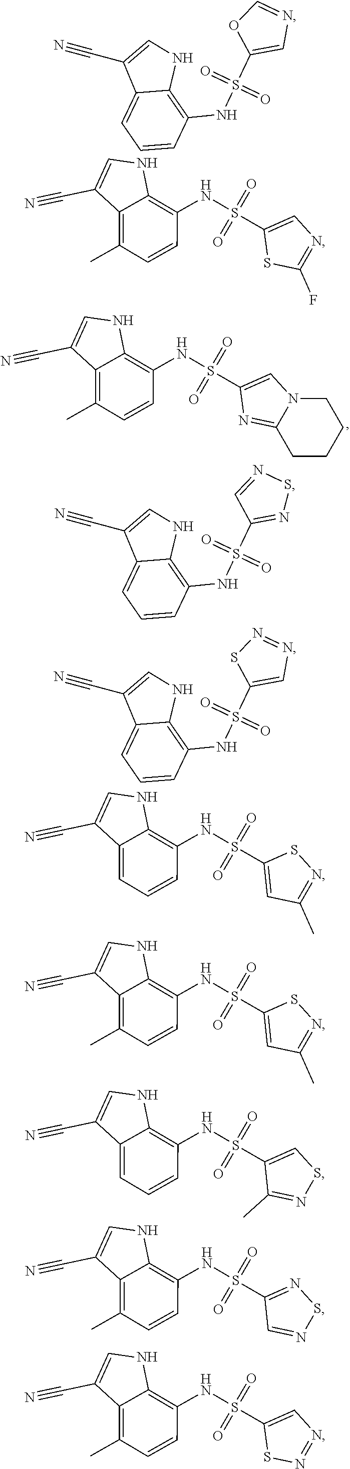

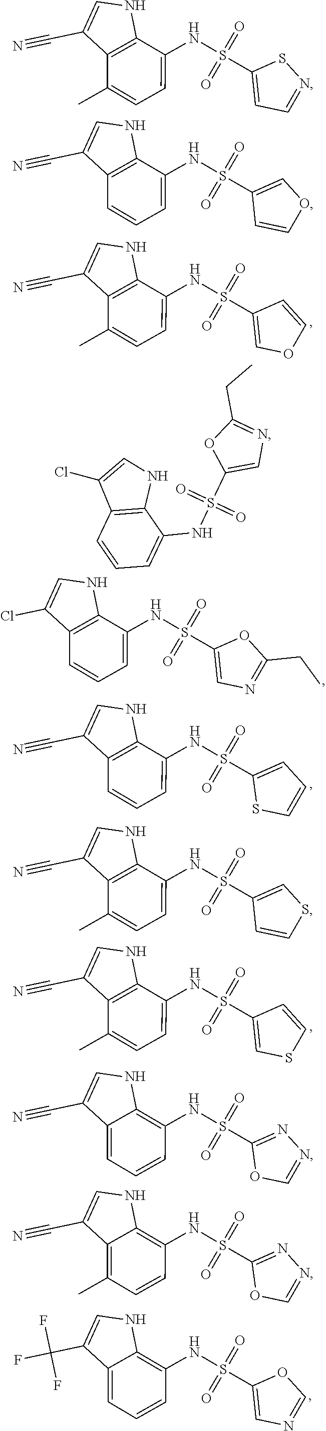

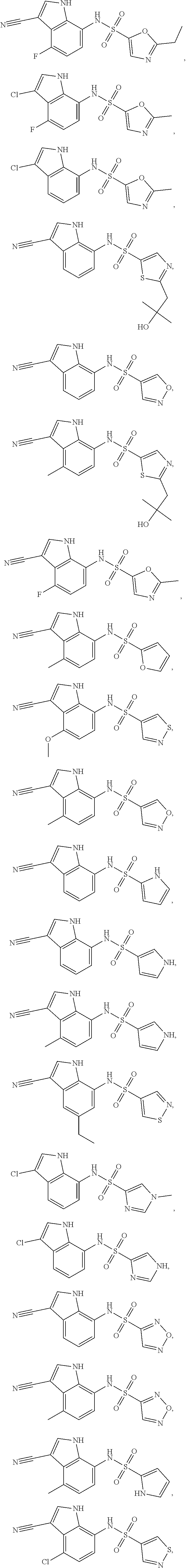

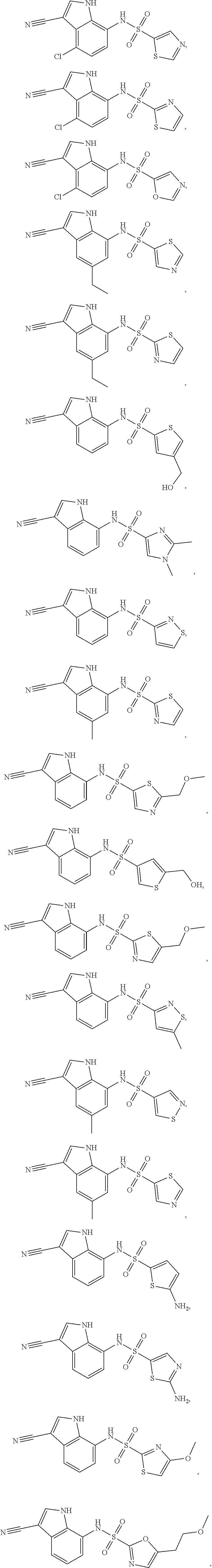

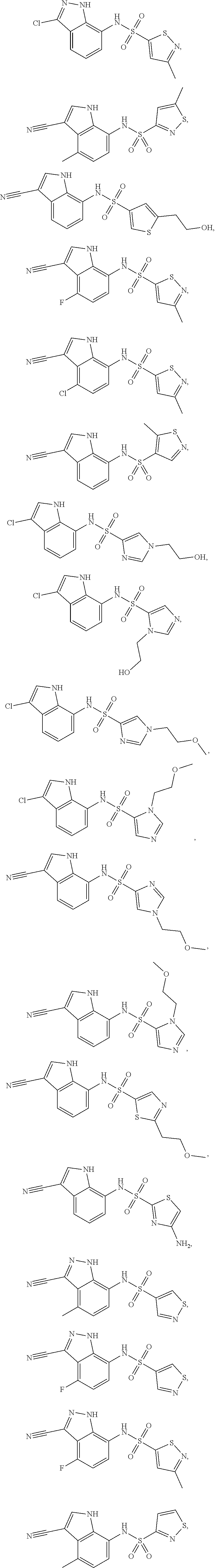

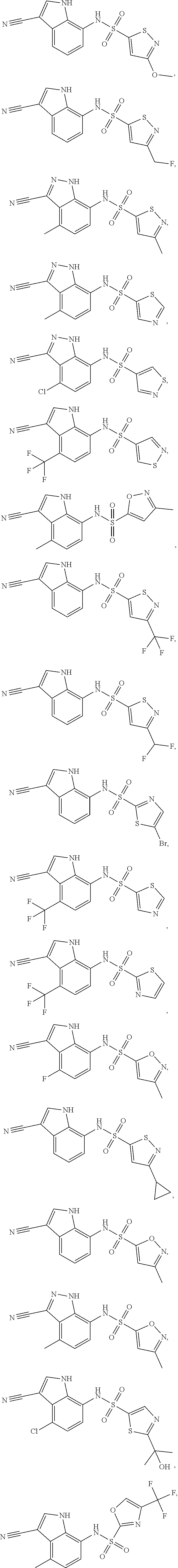

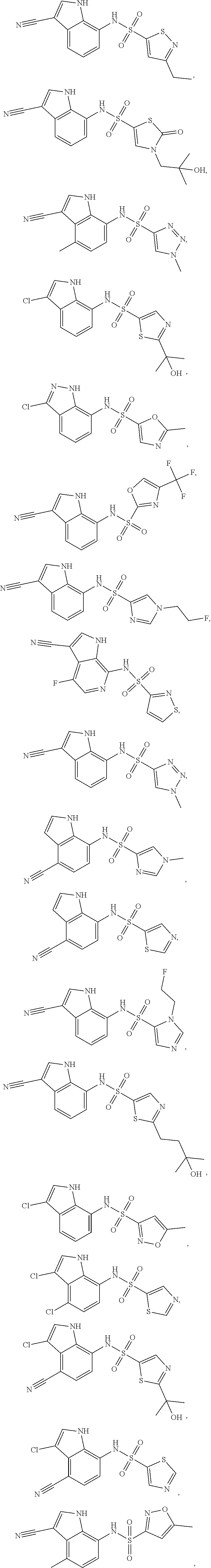

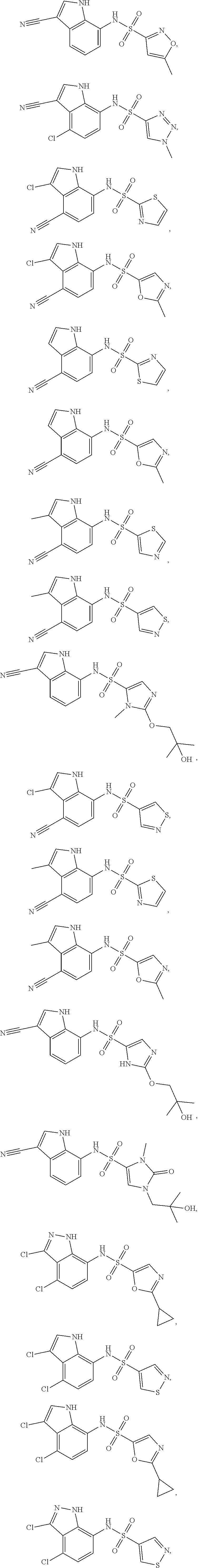

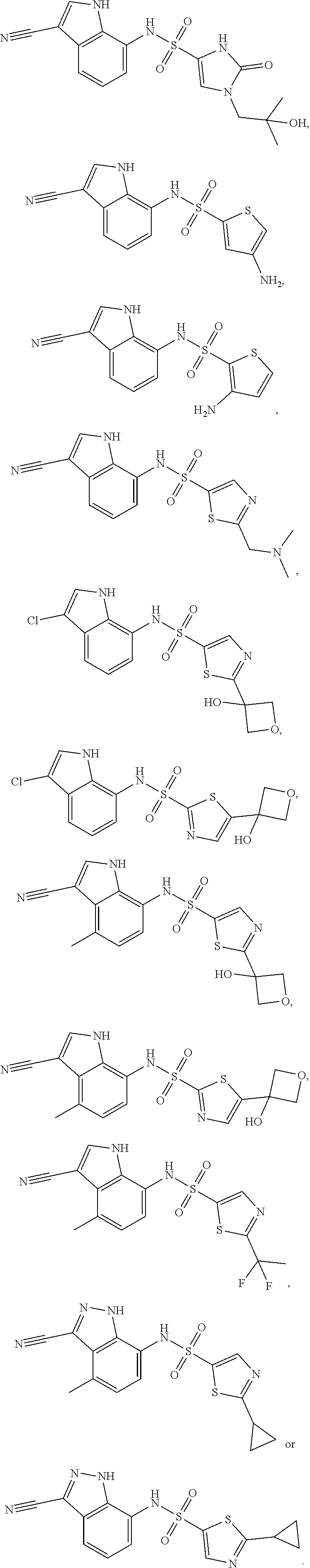

Other embodiments disclosed herein include a pharmaceutical composition comprising a therapeutically effective amount of a compound disclosed herein and a pharmaceutically acceptable excipient.

Other embodiments disclosed herein include method of preventing, treating, or ameliorating one or more cancers in a subject, by administering the compounds disclosed herein, or a pharmaceutically acceptable salt thereof, to a subject in need thereof. The cancers include, but are not limited to, colorectal cancer (CRC), pleural mesothelioma (PM), cutaneous squamous cell carcinoma (CSCC); tumor mutation burden high (TMB-H), Bacillus Calmette-Gudrin bladder cancer, endometrial carcinoma (EC), esophageal squamous cell carcinoma (ESCC), Merkel cell carcinoma (MCC), hepatocellular carcinoma (HCC), primary mediastinal large B cell lymphoma (PMBCL), cervical cancer, urothelial carcinoma, classical Hodgkin's lymphoma, head and neck squamous cell carcinoma, liver cancer, gastric cancer, prostate cancer, sarcoma, melanoma, non-small cell lung cancer (NSCLC), small cell lung cancer, renal cell carcinoma, triple negative breast cancer, luminal B breast cancer, colon cancer, ovarian cancer, pancreatic cancer and glioblastoma.

### BRIEF DESCRIPTION OF THE DRAWINGS

The following drawings and the associated descriptions are provided to illustrate implementations of the present disclosure and do not limit the scope of the claims.

FIG. 1 shows plasma concentrations (ng/mL) of Compound 1 and comparative compound E7820 over 8 hours following oral administration in female BALB/c mice.

FIG. 2. shows telencephalon concentrations of Compound 1 and comparative compound E7820 over 8 hours following oral administration in female BALB/c mice.

FIG. 3. shows plasma concentrations of Compound 1 and Compound 143 over 8 hours following oral administration in female BALB/c mice.

FIG. 4. shows telencephalon concentrations of Compound 1 and Compound 143 over 8 hours following oral administration in female BALB/c mice.

FIG. 5 shows inhibition of cell growth by Compound 1 and Compound 143 in HCT116 colorectal cells.

### DETAILED DESCRIPTION

In some embodiments, provided herein are indole and thiazole-containing sulfonamide compounds that act as modulators of RBM39. Various embodiments of these compounds include compounds having the structure of Formula (T) as described above or pharmaceutically acceptable salts thereof. The structure of Formula (I) encompasses all stereoisomers and racemic mixtures, including the following structures and mixtures thereof:

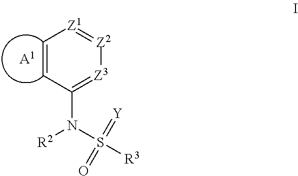

In some embodiments of compounds of Formula (I):
A¹ is selected from

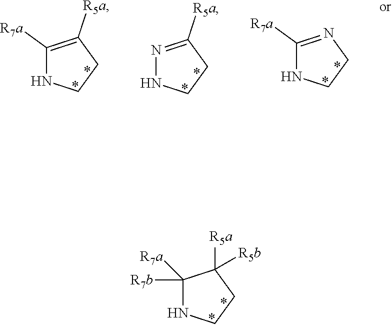

and * represents points of attachment to form a fused bicyclic ring;
Y is O or NH;
Z¹, Z² and Z³ are each independently C(R^{1a}) or N;
each R^{1a} is independently selected from the group consisting of H, halogen, —(C₁-C₆)alkyl and —(C₁-C₆)haloalkyl;
R² is H, —(C₁-C₆)alkyl or —C(O)R⁶;
R³ is a —(C₁-C₆)alkyl, furan, thiophene, a 5-membered monocyclic nitrogen-containing heteroaryl, or a 6-12 membered nitrogen-containing bicyclic heterocyclyl; wherein the —(C₁-C₆)alkyl, furan, thiophene, 5-membered monocyclic nitrogen-containing heteroaryl and the 6-12 membered nitrogen-containing bicyclic heterocyclyl can be optionally substituted with one or two or three substituents selected from R⁴;
each R⁴ is independently selected independently selected from —R^{x1}, —R², —(C₁-C₆)alkyl, —(C₁-C₆)haloalkyl, —(C₁-C₆)alkoxy, —CN, halogen, —NH₂, —N((C₁-C₆)alkyl)₂, —NHC(O)(C₁-C₆)alkyl, —NHBoc, —(CH₂)ₙS(O)₂(C₁-C₆)alkyl and —C(O)R^{z1};
R^{5a} is selected from the group consisting of —H, —CN, halogen, —(C₁-C₆)alkyl, —(C₁-C₆)haloalkyl, —(C₂-C₆)alkenyl and —(C₂-C₆)alkynyl;
R^{5b} is —(C₁-C₆)alkyl; or
R^{5a} is taken together with R^{5b} and the atom to which R^{5a} and R^{5b} are attached to form an optionally substituted 3-7 membered monocyclic cycloalkyl;
R⁶ is H or —(C₁-C₆)alkyl;
R^{7a} and R^{7b} are each independently selected from the group consisting of H, halogen, —CN, —(C₁-C₆)alkyl, —(C₁-C₆)alkoxy, 3-7 membered monocyclic cycloalkyl and —(C₁-C₆)haloalkyl; or R^{7a} is taken together with R^{7b} and the atom to which R^{7a} and R^{7b} are attached to be —C(═O);
R^{x1} is selected from the group consisting of cycloalkyl, heterocyclyl, and heterocyclyl(alkyl), wherein the cycloalkyl, heterocyclyl and heterocyclyl(alkyl) are each optionally substituted with R^{y};
R^{x2} is selected from the group consisting of —(C₁-C₆)alkyl, —(C₁-C₆)alkoxy, alkylamino, and amino; wherein the —(C₁-C₆)alkyl, —(C₁-C₆)alkoxy, alkylamino, and amino are optionally substituted with one or two R^{y2};
R^{y1} is selected from the group consisting of H, —CN, —OH, —C(O)O(C₁-C₆)alkyl, —(C₁-C₆)alkyl, —(C₁-C₆)haloalkyl, —(C₁-C₆)alkoxy, heterocyclyl, BOC, —C(O)(C₁-C₆)alkyl, —S(O)₂(C₁-C₆)alkyl, —CH₂S(O)₂(C₁-C₆)alkyl and —CH₂CN;
each R^{y2} is independently selected from the group consisting of —CN, —OH, —(C₁-C₆)alkyl, —(C₁-C₆)haloalkyl, —(C₁-C₆)alkoxy, —N((C₁-C₆)alkyl)₂, —CH₂CN, —C(O)CH₂CH₂N((C₁-C₆)alkyl)₂, —C(O)(heterocyclyl) and —(CH₂)ₙS(O)₂(C₁-C₆)alkyl;
n is 0, 1, 2, 3 or 4; and
R^{z1} is

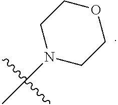 [SMILES: *N1CCOCC1]

In some embodiments, the compound is not

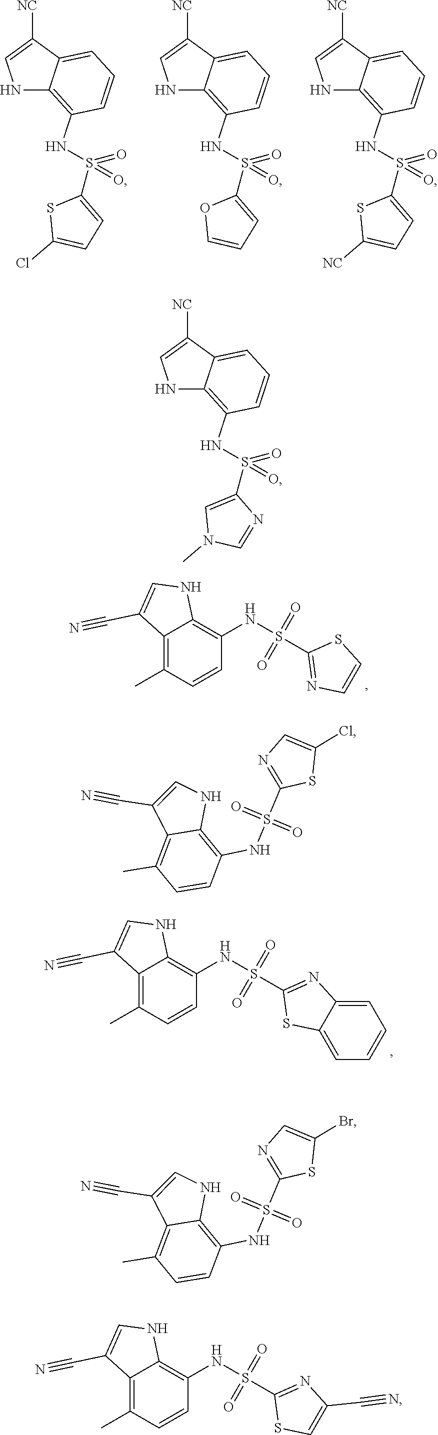

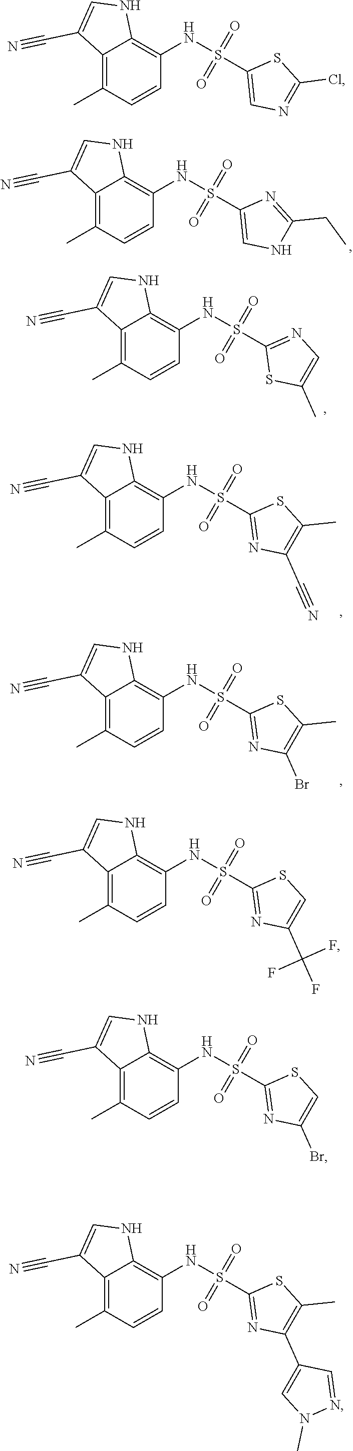

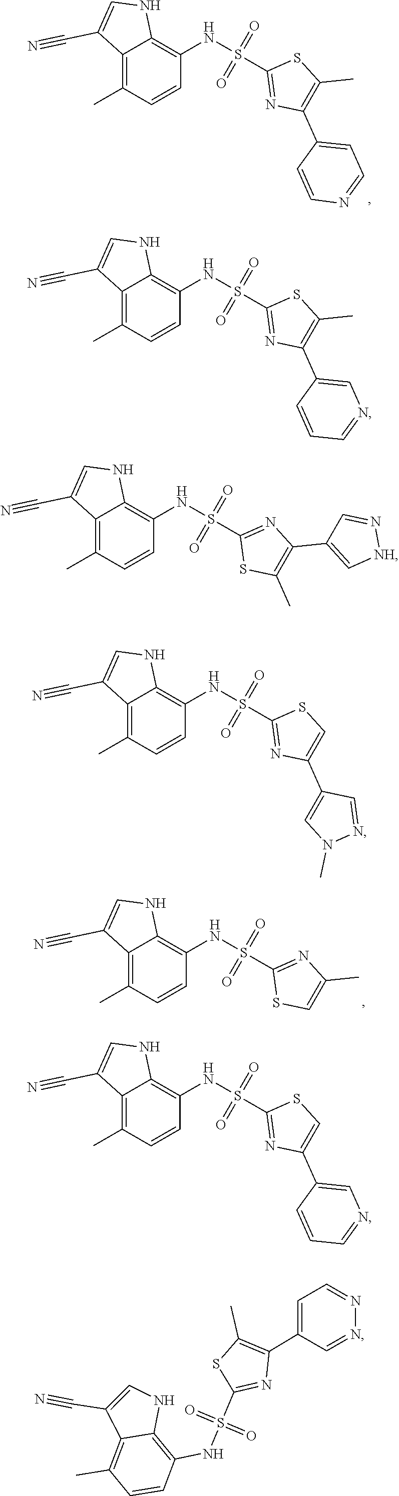

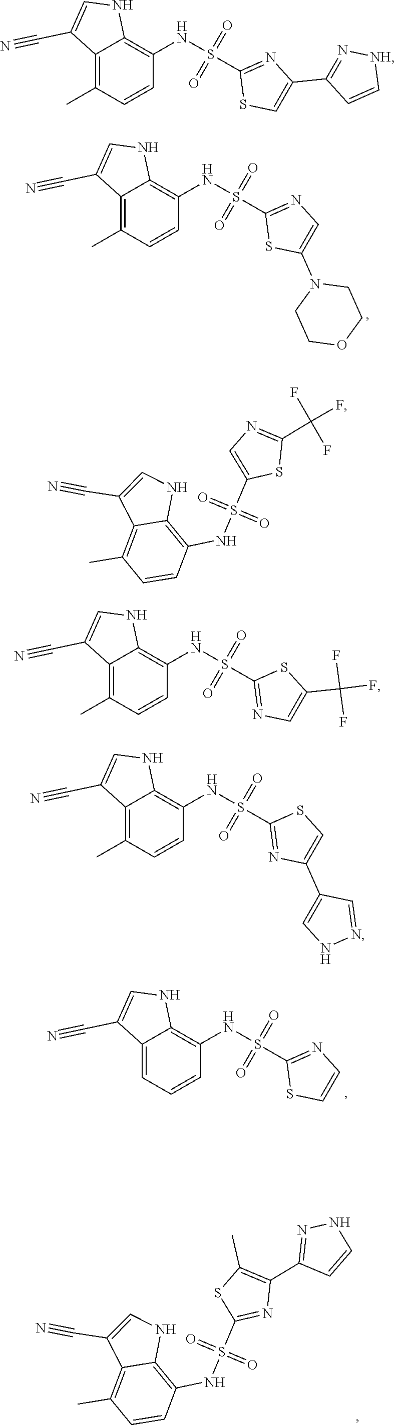

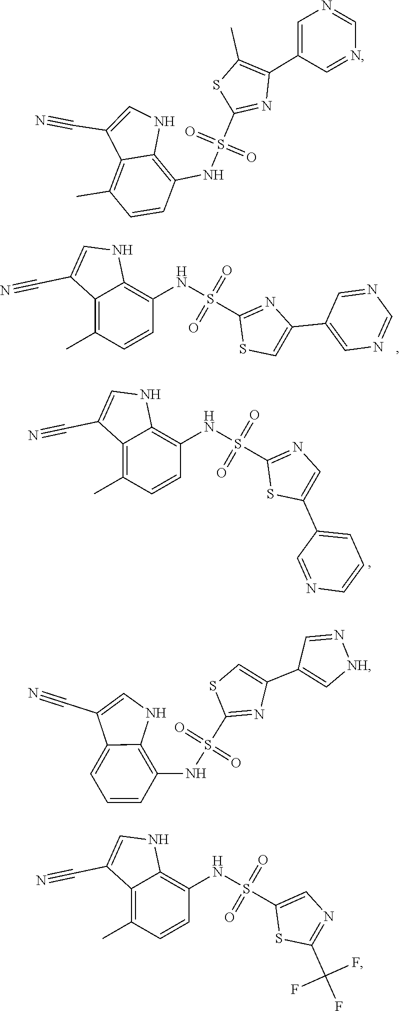

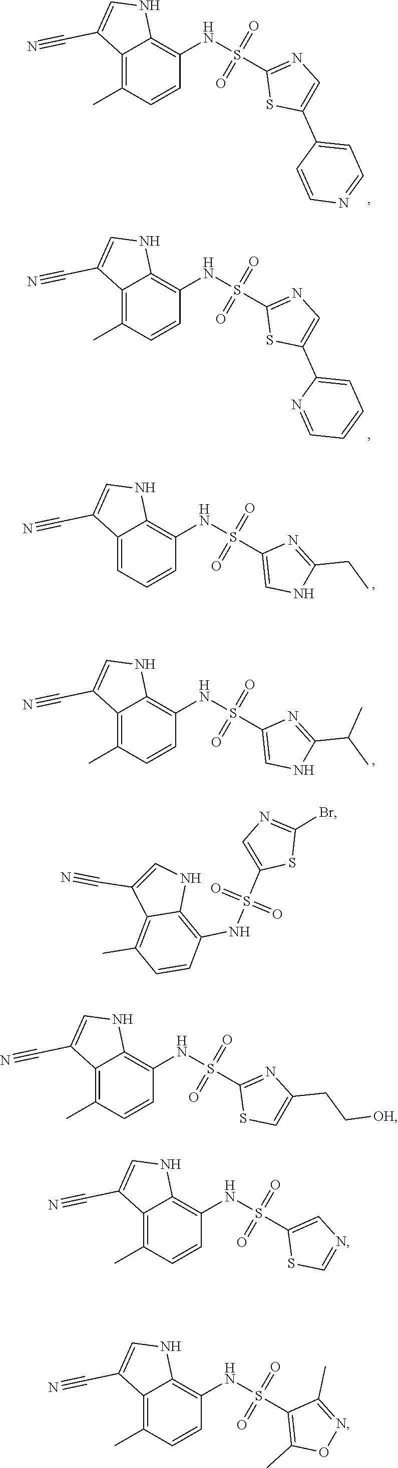

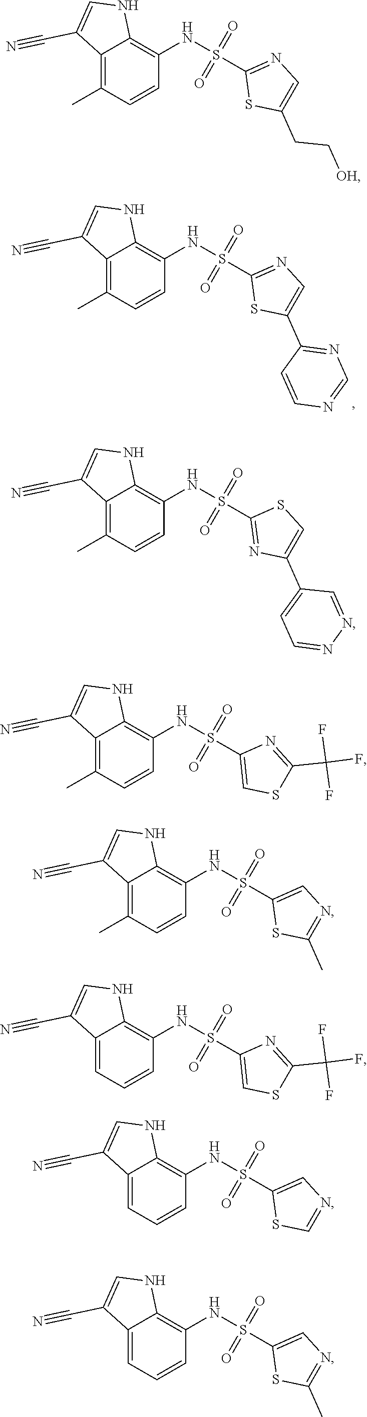

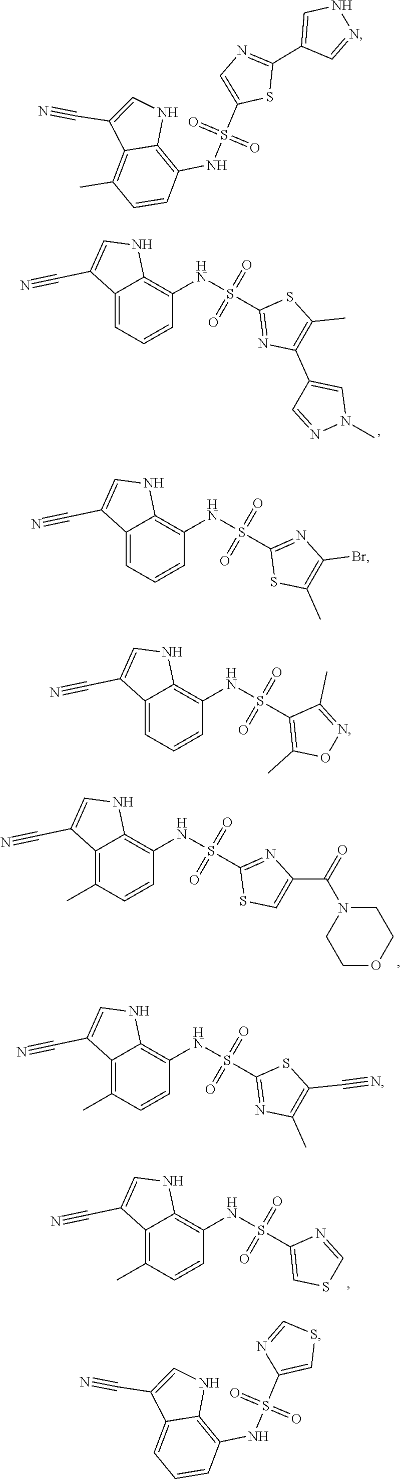

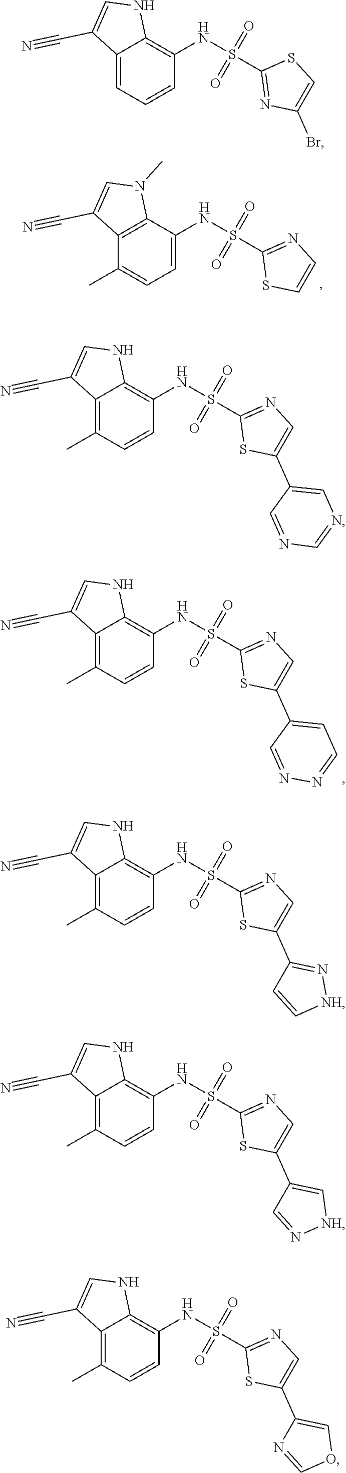

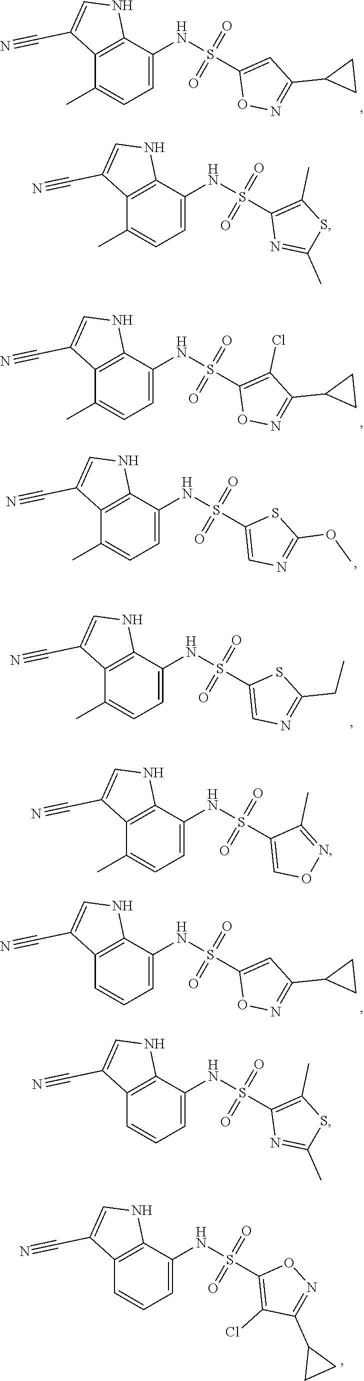

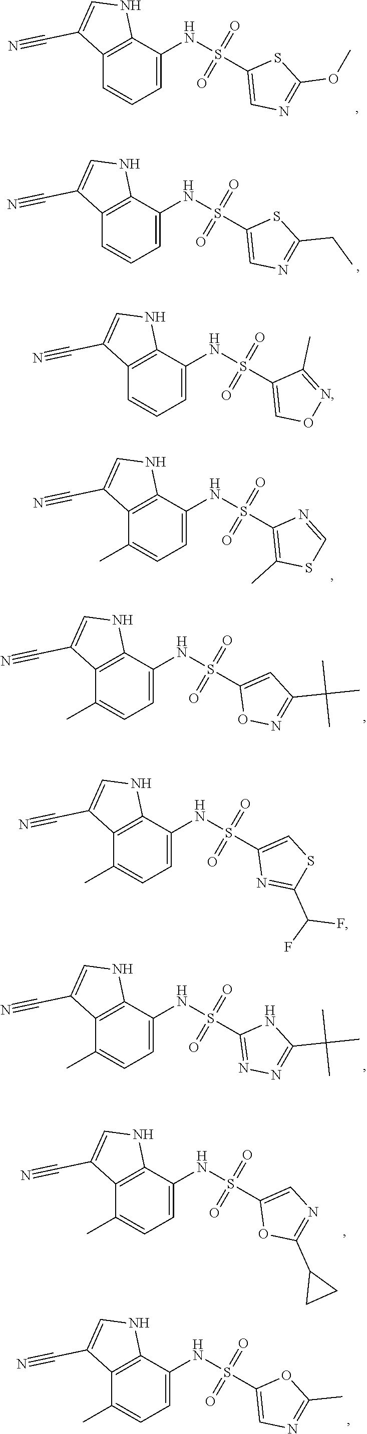

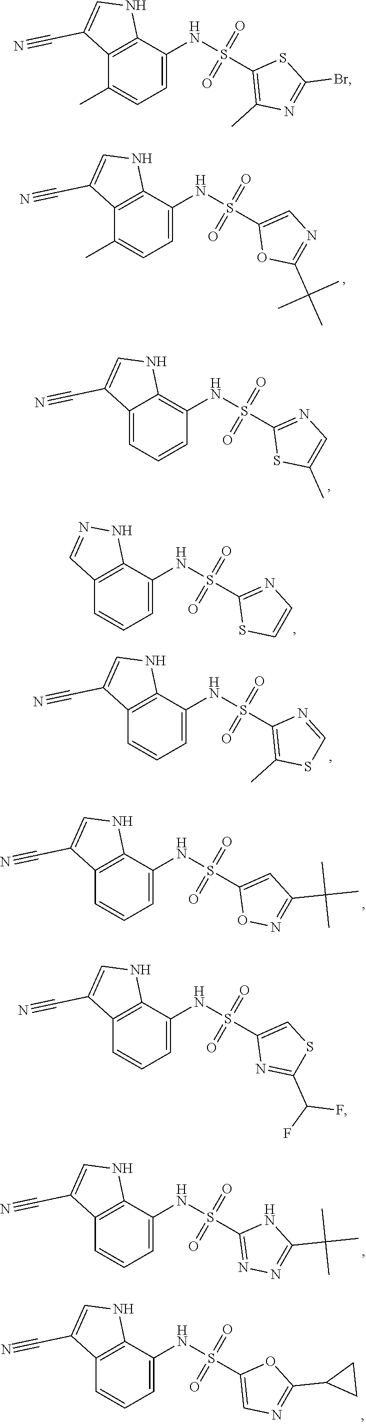

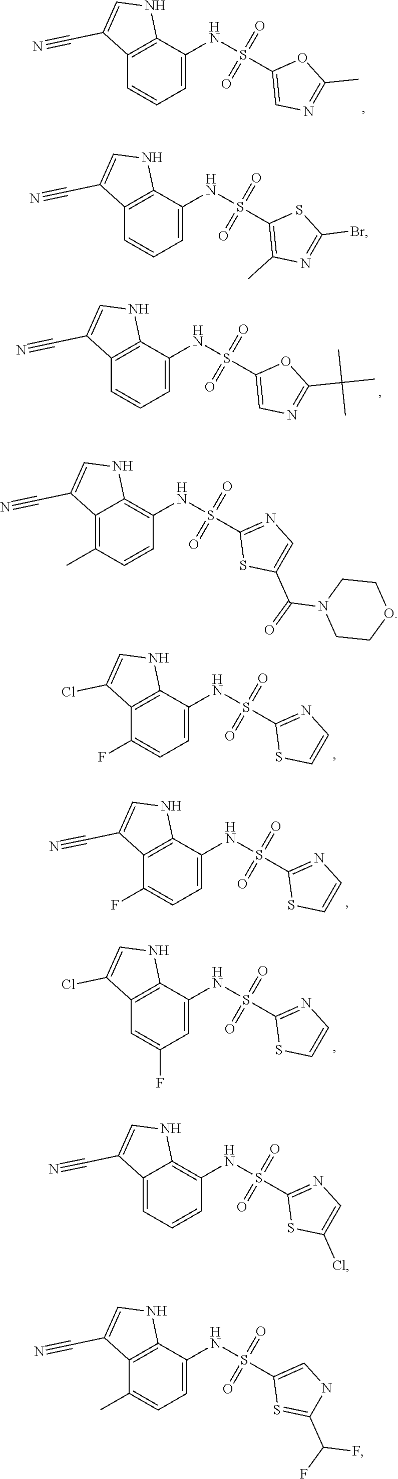

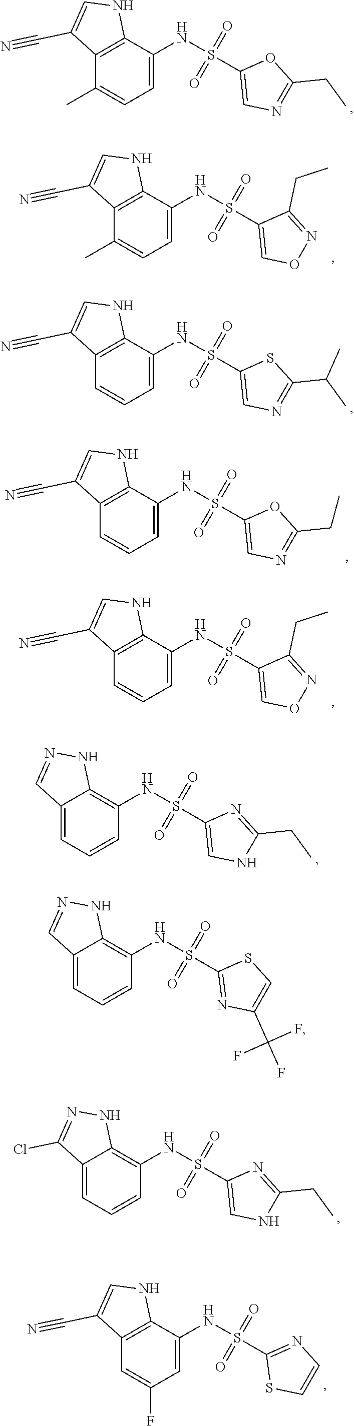

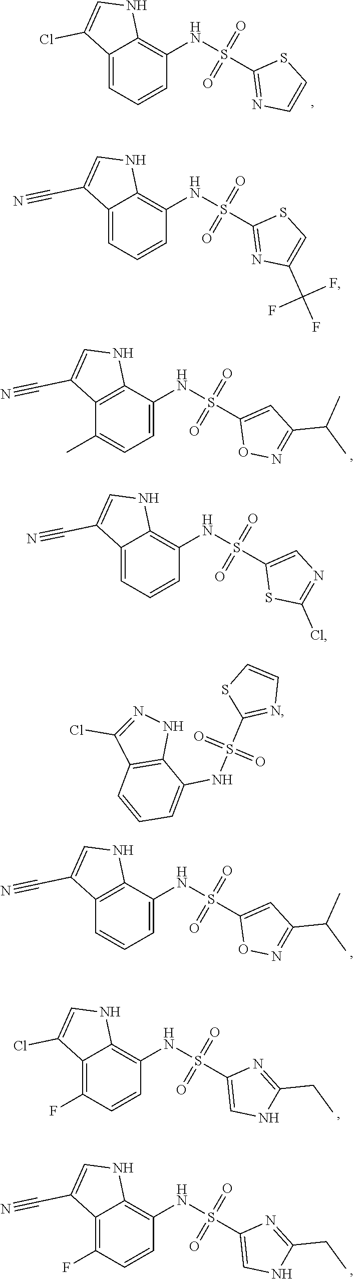

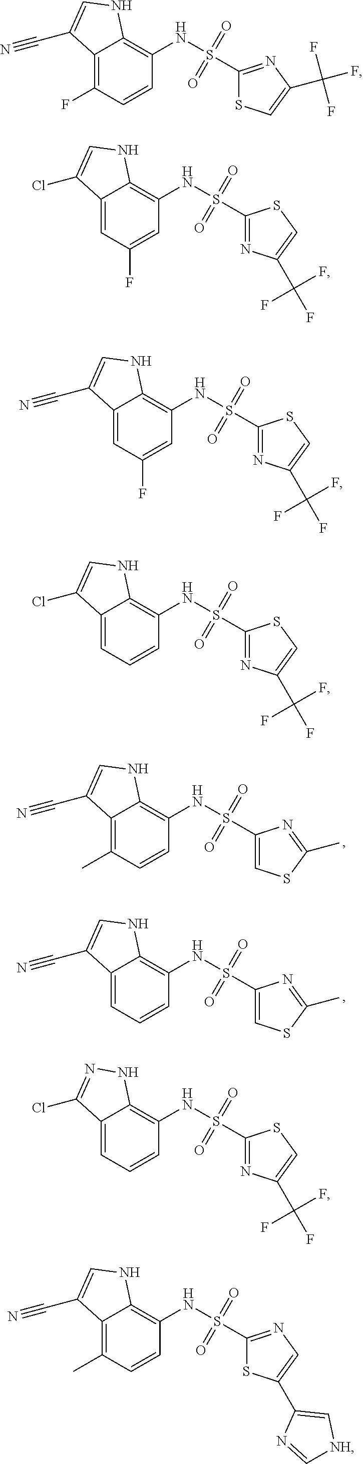

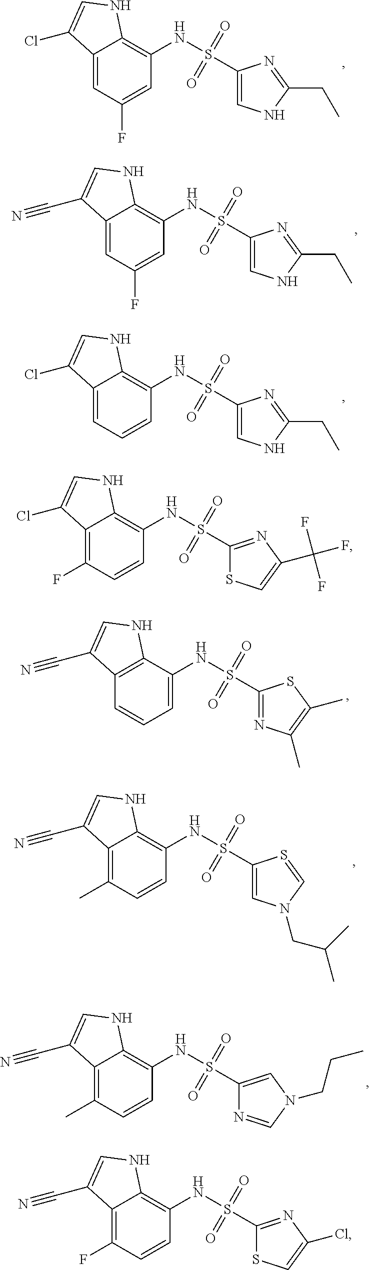

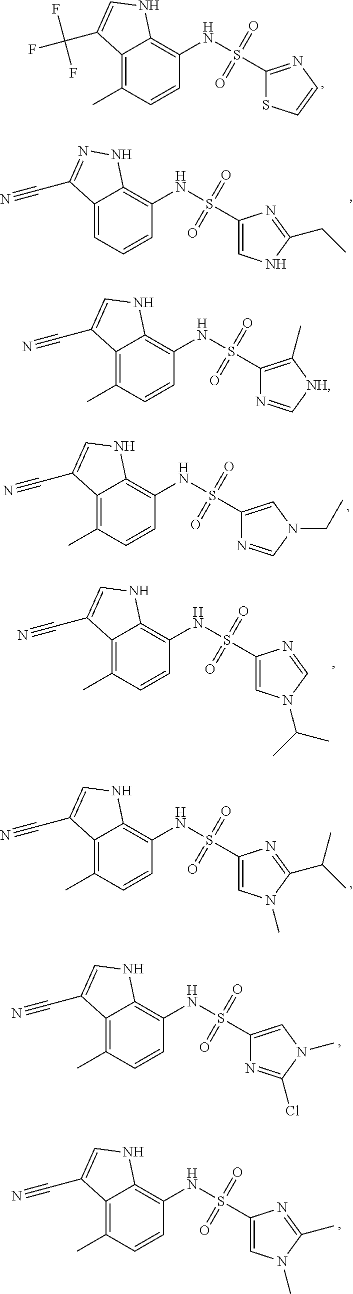

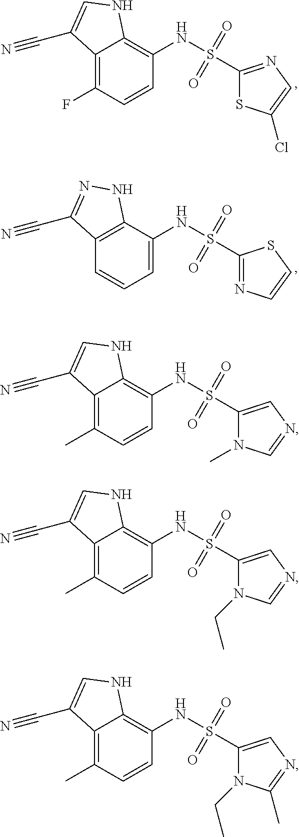 [SMILES: N#Cc1c[nH]c2c(NS(=O)(=O)c3ncc(Cl)s3)ccc(F)c12 ; N#Cc1n[nH]c2c(NS(=O)(=O)c3nccs3)cccc12 ; Cc1ccc(NS(=O)(=O)c2cncn2C)c2[nH]cc(C#N)c12 ; CCn1cncc1S(=O)(=O)Nc1ccc(C)c2c(C#N)c[nH]c12 ; CCn1c(S(=O)(=O)Nc2ccc(C)c3c(C#N)c[nH]c23)cnc1C]

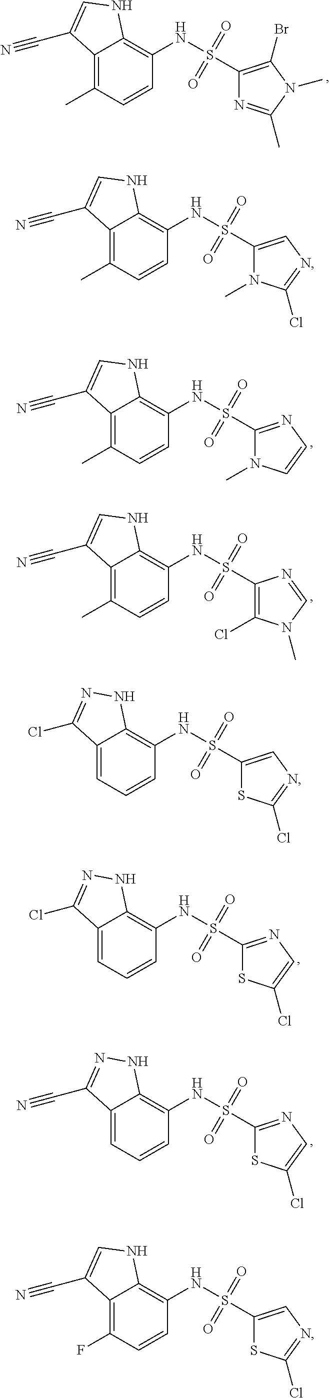

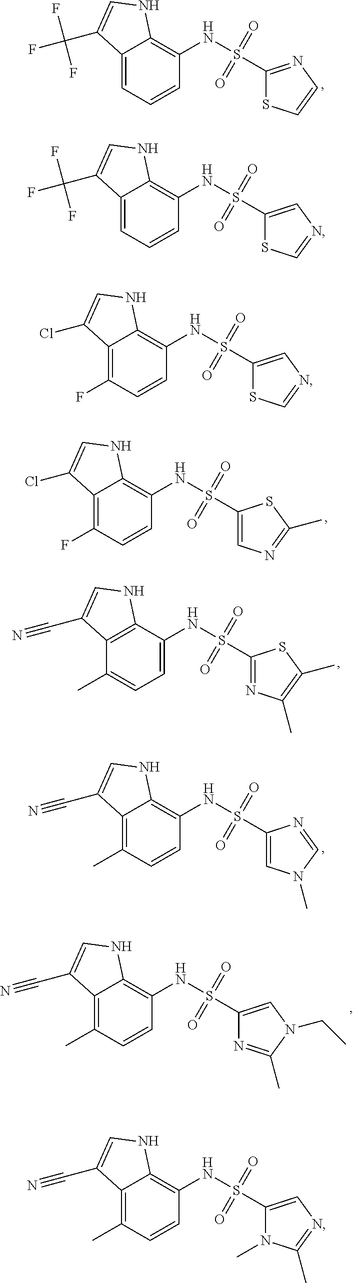

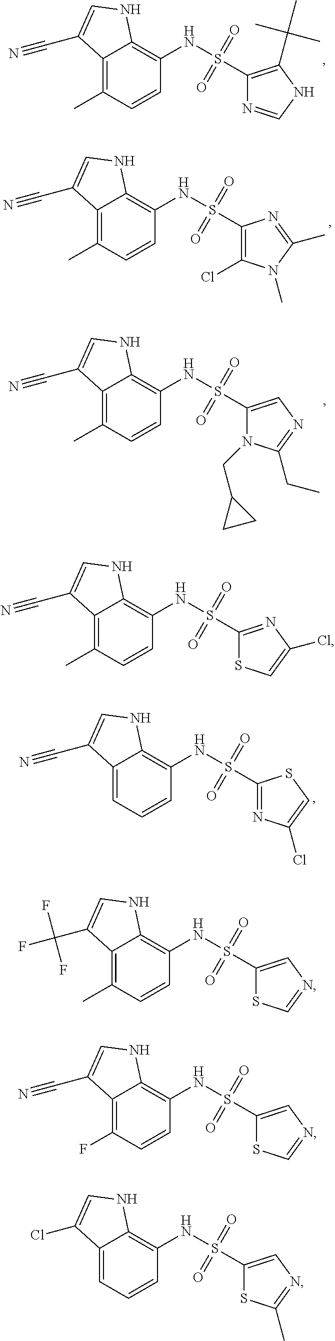

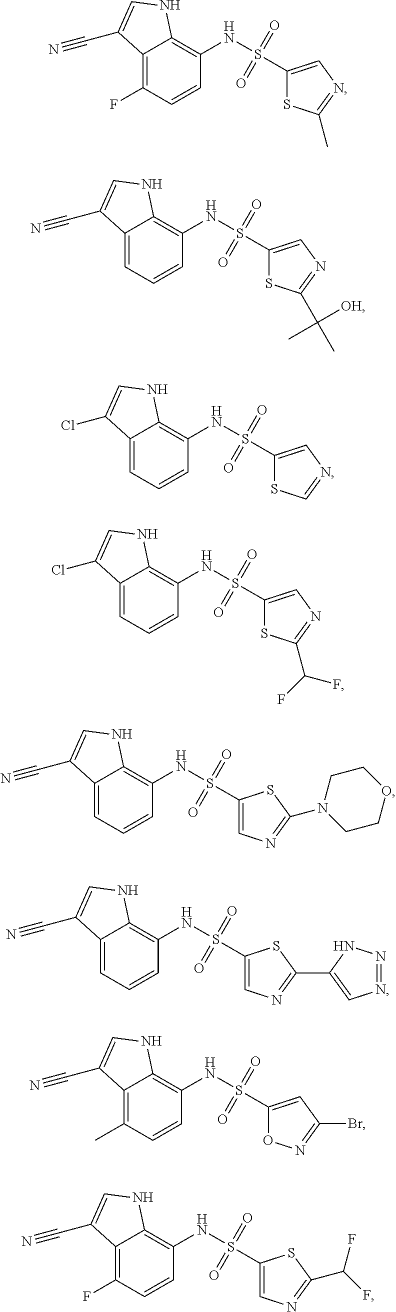

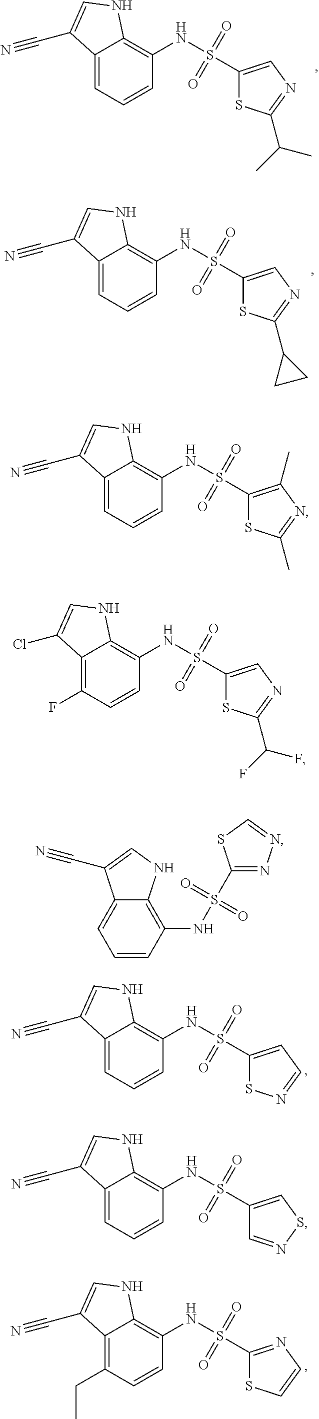

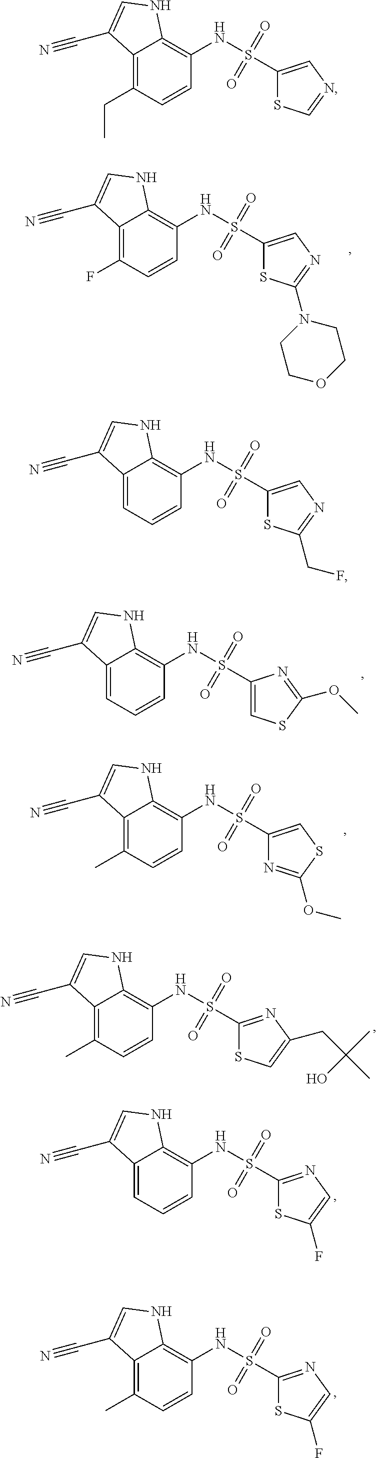

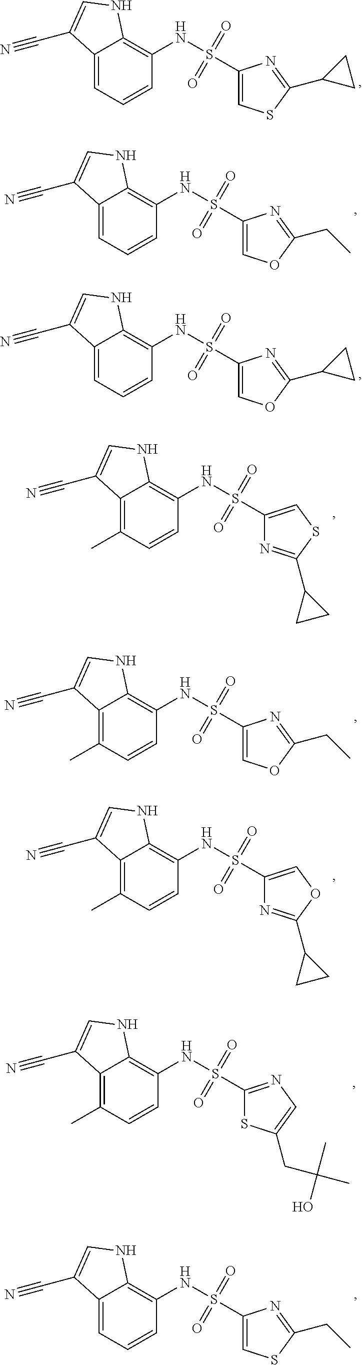

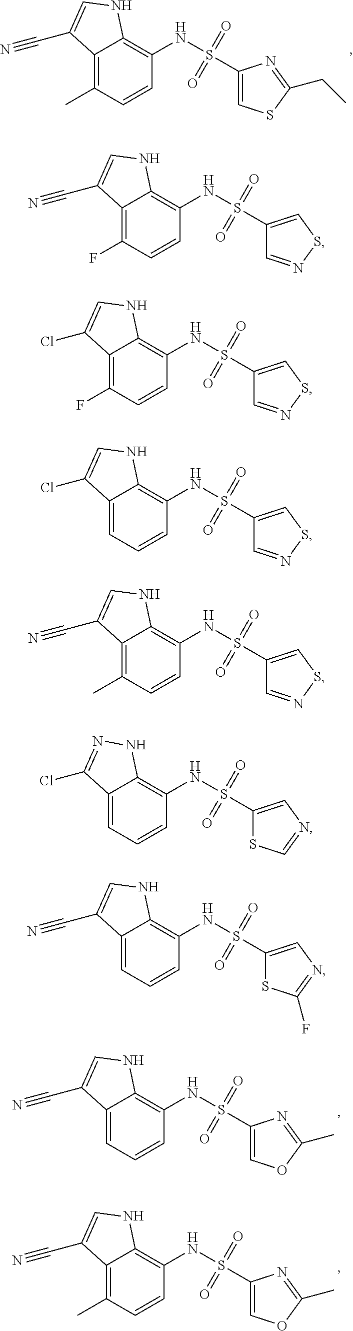

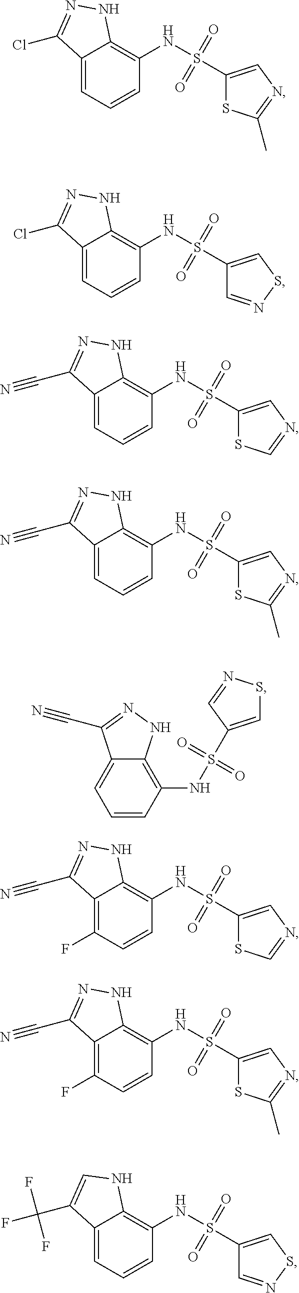

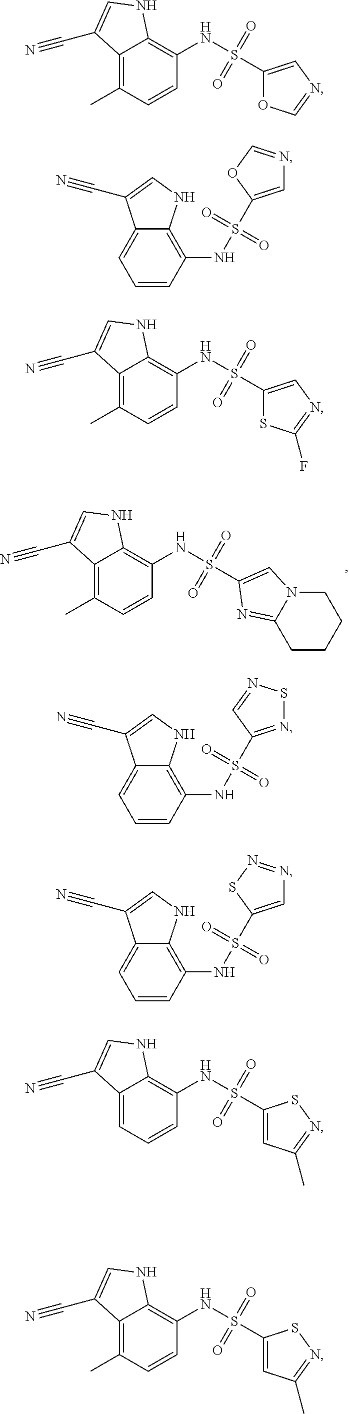

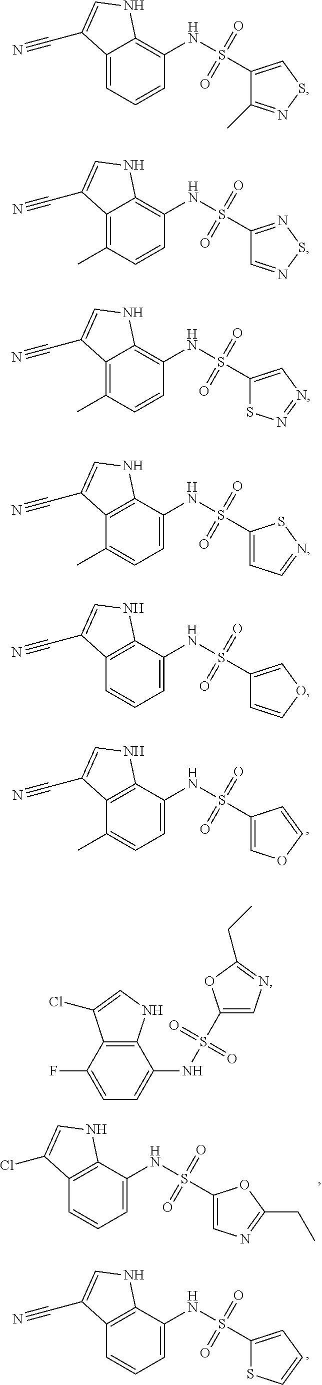

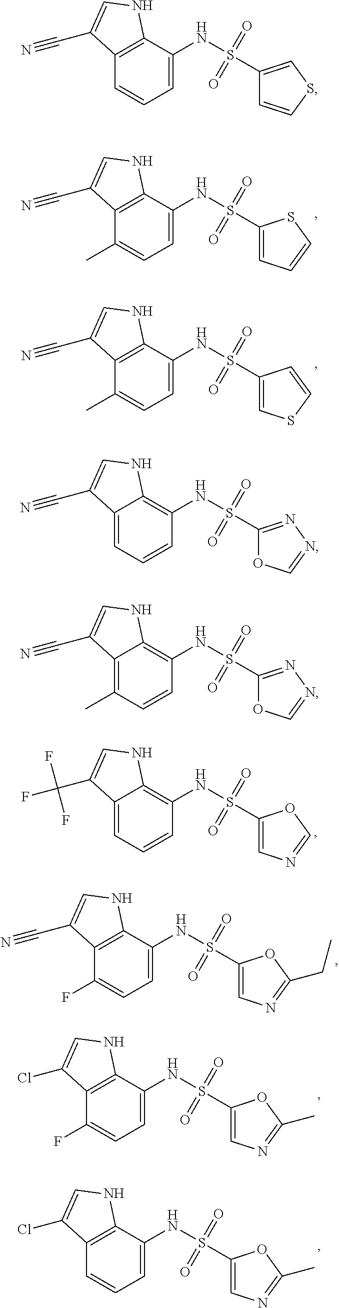

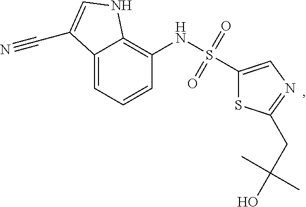 [SMILES: CC(C)(O)Cc1ncc(S(=O)(=O)Nc2cccc3c(C#N)c[nH]c23)s1]

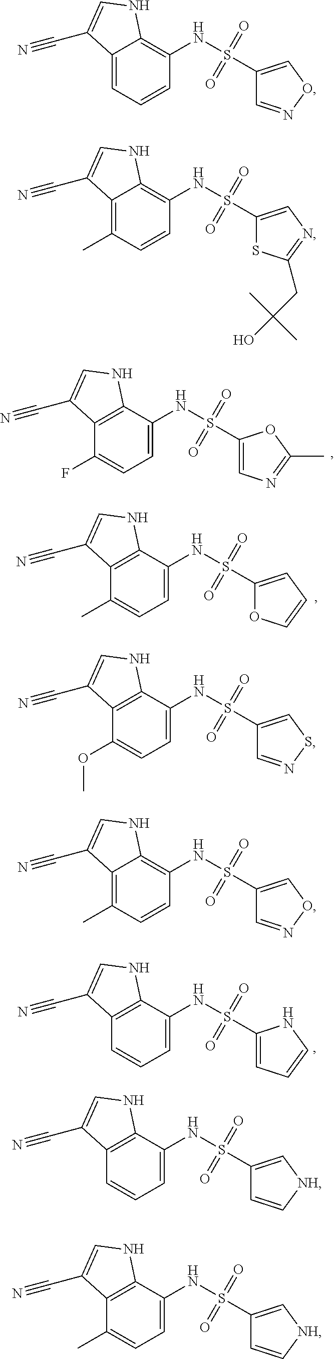

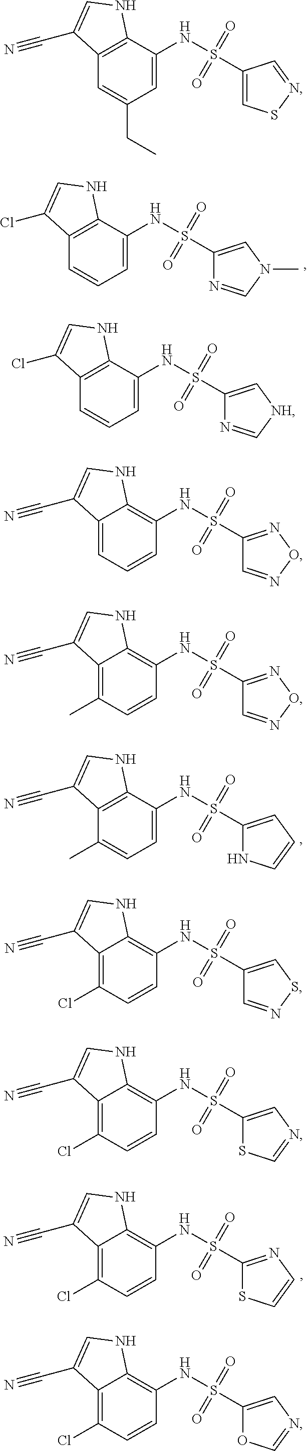

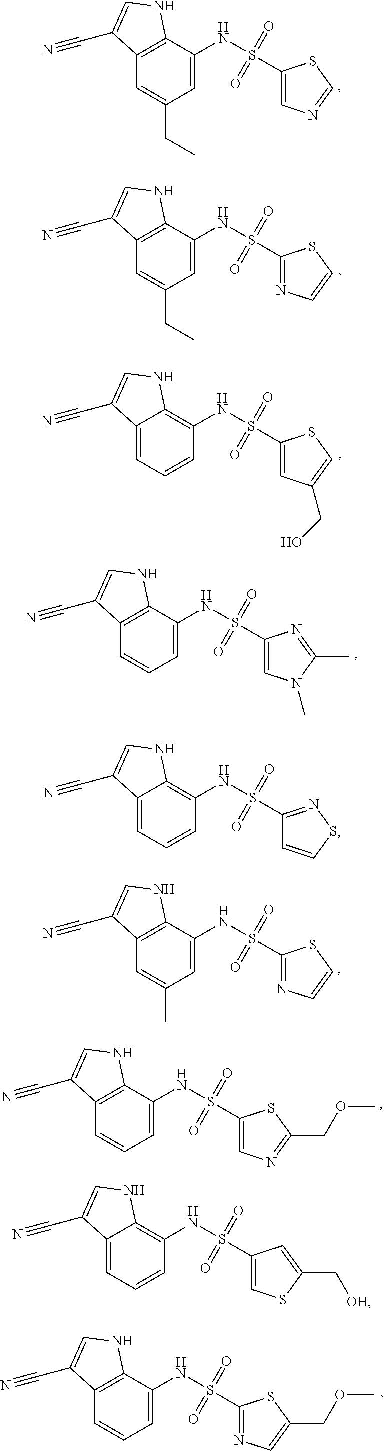

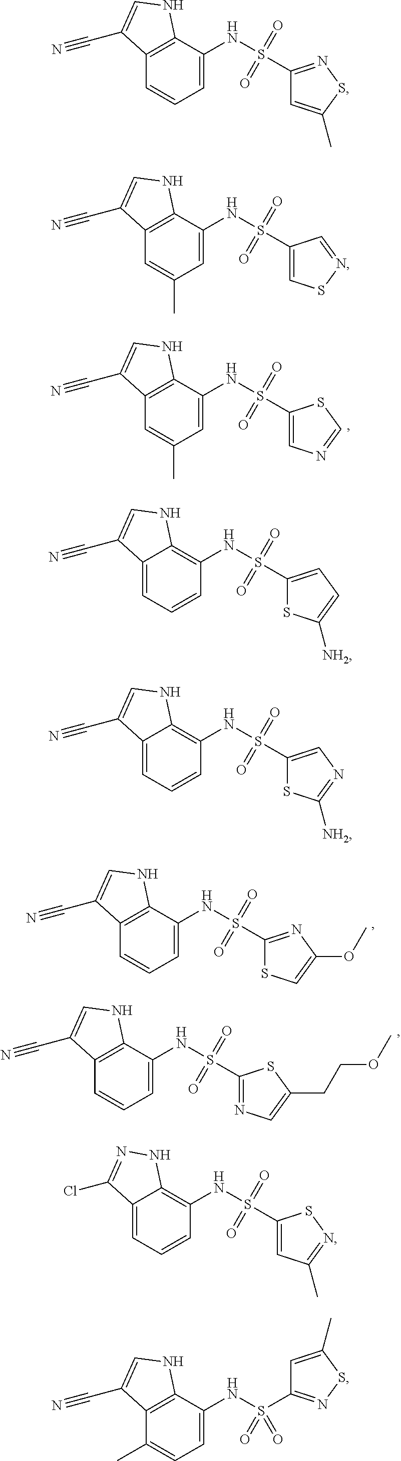

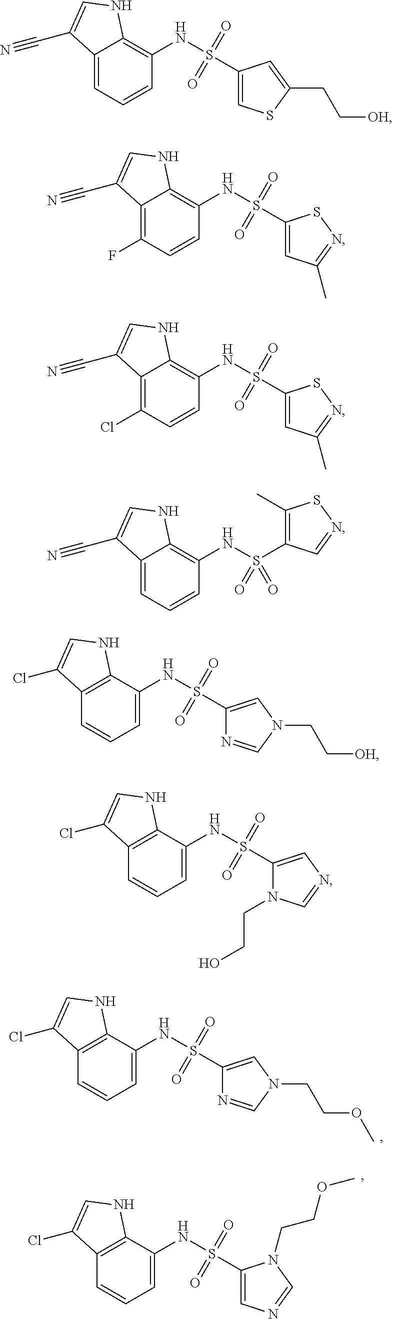

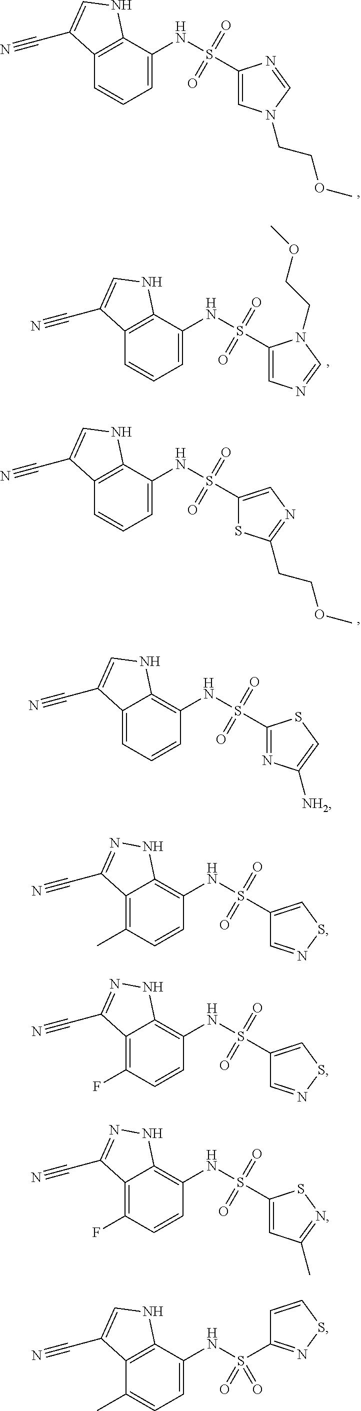

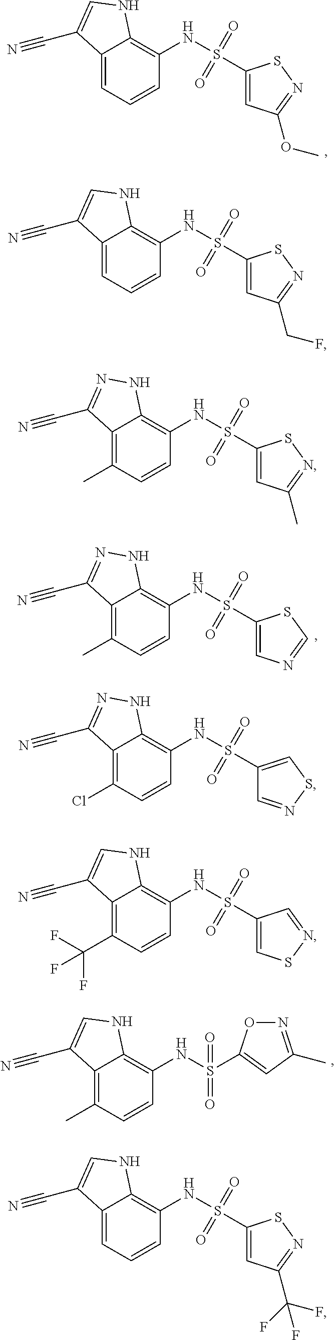

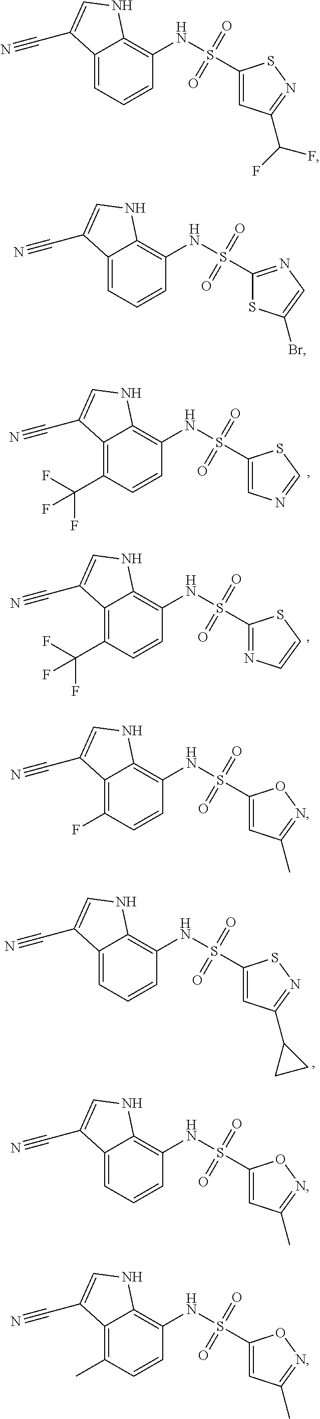

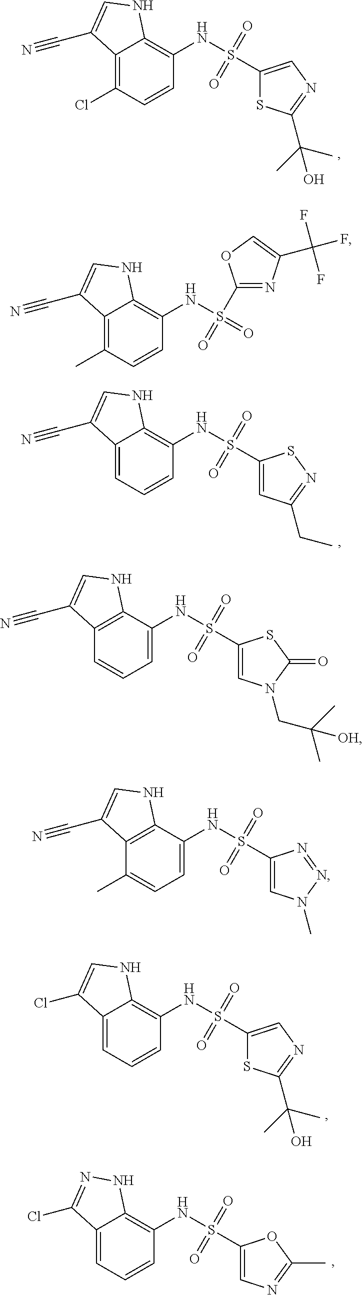

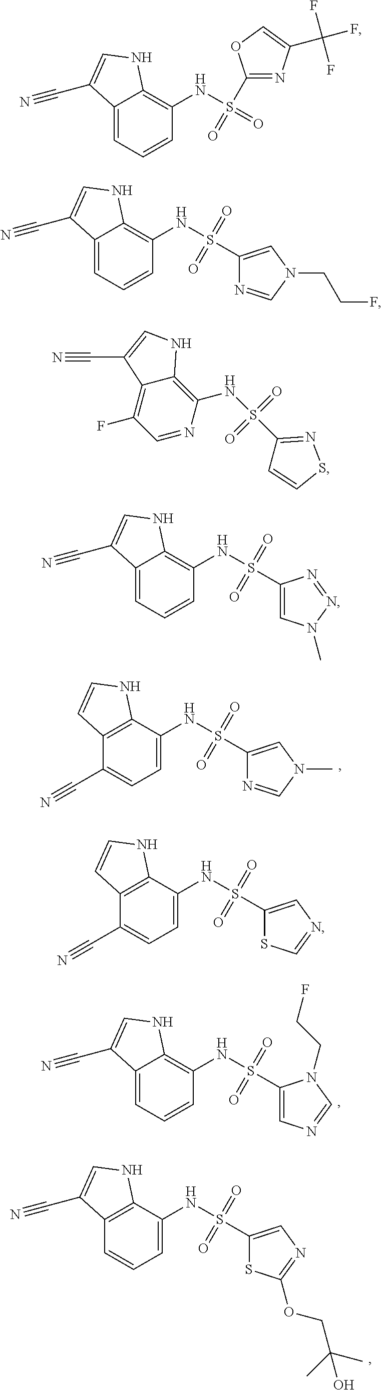

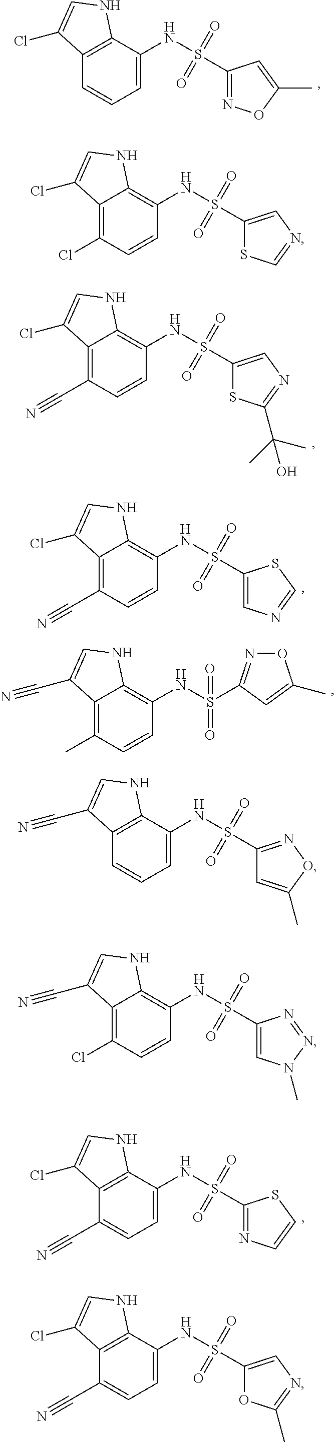

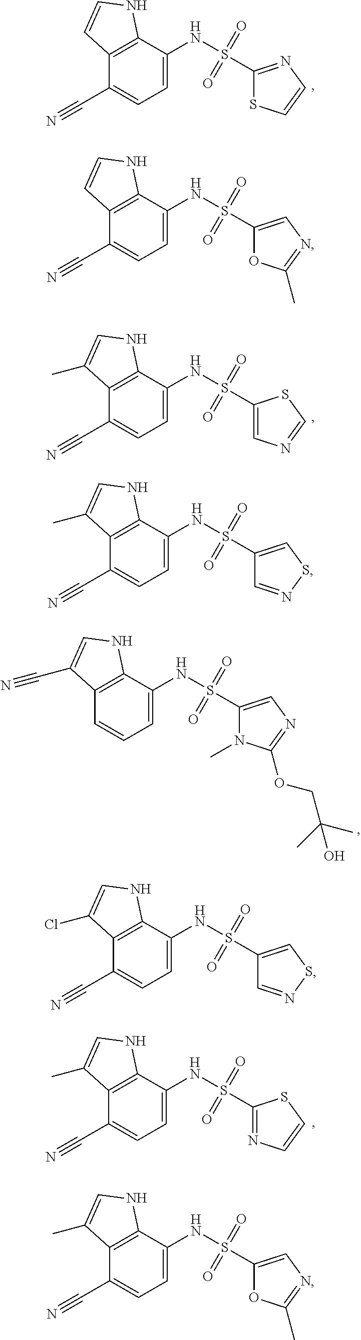

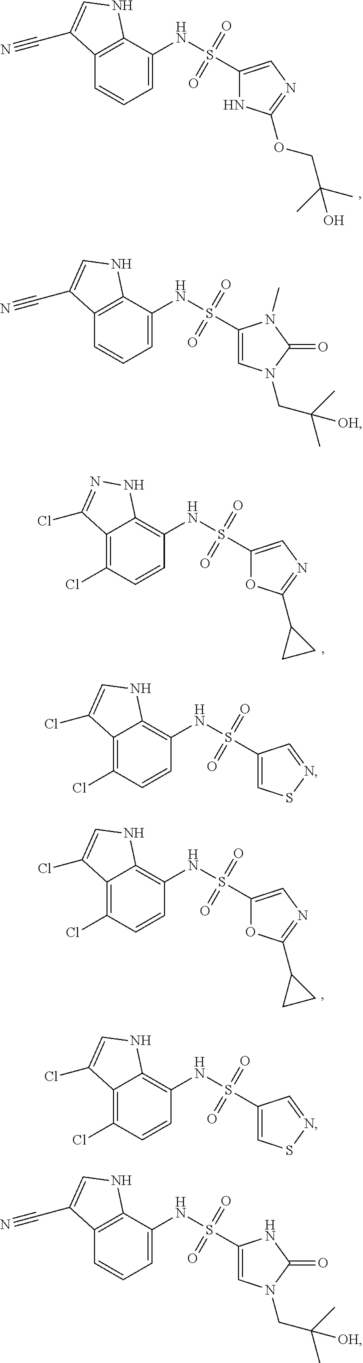

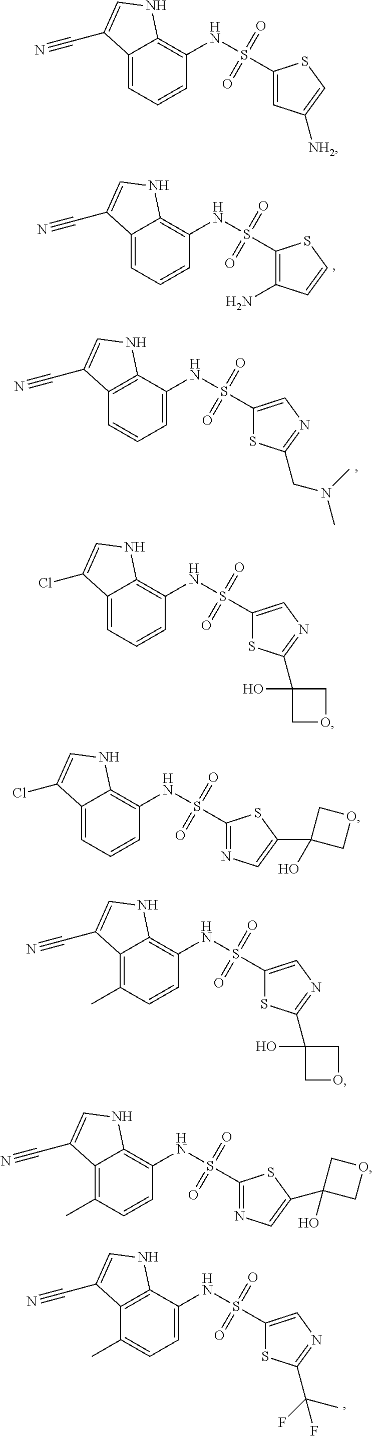

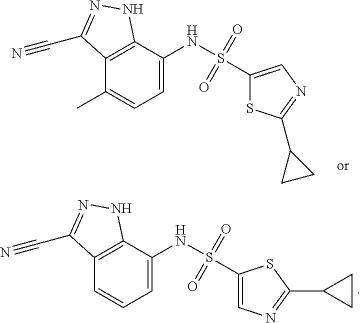 [SMILES: Cc1ccc(NS(=O)(=O)c2cnc(C3CC3)s2)c2[nH]nc(C#N)c12 ; N#Cc1n[nH]c2c(NS(=O)(=O)c3cnc(C4CC4)s3)cccc12]

Some exemplary structures of Formula (I), or pharmaceutically acceptable salts thereof, include those of Formulas (II) through (VI):

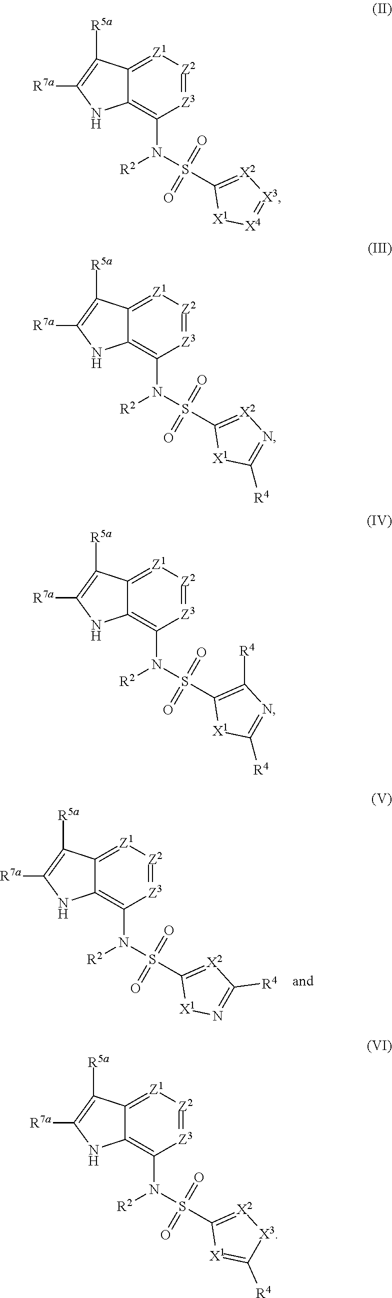

In some embodiments of compounds of Formula (I) or their pharmaceutically acceptable salts, at least one of Z¹, Z² and Z³ can be N. In some embodiments, Z³ can be N. In some embodiments, Z¹ can be N. In other embodiments, Z² can be N. In other embodiments, Z¹ and Z³ can be N.

In some embodiments of compounds of Formula (I) or their pharmaceutically acceptable salts, R^{1a} can be —(C₁-C₆)alkyl. In other embodiments, R^{1a} can be —CH₃. In still other embodiments, R^{1a} can be halogen. In other embodiments, R^{1a} can be —(C₁-C₆)haloalkyl.

In some embodiments of compounds of Formula (I) or their pharmaceutically acceptable salts, R² can be H. In other embodiments, R² can be —(C₁-C₆)alkyl. In other embodiments, R² is —C(O)R⁶. In still other embodiments, R² can be —C(O)(C₁-C₆)alkyl.

In some embodiments of compounds of Formula (I) or their pharmaceutically acceptable salts, R³ can be a 5-membered monocyclic nitrogen-containing heteroaryl optionally substituted with one or two or three substituents selected from R⁴.

In some embodiments of compounds of Formula (I) or their pharmaceutically acceptable salts, R^{5a} can be —CN. In other embodiments, R^{5a} can be halogen. In other embodiments, R^{5a} can be —(C₁-C₆)haloalkyl. In still other embodiments, R^{5a} can be —(C₁-C₆)alkyl.

In some embodiments of compounds of Formula (I) or their pharmaceutically acceptable salts, R^{7a} can be —CN. In other embodiments, R^{7a} can be halogen. In other embodiments, R^{7a} can be —(C₁-C₆)haloalkyl. In still other embodiments, R^{7a} can be —(C₁-C₆)alkyl.

In some embodiments of compounds of Formula (I) or their pharmaceutically acceptable salts, A¹ can be

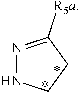

In other embodiments, A¹ can be

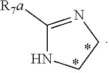

In still other embodiments, A¹ can be

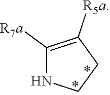

In some embodiments of compounds of Formula (I) or Formula (II) or their pharmaceutically acceptable salts, X¹ can be O, S, or N(R⁴). In some embodiments of compounds of Formula (I), Formula (II), Formula (III), Formula (IV), or Formula (V) or their pharmaceutically acceptable salts, X¹ can be O or S. In some embodiments of compounds of Formula (I), Formula (II), Formula (III), Formula (IV), or Formula (V) or their pharmaceutically acceptable salts, X¹ can be O. In some embodiments of compounds of Formula (I), Formula (II), Formula (III), Formula (IV), or Formula (V) or their pharmaceutically acceptable salts, X¹ can be S. In some embodiments of Formula (I), Formula (II), Formula (III), Formula (V) or Formula (VI) or their pharmaceutically acceptable salts, X² can be N or C(R⁴). In other embodiments of Formula (I), Formula (II), Formula (III), Formula (V) or Formula (VI) or their pharmaceutically acceptable salts, X² can be N. In some embodiments of Formula (I), Formula (II), Formula (III), Formula (V) or Formula (VI) or their pharmaceutically acceptable salts, X² can be C(R⁴). In some embodiments of Formula (I) or Formula (II) or their pharmaceutically acceptable salts, X³ can be C(R⁴) or N. In some embodiments of compounds of Formula (I) or Formula (VI) or their pharmaceutically acceptable salts, X³ can be S or O or N(R⁴). In some embodiments of compounds of Formula (I) or Formula (VI) or their pharmaceutically acceptable salts, X³ can be S. In some embodiments of compounds of Formula (I) or Formula (VI) or their pharmaceutically acceptable salts, X³ can be O.

In some embodiments of compounds of Formula (I) or their pharmaceutically acceptable salts, A¹ can be

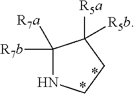

In some embodiments of compounds of Formula (I) or their pharmaceutically acceptable salts, R^{5b} can be —(C₁-C₆)alkyl.

In some embodiments of compounds of Formula (I) or their pharmaceutically acceptable salts, R^{5a} can be taken together with R^{5b} and the atom to which R^{5a} and R^{5b} are attached to form an optionally substituted 3-7 membered monocyclic cycloalkyl. In some embodiments, R^{5a} can be taken together with R^{5b} and the atom to which R^{5a} and R^{5b} are attached to form an optionally substituted cyclopropyl.

In some embodiments of compounds of Formula (I) or their pharmaceutically acceptable salts, R³ can be —(C₁-C₆)alkyl optionally substituted with one or two or three substituents selected from R⁴.

In some embodiments of compounds of Formula (I) or their pharmaceutically acceptable salts, each R⁴ can be independently —H, halogen, —CN, —(C₁-C₆)alkyl, —(C₁-C₆)haloalkyl, —(C₁-C₆)alkoxy, —(CH₂)˜S(O)₂(C₁-C₆)alkyl or —C(O)R^{z}.

In some embodiments of compounds of Formula (I) or their pharmaceutically acceptable salts, R⁴ can be CH₃. In other embodiments, R⁴ can be CD₃. In still other embodiments, R⁴ can be NH₂. In other embodiments, R⁴ can be NHBoc. In yet other embodiments, R⁴ can be NHC(O)(C₁-C₆)alkyl.

In some embodiments of compounds of Formula (I) or their pharmaceutically acceptable salts, R⁴ can be —R^{x1}. In some embodiments, —R^{x}i can be selected from the group consisting of:

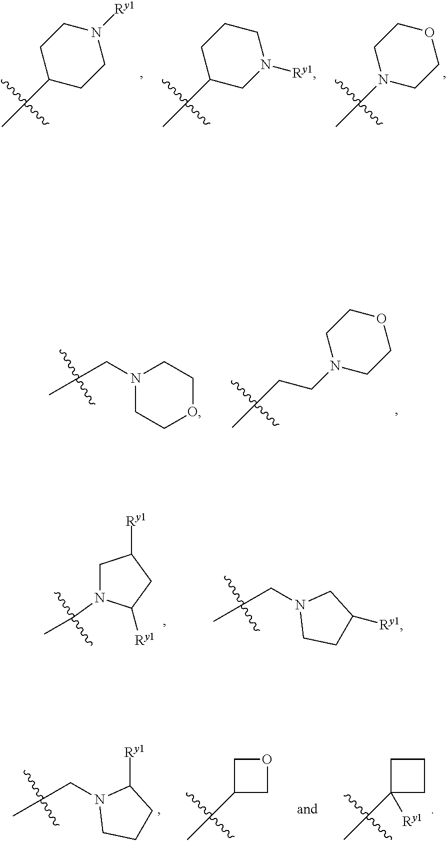

In some embodiments of compounds of Formula (I) or their pharmaceutically acceptable salts, R^{y1} can be —H. In other embodiments, R^{y1} can be —(C₁-C₆)alkyl. In still other embodiments, R¹ can be —CN. In some embodiments, R^{y1} can be CH₂CN. In other embodiments, R^{y1} can be BOC. In other embodiments, R^{y1} can be —C(O)(C₁-C₆)alkyl. In still other embodiments, R^{y1} can be —(CH₂)ₙS(O)₂(C₁-C₆)alkyl. In some embodiments, n can be 0. In other embodiments, n can be 1. In other embodiments, n can be 2. In other embodiments, n can be 3. In other embodiments, n can be 4. In other embodiments, R^{y1} can be heterocyclyl. In some embodiments, R^{y1} can be

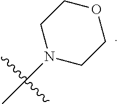 [SMILES: *N1CCOCC1]

In some embodiments of compounds of Formula (I) or their pharmaceutically acceptable salts, R⁴ can be —R^{x2}. In some embodiments, R^{x2} can be selected from the group consisting of:

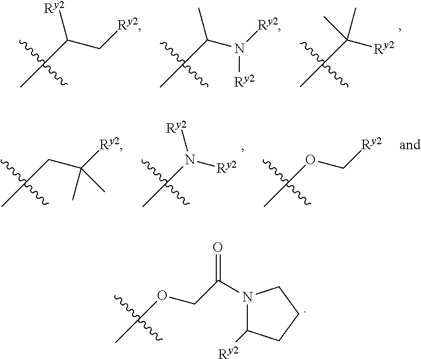

In some embodiments, R^{x2} can be

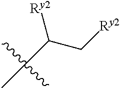

and each R^{y2} can independently be —H, —OH, —CN, C₁-C₆)alkoxy, N((C₁-C₆)alkyl)₂, or (CH₂)ₙS(O)₂(C₁-C₆)alkyl. In other embodiments, R^{x2} can be

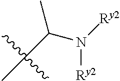

and each R^{y2} can be —(C₁-C₆)alkyl. In some embodiments, each R^{y2} can be —OH. In some embodiments, R^{x2} can be

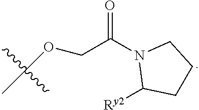

In some embodiments, R^{x2} can be

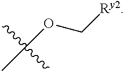

In some embodiments, R^{y2} can be —CN or —CH₂CN. In other embodiments, R^{y2} can be —(CH₂)ₙS(O)₂(C₁-C₆)alkyl. In still other embodiments, R^{x2} can be

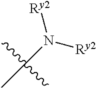

and each R^{y2} can independently be —(C₁-C₆)alkyl, —CH₂CN, —C(O)CH₂CH₂N((C₁-C₆)alkyl)₂, —(CH₂)ₙS(O)₂(C₁-C₆)alkyl, or —CH₂CH₂S(O)₂(C₁-C₆)alkyl.

In some embodiments of compounds of Formula (I) or their pharmaceutically acceptable salts, R³ can be —(C₁-C₆)alkyl, —CH₃. In other embodiments, R³ can be isopropyl. In other embodiments, R³ can be —(C₁-C₆)alkyl substituted with R⁴ and R⁴ can be —R^{x1}. In some embodiments, R^{x1} can be

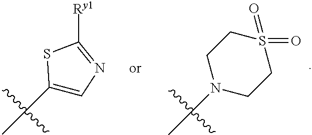

In some embodiments of compounds of Formula (I) or their pharmaceutically acceptable salts, R³ can be a 6-12 membered nitrogen-containing bicyclic heterocyclyl optionally substituted with one or two or three substituents selected from R⁴. In some embodiments, the 6-12 membered nitrogen-containing bicyclic heterocyclyl can be selected from the group consisting of:

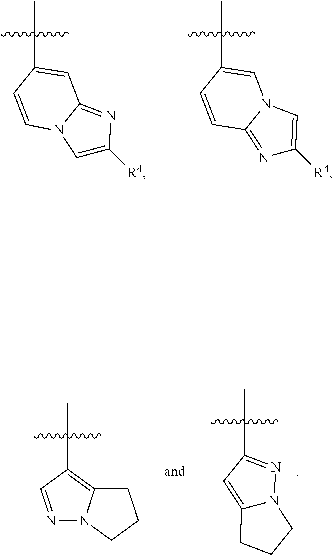

In some embodiments, R⁴ is —CN.

In some embodiments of compounds of Formula (I), the compound can be a compound selected from the group consisting of:

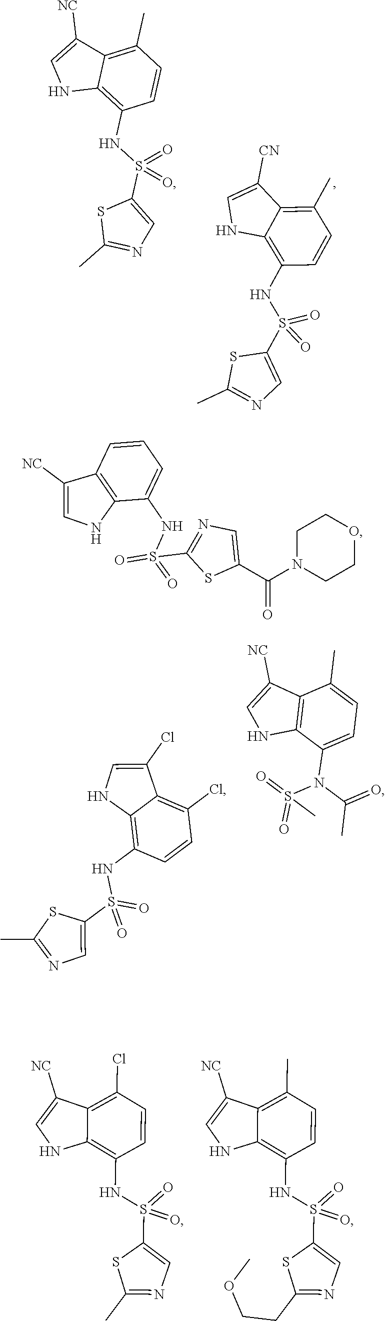

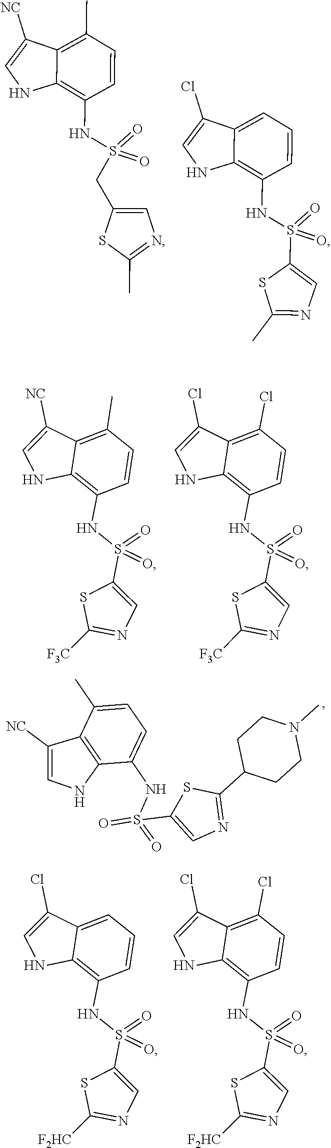

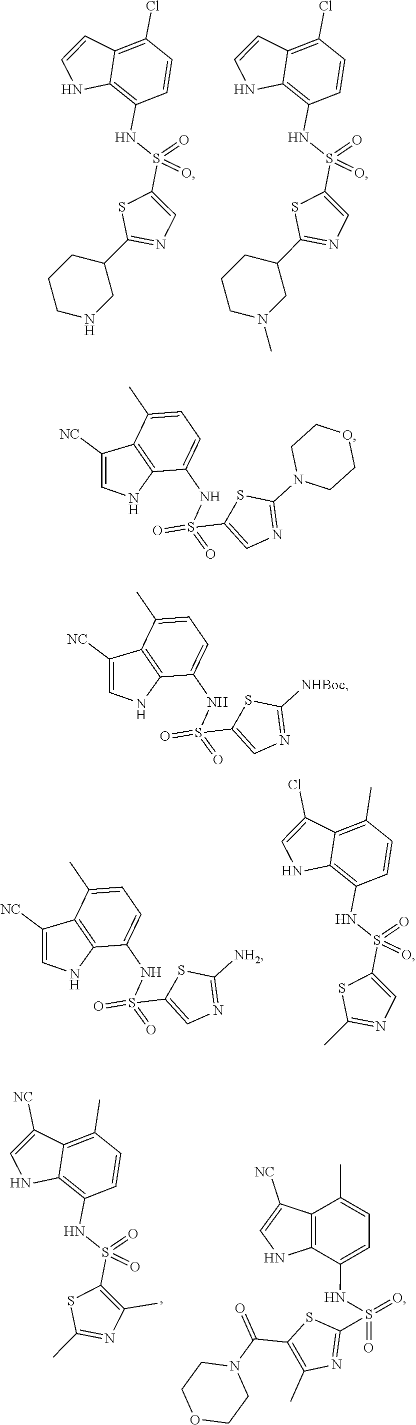

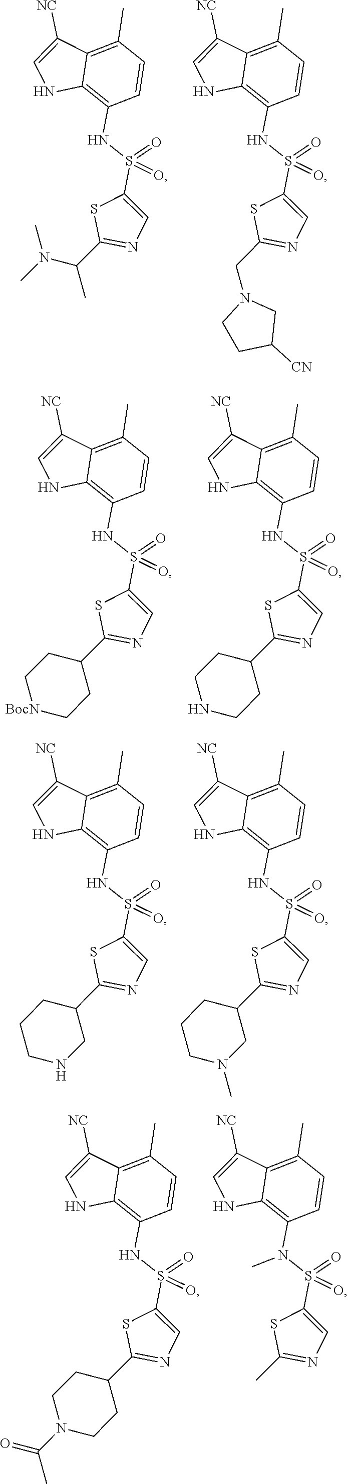

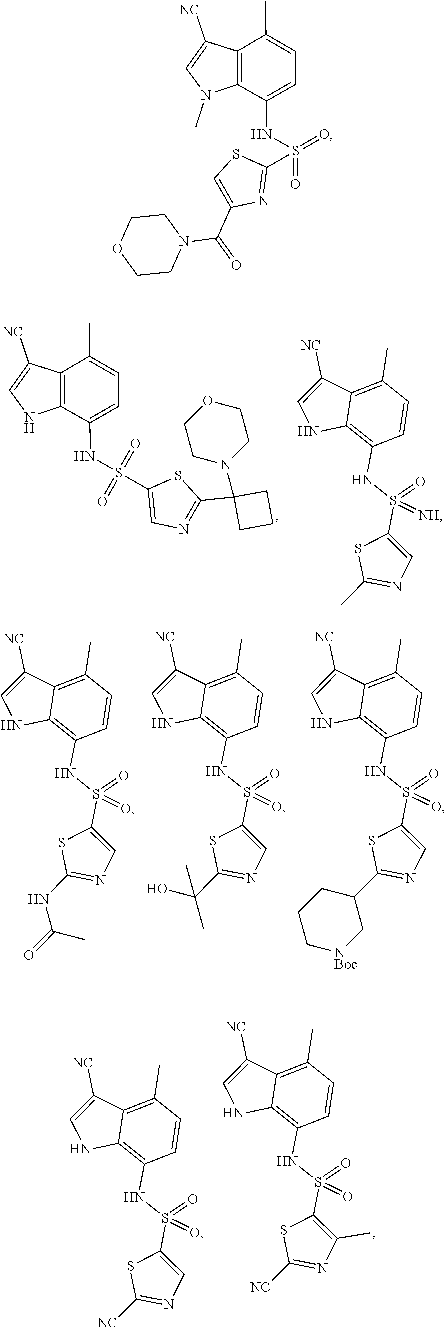

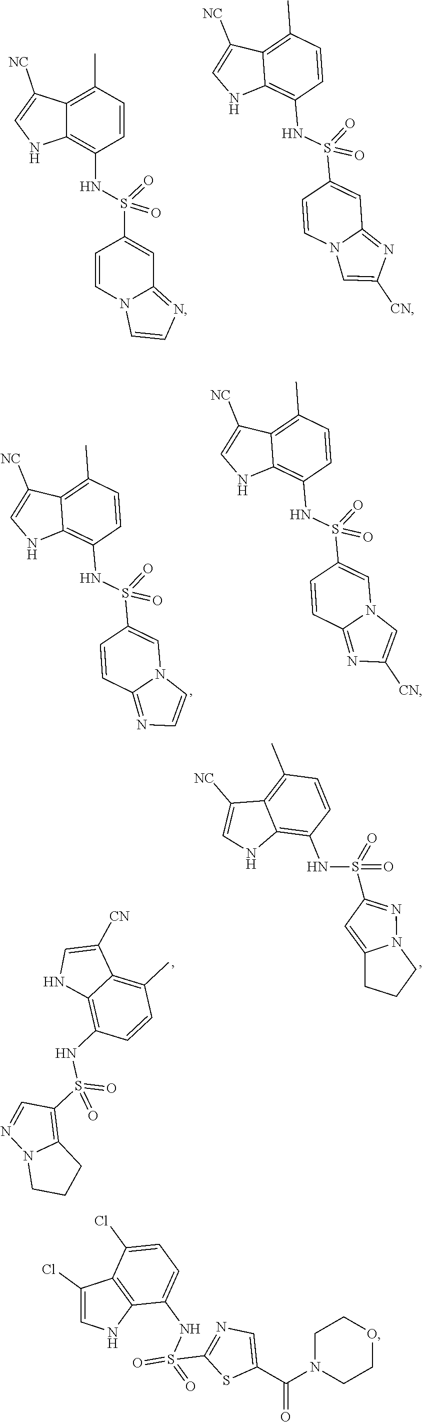

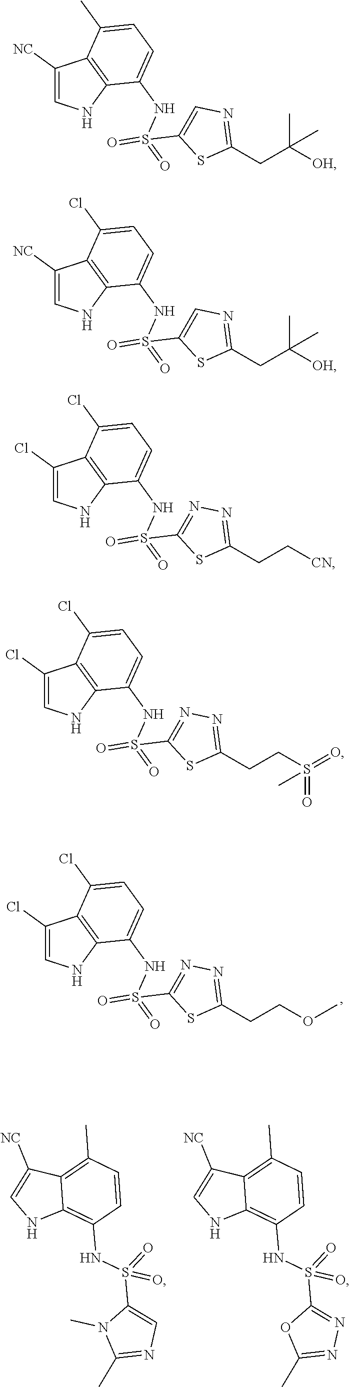

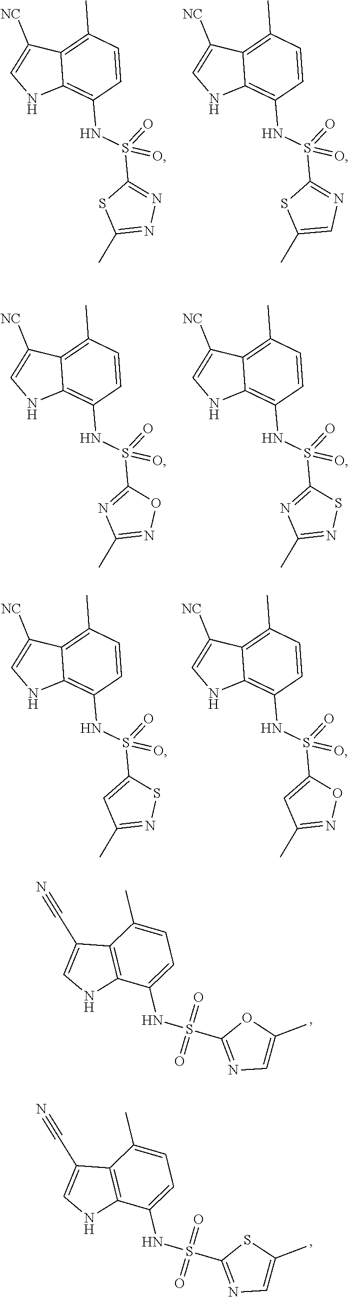

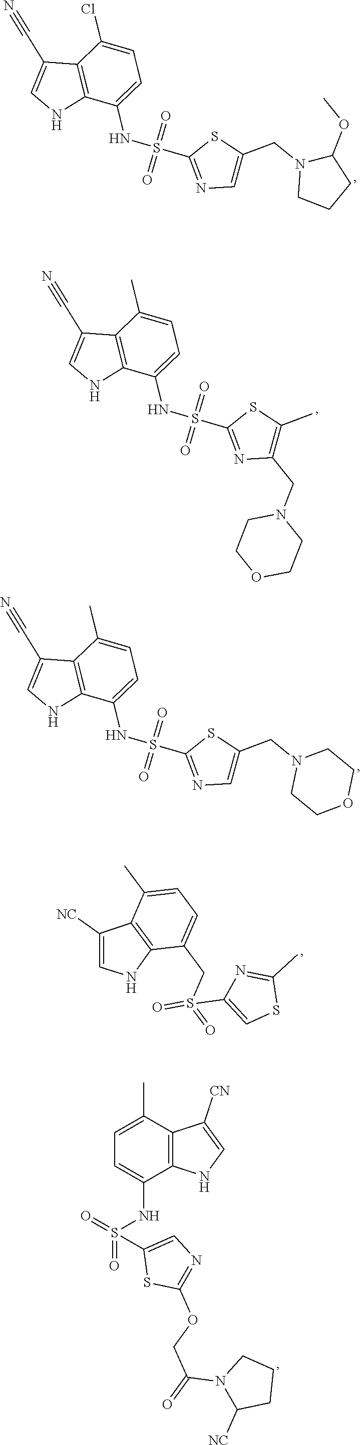 [SMILES: COC1CCCN1Cc1cnc(S(=O)(=O)Nc2ccc(Cl)c3c(C#N)c[nH]c23)s1 ; Cc1sc(S(=O)(=O)Nc2ccc(C)c3c(C#N)c[nH]c23)nc1CN1CCOCC1 ; Cc1ccc(NS(=O)(=O)c2ncc(CN3CCOCC3)s2)c2[nH]cc(C#N)c12 ; Cc1nc(S(=O)(=O)Cc2ccc(C)c3c(C#N)c[nH]c23)cs1 ; Cc1ccc(NS(=O)(=O)c2cnc(OCC(=O)N3CCCC3C#N)s2)c2[nH]cc(C#N)c12]

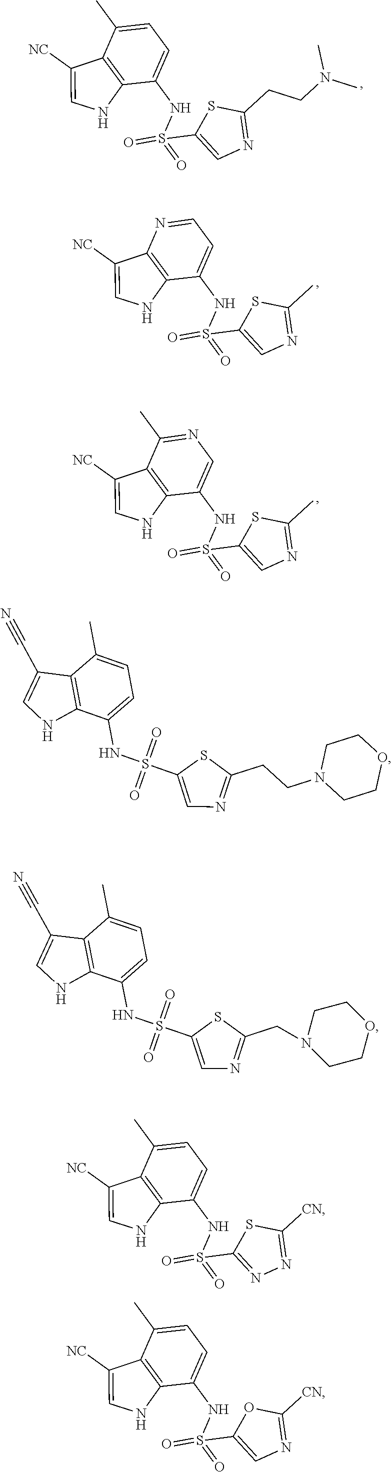

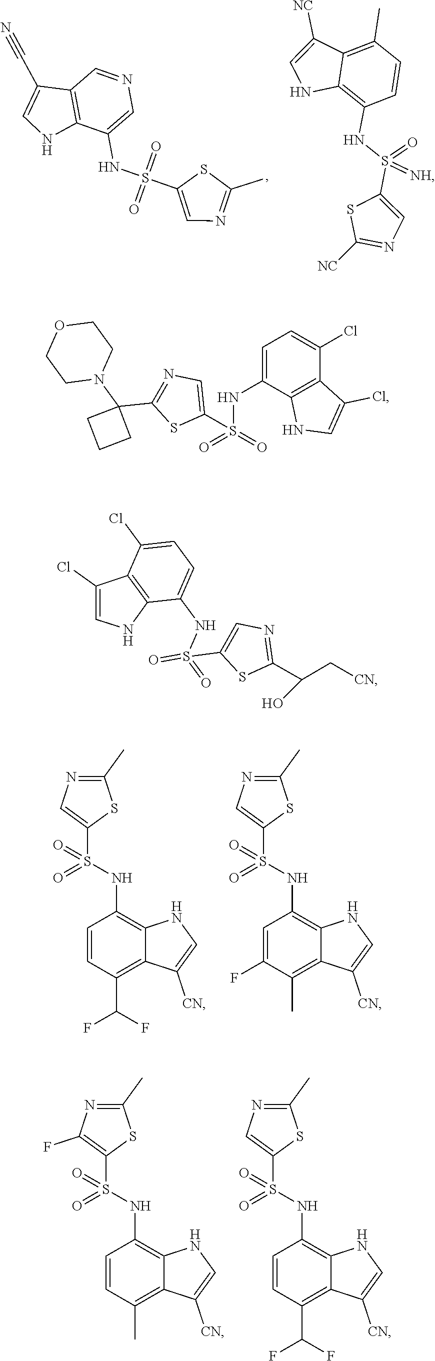

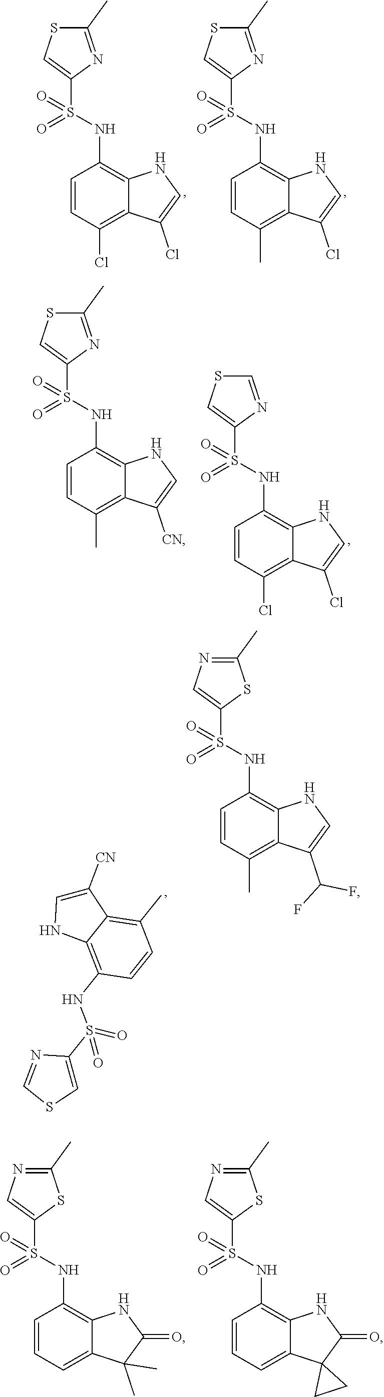

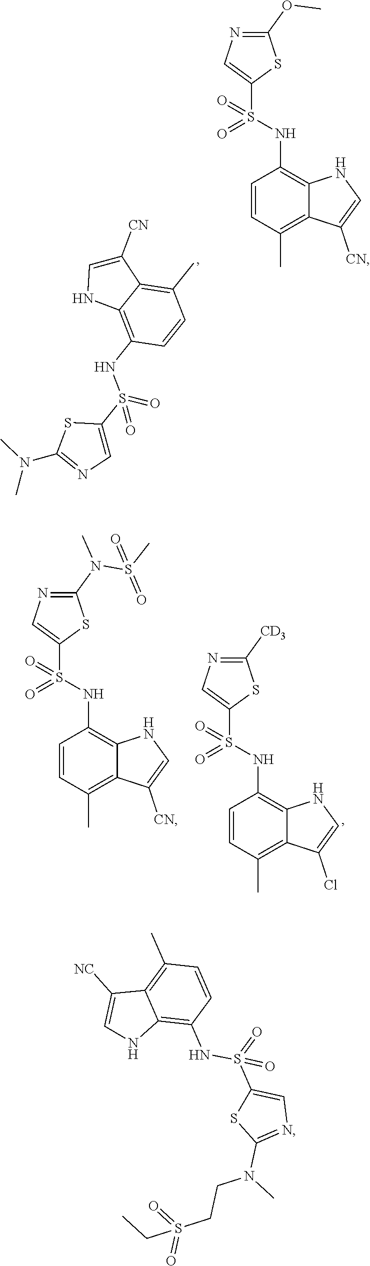

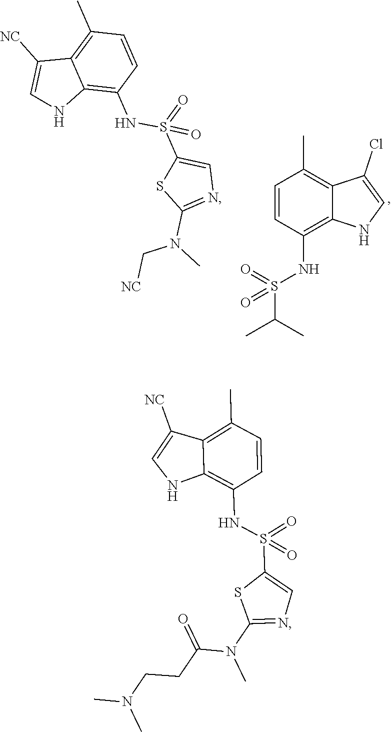

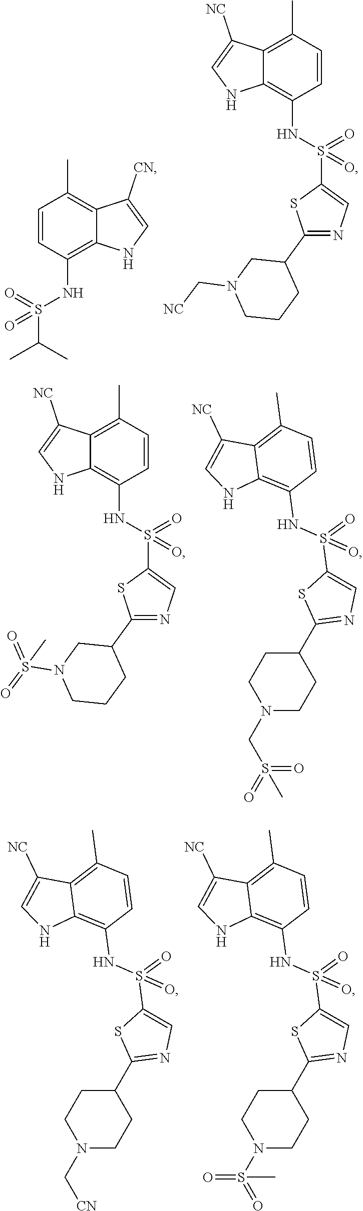

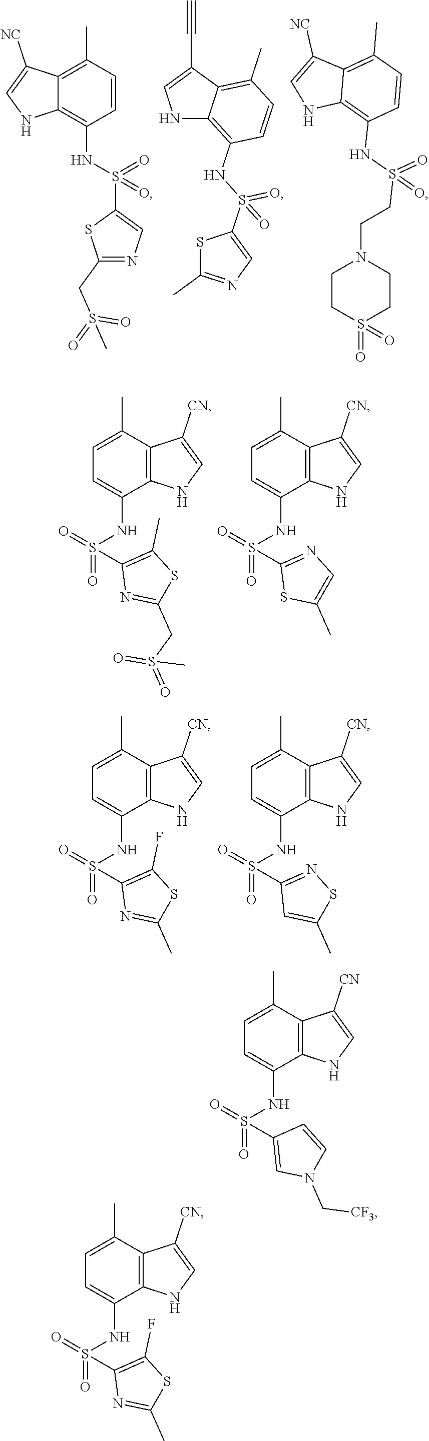

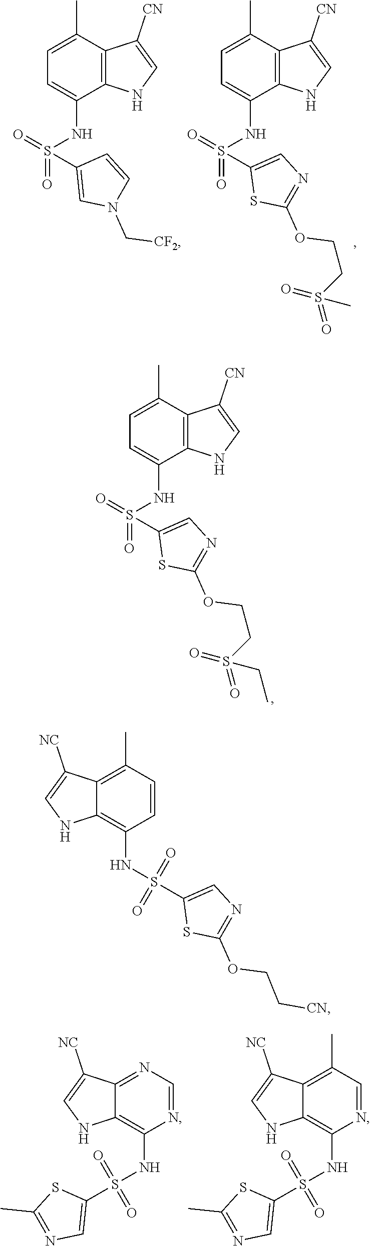

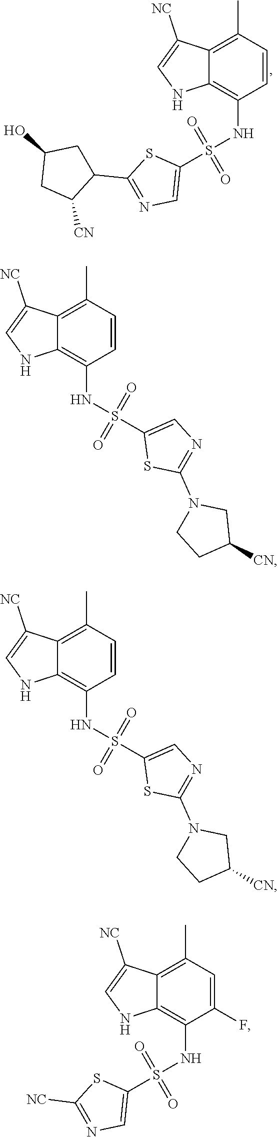

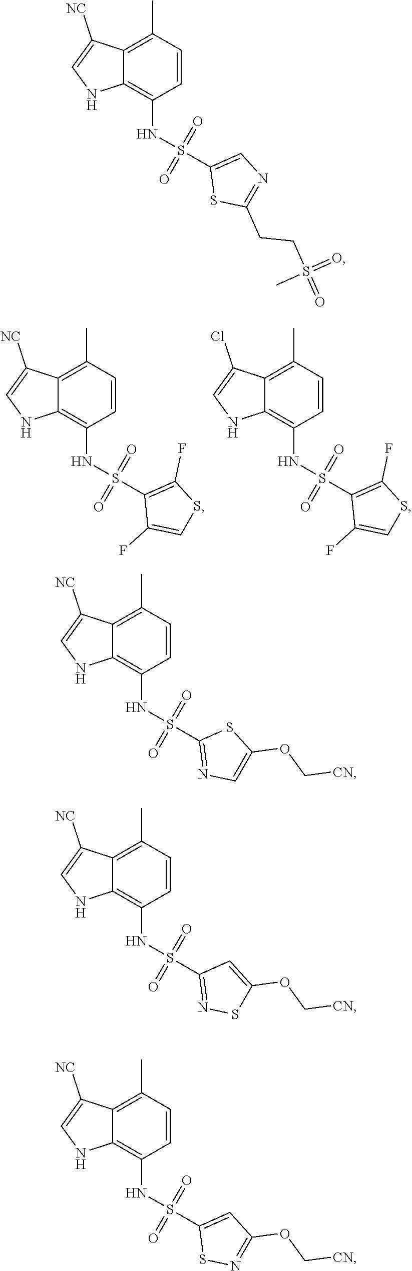

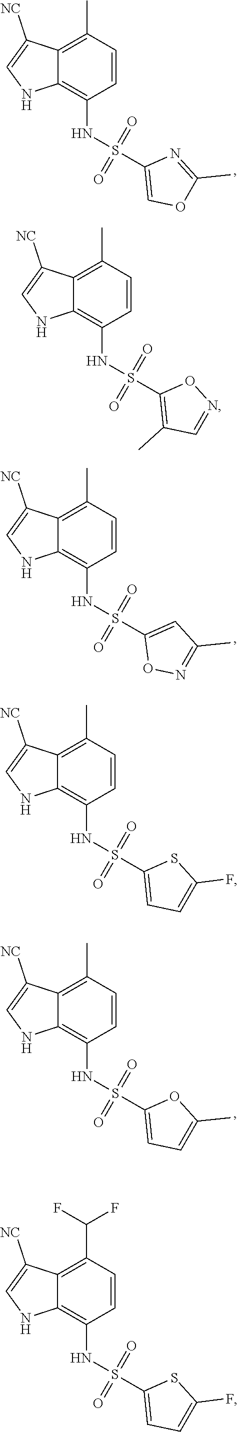 [SMILES: Cc1nc(S(=O)(=O)Nc2ccc(C)c3c(C#N)c[nH]c23)co1 ; Cc1cnoc1S(=O)(=O)Nc1ccc(C)c2c(C#N)c[nH]c12 ; Cc1cc(S(=O)(=O)Nc2ccc(C)c3c(C#N)c[nH]c23)on1 ; Cc1ccc(NS(=O)(=O)c2ccc(F)s2)c2[nH]cc(C#N)c12 ; Cc1ccc(S(=O)(=O)Nc2ccc(C)c3c(C#N)c[nH]c23)o1 ; N#Cc1c[nH]c2c(NS(=O)(=O)c3ccc(F)s3)ccc(C(F)F)c12]

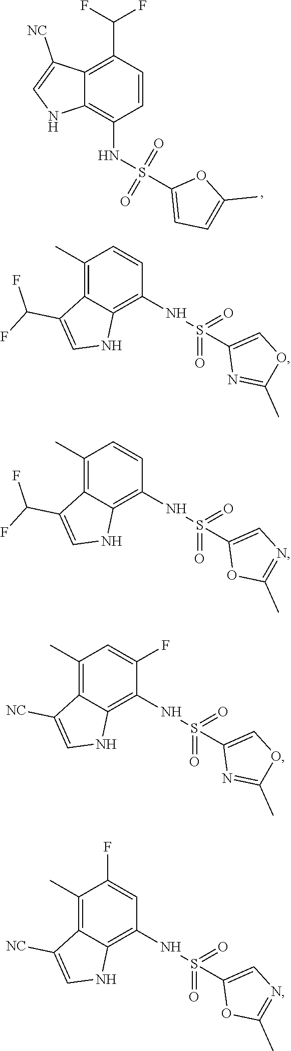 [SMILES: Cc1ccc(S(=O)(=O)Nc2ccc(C(F)F)c3c(C#N)c[nH]c23)o1 ; Cc1nc(S(=O)(=O)Nc2ccc(C)c3c(C(F)F)c[nH]c23)co1 ; Cc1ncc(S(=O)(=O)Nc2ccc(C)c3c(C(F)F)c[nH]c23)o1 ; Cc1nc(S(=O)(=O)Nc2c(F)cc(C)c3c(C#N)c[nH]c23)co1 ; Cc1ncc(S(=O)(=O)Nc2cc(F)c(C)c3c(C#N)c[nH]c23)o1]

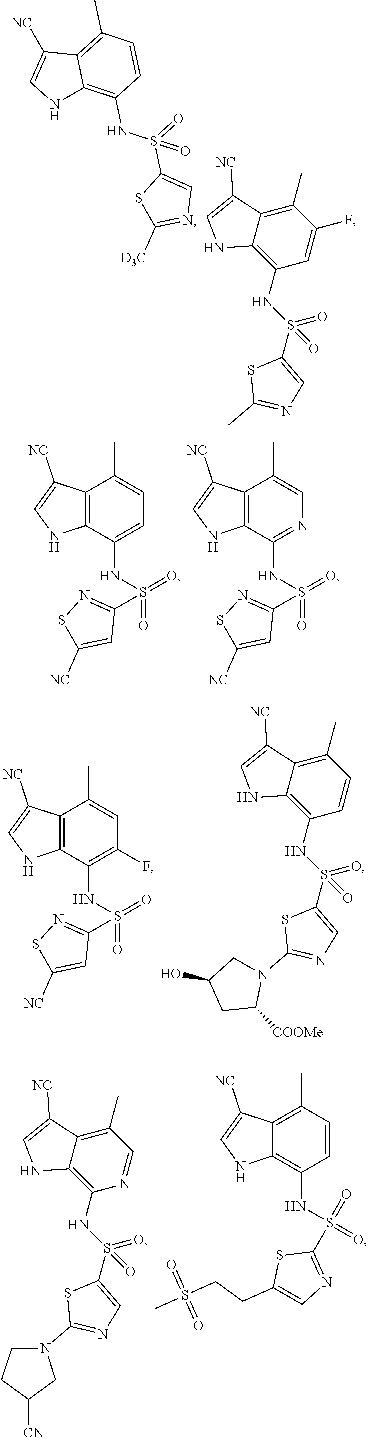

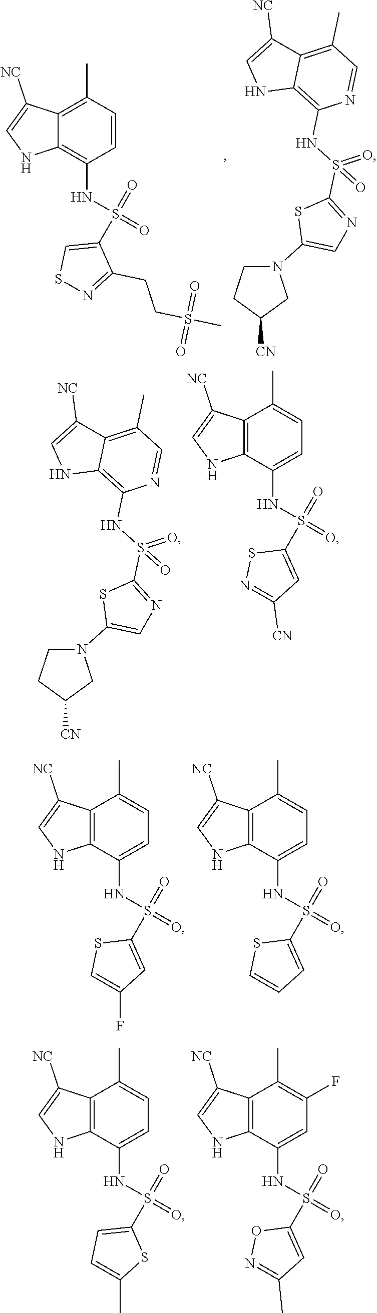

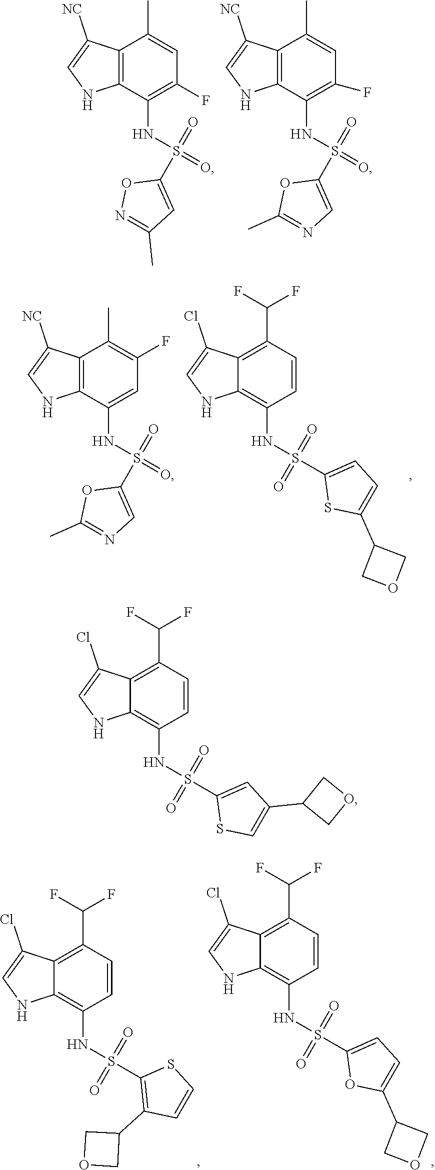

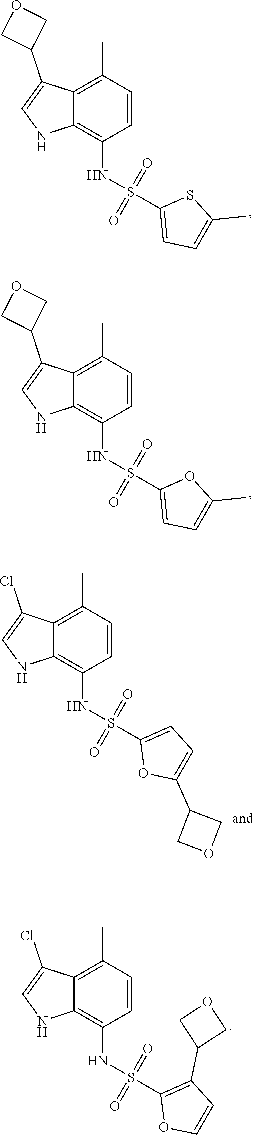 [SMILES: Cc1ccc(S(=O)(=O)Nc2ccc(C)c3c(C4COC4)c[nH]c23)s1 ; Cc1ccc(S(=O)(=O)Nc2ccc(C)c3c(C4COC4)c[nH]c23)o1 ; Cc1ccc(NS(=O)(=O)c2ccc(C3COC3)o2)c2[nH]cc(Cl)c12 ; Cc1ccc(NS(=O)(=O)c2occc2C2COC2)c2[nH]cc(Cl)c12]

or a pharmaceutically acceptable salt of any of the foregoing.

In some embodiments, a compound of Formula (I), or a pharmaceutically acceptable salt thereof, cannot be a compound having the structure:

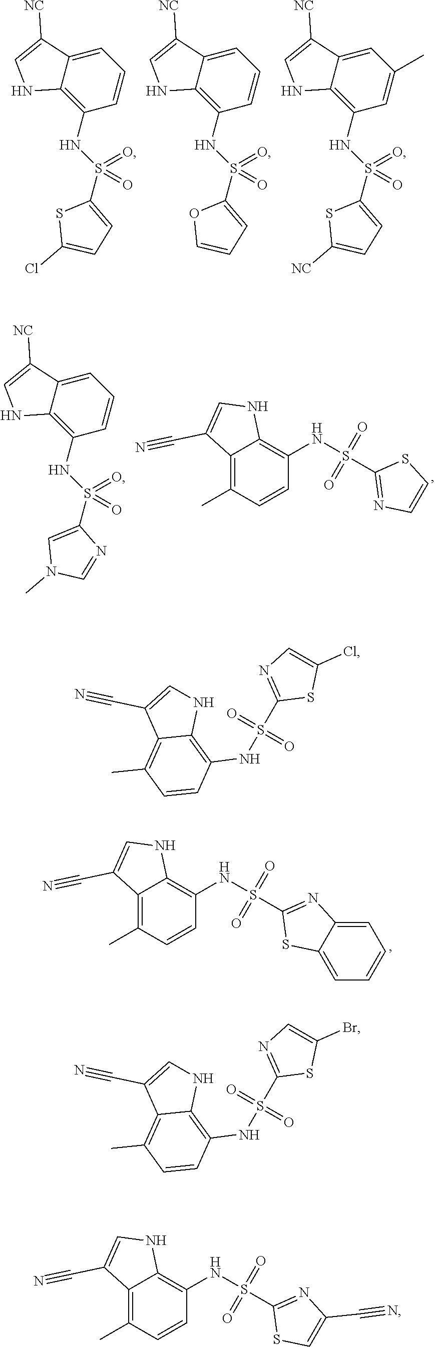

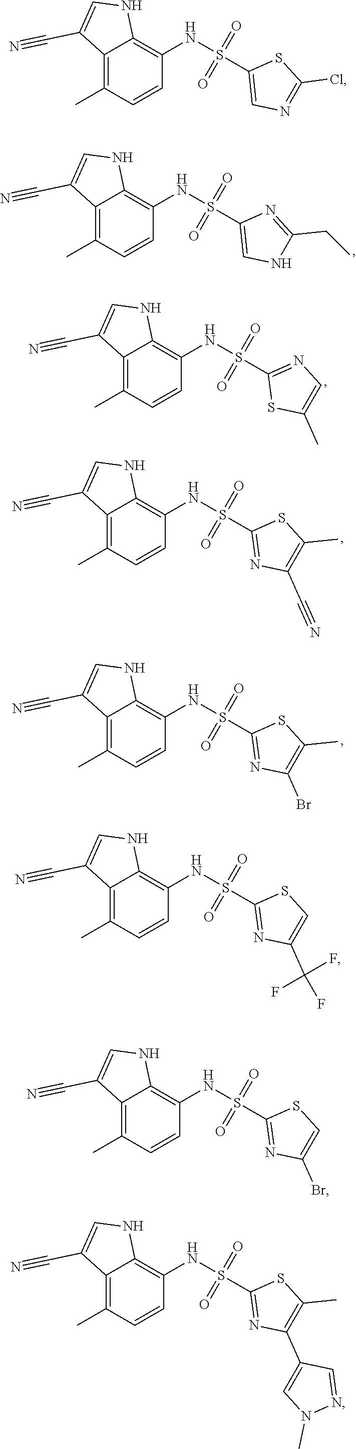

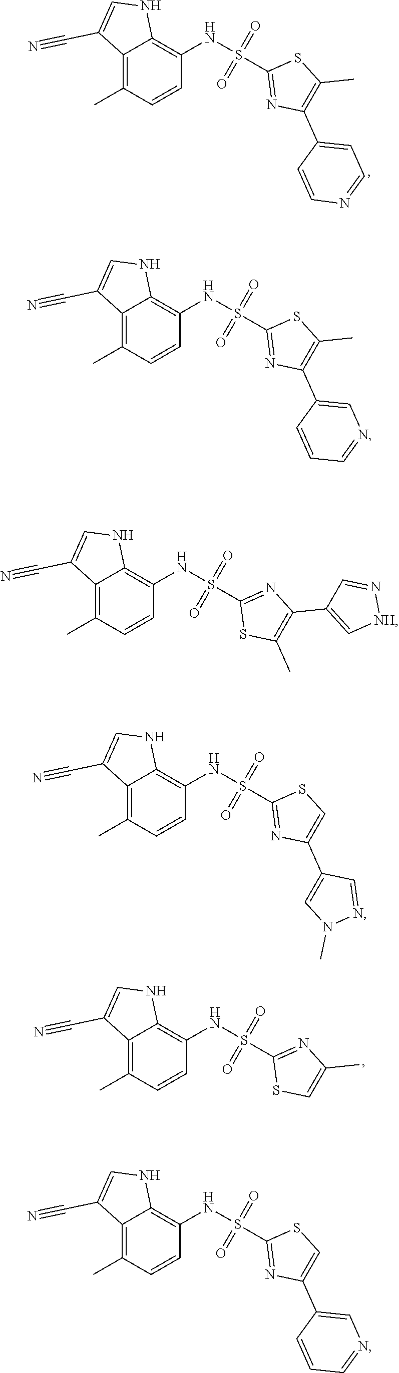 [SMILES: Cc1sc(S(=O)(=O)Nc2ccc(C)c3c(C#N)c[nH]c23)nc1-c1ccncc1 ; Cc1sc(S(=O)(=O)Nc2ccc(C)c3c(C#N)c[nH]c23)nc1-c1cccnc1 ; Cc1sc(S(=O)(=O)Nc2ccc(C)c3c(C#N)c[nH]c23)nc1-c1cn[nH]c1 ; Cc1ccc(NS(=O)(=O)c2nc(-c3cnn(C)c3)cs2)c2[nH]cc(C#N)c12 ; Cc1csc(S(=O)(=O)Nc2ccc(C)c3c(C#N)c[nH]c23)n1 ; Cc1ccc(NS(=O)(=O)c2nc(-c3cccnc3)cs2)c2[nH]cc(C#N)c12]

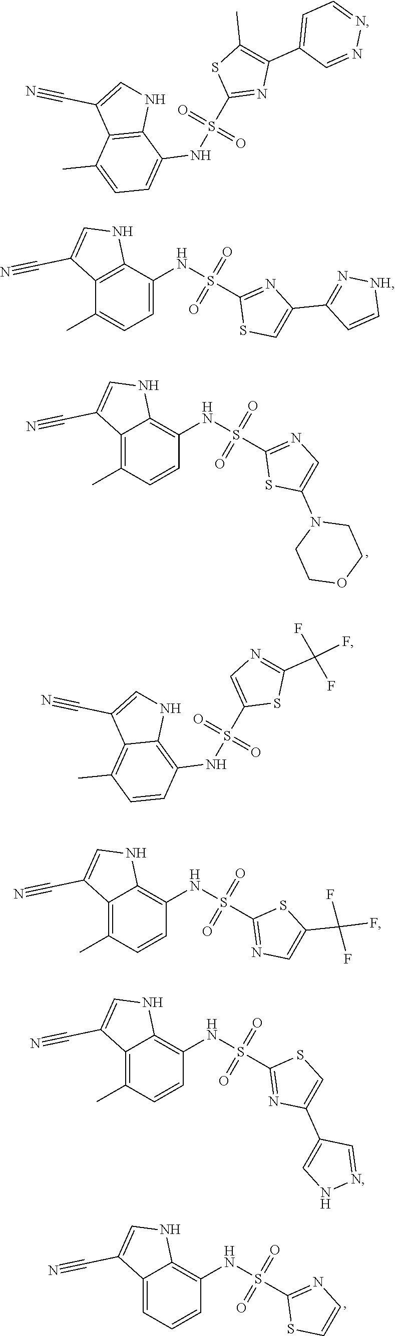

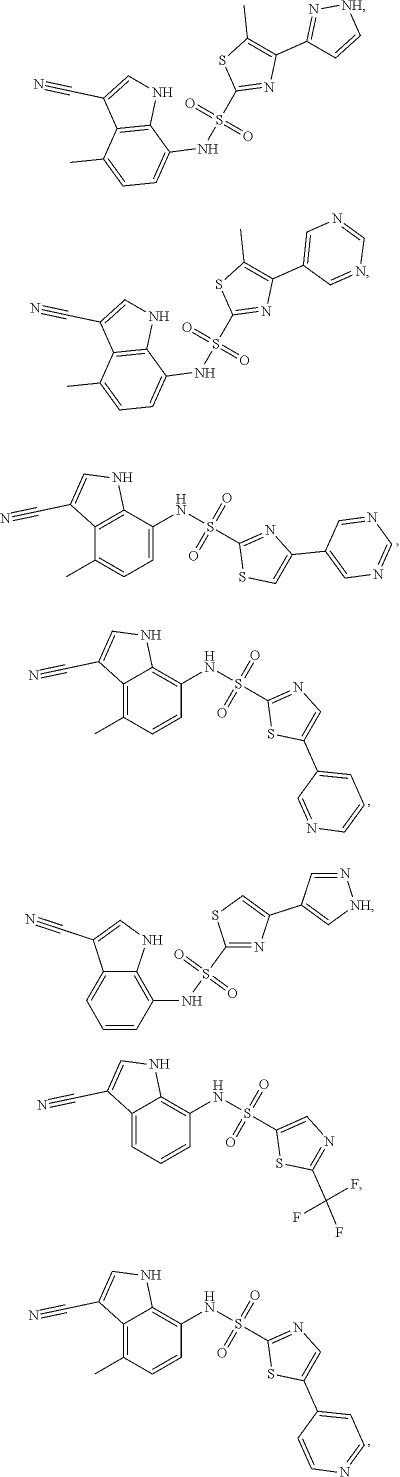

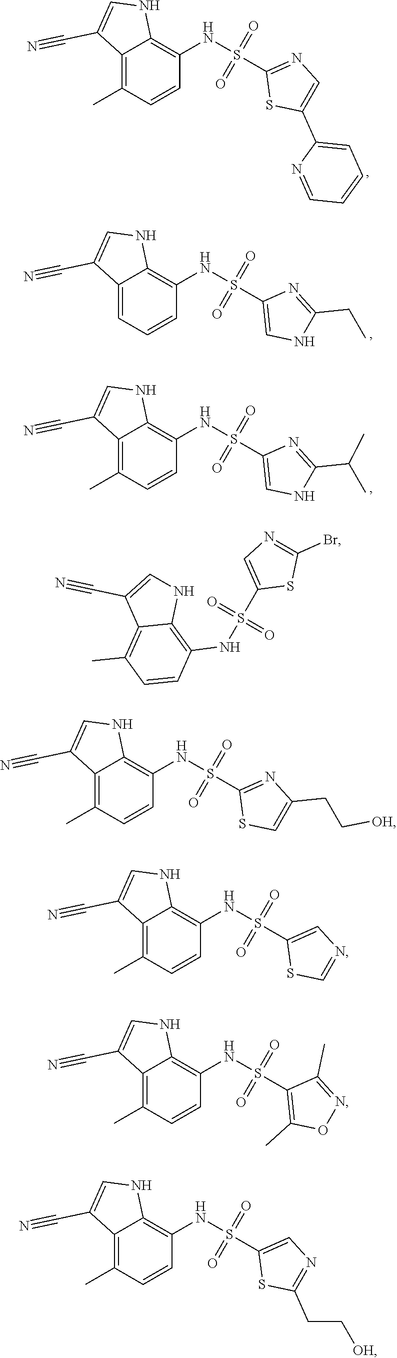

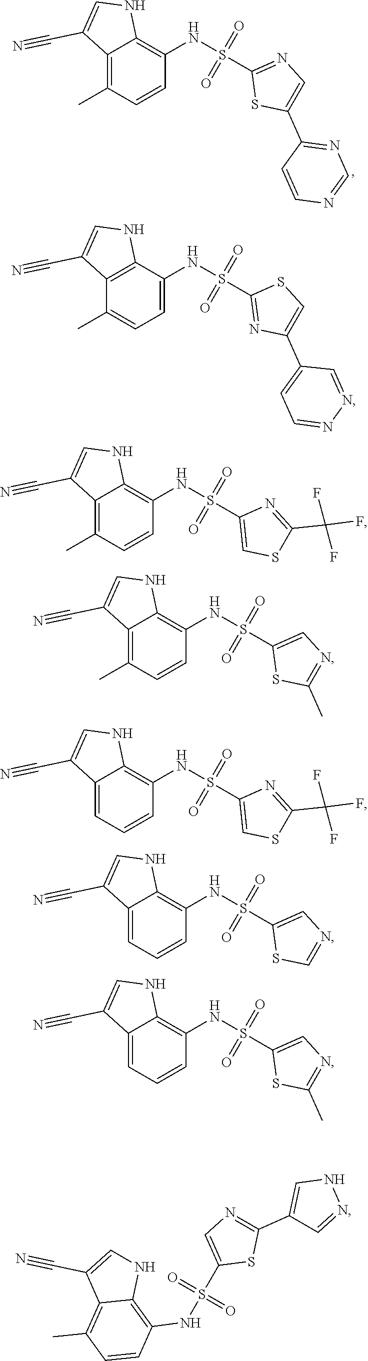

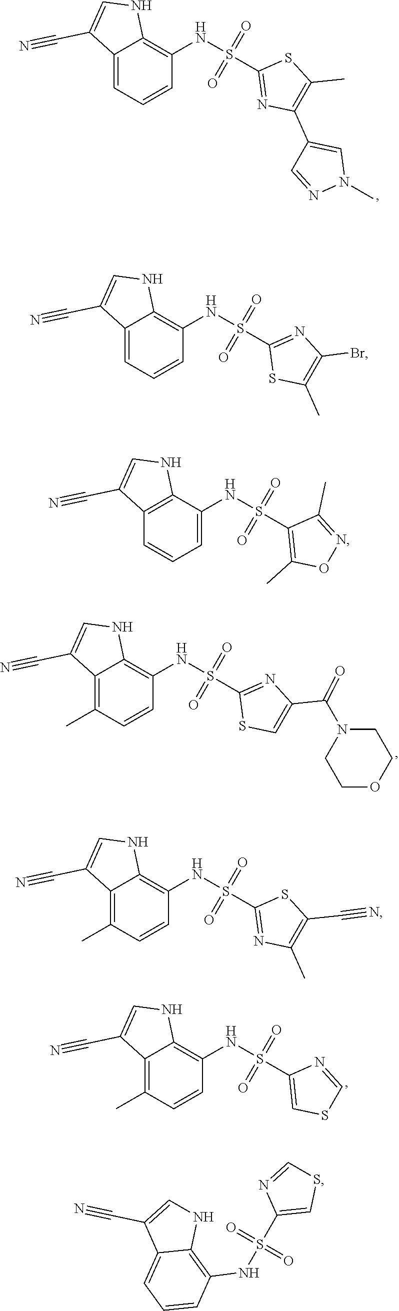

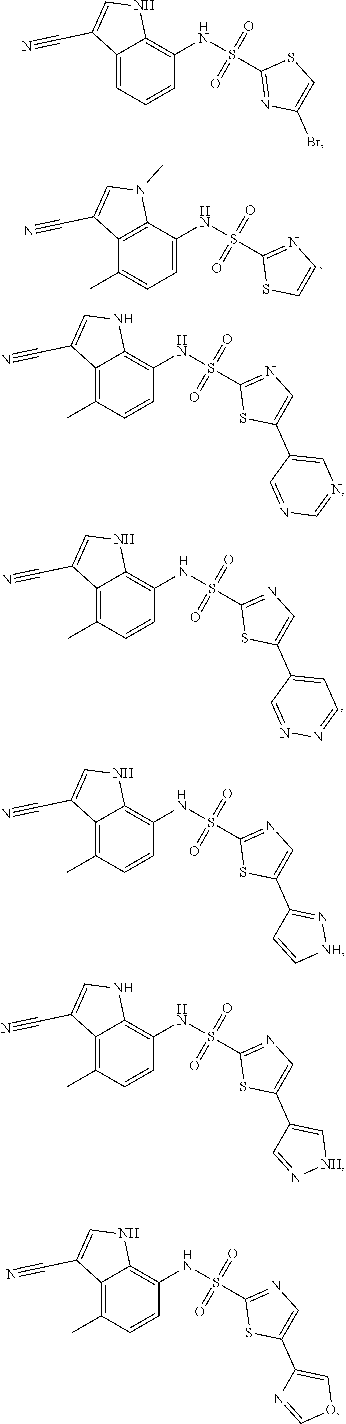

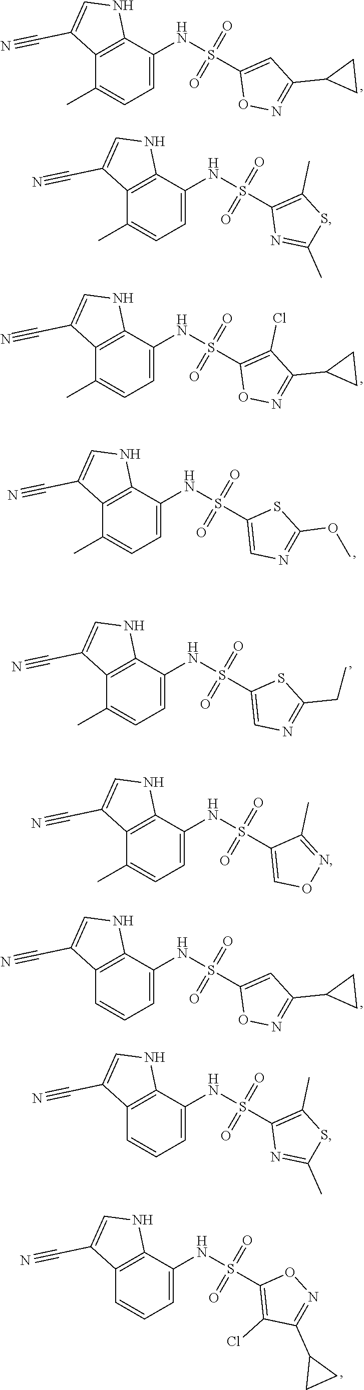

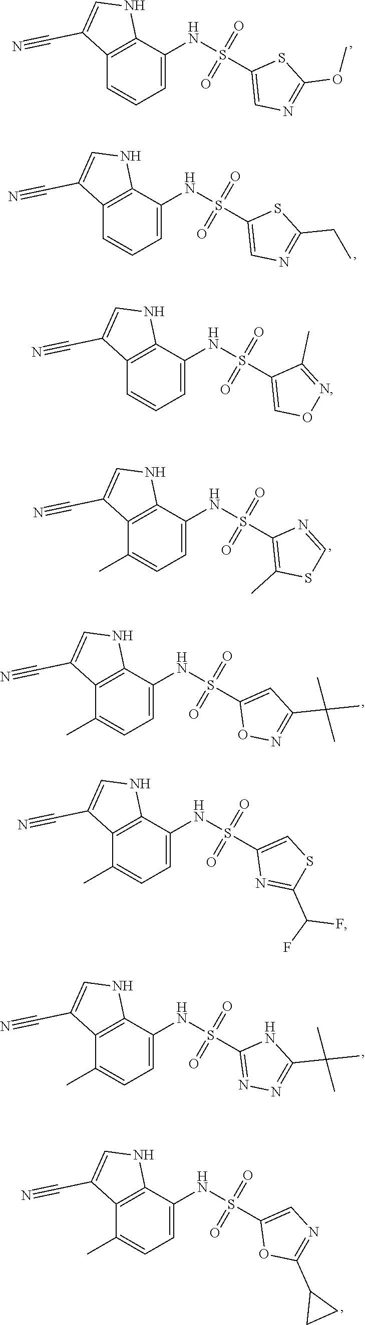

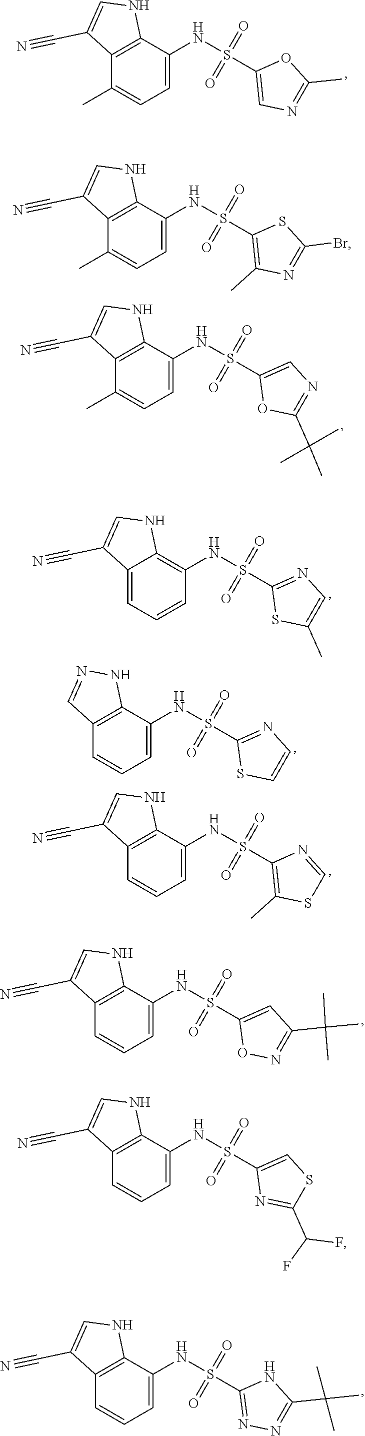

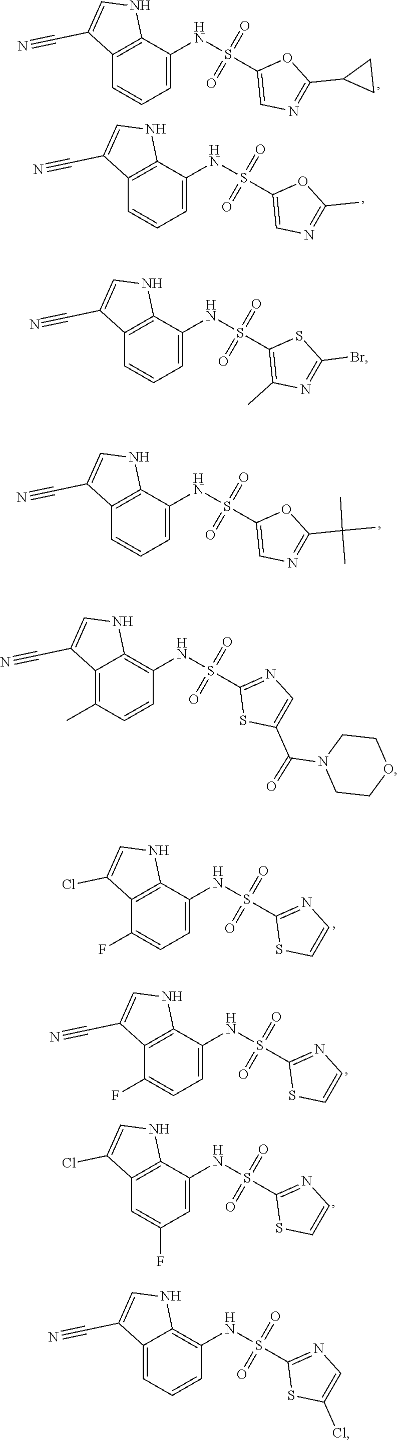

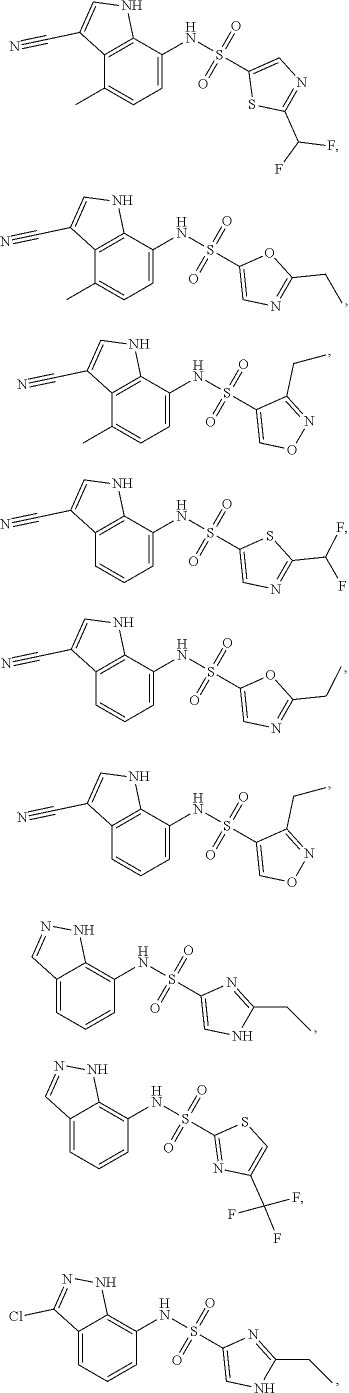

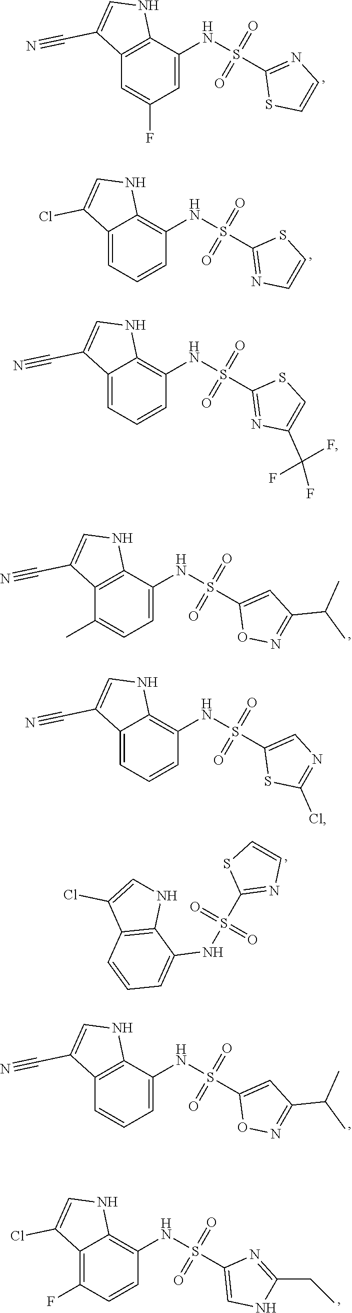

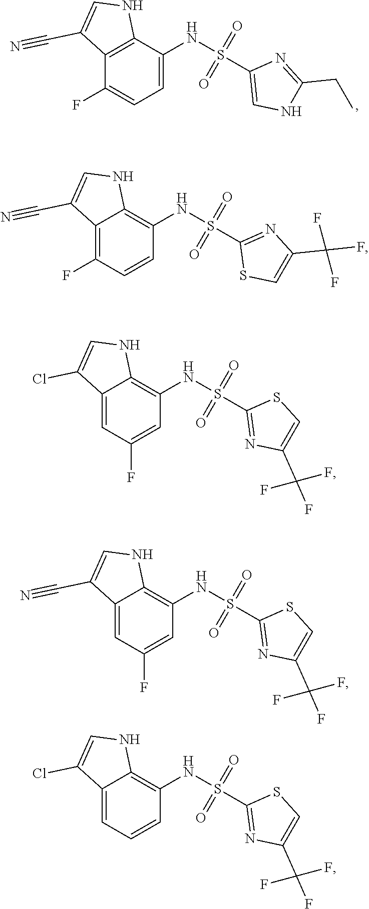 [SMILES: CCc1nc(S(=O)(=O)Nc2ccc(F)c3c(C#N)c[nH]c23)c[nH]1 ; N#Cc1c[nH]c2c(NS(=O)(=O)c3nc(C(F)(F)F)cs3)ccc(F)c12 ; O=S(=O)(Nc1cc(F)cc2c(Cl)c[nH]c12)c1nc(C(F)(F)F)cs1 ; N#Cc1c[nH]c2c(NS(=O)(=O)c3nc(C(F)(F)F)cs3)cc(F)cc12 ; O=S(=O)(Nc1cccc2c(Cl)c[nH]c12)c1nc(C(F)(F)F)cs1]

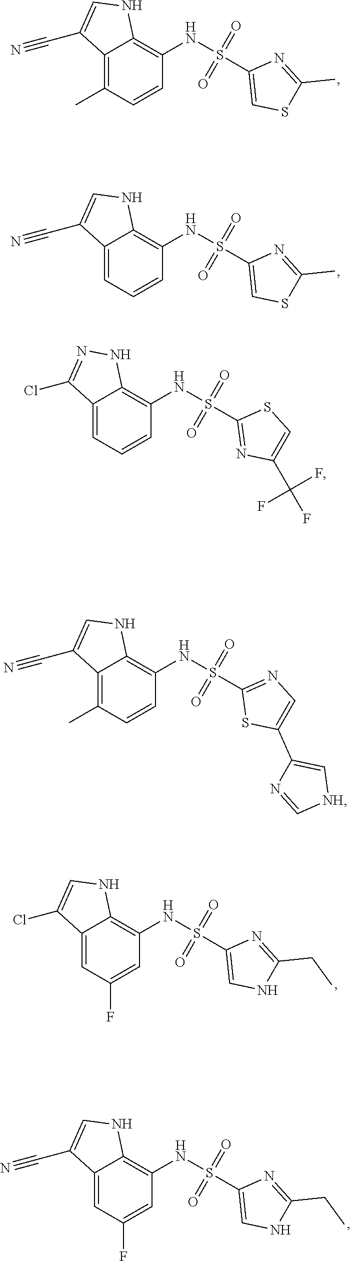 [SMILES: Cc1nc(S(=O)(=O)Nc2ccc(C)c3c(C#N)c[nH]c23)cs1 ; Cc1nc(S(=O)(=O)Nc2cccc3c(C#N)c[nH]c23)cs1 ; O=S(=O)(Nc1cccc2c(Cl)n[nH]c12)c1nc(C(F)(F)F)cs1 ; Cc1ccc(NS(=O)(=O)c2ncc(-c3c[nH]cn3)s2)c2[nH]cc(C#N)c12 ; CCc1nc(S(=O)(=O)Nc2cc(F)cc3c(Cl)c[nH]c23)c[nH]1 ; CCc1nc(S(=O)(=O)Nc2cc(F)cc3c(C#N)c[nH]c23)c[nH]1]

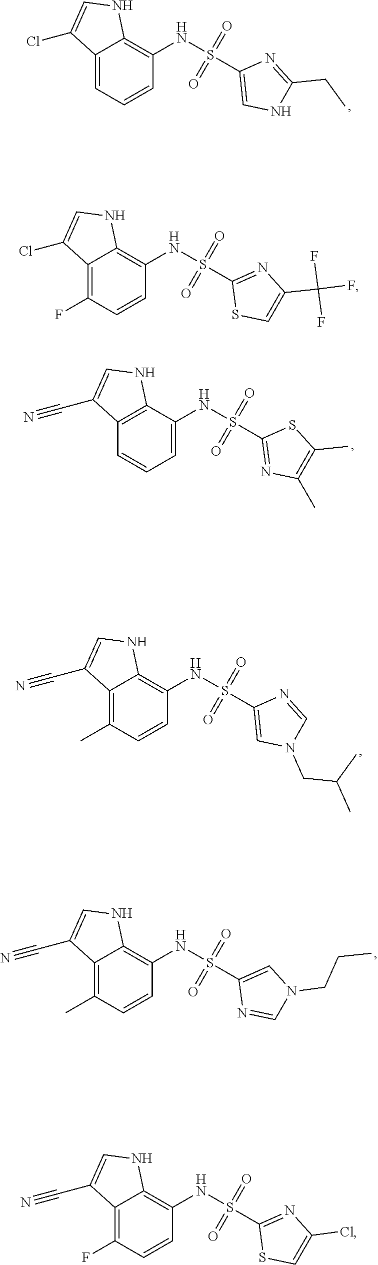 [SMILES: CCc1nc(S(=O)(=O)Nc2cccc3c(Cl)c[nH]c23)c[nH]1 ; O=S(=O)(Nc1ccc(F)c2c(Cl)c[nH]c12)c1nc(C(F)(F)F)cs1 ; Cc1nc(S(=O)(=O)Nc2cccc3c(C#N)c[nH]c23)sc1C ; Cc1ccc(NS(=O)(=O)c2cn(CC(C)C)cn2)c2[nH]cc(C#N)c12 ; CCCn1cnc(S(=O)(=O)Nc2ccc(C)c3c(C#N)c[nH]c23)c1 ; N#Cc1c[nH]c2c(NS(=O)(=O)c3nc(Cl)cs3)ccc(F)c12]

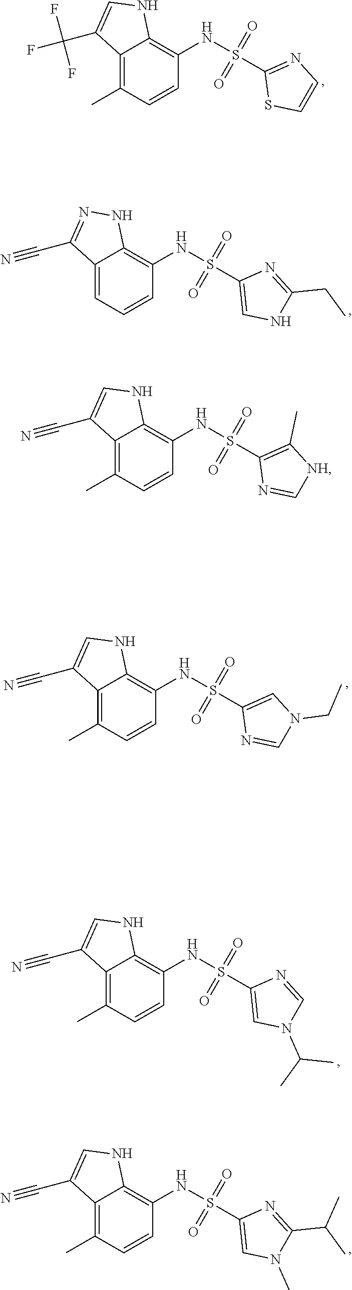 [SMILES: Cc1ccc(NS(=O)(=O)c2nccs2)c2[nH]cc(C(F)(F)F)c12 ; CCc1nc(S(=O)(=O)Nc2cccc3c(C#N)n[nH]c23)c[nH]1 ; Cc1[nH]cnc1S(=O)(=O)Nc1ccc(C)c2c(C#N)c[nH]c12 ; CCn1cnc(S(=O)(=O)Nc2ccc(C)c3c(C#N)c[nH]c23)c1 ; Cc1ccc(NS(=O)(=O)c2cn(C(C)C)cn2)c2[nH]cc(C#N)c12 ; Cc1ccc(NS(=O)(=O)c2cn(C)c(C(C)C)n2)c2[nH]cc(C#N)c12]

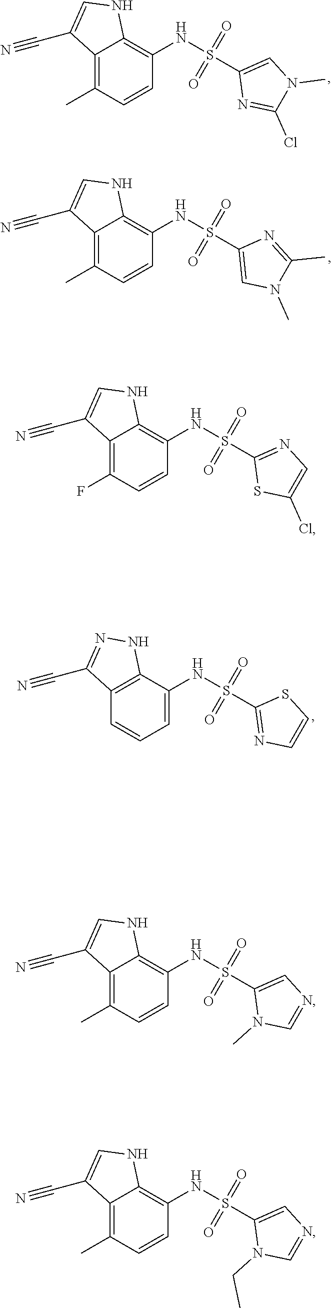 [SMILES: Cc1ccc(NS(=O)(=O)c2cn(C)c(Cl)n2)c2[nH]cc(C#N)c12 ; Cc1ccc(NS(=O)(=O)c2cn(C)c(C)n2)c2[nH]cc(C#N)c12 ; N#Cc1c[nH]c2c(NS(=O)(=O)c3ncc(Cl)s3)ccc(F)c12 ; N#Cc1n[nH]c2c(NS(=O)(=O)c3nccs3)cccc12 ; Cc1ccc(NS(=O)(=O)c2cncn2C)c2[nH]cc(C#N)c12 ; CCn1cncc1S(=O)(=O)Nc1ccc(C)c2c(C#N)c[nH]c12]

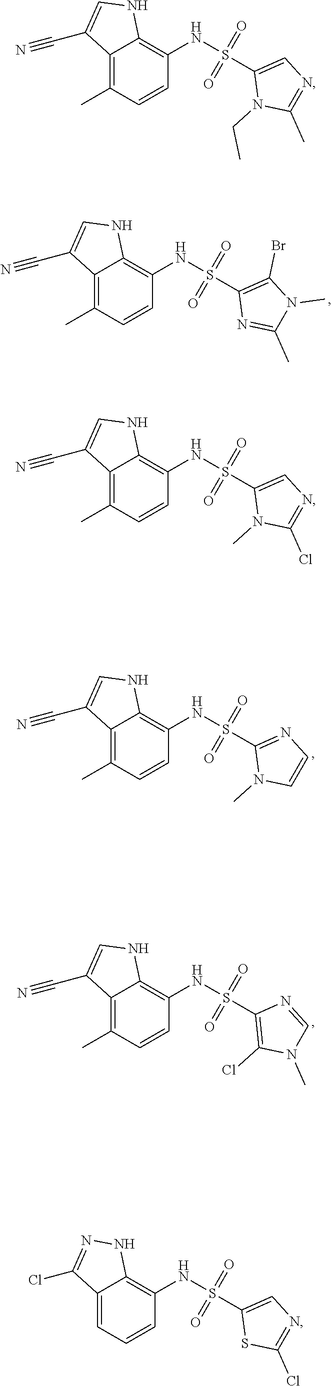 [SMILES: CCn1c(S(=O)(=O)Nc2ccc(C)c3c(C#N)c[nH]c23)cnc1C ; Cc1ccc(NS(=O)(=O)c2nc(C)n(C)c2Br)c2[nH]cc(C#N)c12 ; Cc1ccc(NS(=O)(=O)c2cnc(Cl)n2C)c2[nH]cc(C#N)c12 ; Cc1ccc(NS(=O)(=O)c2nccn2C)c2[nH]cc(C#N)c12 ; Cc1ccc(NS(=O)(=O)c2ncn(C)c2Cl)c2[nH]cc(C#N)c12 ; O=S(=O)(Nc1cccc2c(Cl)n[nH]c12)c1cnc(Cl)s1]

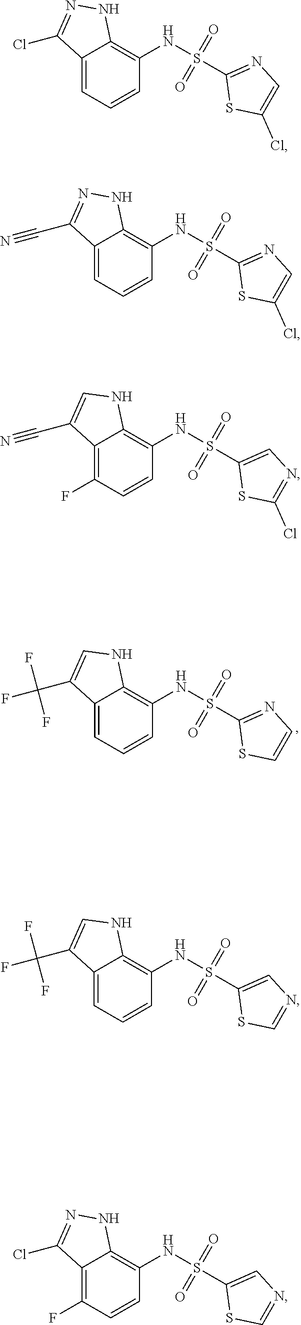 [SMILES: O=S(=O)(Nc1cccc2c(Cl)n[nH]c12)c1ncc(Cl)s1 ; N#Cc1n[nH]c2c(NS(=O)(=O)c3ncc(Cl)s3)cccc12 ; N#Cc1c[nH]c2c(NS(=O)(=O)c3cnc(Cl)s3)ccc(F)c12 ; O=S(=O)(Nc1cccc2c(C(F)(F)F)c[nH]c12)c1nccs1 ; O=S(=O)(Nc1cccc2c(C(F)(F)F)c[nH]c12)c1cncs1 ; O=S(=O)(Nc1ccc(F)c2c(Cl)n[nH]c12)c1cncs1]

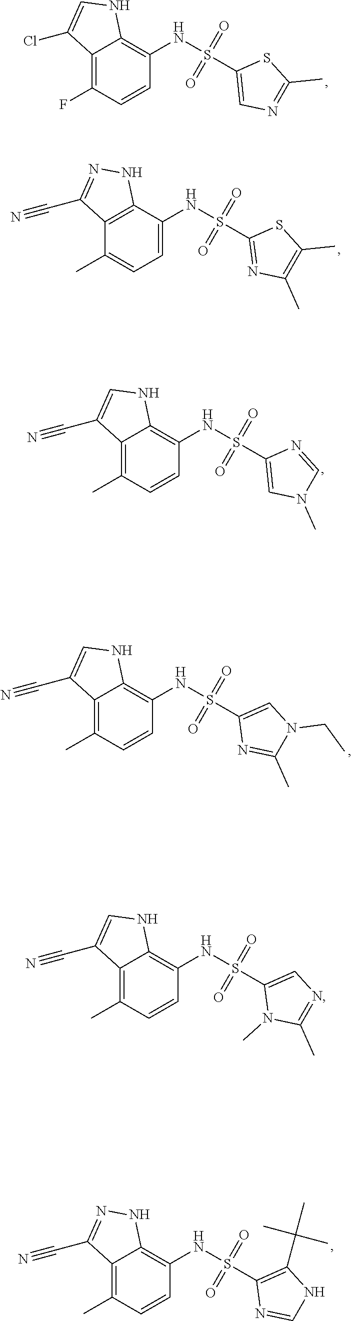 [SMILES: Cc1ncc(S(=O)(=O)Nc2ccc(F)c3c(Cl)c[nH]c23)s1 ; Cc1nc(S(=O)(=O)Nc2ccc(C)c3c(C#N)n[nH]c23)sc1C ; Cc1ccc(NS(=O)(=O)c2cn(C)cn2)c2[nH]cc(C#N)c12 ; CCn1cc(S(=O)(=O)Nc2ccc(C)c3c(C#N)c[nH]c23)nc1C ; Cc1ccc(NS(=O)(=O)c2cnc(C)n2C)c2[nH]cc(C#N)c12 ; Cc1ccc(NS(=O)(=O)c2nc[nH]c2C(C)(C)C)c2[nH]nc(C#N)c12]

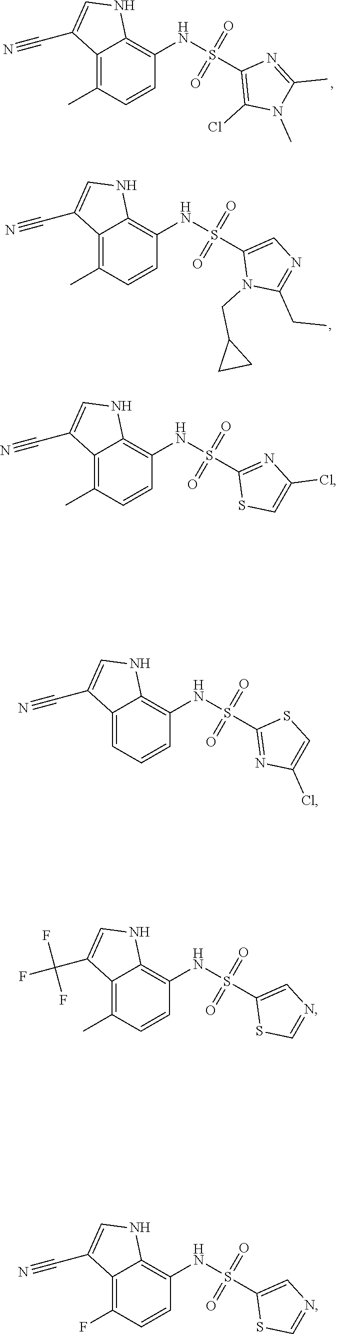 [SMILES: Cc1ccc(NS(=O)(=O)c2nc(C)n(C)c2Cl)c2[nH]cc(C#N)c12 ; CCc1ncc(S(=O)(=O)Nc2ccc(C)c3c(C#N)c[nH]c23)n1CC1CC1 ; Cc1ccc(NS(=O)(=O)c2nc(Cl)cs2)c2[nH]cc(C#N)c12 ; N#Cc1c[nH]c2c(NS(=O)(=O)c3nc(Cl)cs3)cccc12 ; Cc1ccc(NS(=O)(=O)c2cncs2)c2[nH]cc(C(F)(F)F)c12 ; N#Cc1c[nH]c2c(NS(=O)(=O)c3cncs3)ccc(F)c12]

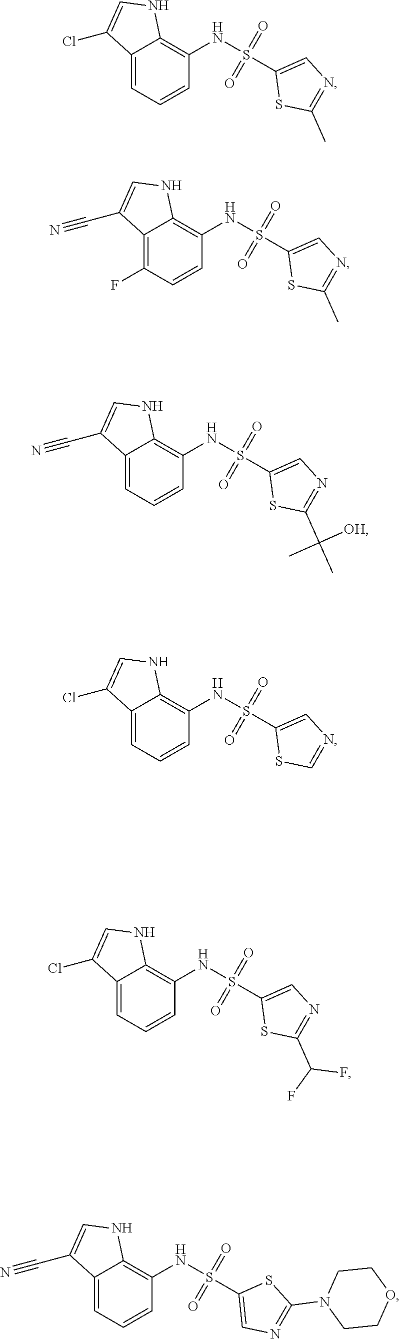 [SMILES: Cc1ncc(S(=O)(=O)Nc2cccc3c(Cl)c[nH]c23)s1 ; Cc1ncc(S(=O)(=O)Nc2ccc(F)c3c(C#N)c[nH]c23)s1 ; CC(C)(O)c1ncc(S(=O)(=O)Nc2cccc3c(C#N)c[nH]c23)s1 ; O=S(=O)(Nc1cccc2c(Cl)c[nH]c12)c1cncs1 ; O=S(=O)(Nc1cccc2c(Cl)c[nH]c12)c1cnc(C(F)F)s1 ; N#Cc1c[nH]c2c(NS(=O)(=O)c3cnc(N4CCOCC4)s3)cccc12]

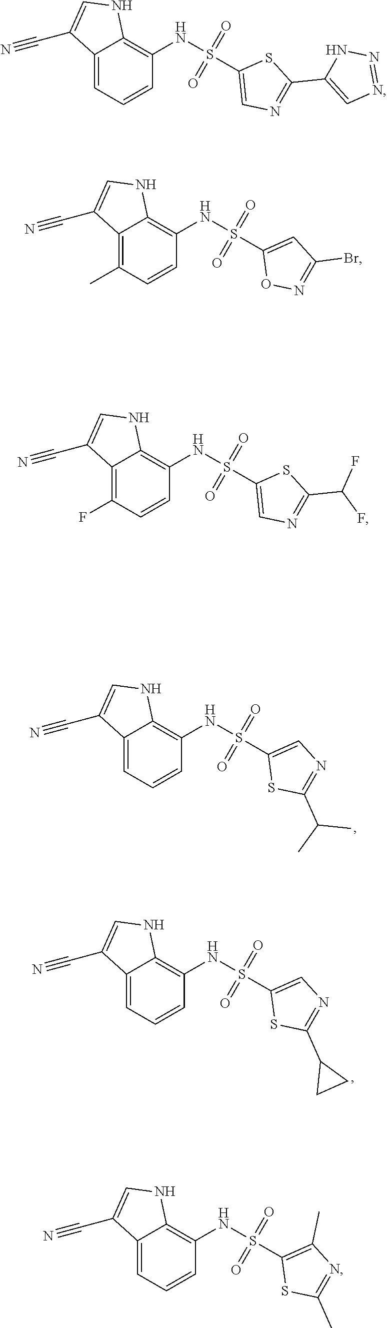 [SMILES: N#Cc1c[nH]c2c(NS(=O)(=O)c3cnc(-c4cnn[nH]4)s3)cccc12 ; Cc1ccc(NS(=O)(=O)c2cc(Br)no2)c2[nH]cc(C#N)c12 ; N#Cc1c[nH]c2c(NS(=O)(=O)c3cnc(C(F)F)s3)ccc(F)c12 ; CC(C)c1ncc(S(=O)(=O)Nc2cccc3c(C#N)c[nH]c23)s1 ; N#Cc1c[nH]c2c(NS(=O)(=O)c3cnc(C4CC4)s3)cccc12 ; Cc1nc(C)c(S(=O)(=O)Nc2cccc3c(C#N)c[nH]c23)s1]

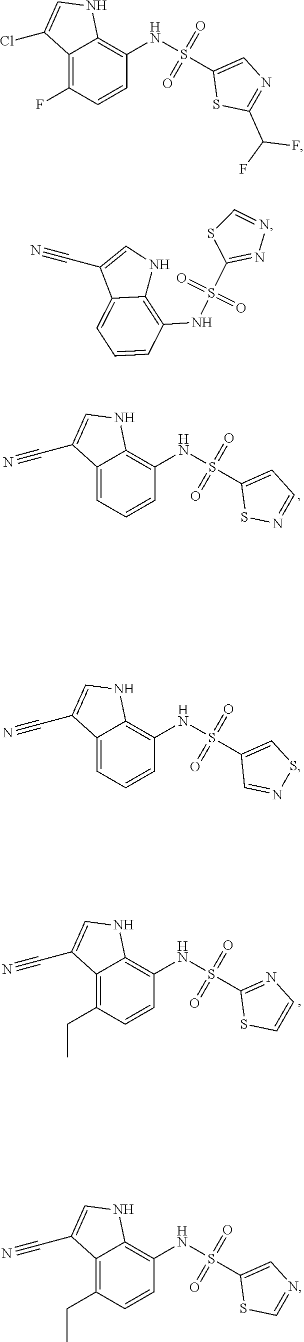

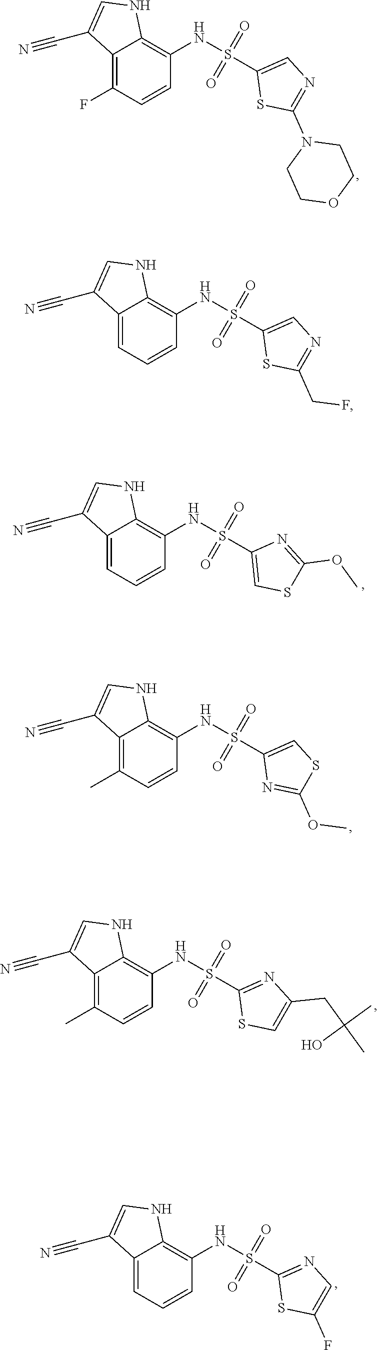 [SMILES: N#Cc1c[nH]c2c(NS(=O)(=O)c3cnc(N4CCOCC4)s3)ccc(F)c12 ; N#Cc1c[nH]c2c(NS(=O)(=O)c3cnc(CF)s3)cccc12 ; COc1nc(S(=O)(=O)Nc2cccc3c(C#N)c[nH]c23)cs1 ; COc1nc(S(=O)(=O)Nc2ccc(C)c3c(C#N)c[nH]c23)cs1 ; Cc1ccc(NS(=O)(=O)c2nc(CC(C)(C)O)cs2)c2[nH]cc(C#N)c12 ; N#Cc1c[nH]c2c(NS(=O)(=O)c3ncc(F)s3)cccc12]

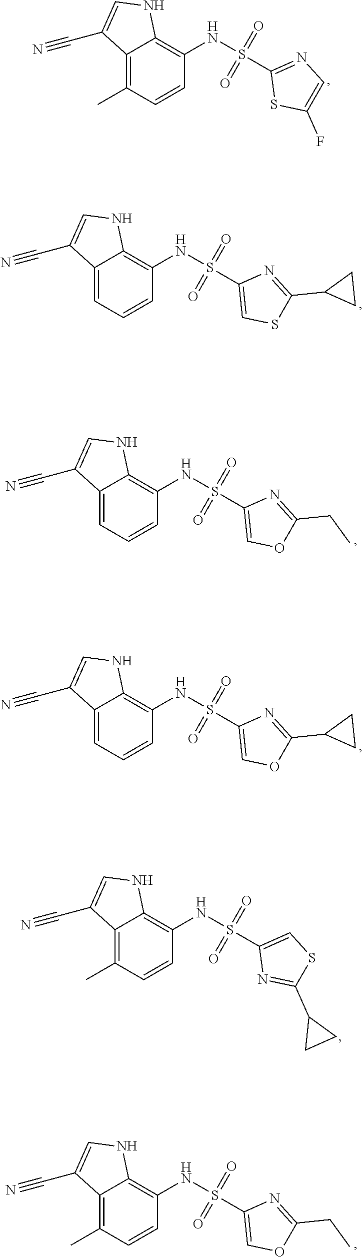 [SMILES: Cc1ccc(NS(=O)(=O)c2ncc(F)s2)c2[nH]cc(C#N)c12 ; N#Cc1c[nH]c2c(NS(=O)(=O)c3csc(C4CC4)n3)cccc12 ; CCc1nc(S(=O)(=O)Nc2cccc3c(C#N)c[nH]c23)co1 ; N#Cc1c[nH]c2c(NS(=O)(=O)c3coc(C4CC4)n3)cccc12 ; Cc1ccc(NS(=O)(=O)c2csc(C3CC3)n2)c2[nH]cc(C#N)c12 ; CCc1nc(S(=O)(=O)Nc2ccc(C)c3c(C#N)c[nH]c23)co1]

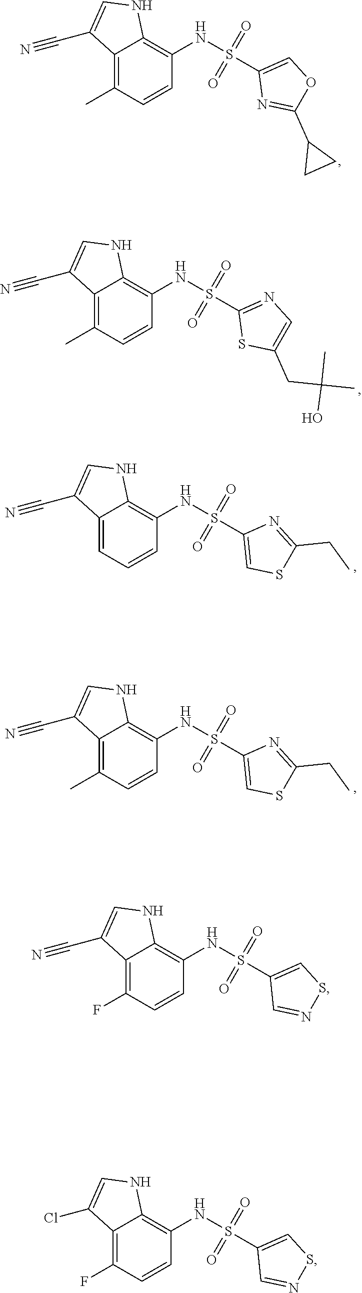 [SMILES: Cc1ccc(NS(=O)(=O)c2coc(C3CC3)n2)c2[nH]cc(C#N)c12 ; Cc1ccc(NS(=O)(=O)c2ncc(CC(C)(C)O)s2)c2[nH]cc(C#N)c12 ; CCc1nc(S(=O)(=O)Nc2cccc3c(C#N)c[nH]c23)cs1 ; CCc1nc(S(=O)(=O)Nc2ccc(C)c3c(C#N)c[nH]c23)cs1 ; N#Cc1c[nH]c2c(NS(=O)(=O)c3cnsc3)ccc(F)c12 ; O=S(=O)(Nc1ccc(F)c2c(Cl)c[nH]c12)c1cnsc1]

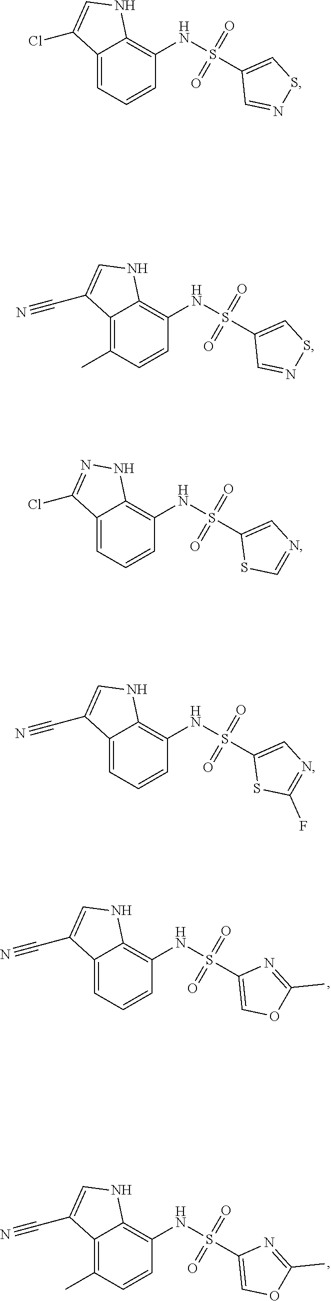 [SMILES: O=S(=O)(Nc1cccc2c(Cl)c[nH]c12)c1cnsc1 ; Cc1ccc(NS(=O)(=O)c2cnsc2)c2[nH]cc(C#N)c12 ; O=S(=O)(Nc1cccc2c(Cl)n[nH]c12)c1cncs1 ; N#Cc1c[nH]c2c(NS(=O)(=O)c3cnc(F)s3)cccc12 ; Cc1nc(S(=O)(=O)Nc2cccc3c(C#N)c[nH]c23)co1 ; Cc1nc(S(=O)(=O)Nc2ccc(C)c3c(C#N)c[nH]c23)co1]

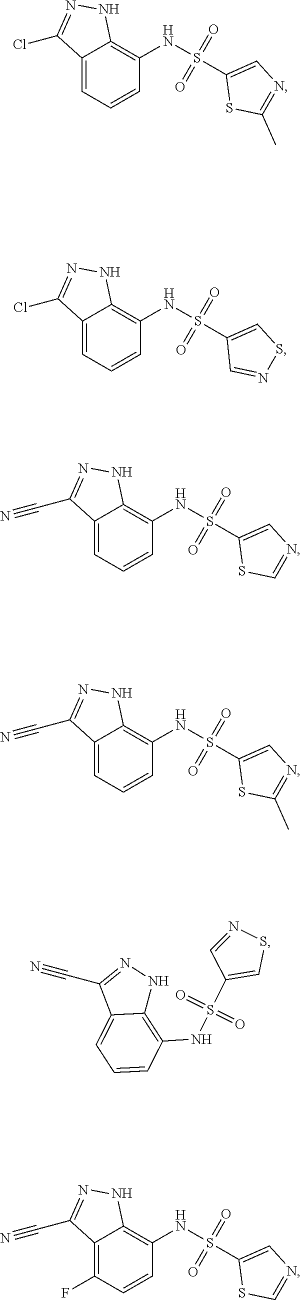 [SMILES: Cc1ncc(S(=O)(=O)Nc2cccc3c(Cl)n[nH]c23)s1 ; O=S(=O)(Nc1cccc2c(Cl)n[nH]c12)c1cnsc1 ; N#Cc1n[nH]c2c(NS(=O)(=O)c3cncs3)cccc12 ; Cc1ncc(S(=O)(=O)Nc2cccc3c(C#N)n[nH]c23)s1 ; N#Cc1n[nH]c2c(NS(=O)(=O)c3cnsc3)cccc12 ; N#Cc1n[nH]c2c(NS(=O)(=O)c3cncs3)ccc(F)c12]

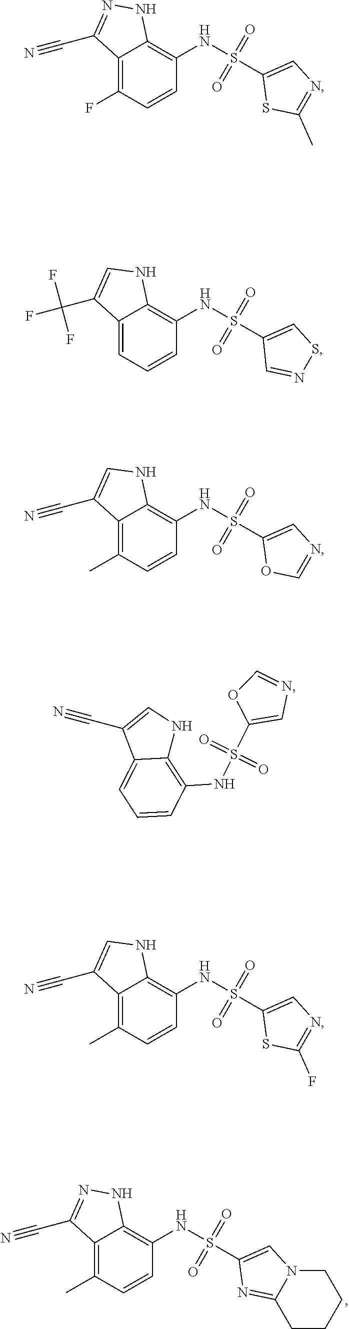 [SMILES: Cc1ncc(S(=O)(=O)Nc2ccc(F)c3c(C#N)n[nH]c23)s1 ; O=S(=O)(Nc1cccc2c(C(F)(F)F)c[nH]c12)c1cnsc1 ; Cc1ccc(NS(=O)(=O)c2cnco2)c2[nH]cc(C#N)c12 ; N#Cc1c[nH]c2c(NS(=O)(=O)c3cnco3)cccc12 ; Cc1ccc(NS(=O)(=O)c2cnc(F)s2)c2[nH]cc(C#N)c12 ; Cc1ccc(NS(=O)(=O)c2cn3c(n2)CCCC3)c2[nH]nc(C#N)c12]

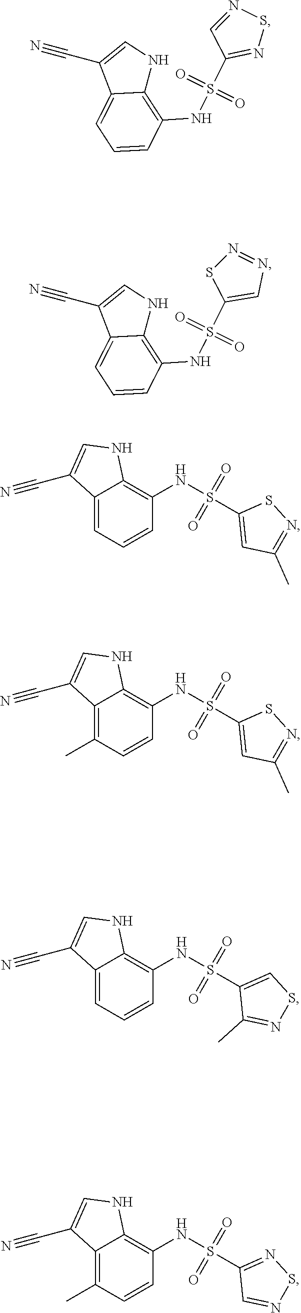 [SMILES: N#Cc1c[nH]c2c(NS(=O)(=O)c3cnsn3)cccc12 ; N#Cc1c[nH]c2c(NS(=O)(=O)c3cnns3)cccc12 ; Cc1cc(S(=O)(=O)Nc2cccc3c(C#N)c[nH]c23)sn1 ; Cc1cc(S(=O)(=O)Nc2ccc(C)c3c(C#N)c[nH]c23)sn1 ; Cc1nscc1S(=O)(=O)Nc1cccc2c(C#N)c[nH]c12 ; Cc1ccc(NS(=O)(=O)c2cnsn2)c2[nH]cc(C#N)c12]

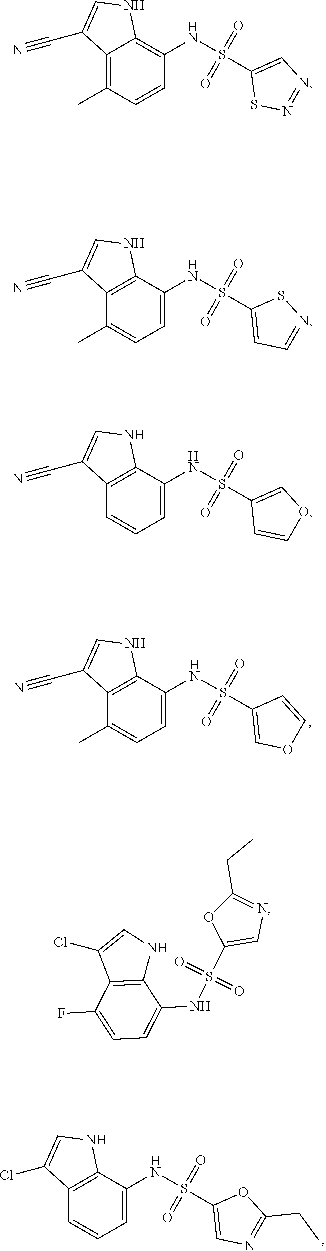 [SMILES: Cc1ccc(NS(=O)(=O)c2cnns2)c2[nH]cc(C#N)c12 ; Cc1ccc(NS(=O)(=O)c2ccns2)c2[nH]cc(C#N)c12 ; N#Cc1c[nH]c2c(NS(=O)(=O)c3ccoc3)cccc12 ; Cc1ccc(NS(=O)(=O)c2ccoc2)c2[nH]cc(C#N)c12 ; CCc1ncc(S(=O)(=O)Nc2ccc(F)c3c(Cl)c[nH]c23)o1 ; CCc1ncc(S(=O)(=O)Nc2cccc3c(Cl)c[nH]c23)o1]

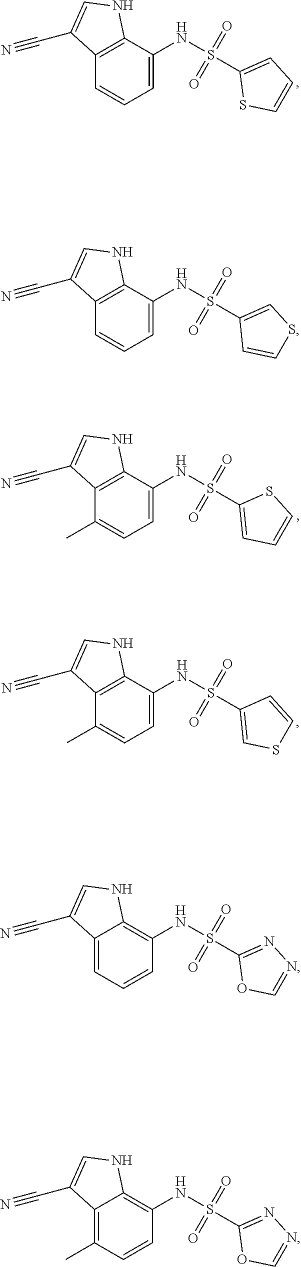 [SMILES: N#Cc1c[nH]c2c(NS(=O)(=O)c3cccs3)cccc12 ; N#Cc1c[nH]c2c(NS(=O)(=O)c3ccsc3)cccc12 ; Cc1ccc(NS(=O)(=O)c2cccs2)c2[nH]cc(C#N)c12 ; Cc1ccc(NS(=O)(=O)c2ccsc2)c2[nH]cc(C#N)c12 ; N#Cc1c[nH]c2c(NS(=O)(=O)c3nnco3)cccc12 ; Cc1ccc(NS(=O)(=O)c2nnco2)c2[nH]cc(C#N)c12]

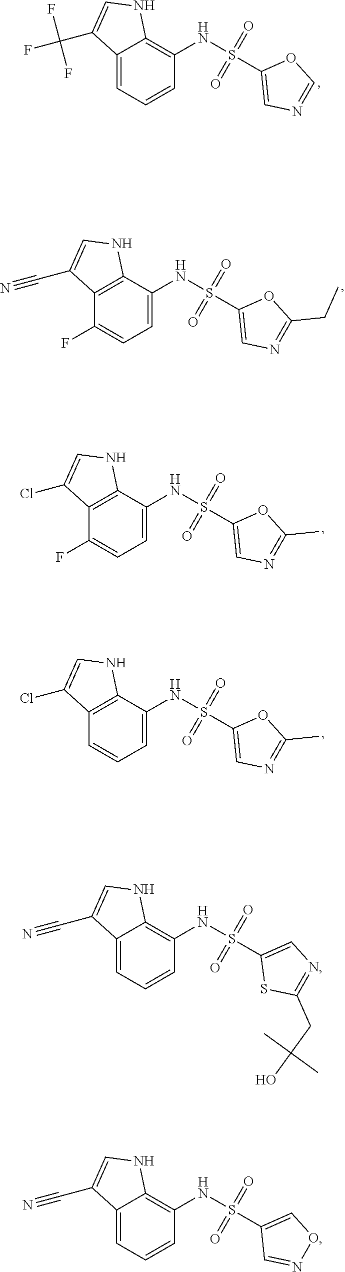 [SMILES: O=S(=O)(Nc1cccc2c(C(F)(F)F)c[nH]c12)c1cnco1 ; CCc1ncc(S(=O)(=O)Nc2ccc(F)c3c(C#N)c[nH]c23)o1 ; Cc1ncc(S(=O)(=O)Nc2ccc(F)c3c(Cl)c[nH]c23)o1 ; Cc1ncc(S(=O)(=O)Nc2cccc3c(Cl)c[nH]c23)o1 ; CC(C)(O)Cc1ncc(S(=O)(=O)Nc2cccc3c(C#N)c[nH]c23)s1 ; N#Cc1c[nH]c2c(NS(=O)(=O)c3cnoc3)cccc12]

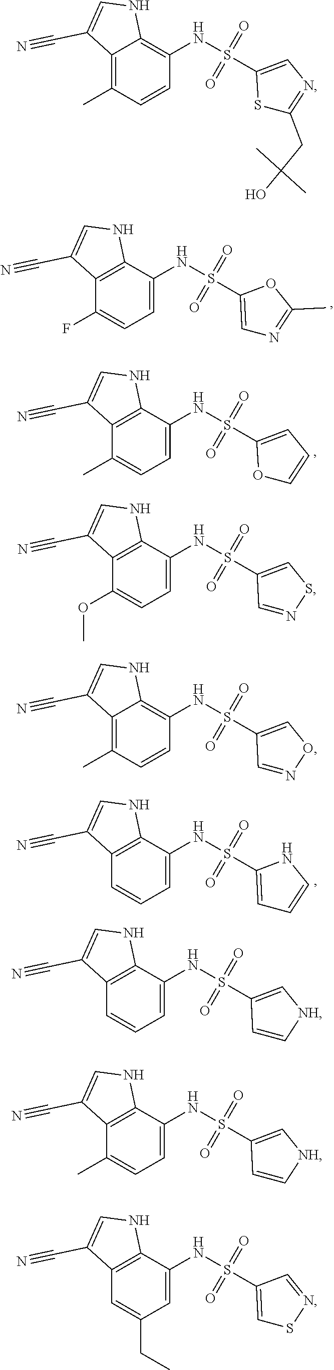

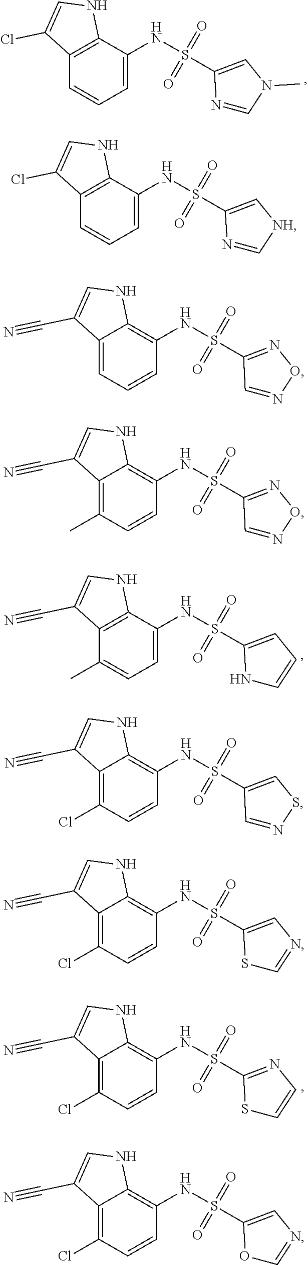

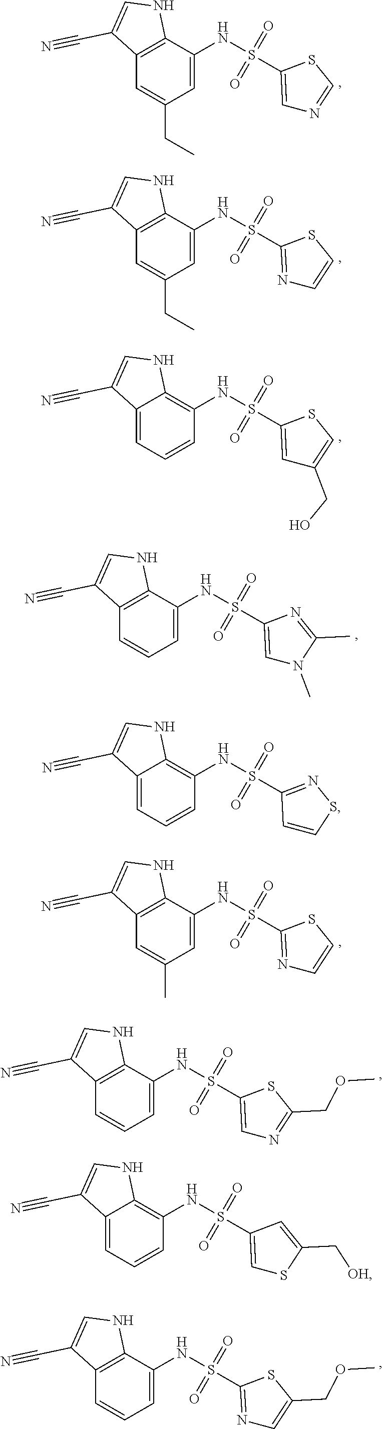

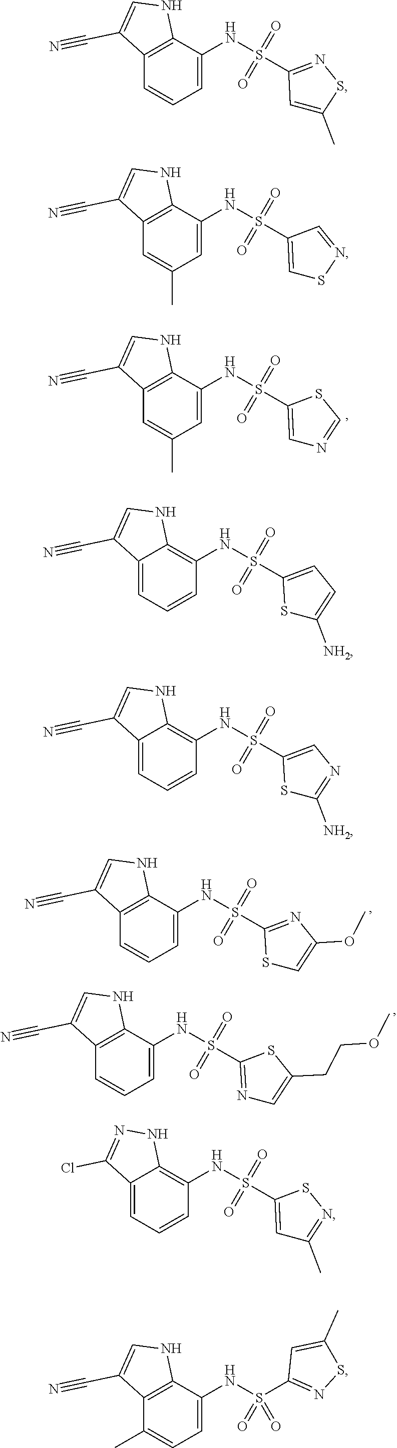

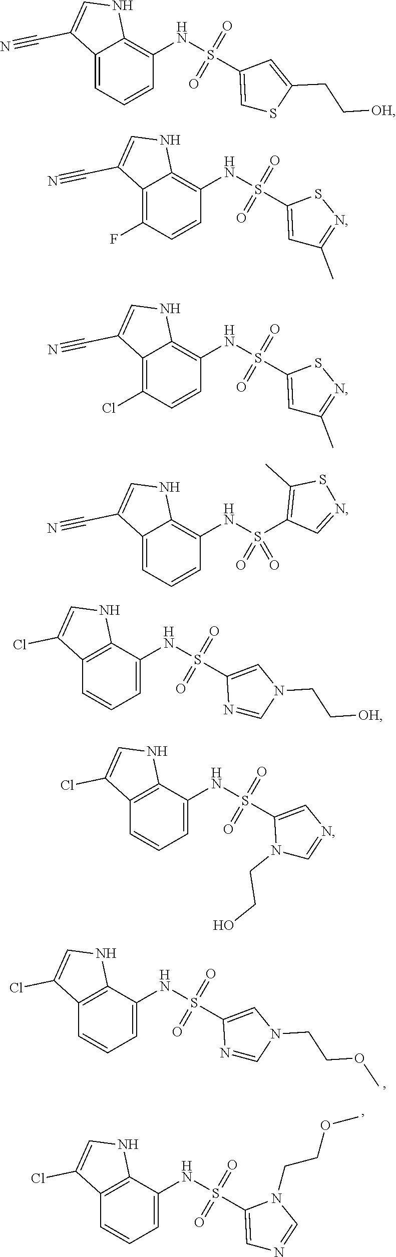

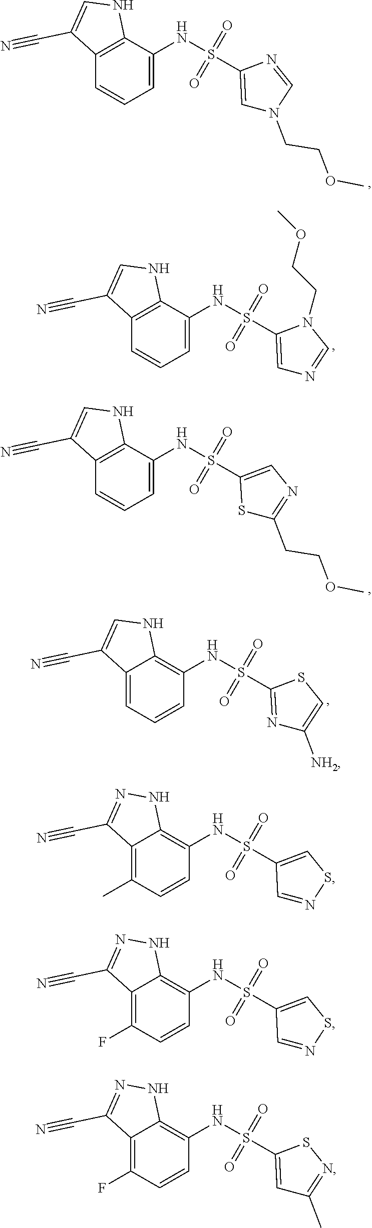

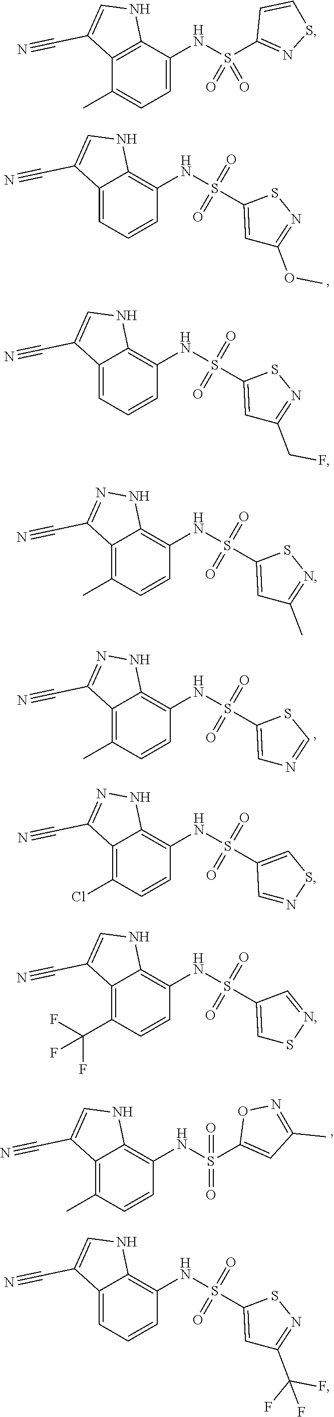

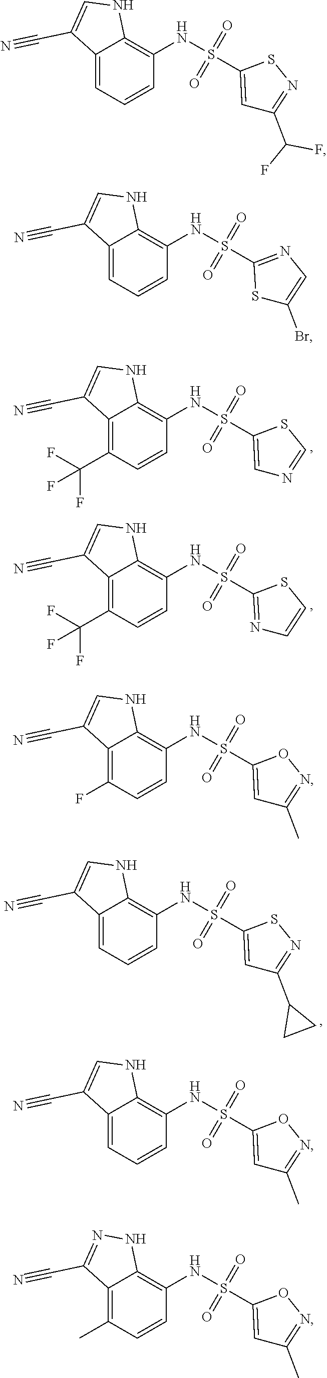

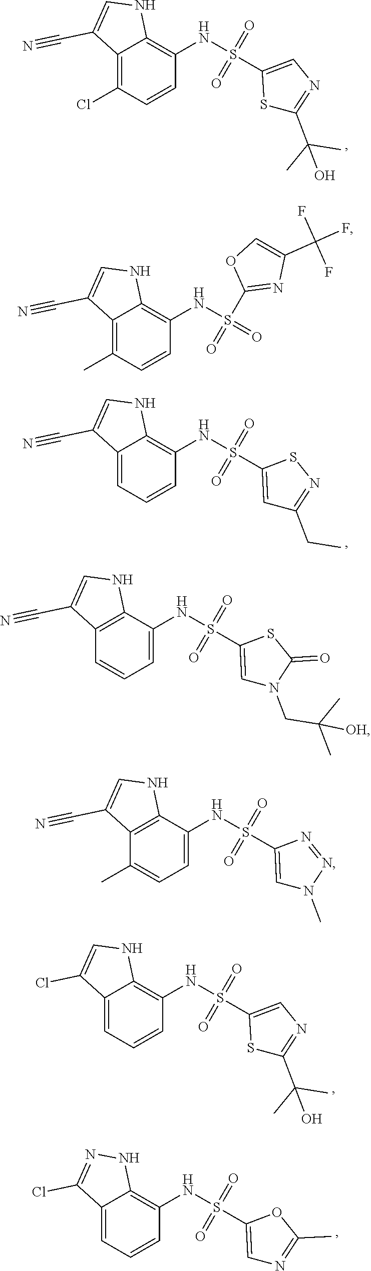

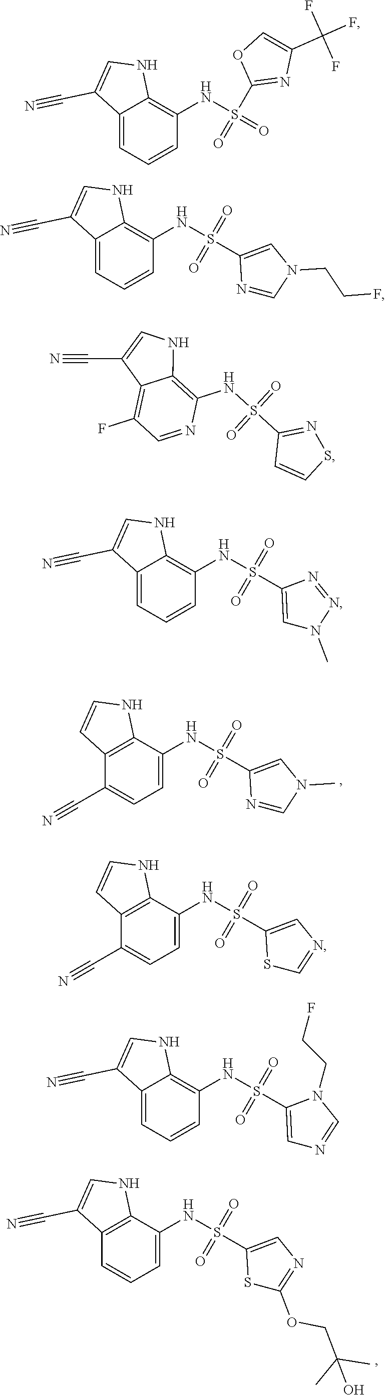

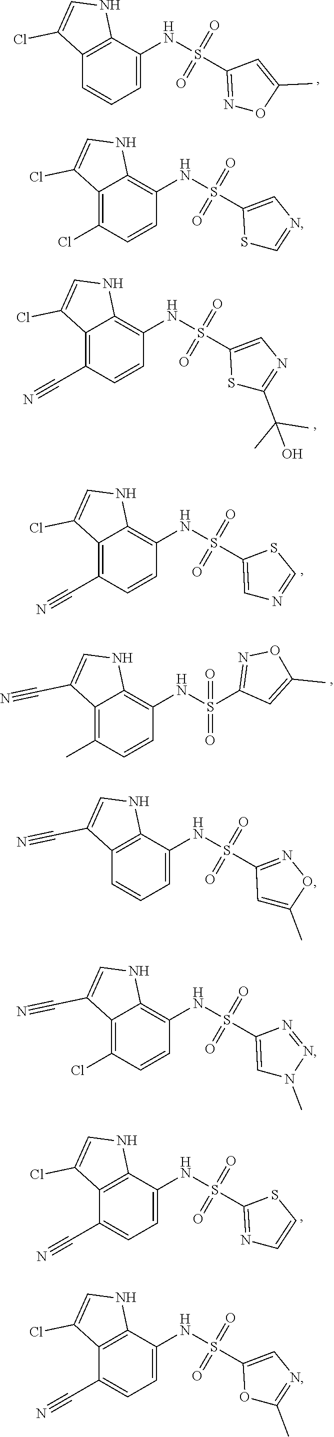

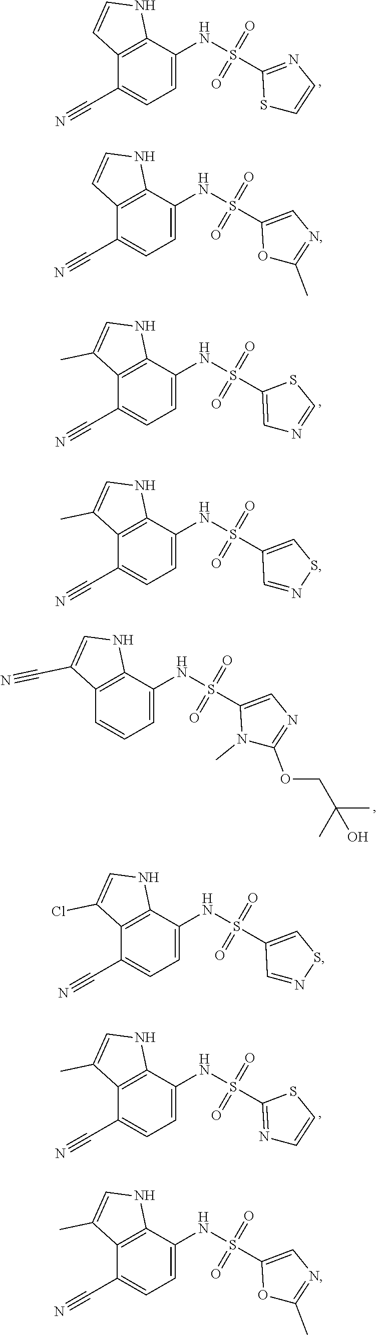

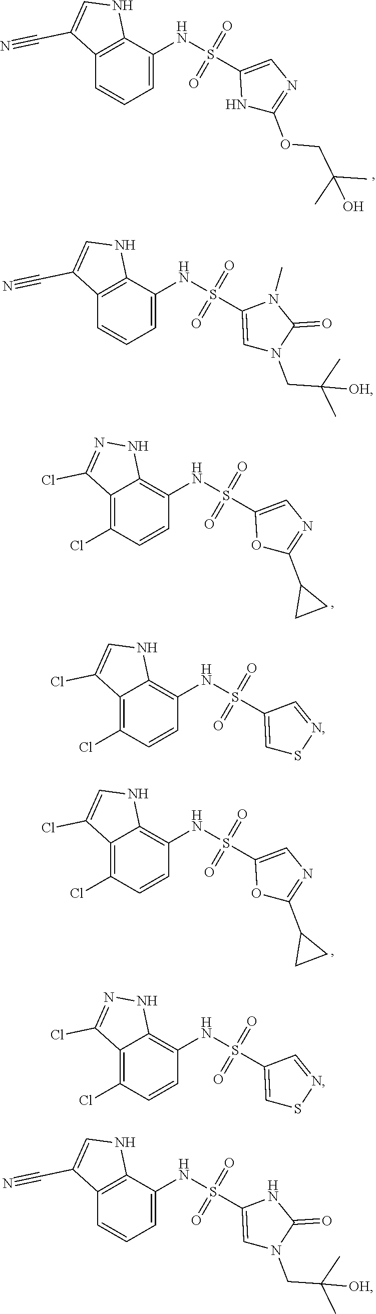

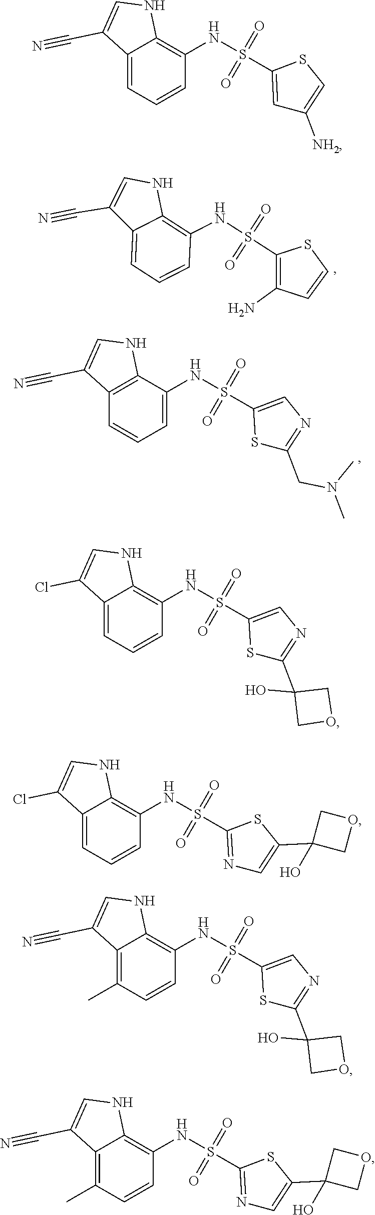

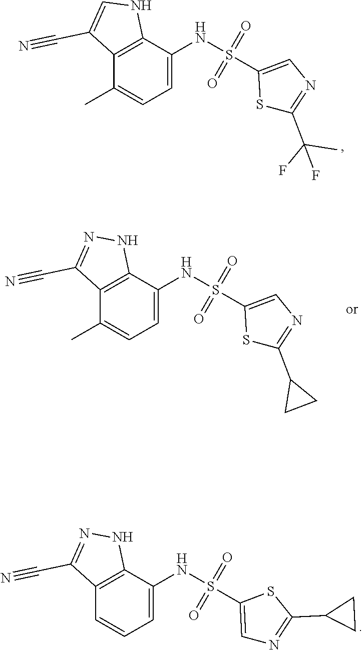 [SMILES: Cc1ccc(NS(=O)(=O)c2cnc(C(C)(F)F)s2)c2[nH]cc(C#N)c12 ; Cc1ccc(NS(=O)(=O)c2cnc(C3CC3)s2)c2[nH]nc(C#N)c12 ; N#Cc1n[nH]c2c(NS(=O)(=O)c3cnc(C4CC4)s3)cccc12]

Where the compounds disclosed herein have at least one chiral center, they may exist as individual enantiomers and diasteromers or a mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise. (including pharmaceutically acceptable salts of any of the foregoing).

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

“Solvate” refers to the compound formed by the interaction of a solvent and a compound described herein or salt thereof. Suitable solvates are pharmaceutically acceptable solvates including hydrates.

The term “pharmaceutically acceptable salt” refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable salts can also be formed using inorganic and organic bases. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts. In some embodiments, treatment of the compounds disclosed herein with an inorganic base results in loss of a labile hydrogen from the compound to afford the salt form including an inorganic cation such as Li⁺, Na⁺, K⁺, Mg²⁺ and Ca²⁺ and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Ca to CC” or “Ca-b” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)—, (CH₃)₂CHCH₂—, and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkylthio” refers to the formula —SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atom, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

As used herein, “alkylene” means a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” means a straight or branched chain di-radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-carbamyl” group refers to an “—N(R_{A})C(═O)ORB” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-thiocarbamyl” group refers to an “—N(R_{A})C(═S)ORB” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₁₋₁₀ aryl, 5-10 membered heteroaryl, and 3-10 membered heterocyclyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Unless otherwise indicated, wherever a group is described as “optionally substituted” that group can be substituted with the above substituents.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

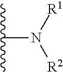

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

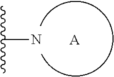

where ring A is a heterocyclyl ring containing the depicted nitrogen.

Similarly, when two “adjacent” R groups are said to form a ring “together with the atoms to which they are attached,” it is meant that the collective unit of the atoms, intervening bonds, and the two R groups are the recited ring. For example, when the following substructure is present:

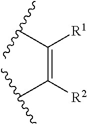

and R¹ and R² are defined as selected from the group consisting of hydrogen and alkyl, or R¹ and R² together with the atoms to which they are attached form an aryl or carbocylyl, it is meant that R¹ and R² can be selected from hydrogen or alkyl, or alternatively, the substructure has structure:

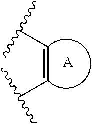

where A is an aryl ring or a carbocylyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

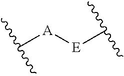

includes the substituent being oriented such that the A is attached at the leftmost attachment point of the molecule as well as the case in which A is attached at the rightmost attachment point of the molecule.

As used herein, “isosteres” of a chemical group are other chemical groups that exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated include —SO₃H, —SO₂HNR, —PO₂(R)₂, —PO₃(R)₂, —CONHNHSO₂R, —COHNSO₂R, and —CONRCN, where R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of carbocyclic and heterocyclic isosteres contemplated. The atoms of said ring structure may be optionally substituted at one or more positions with R as defined above.

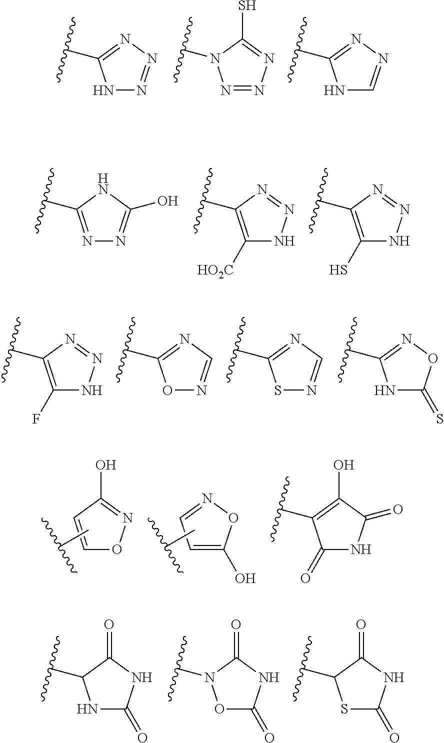

It is also contemplated that when chemical substituents are added to a carboxylic isostere, the compound retains the properties of a carboxylic isostere. It is contemplated that when a carboxylic isostere is optionally substituted with one or more moieties selected from R as defined above, then the substitution and substitution position is selected such that it does not eliminate the carboxylic acid isosteric properties of the compound. Similarly, it is also contemplated that the placement of one or more R substituents upon a carbocyclic or heterocyclic carboxylic acid isostere is not a substitution at one or more atom(s) that maintain(s) or is/are integral to the carboxylic acid isosteric properties of the compound, if such substituent(s) would destroy the carboxylic acid isosteric properties of the compound.

Other carboxylic acid isosteres not specifically exemplified in this specification are also contemplated.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

### Methods of Preparation

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

Scheme A provides a general synthetic scheme for the synthesis of compounds of Formula 1.

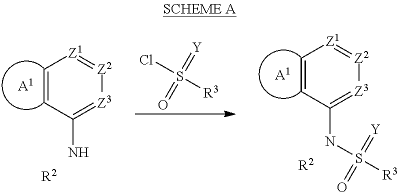

To a solution of substituted heteroaromatic sulfonyl chloride (1.00 eq) in dichloromethane (0.500 mL) is added Pyridine (2.00 eq) and 7-amino-4-substituted-1H-indole-3-carbonitrile or other substitution (1.00 eq). The mixture is stirred at 20° C. for 1 h. The reaction mixture is concentrated under reduced pressure to remove solvent. The crude product is purified by prep-HPLC (column: Phenomenex luna C18 150*25 mm*10 μm; mobile phase: [water(FA)-ACN]; gradient: 43%-73% B over 10 min) and lyophilized to afford the desired heteroaromatic indole sulfonamide.

### Administration and Pharmaceutical Compositions

The compounds are administered at a therapeutically effective dosage. While human dosage levels have yet to be optimized for the compounds described herein, generally, a daily dose may be from about 0.0125 mg/kg to about 120 mg/kg or more of body weight, from about 0.025 mg/kg or less to about 70 mg/kg, from about 0.05 mg/kg to about 50 mg/kg of body weight, or from about 0.075 mg/kg to about 10 mg/kg of body weight. Thus, for administration to a 70 kg person, the dosage range would be from about 0.88 mg per day to about 8000 mg per day, from about 1.8 mg per day or less to about 7000 mg per day or more, from about 3.6 mg per day to about 6000 mg per day, from about 5.3 mg per day to about 5000 mg per day, or from about 11 mg to about 3000 mg per day. The amount of active compound administered will, of course, be dependent on the subject and disease state being treated, the severity of the affliction, the manner and schedule of administration and the judgment of the prescribing physician.

Administration of the compounds disclosed herein, or the pharmaceutically acceptable salts thereof, can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form, however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan. In some embodiments, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day.

### Methods of Treatment

The compounds disclosed herein and/or pharmaceutically acceptable salts thereof can effectively modulate RNA splicing by RBM39. Some embodiments provide pharmaceutical compositions comprising one or more compounds disclosed herein and a pharmaceutically acceptable excipient.

Some embodiments of the present invention include methods of treating cancer with the compounds and compositions comprising compounds described herein. Some methods include administering a compound, composition, pharmaceutical composition described herein to a subject in need thereof. In some embodiments, a subject can be an animal, e.g., a mammal, a human. Example cancers include, but are not limited to, colorectal cancer (CRC), pleural mesothelioma (PM), cutaneous squamous cell carcinoma (CSCC); tumor mutation burden high (TMB-H), Bacillus Calmette-Gudrin bladder cancer, endometrial carcinoma (EC), esophageal squamous cell carcinoma (ESCC), Merkel cell carcinoma (MCC), hepatocellular carcinoma (HCC), primary mediastinal large B cell lymphoma (PMBCL), cervical cancer, urothelial carcinoma, classical Hodgkin's lymphoma, head and neck squamous cell carcinoma, liver cancer, gastric cancer, prostate cancer, sarcoma, melanoma, non-small cell lung cancer (NSCLC), small cell lung cancer, renal cell carcinoma, triple negative breast cancer, luminal B breast cancer, colon cancer, ovarian cancer, pancreatic cancer and glioblastoma.

In some embodiments, the method of administering one or more of the compounds disclosed herein results in the degradation or reduction of RBM39 protein.

In some embodiments, the method of administering one or more of the compounds disclosed herein results in increased expression of immunogenic neoepitopes.

In some embodiments, the method of administering one or more of the compounds disclosed herein results in increased CD8⁺ T cell expansion. In some embodiments, the method includes administering a pharmaceutically acceptable salt thereof of one or more compounds disclosed herein.

In some embodiments, the subject is a human.

Further embodiments include administering a combination of compounds to a subject in need thereof. A combination can include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered i.v.

Some embodiments further include administering surgery, radiation therapy, chemotherapy, targeted therapy, immunotherapy, hormonal therapy, or antiviral therapy. In some embodiments, the immunotherapy includes administration of an immune checkpoint inhibitor.

### Immune Checkpoint Inhibitors

In some embodiments, one or more immune checkpoint inhibitor may be co-administered with a compound of Formula (I). A review describing immune checkpoint pathways and the blockade of such pathways with immune checkpoint inhibitor compounds is provided by Pardoll in Nature Reviews Cancer (April, 2012), pages 252-264, which is incorporated herein by reference in its entirety. Immune check point inhibitor compounds display anti-tumor activity by blocking one or more of the endogenous immune checkpoint pathways that downregulate an anti-tumor immune response. The inhibition or blockade of an immune checkpoint pathway typically involves inhibiting a checkpoint receptor and ligand interaction with an immune checkpoint inhibitor compound to reduce or eliminate the down regulation signal and resulting diminishment of the anti-tumor response.

In some embodiments of the present disclosure, the immune checkpoint inhibitor compound inhibits the signaling interaction between an immune checkpoint receptor and the corresponding ligand of the immune checkpoint receptor. The immune checkpoint inhibitor compound can act by blocking activation of the immune checkpoint pathway by inhibition (antagonism) of an immune checkpoint receptor (some examples of receptors include CTLA-4, PD-1, LAG-3, TIM-3, BTLA, and KIR) or by inhibition of a ligand of an immune checkpoint receptor (some examples of ligands include PD-L1 and PD-L2). In such embodiments, the effect of the immune checkpoint inhibitor compound is to reduce or eliminate down regulation of certain aspects of the immune system anti-tumor response in the tumor microenvironment.

The Programmed Death 1 (PD-1) protein is an inhibitory member of the extended CD28/CTLA-4 family of T cell regulators (Okazaki et al. (2002) Curr Opin Immunol 14: 391779-82; Bennett et al. (2003) J. Immunol. 170:711-8; which are incorporated herein by reference in their entirety). Other members of the CD28 family include CD28, CTLA-4, ICOS and BTLA. PD-1 is suggested to exist as a monomer, lacking the unpaired cysteine residue characteristic of other CD28 family members. PD-1 is expressed on activated B cells, T cells, and monocytes.

The PD-1 gene encodes a 55 kDa type I transmembrane protein (Agata et al. (1996) Int Immunol. 8:765-72, which is incorporated herein by reference in its entirety). Although structurally similar to CTLA-4, PD-1 lacks the MYPPY motif that is important for B7-1 and B7-2 binding. Two ligands for PD-1 have been identified, PD-L1 (B7-H1) and PD-L2 (B7-DC), that have been shown to downregulate T cell activation upon binding to PD-1 (Freeman et al. (2000) J. Exp. Med. 192:1027-34; Carter et al. (2002) Eur. J. Immunol. 32:634-43; which are incorporated herein by reference in their entirety). Both PD-L1 and PD-L2 are B7 homologs that bind to PD-1, but do not bind to other CD28 family members. PD-L1 is abundant in a variety of human cancers (Dong et al. (2002) Nat. Med. 8:787-9, which is incorporated herein by reference in its entirety).

PD-1 is known as an immunoinhibitory protein that negatively regulates TCR signals (Ishida, Y. et al. (1992) EMBO J. 11:3887-3895; Blank, C. et al. (Epub 2006 Dec. 29) Immunol. Immunother. 56(5):739-745; which are incorporated herein by reference in their entirety). The interaction between PD-1 and PD-L1 can act as an immune checkpoint, which can lead to, e.g., a decrease in tumor infiltrating lymphocytes, a decrease in T-cell receptor mediated proliferation, and/or immune evasion by cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100; which are incorporated herein by reference in their entirety). Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66; which are incorporated herein by reference in their entirety).

The immune checkpoint receptor cytotoxic T-lymphocyte associated antigen 4 (CTLA-4) is expressed on T-cells and is involved in signaling pathways that reduce the level of T-cell activation. It is believed that CTLA-4 can downregulate T-cell activation through competitive binding and sequestration of CD80 and CD86. In addition, CTLA-4 has been shown to be involved in enhancing the immunosuppressive activity of T_{Reg} cells.

The immune checkpoint receptor programmed death 1 (PD-1) is expressed by activated T-cells upon extended exposure to antigen. Engagement of PD-1 with its known binding ligands, PD-L1 and PD-L2, occurs primarily within the tumor microenvironment and results in downregulation of anti-tumor specific T-cell responses. Both PD-L1 and PD-L2 are known to be expressed on tumor cells. The expression of PD-L1 and PD-L2 on tumors has been correlated with decreased survival outcomes.

The immune checkpoint receptor T cell membrane protein 3 (TIM-3) is expressed on Th1 and Tc1 cells, but not other T-cells. Interaction of TIM-3 with its ligand, galectin-9, produces a Th1 cell death signal. TIM-3 has been reported to play a role in maintaining T-cell exhaustion and blockade of TIM-3 has been shown to restore activity to exhausted T-cells.

The immune checkpoint receptor B- and T-lymphocyte attenuator (BTLA) receptor is expressed on both resting and activated B-cells and T-cells. Activation of BTLA when combined with its ligand HVEM (herpes virus entry mediator) results in downregulation of both T-cell activation and proliferation. HVEM is expressed by certain tumors (e.g., melanoma) and tumor-associated endothelial cells.

The immune checkpoint receptors known as killer cell immunoglobulin-like receptors (KIR) are a polymorphic family of receptors expressed on NK cells and some T-cells and function as regulators of immune tolerance associated with natural killer (NK) cells. Blocking certain KIR receptors with inhibitor compounds can facilitate the destruction of tumors through the increased activity of NK cells.

In some embodiments of the present disclosure, the immune checkpoint inhibitor compound is a small organic molecule (molecular weight less than 1000 daltons), a peptide, a polypeptide, a protein, an antibody, an antibody fragment, or an antibody derivative. In some embodiments, the immune checkpoint inhibitor compound is an antibody. In some embodiments, the antibody is a monoclonal antibody, specifically a human or a humanized monoclonal antibody.

Monoclonal antibodies, antibody fragments, and antibody derivatives for blocking immune checkpoint pathways can be prepared by any of several methods known to those of ordinary skill in the art, including but not limited to, somatic cell hybridization techniques and hybridoma, methods. Hybridoma generation is described in Antibodies, A Laboratory Manual, Harlow and Lane, 1988, Cold Spring Harbor Publications, New York, which is incorporated herein by reference in its entirety. Human monoclonal antibodies can be identified and isolated by screening phage display libraries of human immunoglobulin genes by methods described for example in U.S. Pat. Nos. 5,223,409, 5,403,484, 5,571,698, 6,582,915, and 6,593,081, which are incorporated herein by reference in their entirety. Monoclonal antibodies can be prepared using the general methods described in U.S. Pat. No. 6,331,415 (Cabilly), which is incorporated herein by reference in its entirety.

As an example, human monoclonal antibodies can be prepared using a XenoMouse™ (Abgenix, Freemont, Calif.) or hybridomas of B cells from a XenoMouse. A XenoMouse is a murine host having functional human immunoglobulin genes as described in U.S. Pat. No. 6,162,963 (Kucherlapati), which is incorporated herein by reference in its entirety.

Methods for the preparation and use of immune checkpoint antibodies are described in the following illustrative publications. The preparation and therapeutic uses of anti-CTLA-4 antibodies are described in U.S. Pat. No. 7,229,628 (Allison), U.S. Pat. No. 7,311,910 (Linsley), and U.S. Pat. No. 8,017,144 (Korman), which are incorporated herein by reference in their entirety. The preparation and therapeutic uses of anti-PD-1 antibodies are described in U.S. Pat. No. 8,008,449 (Korman) and U.S. Patent Application No. 2011/0271358 (Freeman), which are incorporated herein by reference in their entirety. The preparation and therapeutic uses of anti-PD-L1 antibodies are described in U.S. Pat. No. 7,943,743 (Korman), which is incorporated herein by reference in its entirety. The preparation and therapeutic uses of anti-TIM-3 antibodies are described in U.S. Pat. No. 8,101,176 (Kuchroo) and U.S. Pat. No. 8,552,156 (Tagayanagi), which are incorporated herein by reference in their entirety. The preparation and therapeutic uses of anti-LAG-3 antibodies are described in U.S. Patent Application No. 2011/0150892 (Thudium) and International Publication Number WO2014/008218 (Lonberg), which are incorporated herein by reference in their entirety. The preparation and therapeutic uses of anti-KIR antibodies are described in U.S. Pat. No. 8,119,775 (Moretta), which is incorporated herein by reference in its entirety. The preparation of antibodies that block BTLA regulated inhibitory pathways (anti-BTLA antibodies) are described in U.S. Pat. No. 8,563,694 (Mataraza), which is incorporated herein by reference in its entirety.

In some embodiments, the one or more immune checkpoint inhibitor is an inhibitor of PD-1, PD-L1, or CTLA-4. In some embodiments, the immune checkpoint inhibitor is a PD-1 inhibitor. In some embodiments, the immune checkpoint inhibitor is a binding ligand of PD-L1. In some embodiments, the immune checkpoint inhibitor is a PD-L1 inhibitor. In some embodiments, the immune checkpoint inhibitor is a CTLA-4 inhibitor.

In some embodiments, the one or more immune checkpoint inhibitor as described herein includes a first immune checkpoint inhibitor and a second immune checkpoint inhibitor, wherein the first immune checkpoint inhibitor is different from the second immune checkpoint inhibitor. In some embodiments, the first and the second immune checkpoint inhibitor are independently an inhibitor of PD-1, PD-L1 or CTLA-4. In some embodiments, the first immune checkpoint inhibitor is a PD-1 inhibitor, and the second immune checkpoint inhibitor is a CTLA-4 inhibitor.

In some embodiments, the immune checkpoint inhibitor is pembrolizumab, nivolumab, cemiplimab, atezolizumab, avelumab, pembrolizumab, pidilizumab, ipilimumab, BMS 936559, durvalumab, spartalizumab, or any combinations thereof. In some embodiments, the one or more immune checkpoint inhibitor may include an anti-PD-1 HuMAbs can be selected from 17D8, 2D3, 4H1, 5C4 (also referred to herein as nivolumab), 4A1 1, 7D3 and 5F4, all of which are described in U.S. Pat. No. 8,008,449, which is incorporated herein by reference in its entirety. In some embodiments, the anti-PD-1 HuMAbs can be selected from 3G10, 12A4 (also referred to herein as BMS-936559), 10A5, 5F8, 10H10, 1B12, 7H1, 1 1E6, 12B7, and 13G4, all of which are described in U.S. Pat. No. 7,943,743, which is incorporated herein by reference in its entirety.

In some embodiments, the one or more immune checkpoint inhibitor may be incorporated in a pharmaceutically acceptable formulation. In some embodiments, the one or more immune checkpoint inhibitor is incorporated in a pharmaceutically acceptable aqueous formulation. Examples of acceptable aqueous formulations include isotonic buffered and pH 4.5-8 adjusted saline solutions such as Lactated Ringer's Solution and the like.

In some embodiments, the immune checkpoint inhibitor compound is incorporated in a pharmaceutically acceptable liposome formulation, wherein the formulation is a passive or targeted liposome formulation. Examples of methods for the preparation of suitable liposome formulations of antibodies are described U.S. Pat. No. 5,399,331 (Loughrey), U.S. Pat. No. 8,304,565 (Wu) and U.S. Pat. No. 7,780,882 (Chang), which are incorporated herein by reference in their entirety.

In some embodiments, the one or more immune checkpoint inhibitor may be an antibody. In some embodiments, the antibody is a dry, lyophilized solid that is reconstituted with an aqueous reconstitution solvent prior to use. In some embodiments, the antibody is incorporated in a pharmaceutically acceptable formulation and the pharmaceutically acceptable formulation is injected directly into a tumor. In some embodiments, the immune checkpoint inhibitor antibody is incorporated in a pharmaceutically acceptable formulation and the pharmaceutically acceptable formulation is injected into the peritumoral region surrounding a tumor. The peritumoral region may contain antitumor immune cells. In some embodiments, the antibody is incorporated in a pharmaceutically acceptable formulation and the pharmaceutically acceptable formulation is administered by intravenous injection or infusion. In some embodiments, the immune checkpoint inhibitor antibody is incorporated in a pharmaceutically acceptable formulation and the pharmaceutically acceptable formulation is administered by subcutaneous injection or intradermal injection. In some embodiments, the antibody is incorporated in a pharmaceutically acceptable formulation and the pharmaceutically acceptable formulation is administered by intraperitoneal injection or lavage.

The precise amount of immune checkpoint inhibitor compound incorporated in a particular method or therapeutic combination of the disclosure may vary according to factors known in art such as for example, the physical and clinical status of the subject, the method of administration, the content of the formulation, the physical and chemical nature of the immune checkpoint inhibitor compound, the intended dosing regimen or sequence. Those of ordinary skill in the art, however, can readily determine the appropriate amount with due consideration of such factors.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Examples

### General Procedures

It will be apparent to the skilled artisan that methods for preparing precursors and functionality related to the compounds claimed herein are generally described in the literature. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry (incorporated herein by reference in their entirety) and the like. All the intermediate compounds of the present invention were used without further purification unless otherwise specified.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader, and represent preferred methods for making the compounds exemplified herein. These methods are not limiting, and it will be apparent that other routes may be employed to prepare these compounds. Such methods specifically include solid phase-based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

The following abbreviations have the indicated meanings:
Ac₂O=acetic anhydride
ACN=acetonitrile
AcOH=acetic acid
AcOK=potassium acetate
BOC=tert-butoxycarbonyl
BOC₂O=di-tert-butyldicarbonate
Bu=butyl
clogP=calculated partition coefficient
DCM=dichloromethane
DIEA=N,N-diisopropylethylamine
DIPEA=N,N-diisopropylethylamine
DMAP=4-dimethylaminopyridine
DMF=dimethylformamide
DMSO=dimethyl sulfoxide
ESI=electrospray ionization
Et=Ethyl
FA=formic acid
FBS=fetal bovine serum
HATU=hexafluorophosphate benzotriazole tetramethyl uranium
HBSS=Hank's Balanced Salt Solution
HCHO=formaldehyde
HEPES=4-(2-hydroxyethyl)-1-piperazineethanesulfonic acid
HOAc=acetic acid
HPLC=high performance liquid chromatography
LCMS=liquid chromatography mass spectrometry
LHMDS=lithium bis(trimethylsilyl)amide
Log P=octanol/water partition coefficient
MDCK=Madin Darby canine kidney
MDR1=multidrug resistance gene-1
MS=mass spectrometry
MW=molecular weight
NADPH=reduced nicotinamide adenine dinucleotide phosphate
NCS=N-Chlorosuccinimide
NMR=nuclear magnetic resonance
PBS=phosphate-buffered saline
Pd₂(dba)₃=tris(dibenzylideneacetone)dipalladium(0)
Pd(dppf)Cl₂=bis(diphenylphosphino)ferrocene]dichloropalladium(II)
Ph=phenyl
PK=pharmacokinetic
Py=pyridine
TBS-Cl=tert-butylchlorodimethylsilane
TEA=triethylamine
TFA=trifluoracetic acid
THF=tetrahydrofuran
TLC=thin-layer chromatography

The following example schemes are provided for the guidance of the reader, and collectively represent an example method for making the compounds provided herein. Furthermore, other methods for preparing compounds described herein will be readily apparent to the person of ordinary skill in the art in light of the following reaction schemes and examples. Unless otherwise indicated, all variables are as defined above.

### General Example. Preparative HPLC

Preparative HPLC were carried out under one of the following conditions:
1) Column: Welch ultimate C18 150*25 mm*7 μm; mobile phase: [water(FA)-ACN]; gradient: 37%-67% B over 10 min;
2) column: Waters Xbridge 150*25 mm 10 μm; mobile phase: [water(NH₄HCO₃)-ACN]; gradient: 11%-41% B over 18 min; or
3) column: Waters Xbridge C18 150*50 mm*10 μm; mobile phase: [water(NH3H₂O)-ACN]; gradient: 12%-42% B over 10 min.

### Example 1. Preparation of N-(3-cyano-4-methyl-1H-indol-7-yl)-2-methyl-thiazole-5-sulfonamide (Compound 1)

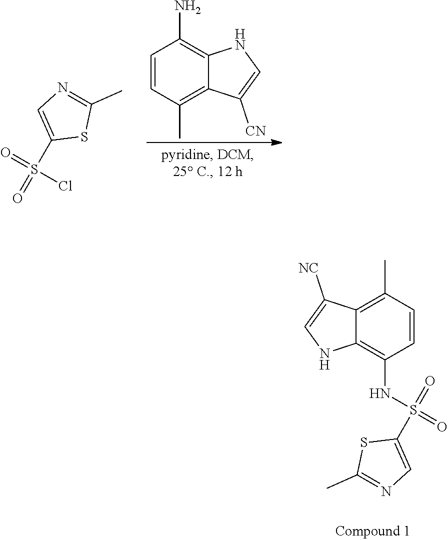

To a solution of 2-methylthiazole-5-sulfonyl chloride (100 mg, 506 μmol, 1.00 eq) and 7-amino-4-methyl-1H-indole-3-carbonitrile (95.3 mg, 557 μmol, 1.10 eq) in Dichloromethane (2.00 mL) was added Pyridine (80.0 mg, 1.01 mmol, 81.7 μL, 2.00 eq). The mixture was stirred at 25° C. for 12 h. The reaction mixture was filtered and collected the filter cake. The crude product was triturated with ethyl acetate (3.00 ml) at 25° C. for 10 min to give the N-(3-cyano-4-methyl-1H-indol-7-yl)-2-methyl-thiazole-5-sulfonamide (48.34 mg, 143.97 μmol, 28.46% yield, 99% purity) as a red solid. MS (ESI) m/z 333.0 [M+H]⁺. ¹H NMR (400 MHz, DMSO-d₆) δ=11.99 (s, 1H), 10.28 (s, 1H), 8.19 (d, J=3.2 Hz, 1H), 7.90 (s, 1H), 6.88 (d, J=7.6 Hz, 1H), 6.72 (d, J=7.6 Hz, 1H), 2.68 (s, 3H), 2.61 (s, 3H).

### Example 2. Synthetic Scheme of Compound 2

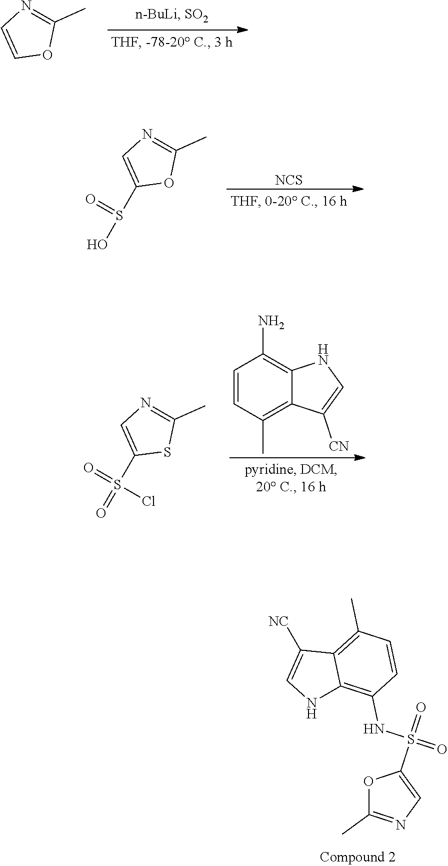

### Example 2.1. Preparation of 2-methyloxazole-5-sulfinic acid

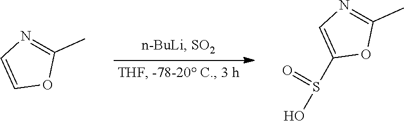

To a solution of 2-methyloxazole (200 mg, 2.41 mmol, 1.00 eq) in tetrahydrofuran (2.00 mL) was added n-Butyllithium (2.5 M, 1.44 mL, 1.50 eq) dropwise at −78° C. and stirred 30 min. under nitrogen, and then sulfur dioxide (154 mg, 2.41 mmol, 1.00 eq) was bubbled into at −65° C. for 30 min. The reaction mixture was warmed to 20° C. slowly and stirred for 2 h. A solution of 2-methyloxazole-5-sulfinic acid (400 mg, crude) in tetrahydrofuran (2.00 mL) was obtained as a yellow liquid and it used into next step directly.

### Example 2.2. Preparation of 2-methyloxazole-5-sulfonyl chloride

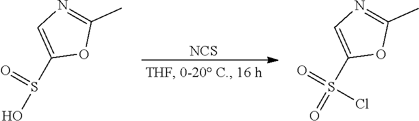

To a solution of 2-methyloxazole-5-sulfinic acid (395 mg, 2.42 mmol, 1.00 eq) in tetrahydrofuran (3.00 mL) was added 1-chloropyrrolidine-2,5-dione (969 mg, 7.26 mmol, 3.00 eq) at 0° C. The mixture was stirred at 20° C. for 16 h. The mixture was poured into water (30.0 mL) and extracted with ethyl acetate (30.0 mL). The combined organic layer was dried over anhydrous sodium sulfate, filtered and concentrated under vacuum to give a residue. The residue was purified by column chromatography (SiO₂, petroleum ether/ethyl acetate=3/1) to give 2-methyloxazole-5-sulfonyl chloride (180 mg, 912 μmol, 37% yield, 92% purity) as a yellow oil. ¹H NMR (400 MHz, CDCl₃) δ=7.75 (s, 1H), 2.66 (s, 3H).

### Example 2.3. Preparation of N-(3-cyano-4-methyl-1H-indol-7-yl)-2-methyl-oxazole-5-sulfonamide (Compound 2)

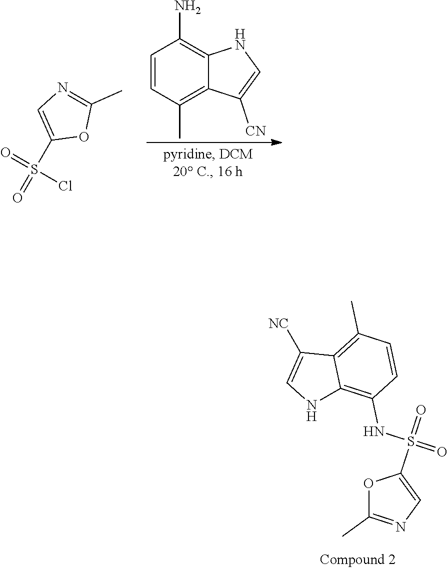

To a solution of 2-methyloxazole-5-sulfonyl chloride (50.0 mg, 275 mol, 1.00 eq) in dichloromethane (1.00 mL) was added pyridine (43.6 mg, 551 μmol, 44.5 μL, 2.00 eq) and 7-amino-4-methyl-1H-indole-3-carbonitrile (47.1 mg, 275 μmol, 1.00 eq). The mixture was stirred at 20° C. for 16 h. The mixture was concentrated under vacuum to give a residue. The residue was purified by prep-HPLC (column: Phenomenex luna C18 150*25 mm*10 μm; mobile phase: [water(FA)-ACN]; gradient: 22%-52% B over 8 min) and lyophilized to afford N-(3-cyano-4-methyl-1H-indol-7-yl)-2-methyl-oxazole-5-sulfonamide (30.66 mg, 95.66 μmol, 34.74% yield, 98.7% purity) as a purple solid. MS (ESI) m/z 317.2 [M+H]⁺. ¹H NMR (400 MHz, acetone) δ=11.39-11.05 (m, 1H), 8.15 (s, 1H), 7.36 (s, 1H), 7.01-6.81 (m, 2H), 2.71 (s, 3H), 2.51 (s, 3H).

### Example 3. Synthetic Scheme of Compound 3

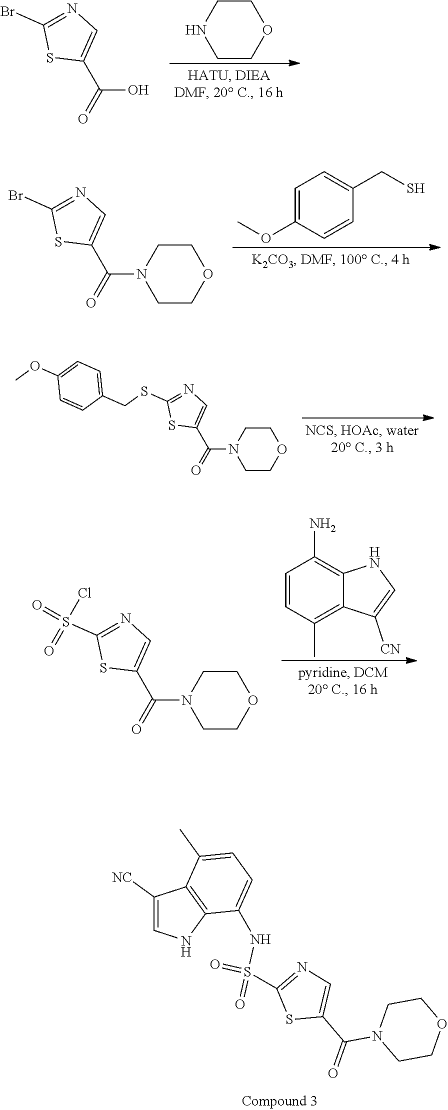

### Example 3.1. Preparation of 2-(2-bromothiazol-5-yl)-morpholino-methanone

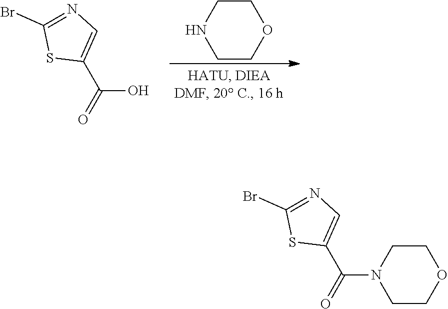 [SMILES: O=C(O)c1cnc(Br)s1 ; C1COCCN1 ; O=C(c1cnc(Br)s1)N1CCOCC1]

To a solution of 2-bromothiazole-5-carboxylic acid (1.50 g, 7.21 mmol, 1.00 eq) in dimethylformamide (15.0 mL) was added morpholine (690 mg, 7.93 mmol, 697 L, 1.10 eq), O-(7-azabenzotriazol-1-yl)-N,N,N,N-tetramethyluroniumhexafluorophosphate (2.74 g, 7.21 mmol, 1.00 eq) and N,N-diisopropylethylamine (1.86 g, 14.4 mmol, 2.51 mL, 2.00 eq) at 20° C. The mixture was stirred at 20° C. for 16 h. The mixture was poured into water (30.0 mL), extracted with dichloromethane (70 mL×3), dried over sodium sulfate, filtered and concentrated under reduced pressure to give a residue. The residue was purified by column chromatography (SiO₂, Petroleum ether/Ethyl acetate=100/1 to 5/1), TLC(Petroleum ether/Ethyl acetate=1:1, R=0.5, P=0.3). (2-Bromothiazol-5-yl)-morpholino-methanone (1.00 g, 3.57 mmol, 49% yield, 98% purity) was obtained as a white solid. ¹H NMR (400 MHz, DMSO-d₆) δ=8.02 (s, 1H), 3.63 (s, 8H).

### Example 3.2. Preparation of 3-[2-[(4-methoxyphenyl)methylsulfanyl]thiazol-5-yl]-morpholino-methanone

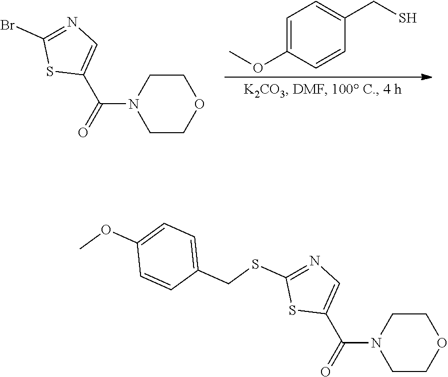

To a solution of (2-bromothiazol-5-yl)-morpholino-methanone (500 mg, 1.80 mmol, 1.00 eq) in dimethylformamide (5.00 mL) was added (4-methoxyphenyl)methanethiol (306 mg, 1.98 mmol, 276 μL, 1.10 eq) and potassium carbonate (498 mg, 3.61 mmol, 2.00 eq) at 20° C. The mixture was stirred at 100° C. for 4 h. The mixture was poured into water (30.0 mL), extracted with dichloromethane (70.0 mL×3), dried over sodium sulfate, filtered and concentrated under reduced pressure to give a residue. The residue was purified by column chromatography (SiO₂, Petroleum ether/Ethyl acetate=100/1 to 1/1), TLC (Petroleum ether/Ethyl acetate=0:1, Rf(R=0.5, P=0.3)). [2-[(4-Methoxyphenyl)methylsulfanyl]thiazol-5-yl]-morpholino-methanone (600 mg, crude) was obtained as a white solid. MS (ESI) m/z 351.2 [M+H]⁺.

### Example 3.3. Preparation of 5-(morpholine-4-carbonyl)thiazole-2-sulfonyl chloride

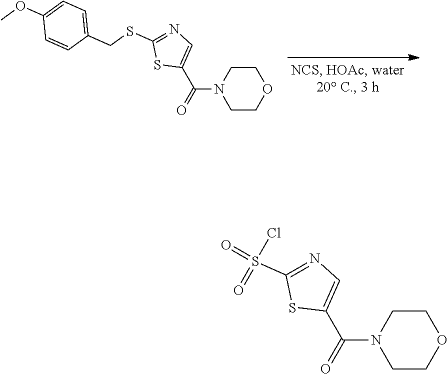 [SMILES: COc1ccc(CSc2ncc(C(=O)N3CCOCC3)s2)cc1 ; O=C(c1cnc(S(=O)(=O)Cl)s1)N1CCOCC1]

To a solution of [2-[(4-methoxyphenyl)methylsulfanyl]thiazol-5-yl]-morpholino-methanone (200 mg, 570 μmol, 1.00 eq) in acetic acid (3.00 mL) and water (1.00 mL) was added 1-chloropyrrolidine-2,5-dione (333 mg, 2.50 mmol, 4.37 eq) at 20° C. The mixture was stirred at 20° C. for 3 h. The mixture was poured into water(20.0 mL), extracted with dichloromethane (50 mL×3), dried over sodium sulfate, filtered and concentrated under reduced pressure to give a residue. 5-(morpholine-4-carbonyl)thiazole-2-sulfonyl chloride (155 mg, crude) was obtained as a white oil. MS (ESI) m/z 297.0/289.9 [M+H]⁺.

### Example 3.4. Preparation of N-(3-cyano-4-methyl-1H-indol-7-yl)-5-(morpholine-4-carbonyl)thiazole-2-sulfonamide (Compound 3)

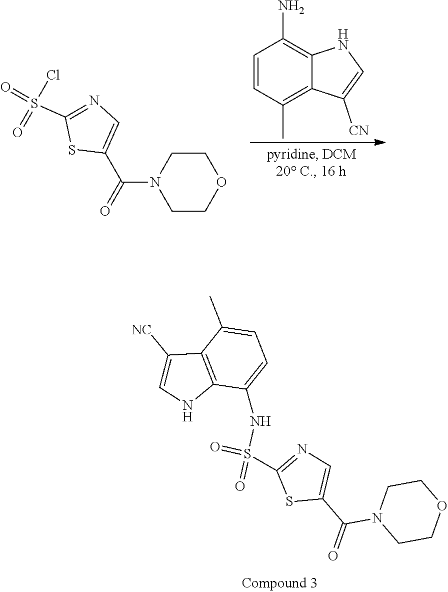 [SMILES: O=C(c1cnc(S(=O)(=O)Cl)s1)N1CCOCC1 ; Cc1ccc(N)c2[nH]cc(C#N)c12 ; Cc1ccc(NS(=O)(=O)c2ncc(C(=O)N3CCOCC3)s2)c2[nH]cc(C#N)c12]

To a solution of 7-amino-4-methyl-1H-indole-3-carbonitrile (30.0 mg, 175 μmol, 1.10 eq) in dichloromethane (1.00 mL) was added PYRIDINE (25.2 mg, 318 μmol, 25.7 L, 2.00 eq) and 5-(morpholine-4-carbonyl)thiazole-2-sulfonyl chloride (47.2 mg, 159 μmol, 1.00 eq) at 20° C. The mixture was stirred at 20° C. for 16 h. The mixture was poured into water (10.0 mL), and extracted with dichloromethane (20.0 mL×3), dried over sodium sulfate, filtered and concentrated under reduced pressure to give a residue. The crude product was purified by prep-HPLC (column: Phenomenex luna C18 150*25 mm*10 μm; mobile phase: [water(FA)-ACN]; gradient: 24%-54% B over 15 min). N-(3-cyano-4-methyl-1H-indol-7-yl)-5-(morpholine-4-carbonyl)thiazole-2-sulfonamide (5.86 mg, 13.38 μmol, 8.40% yield, 98.55% purity) was obtained as pink solid. MS (ESI) m/z 432.2 [M+H]⁺. ¹H NMR (400 MHz, DMSO-d₆)=12.12-12.03 (m, 1H), 10.87 (s, 1H), 8.37-8.26 (m, 1H), 8.19 (d, J=1.6 Hz, 1H), 6.84 (d, J=7.6 Hz, 1H), 6.73 (d, J=8.0 Hz, 1H), 3.60 (dd, J=4.8, 18.4 Hz, 8H), 2.60 (s, 3H).

### Example 4. Preparation of N-(3,4-dichloro-1H-indol-7-yl)-2-methyl-thiazole-5-sulfonamide (Compound 4)

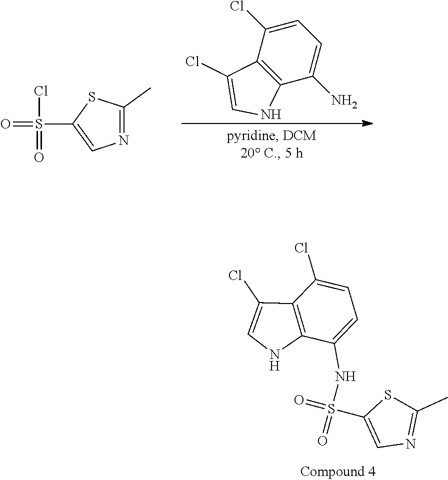

To a solution of 2-methylthiazole-5-sulfonyl chloride (30.0 mg, 152 mol, 1.20 eq) 3,4-dichloro-1H-indol-7-amine (30.0 mg, 126 μmol, 1.00 eq, HCl) in dichloromethane (0.500 mL) was added Pyridine (20.0 mg, 253 mol, 20.4 μL, 2.00 eq). The mixture was stirred at 25° C. for 5 h. The reaction mixture was concentrated under reduced pressure to remove solvent. The crude product was purified by prep-HPLC (column: Welch ultimate C18 150*25 mm*7 μm; mobile phase: [water(FA)-ACN]; gradient: 37%-67% B over 10 min) and desired fraction was collected and lyophilized to give the N-(3,4-dichloro-1H-indol-7-yl)-2-methyl-thiazole-5-sulfonamide (25.7 mg, 70.8 μmol, 56.1% yield, 100% purity) as a pink solid. MS (ESI) m/z 362.1/364.1/363.1 [M+H]⁺. ¹H NMR (400 MHz, DMSO-d₆) δ=11.43 (s, 1H), 10.53-10.21 (m, 1H), 7.93 (s, 1H), 7.53 (d, J=2.0 Hz, 1H), 6.99 (d, J=8.0 Hz, 1H), 6.79 (d, J=8.0 Hz, 1H), 2.66 (s, 3H).

### Example 5. Preparation of N-(3-(difluoromethyl)-4-methyl-1H-indol-7-yl)-2-methylthiazole-5-sulfonamide (Compound 5)

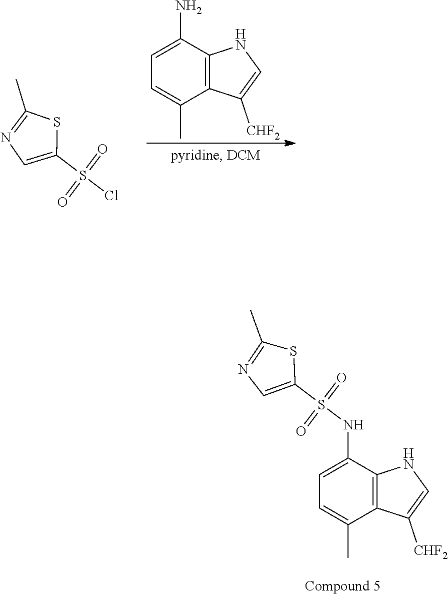

To a solution of 3-(difluoromethyl)-4-methyl-1H-indol-7-amine (28.4 mg, 145 mol, 0.450 eq) and pyridine (50.9 mg, 644 mol, 51.9 μL, 2.00 eq) in dichloromethane (0.800 mL) was added dropwise a solution of 2-methylthiazole-5-sulfonyl chloride (65 mg, 322 mol, 1.00 eq) in dichloromethane (0.200 mL) at 0° C. The mixture was stirred at 0° C. for 1 h. The mixture was poured into water (10.0 mL) and extracted with dichloromethane (10.0 ml×3). The combined organic phase was washed with brine (20.0 mL), dried with anhydrous sodium sulfate, filtered and concentrated in vacuum. The residue was purified by prep-HPLC (column: Phenomenex luna C18 150*25 mm*10 μm; mobile phase: [water(FA)-ACN]; gradient: 36%-66% B over 8 min). N-(3-(difluoromethyl)-4-methyl-1H-indol-7-yl)-2-methylthiazole-5-sulfonamide (7.24 mg, 15.03 mol, 4.67% yield, 98.67% purity) was obtained as a gray solid. MS (ESI) m/z 475.1/477.1/476.1 [M+H]⁺. ¹H NMR (400 MHz, CDCl₃) δ=11.3 (s, 1H), 10.2 (s, 1H), 7.9 (s, 1H), 7.7 (s, 1H), 7.38-7.10 (m, 1H), 6.8 (d, J=8.4 Hz, 1H), 6.70 (d, J=7.6 Hz, 1H), 2.67 (s, 3H), 2.51 (s, 3H).

### Example 6. Synthetic Scheme of Compound 6

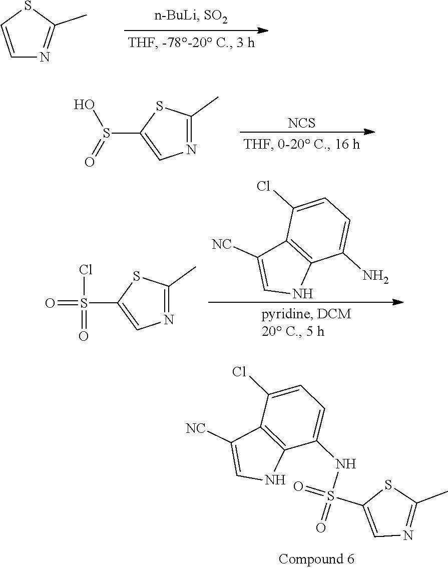

### Example 6.1. Preparation of 2-methylthiazole-5-sulfinic acid

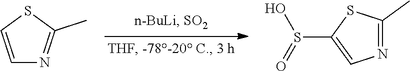

To a solution of 2-methylthiazole (1.00 g, 10.1 mmol, 1.00 eq) in tetrahydrofuran (20.0 mL) was added n-butyllithium (2.5 M, 6.05 mL, 1.50 eq) dropwise at −78° C. and stirred 30 min under nitrogen, and then sulfur dioxide (646 mg, 10.1 mmol, 1.00 eq) was bubbled into at −65° C. for 30 min. The reaction mixture was warmed to 20° C. slowly and stirred for 2 h. The crude product 2-methylthiazole-5-sulfinic acid (2.00 g, crude) in tetrahydrofuran (20.0 mL) was obtained as a yellow liquid. The mixture was used into the next step without further purification.

### Example 6.2. Preparation of 2-methylthiazole-5-sulfonyl chloride

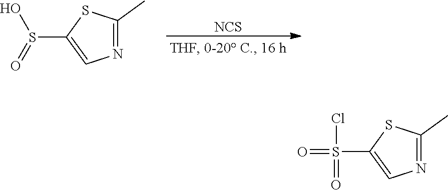

To a solution of 2-methylthiazole-5-sulfinic acid (2.00 g, 11.2 mmol, 1.00 eq) in tetrahydrofuran (20.0 mL) was added N-chloro-succinimide (4.47 g, 33.5 mmol, 3.00 eq) at 0° C. and stirred another 12 h at 20° C. The reaction mixture was quenched by addition water (20.0 mL) at 0° C., and then extracted with ethyl acetate (30 mL). The combined organic layers were washed with brine (20.0 mL), dried over sodium sulfate, filtered and concentrated under reduced pressure to give a residue. The residue was purified by column chromatography (SiO₂, Petroleum ether/Ethyl acetate=1/0 to 3/1) and concentrated under reduced pressure to give the 2-methylthiazole-5-sulfonyl chloride (1.30 g, 6.25 mmol, 56% yield, 95% purity) as a yellow oil. MS (ESI) m/z 198.0/200.0 [M+H]⁺. ¹H NMR (400 MHz, CDCl₃-d) 6=8.32 (s, 1H), 2.85 (s, 3H).

### Example 6.3. Preparation of N-(4-chloro-3-cyano-1H-indol-7-yl)-2-methyl-thiazole-5-sulfonamide (Compound 6)

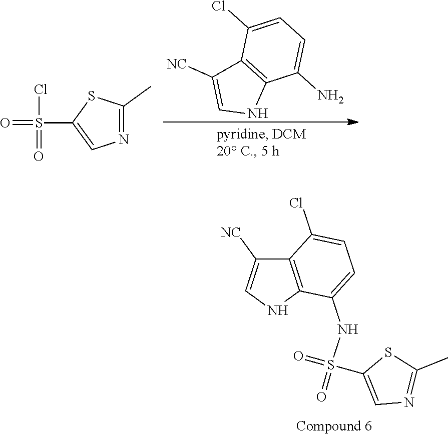

To a solution of 2-methylthiazole-5-sulfonyl chloride (37.1 mg, 188 mol, 1.20 eq) and 7-amino-4-chloro-1H-indole-3-carbonitrile (30.0 mg, 157 μmol, 1.00 eq) in dichloromethane (0.500 mL) was added pyridine (24.8 mg, 313 mol, 25.3 μL, 2.00 eq). The mixture was stirred at 25° C. for 5 h. The reaction mixture was concentrated under reduced pressure to remove solvent. The crude product was purified by prep-HPLC (column: Waters xbridge 150*25 mm 10 μm; mobile phase: [water(NH₄HCO₃)-ACN]; gradient: 11%-41% B over 18 min) and lyophilized to give the N-(4-chloro-3-cyano-1H-indol-7-yl)-2-methyl-thiazole-5-sulfonamide (9.20 mg, 26.0 μmol, 16.6% yield, 99.8% purity) as a white solid. MS (ESI) m/z 353.0/354.9/354.1 [M+H]⁺. ¹H NMR (400 MHz, DMSO-d₆) δ=12.34 (s, 1H), 10.50 (s, 1H), 8.33 (s, 1H), 7.93 (s, 1H), 7.18 (d, J=8.0 Hz, 1H), 6.91-6.79 (m, 1H), 2.68 (s, 3H).

### Example 7. Synthetic Scheme of Compound 7

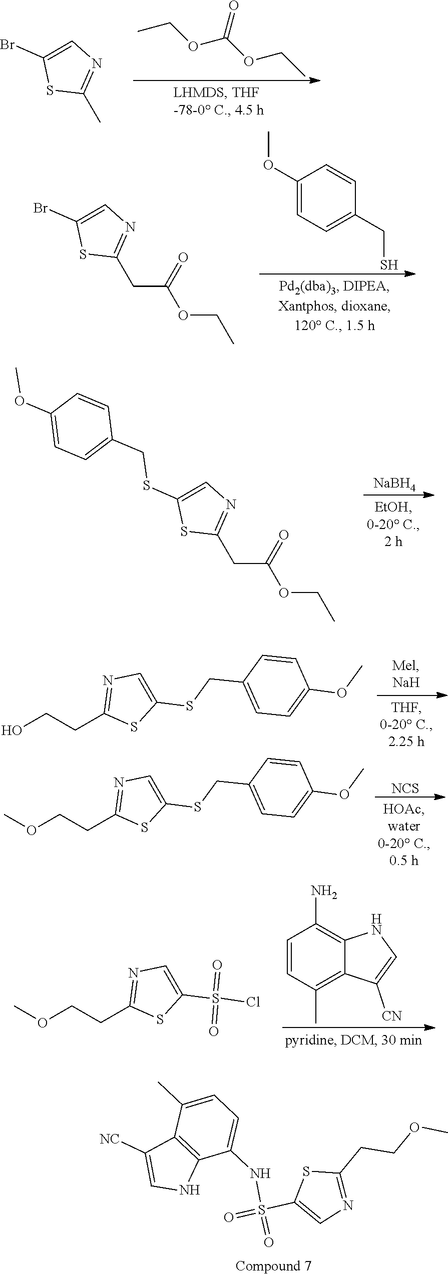

### Example 7.1. Preparation of ethyl 2-(5-bromothiazol-2-yl)acetate

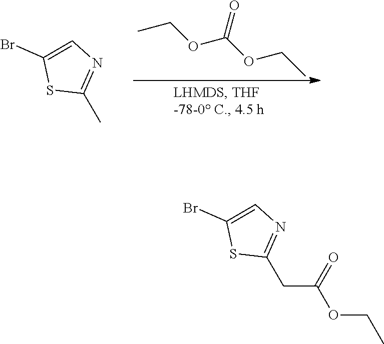

To a solution of 5-bromo-2-methyl-thiazole (1.50 g, 8.42 mmol, 1.00 eq) in tetrahydrofuran (15.0 mL) was added lithium bis(trimethylsilyl)amide (1 M, 18.5 mL, 2.20 eq). The mixture was stirred at −78° C. for 0.5 h. Diethyl carbonate (1.20 g, 10.2 mmol, 1.23 mL, 1.21 eq) was added and stirred at 0° C. for 4 h. After the end of the reaction, the reaction mixture was quenched with saturated aqueous ammonium chloride aqueous solution (50.0 mL) and extracted with ethyl acetate (50 mL×3). The organic was concentrated under vacuum to give a residue. The residue was purified by column chromatography (SiO₂, petroleum ether/ethyl acetate=3/1) to give ethyl 2-(5-bromothiazol-2-yl)acetate (550 mg, 2.20 mmol, 26% yield) as a yellow oil. ¹H NMR (400 MHz, DMSO-d₆) δ=7.87-7.74 (m, 1H), 4.20-4.10 (m, 4H), 1.21 (t, 1=7.2 Hz, 3H).

### Example 7.2. Preparation of 2-[5-[(4-methoxyphenyl)methylsulfanyl]thiazol-2-yl]acetate

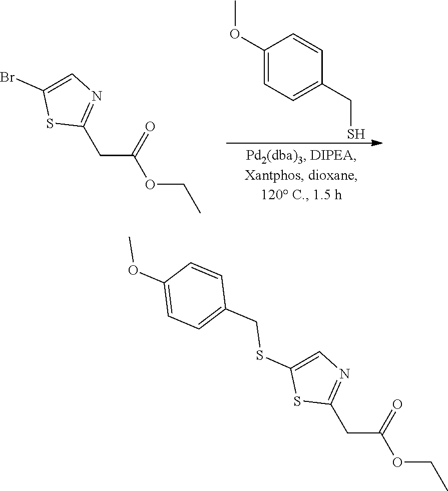

Ethyl 2-(5-bromothiazol-2-yl)acetate (500 mg, 2.00 mmol, 1.00 eq), (4-methoxyphenyl)methanethiol (462 mg, 3.00 mmol, 417 μL, 1.50 eq), 4,5-bis(diphenylphosphino)-9,9-dimethylxanthene (116 mg, 200 mol, 0.100 eq), diisopropylethylamine (775 mg, 6.00 mmol, 1.04 mL, 3.00 eq) and tris(dibenzylideneacetone)dipalladium(0) (183 mg, 200 μmol, 0.100 eq) were taken up into a microwave tube in dimethylformamide (5.00 mL). The sealed tube was heated at 120° C. for 1.5 h under microwave. The mixture was poured into water (30.0 mL) and extracted with ethyl acetate (3×5 mL). The combined organic layer was dried over anhydrous sodium sulfate, filtered and concentrated under vacuum to give a residue. The residue was purified by column chromatography (SiO₂, petroleum ether/ethyl acetate=3/1) to give ethyl 2-[5-[(4-methoxyphenyl)methylsulfanyl]thiazol-2-yl]acetate (490 mg, 1.52 mmol, 75% yield) as a yellow oil. MS (ESI) m/z 324.1 [M+H]⁺.

### Example 7.3. Preparation of 2-[5-[(4-methoxyphenyl)methylsulfanyl]thiazol-2-yl]ethanol

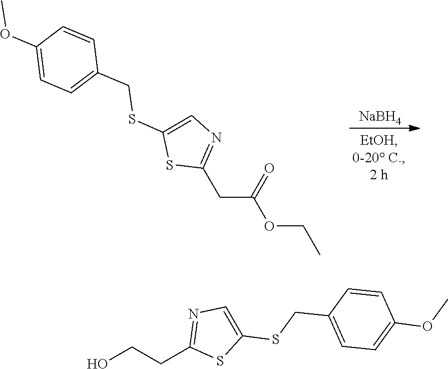

To a solution of ethyl 2-[5-[(4-methoxyphenyl)methylsulfanyl]thiazol-2-yl]acetate (220 mg, 680 mol, 1.00 eq) in ethanol (3.00 mL) was added sodium tetrahydroborate (66.9 mg, 1.77 mmol, 2.60 eq) in portion at 0° C. The mixture was warmed to 20° C. and stirred at 20° C. for 2 h. The mixture was quenched by adding saturated ammonium chloride (10.0 mL) and extracted with ethyl acetate (10.0 mL×3). The organic layer was concentrated under vacuum to give a crude product. The residue was purified by column chromatography (SiO₂, petroleum ether/ethyl acetate=3/1) to give 2-[5-[(4-methoxyphenyl)methylsulfanyl]thiazol-2-yl]ethanol (150 mg, 533 μmol, 78% yield) as a yellow oil. ¹H NMR (400 MHz, CDCl₃) δ=7.44 (s, 1H), 7.10 (d, J=8.4 Hz, 2H), 6.82 (d, J=8.4 Hz, 2H), 4.00 (t, J=5.6 Hz, 2H), 3.90 (s, 2H), 3.80 (s, 3H), 3.15 (t, J=5.6 Hz, 2H).

### Example 7.4. Preparation of 2-(2-methoxyethyl)-5-[(4-methoxyphenyl)methylsulfanyl]thiazole

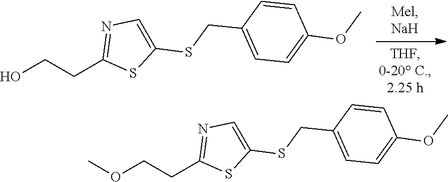

To a solution of 2-[5-[(4-methoxyphenyl)methylsulfanyl]thiazol-2-yl]ethanol (100 mg, 355 μmol, 1.00 eq) in tetrahydrofuran (2.00 mL) was added sodium hydride (15.6 mg, 391 μmol, 60% purity, 1.10 eq) at 0° C. The mixture was stirred at 0° C. for 10 min. Methyl iodide (60.5 mg, 426 μmol, 26.6 μL, 1.20 eq) was added to the mixture at 0° C. and stirred at 20° C. for 2 h. The mixture was poured in water (20.0 mL) and extracted with ethyl acetate (10.0 mL×2). The organic phase was concentrated under vacuum to give a residue. The residue was purified by column chromatography (SiO₂, petroleum ether/ethyl acetate=3/1) to give 2-(2-methoxyethyl)-5-[(4-methoxyphenyl)methylsulfanyl]thiazole (40.0 mg, 130 mol, 36% yield, 96% purity) as a yellow oil. MS (ESI) m/z 296.2 [M+H]⁺.

### Example 7.5. Preparation of 2-(2-methoxyethyl)thiazole-5-sulfonyl chloride

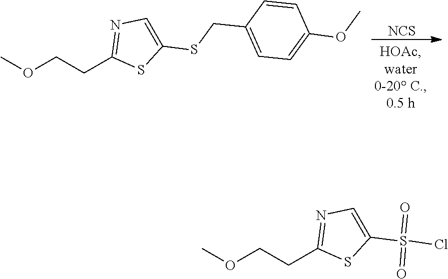

To a solution of 2-(2-methoxyethyl)-5-[(4-methoxyphenyl)methylsulfanyl]thiazole (22 mg, 74.5 μmol, 1.00 eq) in acetic acid (0.300 mL) and water (0.100 mL) was added N-chlorosuccinimide (39.8 mg, 298 mol, 4.00 eq) at 0° C. The mixture was stirred at 20° C. for 0.5 h. The mixture was poured into water (2.00 mL) and extracted with dichloromethane (0.500 mL). A solution of 2-(2-methoxyethyl)thiazole-5-sulfonyl chloride (18.0 mg, 38.0 mol, 51% yield, 51% purity) in dichloromethane (0.5 mL) was obtained as a yellow liquid. The solution was used to next step directly. MS (ESI) m/z 242.1 [M+H]⁺.

### Example 7.6. Preparation of N-(3-cyano-4-methyl-1H-indol-7-yl)-2-(2-methoxyethyl)thiazole-5-sulfonamide (Compound 7)

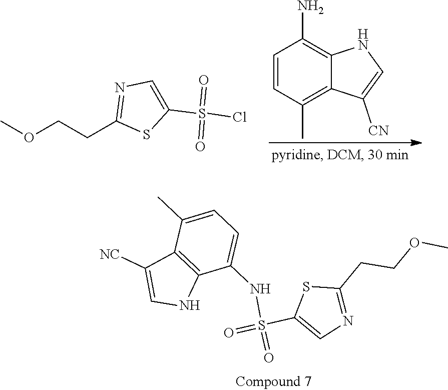

To a solution of 2-(2-methoxyethyl)thiazole-5-sulfonyl chloride (18.0 mg, 74.5 μmol, 1.00 eq) in dichloromethane (0.500 mL) was added pyridine (17.7 mg, 223 μmol, 18.0 μL, 3.00 eq) and 7-amino-4-methyl-1H-indole-3-carbonitrile (12.8 mg, 74.5 mol, 1.00 eq). The mixture was stirred at 20° C. for 0.5 h. The mixture was concentrated under vacuum to give a residue. The residue was purified by prep-HPLC (column: Phenomenex luna C18 150*25 mm*10 μm; mobile phase: [water(FA)-ACN]; gradient: 30%-60% B over 8 min) and lyophilized to afford N-(3-cyano-4-methyl-1H-indol-7-yl)-2-(2-methoxyethyl)thiazole-5-sulfonamide (9.32 mg, 24.11 mol, 32.38% yield, 97.4% purity) as a yellow solid. MS (ESI) m/z 377.1 [M+H]⁺. ¹H NMR (400 MHz, DMSO-d₆) δ=11.97 (s, 1H), 10.26 (d, J=1.2 Hz, 1H), 8.18 (d, J=2.4 Hz, 1H), 7.93 (s, 1H), 6.87 (d, J=7.6 Hz, 1H), 6.71 (d, J=7.6 Hz, 1H), 3.62 (t, J=6.0 Hz, 2H), 3.28-3.14 (m, 5H), 2.60 (s, 3H).

### Example 8. Synthetic Scheme of Compound 8

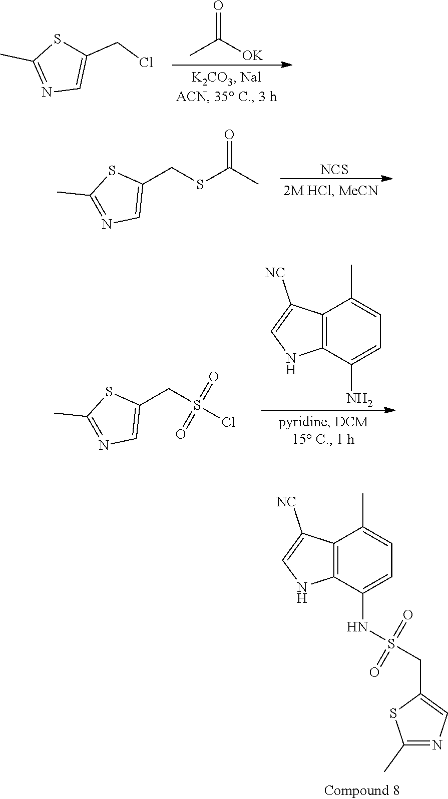

### Example 8.1. Preparation of 2-S-((2-methylthiazol-5-yl)methyl) ethanethioate

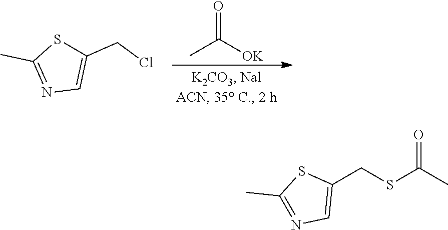

To a solution of 5-(chloromethyl)-2-methyl-thiazole (400 mg, 2.71 mmol, 1.00 eq) in acetonitrile (6.00 mL) was added ethanethioic S-acid (227 mg, 2.98 mmol, 213 μL, 1.10 eq), potassium carbonate (749 mg, 5.42 mmol, 2.00 eq) and sodium iodide (40.6 mg, 271 mol, 0.100 eq) at 16° C. The mixture was stirred at 35° C. for 2 h. The reaction mixture was quenched by addition water (30 mL), and then extracted with ethyl acetate (30 mL×3). The combined organic layers were washed with water (50 mL×2), dried over sodium sulphate, filtered and concentrated under reduced pressure to give a residue. The residue was purified by flash silica gel chromatography (ISCO®; 20 g SepaFlash® Silica Flash Column, Eluent of 0-10% Ethyl acetate/Petroleum ether gradient @50 mL/min). S-[(2-methylthiazol-5-yl)methyl]ethanethioate (450 mg, 2.40 mmol, 88% yield) was obtained as a yellow oil. ¹H NMR (400 MHz, DMSO-d₆) δ=7.48 (s, 1H), 4.30 (s, 2H), 2.58 (s, 3H), 2.37 (s, 3H).

### Example 8.2. Preparation of 3-(2-methylthiazol-5-yl)methanesulfonyl chloride

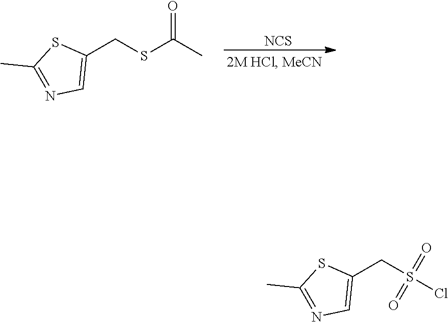

1-chloropyrrolidine-2,5-dione (185 mg, 1.38 mmol, 3.70 eq) was solved in acetonitrile (0.840 mL) and hydrochloric acid (2.00 M, 93.4 μL, 0.500 eq) at 0° C. S-[(2-methylthiazol-5-yl)methyl]ethanethioate (70.0 mg, 373 mol, 1.00 eq) was solved in acetonitrile (0.84 mL), which was added to the mixture at 0° C. for 5 min. The mixture was stirred at 15° C. for 10 min. The mixture was poured into water (10 mL), separated the organic phase and the aqueous phase was extracted with dichloromethane (3×5 mL). The organic layers were combined and washed with brine (10 mL), dried over anhydrous sodium sulfate and concentrated to give a residue. Model LCMS showed desired mass detected. The crude compound was used into the next step without further purification. (2-methylthiazol-5-yl)methanesulfonyl chloride (80 mg, crude) was obtained as a yellow solid.

### Example 8.3. Preparation of N-(3-cyano-4-methyl-1H-indol-7-yl)-1-(2-methylthiazol-5-yl)methanesulfonamide (Compound 8)

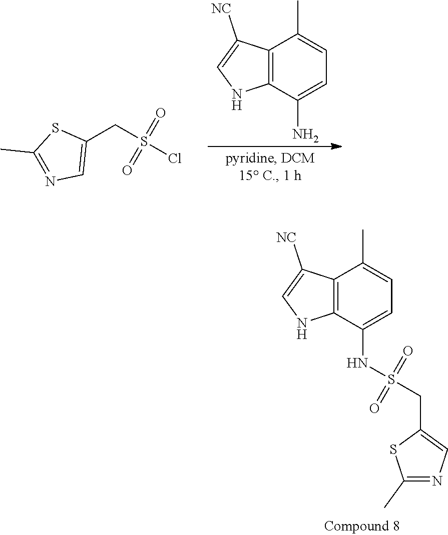 [SMILES: Cc1ncc(CS(=O)(=O)Cl)s1 ; Cc1ccc(N)c2[nH]cc(C#N)c12 ; Cc1ncc(CS(=O)(=O)Nc2ccc(C)c3c(C#N)c[nH]c23)s1]

To a solution of (2-methylthiazol-5-yl)methanesulfonyl chloride (51.4 mg, 170 μmol, 1.00 eq) in dichloromethane (5 mL) was added PYRIDINE (26.9 mg, 340 μmol, 27.5 μL, 2.00 eq). 7-amino-4-methyl-1H-indole-3-carbonitrile (29.1 mg, 170 mol, 1.00 eq) was added to the mixture at 0° C. The mixture was stirred at 15° C. for 2 h. The mixture was poured into water (10 mL), separated the organic phase and the aqueous phase was extracted with dichloromethane (3×5 mL). The organic layers were combined and washed with brine (10 mL), dried over anhydrous sodium sulfate and concentrated to give a residue. The residue was purified by prep-HPLC (Column: Waters xbridge 150*25 mm 10 um; Condition: water(NH₄HCO₃)-ACN; B %: 10%-40%; FlowRate (ml/min): 25.) to give desired compound. N-(3-cyano-4-methyl-1H-indol-7-yl)-1-(2-methylthiazol-5-yl)methanesulfonamide (21.46 mg, 61.95 mol, 36.43% yield, 100% purity) was obtained as a brown solid. MS (ESI) m/z 347.1 [M+H]⁺. ¹H NMR (400 MHz, DMSO-d₆) δ=11.99-11.75 (m, 1H), 9.64 (s, 1H), 8.20 (s, 1H), 7.48 (s, 1H), 7.12 (d, J=7.6 Hz, 1H), 6.96 (d, J=7.6 Hz, 1H), 4.71 (s, 2H), 2.64 (s, 3H), 2.61 (s, 3H).

### Example 9. Synthetic Scheme of Compound 9

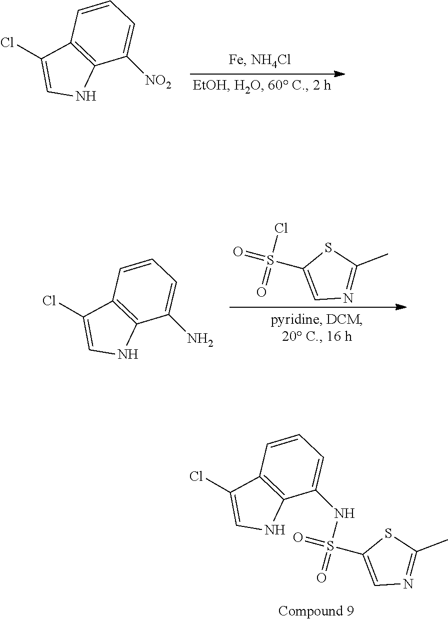

### Example 9.1. Preparation of 3-chloro-1H-indol-7-amine

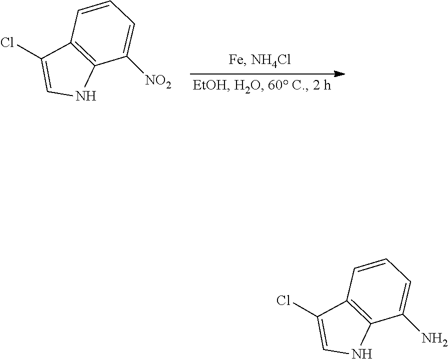 [SMILES: O=[N+]([O-])c1cccc2c(Cl)c[nH]c12 ; Nc1cccc2c(Cl)c[nH]c12]

To a solution of 3-chloro-7-nitro-1H-indole (200 mg, 1.02 mmol, 1.00 eq) and iron (227 mg, 4.07 mmol, 4.00 eq) in ethanol (2.00 mL) was added ammonium chloride (435 mg, 8.14 mmol, 8.00 eq) and water (2.00 mL), the mixture was stirred at 60° C. for 2 hours. The reaction mixture was filtered and concentrated under reduced pressure to give a residue. Without purification, the residue was directly used to the next step reaction. 3-chloro-1H-indol-7-amine (200 mg, crude) was obtained as a black oil. MS (ESI) m/z 167.3/169.3 [M+H]⁺.

### Example 9.2. Preparation of N-(3-chloro-1H-indol-7-yl)-2-methylthiazole-5-sulfonamide (Compound 9)

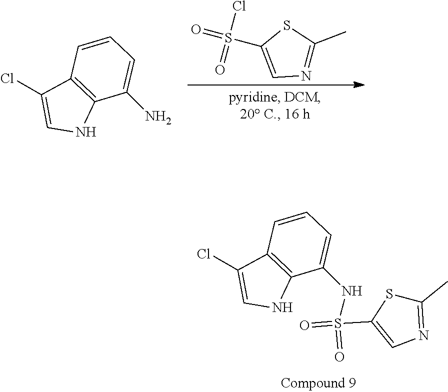

To a solution of 3-chloro-1H-indol-7-amine (50.0 mg, 300 mol, 1.00 eq) and 2-methylthiazole-5-sulfonyl chloride (59.6 mg, 300 mol, 1.00 eq) in dichloromethane (0.500 mL) was added pyridine (490 mg, 6.19 mmol, 0.50 mL, 20.6 eq), the mixture was stirred at 20° C. for 16 hours. The reaction mixture was concentrated under reduced pressure to give a residue. The residue was purified by prep-HPLC (column: Phenomenex luna C18 150*25 mm*10 um; mobile phase: [water(FA)-ACN]; gradient: 30%-60% B over 10 min). N-(3-chloro-1H-indol-7-yl)-2-methylthiazole-5-sulfonamide (11.95 mg, 36.12 μmol, 12.03% yield, 99.07% purity) was obtained as a yellow solid. MS (ESI) m/z 328.2/330.1 [M+H]⁺. ¹H NMR (400 MHz, DMSO-d₆) δ=11.05 (s, 1H), 10.61-10.01 (m, 1H), 7.93 (s, 1H), 7.47 (s, 1H), 7.31 (d, J=7.2 Hz, 1H), 7.11-6.97 (m, 1H), 6.91 (d, J=7.2 Hz, 1H), 2.65 (s, 3H).

### Example 10. Synthetic Scheme of Compound 10

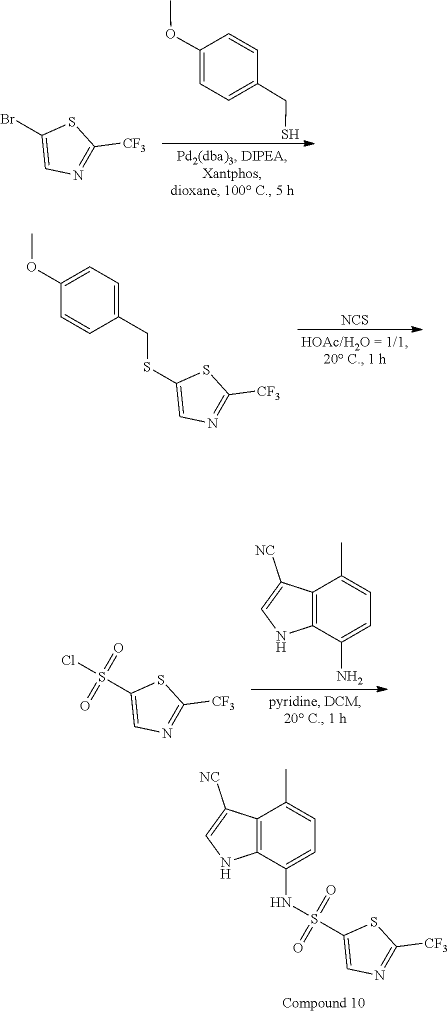

### Example 10.1. Preparation of 5-[(4-methoxyphenyl)methylsulfanyl]-2-(trifluoromethyl)thiazole

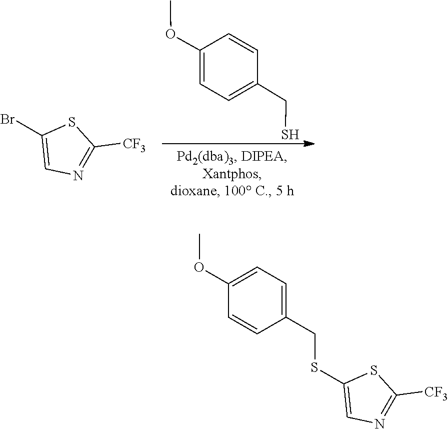

A mixture of 5-bromo-2-(trifluoromethyl)thiazole (450 mg, 1.94 mmol, 1.00 eq), (4-methoxyphenyl)methanethiol (359 mg, 2.33 mmol, 324 L, 1.20 eq), tris(dibenzylideneacetone)dipalladium(0) (178 mg, 194 μmol, 0.100 eq), 4,5-bis(diphenylphosphino)-9,9-dimethylxanthene (112 mg, 194 mol, 0.100 eq) and N,N-diisopropylethylamine (752 mg, 5.82 mmol, 1.01 mL, 3.00 eq) in dioxane (9.00 mL) was degassed and purged with nitrogen for 3 times, and then the mixture was stirred at 100° C. for 5 h under nitrogen atmosphere. The mixture was extracted with ethyl acetate (15.0 mL), washed with brine (15.0 mL), dried with sodium sulfate, filtered and concentrated. The residue was purified by column chromatography (SiO₂, Petroleum ether/Ethyl acetate=1/0 to 20/1) and concentrated under reduced pressure to remove solvent to give the 5-[(4-methoxyphenyl)methylsulfanyl]-2-(trifluoromethyl)thiazole (550 mg, 1.71 mmol, 88% yield, 95% purity) as an off-white oil. ¹H NMR (400 MHz, DMSO-d6) δ=7.99 (s, 1H), 7.19 (d, J=8.4 Hz, 2H), 6.90-6.85 (m, 2H), 4.21 (s, 2H), 3.73 (s, 3H).

### Example 10.2. Preparation of 2-(trifluoromethyl)thiazole-5-sulfonyl chloride

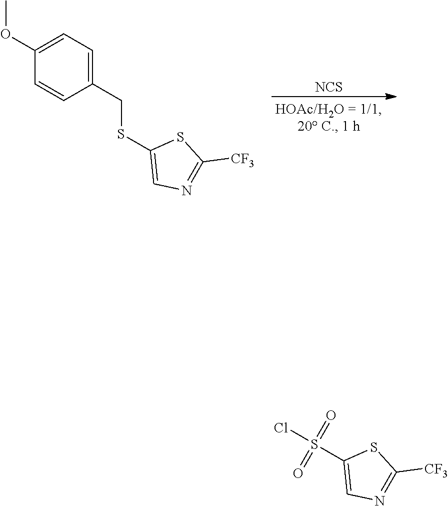 [SMILES: COc1ccc(CSc2cnc(C(F)(F)F)s2)cc1 ; "NCS" ; O=S(=O)(Cl)c1cnc(C(F)(F)F)s1]

To a solution of 5-[(4-methoxyphenyl)methylsulfanyl]-2-(trifluoromethyl)thiazole (50.0 mg, 164 μmol, 1.00 eq) in water (0.250 mL) was added 1-chloropyrrolidine-2,5-dione (65.6 mg, 491 μmol, 3.00 eq) and acetic acid (262 mg, 4.37 mmol, 0.250 mL, 26.7 eq). The mixture was stirred at 20° C. for 1 h. The reaction mixture was concentrated under reduced pressure to remove solvent to give the 2-(trifluoromethyl)thiazole-5-sulfonyl chloride (30.0 mg, crude) as a white solid.

### Example 10.3. Preparation of N-(3-cyano-4-methyl-1H-indol-7-yl)-2-(trifluoromethyl)thiazole-5-sulfonamide (Compound 10)

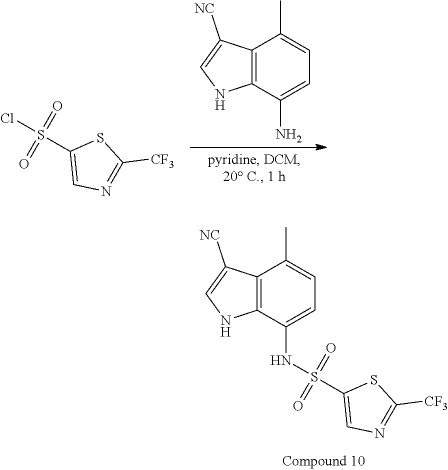 [SMILES: O=S(=O)(Cl)c1cnc(C(F)(F)F)s1 ; Cc1ccc(N)c2[nH]cc(C#N)c12 ; Cc1ccc(NS(=O)(=O)c2cnc(C(F)(F)F)s2)c2[nH]cc(C#N)c12]

To a solution of 2-(trifluoromethyl)thiazole-5-sulfonyl chloride (30.0 mg, 119 μmol, 1.00 eq) in dichloromethane (0.500 mL) was added Pyridine (18.9 mg, 238 μmol, 19.2 μL, 2.00 eq) and 7-amino-4-methyl-1H-indole-3-carbonitrile (20.4 mg, 119 μmol, 1.00 eq). The mixture was stirred at 20° C. for 1 h. The reaction mixture was concentrated under reduced pressure to remove solvent. The crude product was purified by prep-HPLC (column: Phenomenex luna C18 150*25 mm*10 μm; mobile phase: [water(FA)-ACN]; gradient: 43%-73% B over 10 min) and lyophilized to afford the N-(3-cyano-4-methyl-1H-indol-7-yl)-2-(trifluoromethyl)thiazole-5-sulfonamide (6.08 mg, 15.0 μmol, 12.5% yield, 95.0% purity) as a yellow solid. MS (ESI) m/z 387.1 [M+H]⁺. ¹H NMR (400 MHz, DMSO-d₆) δ=12.09 (s, 1H), 10.72 (s, 1H), 8.40-8.31 (m, 1H), 8.21-8.10 (m, 1H), 6.86 (d, J=7.6 Hz, 1H), 6.74 (d, J=7.6 Hz, 1H), 2.60 (s, 3H).

### Example 11. Preparation of N-(3,4-dichloro-1H-indol-7-yl)-2-(trifluoromethyl)thiazole-5-sulfonamide (Compound 11)

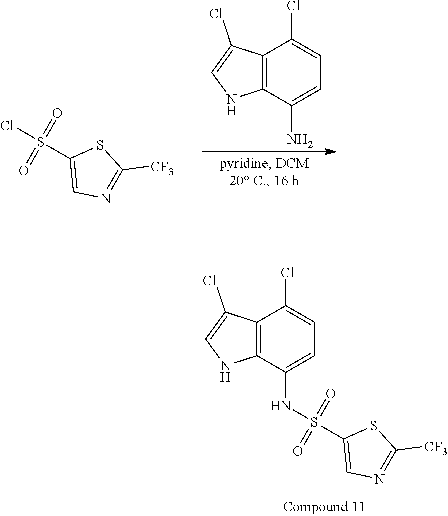 [SMILES: O=S(=O)(Cl)c1cnc(C(F)(F)F)s1 ; Nc1ccc(Cl)c2c(Cl)c[nH]c12 ; O=S(=O)(Nc1ccc(Cl)c2c(Cl)c[nH]c12)c1cnc(C(F)(F)F)s1]

To a solution of 2-(trifluoromethyl)thiazole-5-sulfonyl chloride (30.0 mg, 119 mol, 1.00 eq) in dichloromethane (0.500 mL) was added pyridine (18.9 mg, 238 mol, 19.3 μL, 2.00 eq) and 3,4-dichloro-1H-indol-7-amine (23.9 mg, 119 mol, 1.00 eq). The mixture was stirred at 20° C. for 0.5 h. The reaction mixture was concentrated under reduced pressure to remove solvent. The crude product was purified by prep-HPLC (column: Waters xbridge 150*25 mm 10p m; mobile phase: [water(NH₄HCO₃)-ACN]; gradient: 25%-55% B over 14 min) and lyophilized to give the N-(3,4-dichloro-1H-indol-7-yl)-2-(trifluoromethyl)thiazole-5-sulfonamide (4.65 mg, 11.2 mol, 9.40% yield, 99.8% purity) as a brown solid. MS (ESI) m/z 413.9 [M+H]⁺. ¹H NMR (400 MHz, DMSO-d₆) δ=11.51 (s, 1H), 10.89-10.64 (m, 1H), 8.34 (s, 1H), 7.46 (s, 1H), 6.95 (d, J=8.0 Hz, 1H), 6.81 (d, J=8.0 Hz, 1H).

### Example 12. Preparation of N-(3-cyano-4-methyl-1H-indol-7-yl)-2-(1-methyl-4-piperidyl)thiazole-5-sulfonamide (Compound 12)

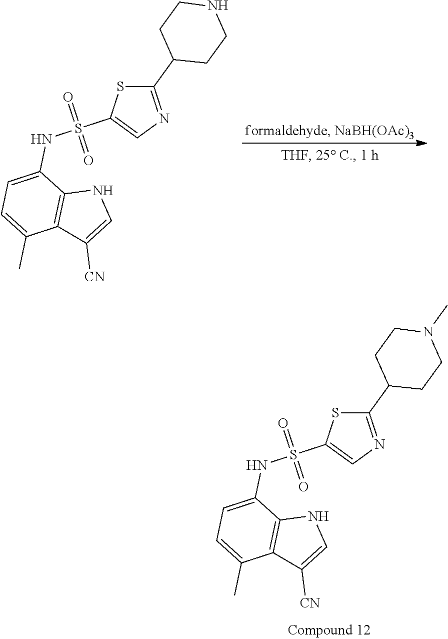 [SMILES: Cc1ccc(NS(=O)(=O)c2cnc(C3CCNCC3)s2)c2[nH]cc(C#N)c12 ; Cc1ccc(NS(=O)(=O)c2cnc(C3CCN(C)CC3)s2)c2[nH]cc(C#N)c12]

To a solution of N-(3-cyano-4-methyl-1H-indol-7-yl)-2-(4-piperidyl)thiazole-5-sulfonamide (20.0 mg, 49.8 μmol, 1.00 eq) in tetrahydrofuran (2.00 mL) was added formaldehyde (2.99 mg, 99.6 mol, 2.74 μL, 2.00 eq) and sodium triacetoxyhydroborate (31.7 mg, 149 μmol, 3.00 eq) at 16° C. The mixture was stirred at 25° C. for 1 h. The mixture was concentrated under reduced pressure to give a residue. The crude product was purified by prep-HPLC (column: Waters xbridge 150*25 mm 10 μm; mobile phase: [water(NH₄HCO₃)-ACN]; gradient: 9%-39% B over 10 min). N-(3-cyano-4-methyl-1H-indol-7-yl)-2-(1-methyl-4-piperidyl)thiazole-5-sulfonamide (8.81 mg, 21.05 mol, 42.27% yield, 99.30% purity) was obtained as a white solid. MS (ESI) m/z 461.2 [M+H]⁺. ¹H NMR (400 MHz, DMSO-d₆) δ=12.05-11.68 (m, 1H), 8.02 (s, 1H), 7.87 (s, 1H), 6.87-6.67 (m, 2H), 6.16-6.14 (m, 1H), 3.10-2.99 (m, 3H), 2.55 (s, 3H), 2.42 (s, 5H), 2.11-1.99 (m, 2H), 1.80-1.67 (m, 2H).

### Example 13. Synthetic Scheme of Compound 13

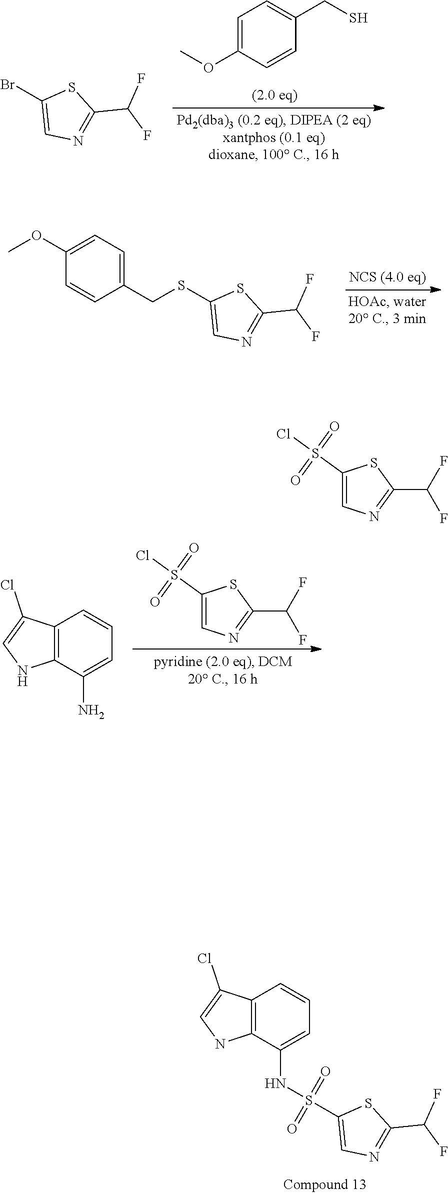

### Example 13.1. Preparation of 2-(difluoromethyl)-5-((4-methoxybenzyl)thio)thiazole

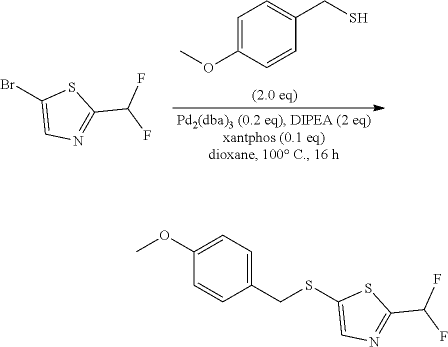

To a solution of 5-bromo-2-(difluoromethyl)thiazole (300 mg, 1.40 mmol, 1.00 eq) in dioxane (6.00 mL) was added (4-methoxyphenyl)methanethiol (432 mg, 2.80 mmol, 390 L, 2.00 eq), N,N-diisopropylethylamine (362 mg, 2.80 mmol, 488 μL, 2.00 eq), tris(dibenzylideneacetone)dipalladium(0) (256 mg, 280 mol, 0.200 eq) and 4,5-bis(diphenylphosphino)-9,9-dimethylxanthene (81.1 mg, 140 μmol, 0.100 eq) at 20° C., the mixture was stirred at 100° C. for 16 h. The mixture was diluted with water (50 mL). And then extracted with ethyl acetate (2×50 mL). The combined organic layers were dried over sodium sulfate, and concentrated in vacuum to give a residue. The crude product was purified by prep-HPLC(column: Phenomenex luna C18 150*25 mm*10 μm; mobile phase: [water(FA)-ACN]; gradient: 56%-76% B over min), which was determined by LCMS. The desired fraction was lyophilized. 2-(difluoromethyl)-5-((4-methoxybenzyl)thio)thiazole (200 mg, 696 mol, 49% yield) was obtained as a yellow liquid. MS (ESI) m/z 288.2. [M+H]⁺. 4H NMR (400 MHz, chloroform) 6 ppm 7.58-7.63 (m, 1H), 7.12 (d, J=8.3 Hz, 2H), 6.80-6.91 (m, 2H), 6.58-6.76 (m, 1H), 3.95-4.02 (m, 2H), 3.77-3.85 (m, 3H).

### Example 13.2. Preparation of 2-(difluoromethyl)thiazole-5-sulfonyl chloride

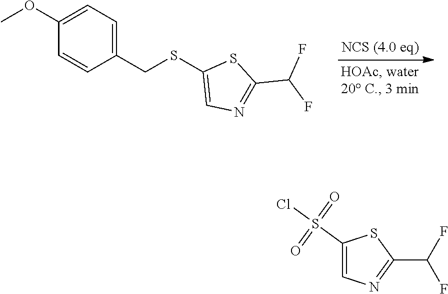 [SMILES: COc1ccc(CSc2cnc(C(F)F)s2)cc1 ; O=S(=O)(Cl)c1cnc(C(F)F)s1]

To a solution of 2-(difluoromethyl)-5-((4-methoxybenzyl)thio)thiazole (100 mg, 348 μmol, 1.00 eq) in acetic acid (1.5 mL) and water (0.5 mL) was added 1-chloropyrrolidine-2,5-dione (185 mg, 1.39 mmol, 4.00 eq) at 0° C., the mixture was stirred at 25° C. for 1 h. The mixture was diluted with ice water (20 mL). And then extracted with ethyl acetate (2×20 mL). The combined organic layers were dried over sodium sulfate, and concentrated in vacuum to give a residue. It was not purified and used for the next step. 2-(difluoromethyl)thiazole-5-sulfonyl chloride (80 mg, crude) was obtained as a yellow liquid.

### Example 13.3. Preparation of N-(3-chloro-1H-indol-7-yl)-2-(difluoromethyl)thiazole-5-sulfonamide (Compound 13)

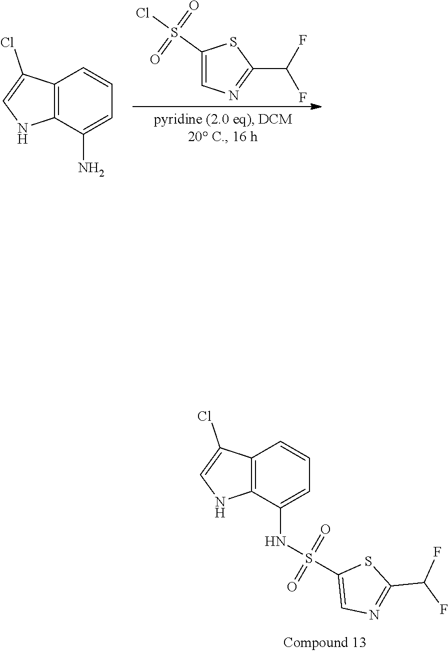 [SMILES: Nc1cccc2c(Cl)c[nH]c12 ; O=S(=O)(Cl)c1cnc(C(F)F)s1 ; O=S(=O)(Nc1cccc2c(Cl)c[nH]c12)c1cnc(C(F)F)s1]

To a solution of 3-chloro-1H-indol-7-amine (34.2 mg, 205 μmol, 1.20 eq) in dichloromethane (1.00 mL) was added pyridine (27.0 mg, 342 μmol, 27.6 μL, 2.00 eq) and 2-(difluoromethyl)thiazole-5-sulfonyl chloride (40.0 mg, 171 mol, 1.00 eq) at 0° C., the mixture was stirred at 20° C. for 10 min. The mixture was concentrated in vacuum to give a crude product. The crude product was purified by prep-HPLC(column: Waters Xbridge C18 150*50 mm*10 μm; mobile phase: [water(NH3H2O)-ACN]; gradient: 8%-38% B over 10 min). The desired fraction was lyophilized. N-(3-chloro-1H-indol-7-yl)-2-(difluoromethyl)thiazole-5-sulfonamide (23.57 mg, 62.85 mol, 36.71% yield, 97% purity) was obtained as a yellow solid. MS (ESI) m/z 363.9. [M+H]⁺. ¹H NMR (400 MHz, DMSO-d₆) δ ppm 11.17 (s, 1H), 10.60 (s, 1H), 8.31 (s, 1H), 7.50 (d, J=2.4 Hz, 1H), 7.19-7.42 (m, 2H), 7.05 (t, J=8.0 Hz, 1H), 7.05 (t, J=8.0 Hz, 1H), 6.85 (d, J=7.2 Hz, 1H).

### Example 14. Preparation of N-(3,4-dichloro-1H-indol-7-yl)-2-(difluoromethyl)thiazole-5-sulfonamide (Compound 14)

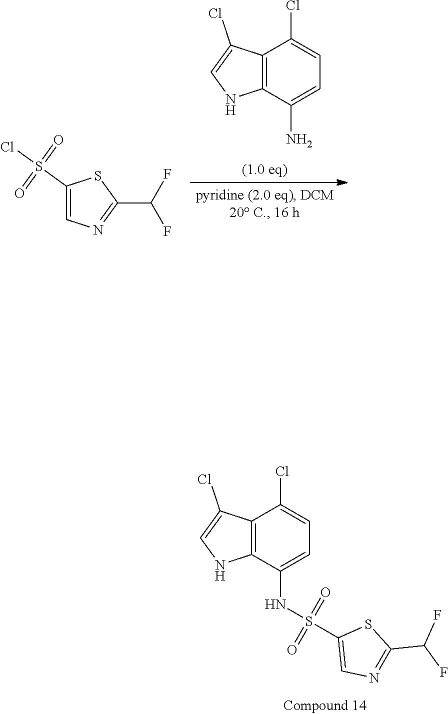 [SMILES: Nc1ccc(Cl)c2c(Cl)c[nH]c12 ; O=S(=O)(Cl)c1cnc(C(F)F)s1 ; O=S(=O)(Nc1ccc(Cl)c2c(Cl)c[nH]c12)c1cnc(C(F)F)s1]

To a solution of 3,4-dichloro-1H-indol-7-amine (40.66 mg, 171 μmol, 1.00 eq, hydrochloric acid) in dichloromethane (1.00 mL) was added pyridine (27.0 mg, 342 mol, 27.6 μL, 2.00 eq) and 2-(difluoromethyl)thiazole-5-sulfonyl chloride (40.0 mg, 171 μmol, 1.00 eq) at 0° C., the mixture was stirred at 20° C. for 10 min. The mixture was concentrated in vacuum to give a crude product. The crude product was purified by prep-HPLC (column: Waters Xbridge C18 150*50 mm*10 μm; mobile phase: [water(NH3H2O)-ACN]; gradient: 12%-42% B over 10 min). The desired fraction was lyophilized. N-(3,4-dichloro-1H-indol-7-yl)-2-(difluoromethyl)thiazole-5-sulfonamide (24.95 mg, 62.02 mol, 36.23% yield, 99% purity) was obtained as a yellow solid. MS (ESI) m/z 416.0/414.1 [M+H]⁺. ¹H NMR (400 MHz, DMSO-d₆) δ ppm 11.58 (s, 1H), 10.65 (s, 1H), 8.32 (s, 1H), 7.59 (d, J=2.4 Hz, 1H), 7.38 (t, J=53.6 Hz, 1H), 7.04 (d, J=8.0 Hz, 1H), 6.76 (d, J=8.0 Hz, 1H).

### Examples 15 and 16. Synthetic Scheme of Compound 15 and Compound 16

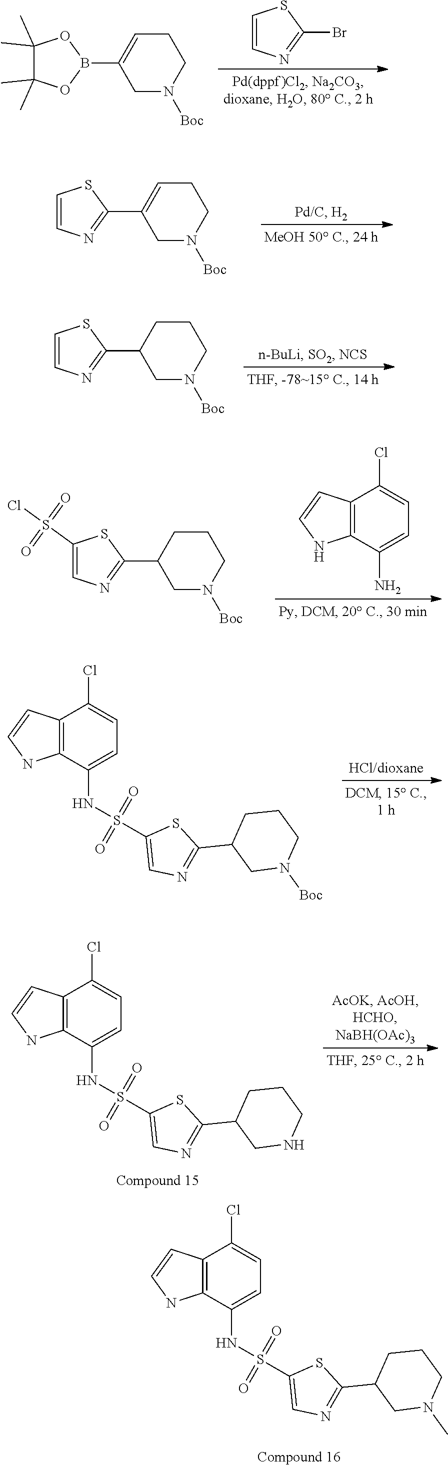

### Example 15.1. Preparation of tert-butyl 5-(thiazol-2-yl)-3,6-dihydropyridine-1(2H)-carboxylate

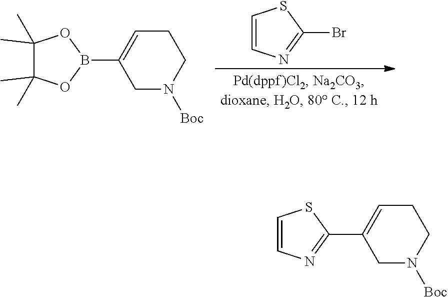 [SMILES: CC(C)(C)OC(=O)N1CCC=C(B2OC(C)(C)C(C)(C)O2)C1 ; Brc1nccs1 ; CC(C)(C)OC(=O)N1CCC=C(c2nccs2)C1]

A mixture of tert-butyl 5-(4,4,5,5-tetramethyl-1,3,2-dioxaborolan-2-yl)-3,6-dihydropyridine-1(2H)-carboxylate (3.00 g, 9.70 mmol, 1.00 eq), 2-bromothiazole (1.59 g, 9.70 mmol, 874 μL, 1.00 eq), sodium carbonate (2.57 g, 24.3 mmol, 2.50 eq) and [1,1-bis(diphenylphosphino)ferrocene]dichloropalladium(II) (710 mg, 970 mol, 0.100 eq) in dioxane (50.0 mL) and water (10.0 mL) was degassed and purged with nitrogen for 3 times, and then the mixture was stirred at 80° C. for 12 h under nitrogen atmosphere. The mixture was diluted with water (100 mL) and extracted with ethyl acetate (100 mL×2). The combined organic layers were washed with brine (100 mL) and dried with sodium sulfate solid and filtered. Then the filtrate was concentrated under reduced pressure to dryness. The residue was purified by flash silica gel chromatography (ISCO®; 80 g Sepa Flash® Silica Flash Column, Eluent of 0-10% Ethyl acetate/Petroleum ether gradient @100 mL/min). Compound tert-butyl 5-(thiazol-2-yl)-3,6-dihydropyridine-1(2H)-carboxylate (1.60 g, 5.59 mmol, 57% yield, 93% purity) was obtained as a white solid. ¹H NMR (400 MHz, CDCl₃) δ=7.75 (d, J=3.2 Hz, 1H), 7.21 (d, J=3.2 Hz, 1H), 6.72 (s, 1H), 4.44 (s, 2H), 3.59 (t, J=5.6 Hz, 2H), 2.36 (s, 2H), 1.50 (s, 9H).

### Example 15.2. Preparation of tert-butyl 3-(thiazol-2-yl)piperidine-1-carboxylate

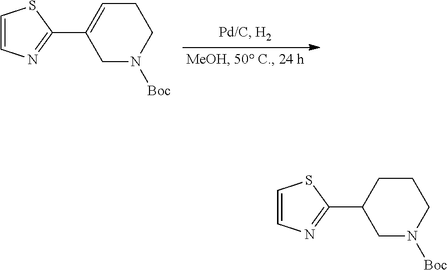 [SMILES: CC(C)(C)OC(=O)N1CCC=C(c2nccs2)C1 ; CC(C)(C)OC(=O)N1CCCC(c2nccs2)C1]

To a solution of tert-butyl 5-(thiazol-2-yl)-3,6-dihydropyridine-1(2H)-carboxylate (1.60 g, 6.01 mmol, 1.00 eq) in methanol (30.0 mL) was added Pd/C (639 mg, 600 μmol, 10% purity, 0.100 eq) under nitrogen. The suspension was degassed under vacuum and purged with hydrogen several times. The mixture was stirred under hydrogen (15 Psi) at 50° C. for 24 h. The suspension was filtered through a pad of Celite and filter cake was washed with methanol (50.0 mL×2). The combined filtrates were concentrated to dryness. The residue was purified by flash silica gel chromatography (ISCO®; 20 g Sepa Flash® Silica Flash Column, Eluent of 0-15% Ethyl acetate/Petroleum ether gradient @100 mL/min). Compound tert-butyl 3-(thiazol-2-yl)piperidine-1-carboxylate (860 mg, 3.11 mmol, 51.7% yield, 97% purity) was obtained as a white solid. ¹H NMR (400 MHz, CDCl₃) δ=7.72 (d, J=3.2 Hz, 1H), 7.23 (d, J=3.2 Hz, 1H), 4.49-4.22 (m, 1H), 4.03 (d, J=12.0 Hz, 1H), 3.27-3.14 (m, 1H), 3.07 (s, 1H), 2.95-2.80 (m, 1H), 2.22 (dd, J=3.6, 9.2 Hz, 1H), 1.89-1.72 (m, 2H), 1.70-1.55 (m, 1H), 1.47 (s, 9H).

### Example 15.3. Preparation of tert-butyl 3-(5-(chlorosulfonyl)thiazol-2-yl)piperidine-1-carboxylate

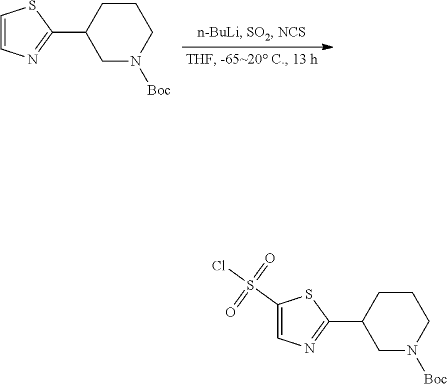 [SMILES: CC(C)(C)OC(=O)N1CCCC(c2nccs2)C1 ; CC(C)(C)OC(=O)N1CCCC(c2ncc(S(=O)(=O)Cl)s2)C1]

To a solution of tert-butyl 3-(thiazol-2-yl)piperidine-1-carboxylate (860 mg, 3.20 mmol, 1.00 eq) in tetrahydrofuran (10.0 mL) was added n-butyllithium (2.50 M, 1.92 mL, 1.50 eq) dropwise at −78° C. and stirred 30 min under nitrogen, and then sulfur dioxide (205 mg, 3.20 mmol, 1.00 eq) was bubbled into at −65° C. for 30 min. The reaction mixture was warmed to 15° C. slowly and stirred at 20° C. for 1 h. N-chloro-succinimide (1.29 g, 9.66 mmol, 3.00 eq) was added to the mixture at 0° C. The mixture was stirred at 15° C. for 12 h. The reaction mixture was poured into water and extracted with dichloromethane (50.0 mL×3). The organic layer was concentrated under reduced pressure to give a residue. The residue was purified by flash silica gel chromatography (ISCO®; 12 g Sepa Flash® Silica Flash Column, Eluent of 0-15% Ethyl acetate/Petroleum ether gradient @40 mL/min). Compound tert-butyl 3-(5-(chlorosulfonyl)thiazol-2-yl)piperidine-1-carboxylate (930 mg, 2.41 mmol, 74.8% yield, 95% purity) was obtained as a colorless oil. MS (ESI) m/z 367.0 [M+H]⁺.

### Example 15.4. Preparation of tert-butyl 3-(5-(N-(4-chloro-1H-indol-7-yl)sulfamoyl)thiazol-2-yl)piperidine-1-carboxylate

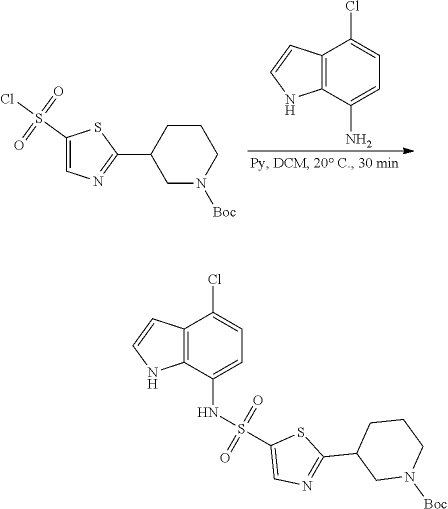 [SMILES: CC(C)(C)OC(=O)N1CCCC(c2ncc(S(=O)(=O)Cl)s2)C1 ; Nc1ccc(Cl)c2cc[nH]c12 ; CC(C)(C)OC(=O)N1CCCC(c2ncc(S(=O)(=O)Nc3ccc(Cl)c4cc[nH]c34)s2)C1]

To a solution of tert-butyl 3-(5-(chlorosulfonyl)thiazol-2-yl)piperidine-1-carboxylate (80 mg, 218 mol, 1.00 eq) in dichloromethane (1.00 mL) was added pyridine (86.2 mg, 1.09 mmol, 88.0 μL, 5.00 eq) and 4-chloro-1H-indol-7-amine (36.3 mg, 218 μmol, 1.00 eq). The mixture was stirred at 20° C. for 0.5 h. The mixture was diluted with water (5.00 mL) and extracted with dichloromethane (5.00 mL×2). The combined organic layers were dried with sodium sulfate solid and filtered. Then the filtrate was concentrated under reduced pressure to dryness. The residue was purified by flash silica gel chromatography (ISCO®; 4 g Sepa Flash® Silica Flash Column, Eluent of 0-10% Ethyl acetate/Petroleum ether gradient @18 mL/min). Compound tert-butyl 3-(5-(N-(4-chloro-1H-indol-7-yl)sulfamoyl)thiazol-2-yl)piperidine-1-carboxylate (90 mg, 173 mol, 79.7% yield, 96% purity) was obtained as a white solid. MS (ESI) m/z 497.2 [M+H]⁺.

### Example 15.5. Preparation of N-(4-chloro-1H-indol-7-yl-2-(piperidin-3-yl)thiazole-5-sulfonamide (Compound 15)

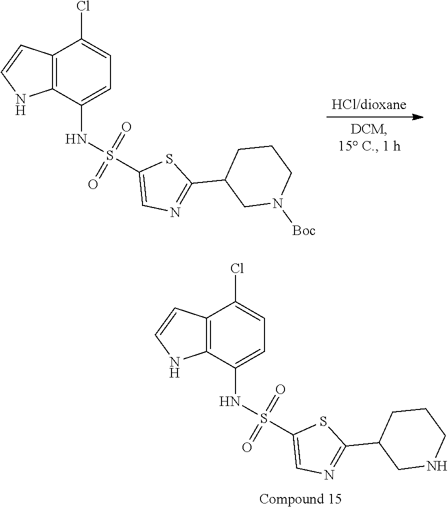 [SMILES: CC(C)(C)OC(=O)N1CCCC(c2ncc(S(=O)(=O)Nc3ccc(Cl)c4cc[nH]c34)s2)C1 ; O=S(=O)(Nc1ccc(Cl)c2cc[nH]c12)c1cnc(C2CCCNC2)s1]

To a solution of tert-butyl 3-(5-(N-(4-chloro-1H-indol-7-yl)sulfamoyl)thiazol-2-yl)piperidine-1-carboxylate (90 mg, 181 mol, 1.00 eq) in dichloromethane (1.00 mL) was added hydrochloric acid/dioxane (4.00 M, 1.00 mL, 22.0 eq). The mixture was stirred at 20° C. for 1 h. The mixture was concentrated in vacuum to get a crude product. The residue was purified by prep-HPLC (column: Phenomenex luna C18 150*25 mm*10 μm; mobile phase: [water(FA)-ACN]; gradient: 15%-45% B over 15 min). The aqueous solution was lyophilized in vacuo to get N-(4-chloro-1H-indol-7-yl)-2-(piperidin-3-yl)thiazole-5-sulfonamide (4.95 mg, 11.1 mol, 6.10% yield, 98.9% purity, formic acid salt) as a white solid. MS (ESI) m/z 397.1 [M+H]⁺. ¹H NMR (400 MHz, DMSO-d₆+D₂O) d=8.19 (s, 1H), 7.81 (s, 1H), 7.16 (d, J=3.2 Hz, 1H), 6.83-6.77 (m, 1H), 6.74-6.68 (m, 1H), 6.26 (d, J=3.2 Hz, 1H), 3.35-3.23 (m, 2H), 3.14 (d, J=12.0 Hz, 1H), 3.04-2.95 (m, 1H), 2.81 (dt, J=3.2, 12.0 Hz, 1H), 2.10-2.00 (m, 1H), 1.84-1.72 (m, 1H), 1.71-1.54 (m, 2H).

### Example 16.1. Preparation of N-(4-chloro-1H-indol-7-yl)-2-(1-methylpiperidin-3-yl)thiazole-5-sulfonamide (Compound 16)

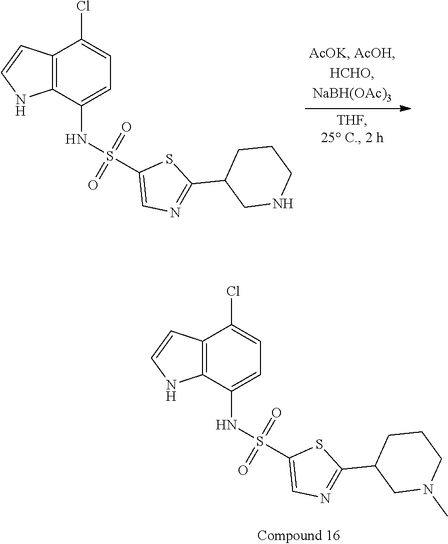 [SMILES: O=S(=O)(Nc1ccc(Cl)c2cc[nH]c12)c1cnc(C2CCCNC2)s1 ; CN1CCCC(c2ncc(S(=O)(=O)Nc3ccc(Cl)c4cc[nH]c34)s2)C1]

To a solution of N-(4-chloro-1H-indol-7-yl)-2-(piperidin-3-yl)thiazole-5-sulfonamide (40.0 mg, 101 mol, 1.00 eq) in tetrahydrofuran (0.500 mL) was added potassium acetate (29.6 mg, 303 μmol, 3.00 eq), acetic acid (18.1 mg, 303 μmol, 17.3 μL, 3.00 eq) and formaldehyde (16.3 mg, 202 μmol, 15.0 μL, 2.00 eq). The mixture was stirred at 25° C. for 1 h. sodium triacetoxyhydroborate (21.3 mg, 101 μmol, 1.00 eq) was added and the mixture was stirred at 25° C. for 1 h. The mixture was blown dry with nitrogen. The residue was purified by prep-HPLC (column: Waters xbridge 150*25 mm 10 μm; mobile phase: [water(NH₄HCO₃)-ACN]; gradient: 18%-48% B over 10 min). The aqueous solution was lyophilized in vacuo. Compound N-(4-chloro-1H-indol-7-yl)-2-(1-methylpiperidin-3-yl)thiazole-5-sulfonamide (8.63 mg, 21.00 μmol, 20.84% yield) was obtained as a yellow gum. MS (ESI) m/z 411.1 [M+H]⁺. ¹H NMR (400 MHz, DMSO-d6) δ=11.07 (s, 1H), 10.39-9.75 (m, 1H), 7.95 (s, 1H), 7.36 (s, 1H), 6.94 (d, J=8.0 Hz, 1H), 6.78 (d, J=8.0 Hz, 1H), 6.41 (s, 1H), 3.29-3.23 (m, 2H), 2.93-2.73 (m, 1H), 2.42 (d, J=8.0 Hz, 2H), 2.26 (s, 3H), 1.86 (d, J=6.4 Hz, 1H), 1.67-1.39 (m, 3H).

### Example 17. Synthetic Scheme of Compound 17

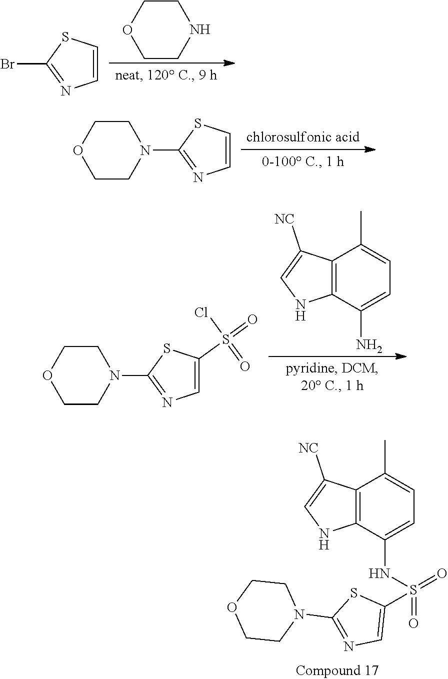

### Example 17.1. Preparation of 4-thiazol-2-ylmorpholine

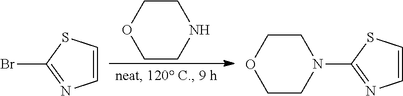

To a solution of 2-bromothiazole (500 mg, 3.05 mmol, 275 μL, 1.00 eq) in morpholine (4.95 g, 56.8 mmol, 5.00 mL, 18.6 eq). The mixture was stirred at 120° C. for 9 h. The reaction mixture was quenched by addition concentrated hydrochloric acid (1 M 10.0 mL at 0° C.), and then extracted with ethyl acetate (20.0 mL). The combined organic layers were washed with brine (15.0 mL), dried over sodium sulfate, filtered and concentrated under reduced pressure to give a residue. The residue was purified by column chromatography (SiO₂, Petroleum ether/Ethyl acetate=1/0 to 3/1) and concentrated under reduced pressure to give the 4-thiazol-2-ylmorpholine (500 mg, 2.79 mmol, 91% yield, 95% purity) as a yellow oil. ¹H NMR (400 MHz, DMSO-d₆) δ=7.19 (d, J=3.6 Hz, 1H), 6.88 (d, J=3.6 Hz, 1H), 3.73-3.67 (m, 4H), 3.38-3.32 (m, 4H).

### Example 17.2. Preparation of 2-morpholinothiazole-5-sulfonyl chloride

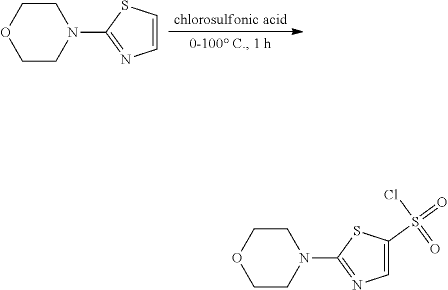 [SMILES: c1csc(N2CCOCC2)n1 ; O=S(=O)(Cl)c1cnc(N2CCOCC2)s1]

A mixture of 4-thiazol-2-ylmorpholine (100 mg, 587 mol, 1.00 eq) in sulfurochloridic acid (1.75 g, 15.0 mmol, 1.00 mL, 25.6 eq) was degassed and purged with nitrogen for 3 times, and then the mixture was stirred at 100° C. for 1 h under nitrogen atmosphere. The reaction mixture was quenched by addition water (10.0 mL) at 0° C., and then extracted with ethyl acetate (20.0 mL). The combined organic layers were dried over sodium sulfate, filtered and concentrated under reduced pressure to give the 2-morpholinothiazole-5-sulfonyl chloride (50 mg, crude) was obtained as a white solid.

### Example 17.3. Preparation of N-(3-cyano-4-methyl-1H-indol-7-yl)-2-morpholino-thiazole-5-sulfonamide (Compound 17)

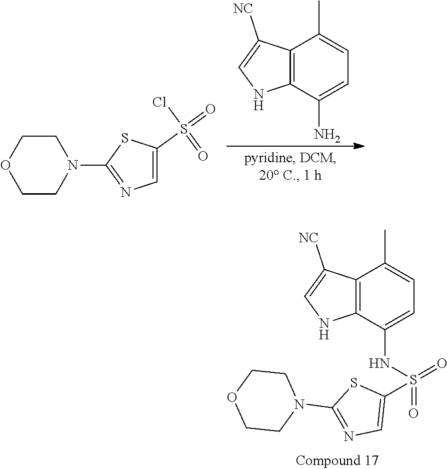 [SMILES: O=S(=O)(Cl)c1cnc(N2CCOCC2)s1 ; Cc1ccc(N)c2[nH]cc(C#N)c12 ; Cc1ccc(NS(=O)(=O)c2cnc(N3CCOCC3)s2)c2[nH]cc(C#N)c12]

To a solution of 2-morpholinothiazole-5-sulfonyl chloride (40.0 mg, 149 μmol, 1.00 eq) in dichloromethane (0.500 mL) was added Pyridine (11.8 mg, 149 mol, 12.0 μL, 1.00 eq) and 7-amino-4-methyl-1H-indole-3-carbonitrile (25.5 mg, 149 μmol, 1.0 eq). The mixture was stirred at 20° C. for 1 h. The reaction mixture was concentrated under reduced pressure to remove solvent. The crude product was purified by prep-HPLC (column: Phenomenex luna C18 150*25 mm*10 μm; mobile phase: [water(FA)-ACN]; gradient: 26%-56% B over 10 min) and lyophilized to give the N-(3-cyano-4-methyl-1H-indol-7-yl)-2-morpholino-thiazole-5-sulfonamide (7.40 mg, 18.2 μmol, 12.2% yield, 99.1% purity) as a pink solid. MS (ESI) m/z 404.3 [M+H]⁺. ¹H NMR (400 MHz, DMSO-d₆) 3=12.09-11.72 (m, 1H), 8.12 (s, 1H), 7.44 (s, 1H), 6.91-6.77 (m, 2H), 3.67 (t, J=4.8 Hz, 4H), 3.42-3.39 (m, 4H), 2.58 (s, 3H).

### Examples 18 and 19. Synthetic Scheme of Compound 18 and Compound 19

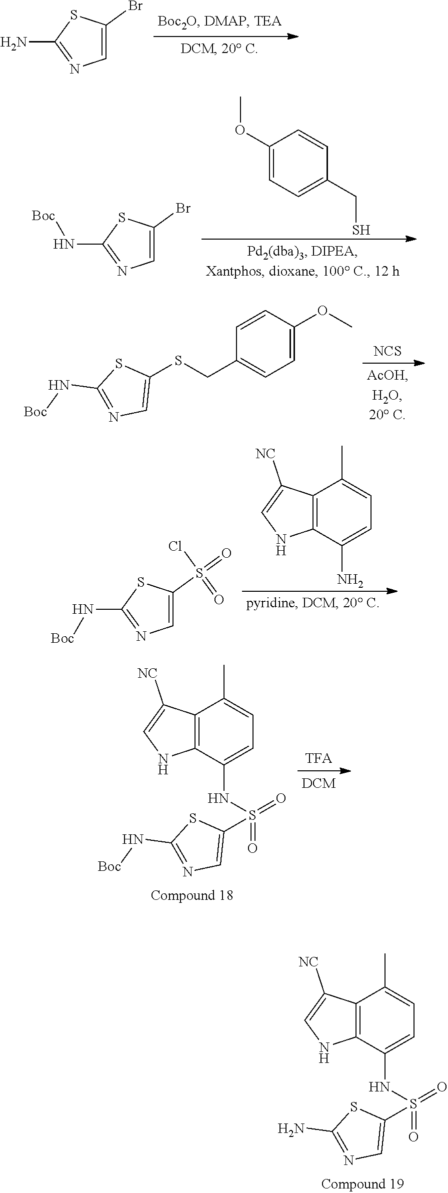

### Example 18.1. Preparation of tert-butyl (5-bromothiazol-2-yl)carbamate

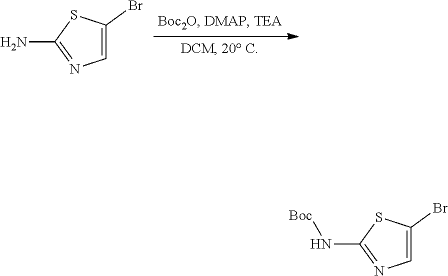 [SMILES: Nc1ncc(Br)s1 ; CC(C)(C)OC(=O)Nc1ncc(Br)s1]

To a solution of 5-bromothiazol-2-amine (5.00 g, 27.9 mmol, 1.00 eq) in dichloromethane (60.0 mL) was added 4-dimethylaminopyridine (341 mg, 2.79 mmol, 0.100 eq) and di-tert-butyldicarbonate (9.14 g, 41.8 mmol, 9.62 mL, 1.50 eq) at 16° C. The mixture was stirred at 16° C. for 2 h. The mixture was poured into water (100 mL) and extracted with dichloromethane (100 mL×3). The combined organic layer was concentrated in vacuum. The residue was purified by column chromatography (SiO₂, Petroleum ether/Ethyl acetate=100/1 to 100/1, TLC(petroleum ether: ethyl acetate=10:1), Rf(R=0.2, P=0.6)). The crude product was triturated with Petroleum ether at 20° C. for 15 min. The mixture was filtered and the filter cake was collected. tert-butyl (5-bromothiazol-2-yl)carbamate (4.7 g, 16.84 mmol, 60.29% yield) was obtained as a white solid. ¹H NMR (400 MHz, DMSO-d₆)=11.73 (s, 1H), 7.43 (s, 1H), 1.48 (s, 9H).

### Example 18.2. Preparation of tert-butyl (5-((4-methoxybenzyl)thio)thiazol-2-yl)carbamate

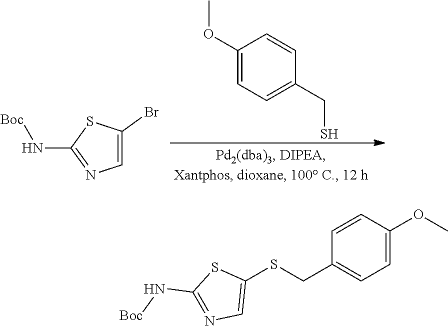

To a solution of tert-butyl (5-bromothiazol-2-yl)carbamate (1.00 g, 3.58 mmol, 1.00 eq) and (4-methoxyphenyl)methanethiol (662 mg, 4.30 mmol, 598 μL, 1.20 eq) in dioxane (10.0 mL) was added N,N-diisopropylethylamine (462 mg, 3.58 mmol, 623 μL, 1.00 eq), 4,5-bis(diphenylphosphino)-9,9-dimethylxanthene (414 mg, 716 μmol, 0.200 eq) and tris(dibenzylideneacetone)dipalladium(0) (205 mg, 358 μmol, 0.100 eq) at 16° C. The mixture was stirred at 100° C. for 16 h. The mixture was stirred at 100° C. for 16 h. The mixture was poured into water (50 mL) and extracted with dichloromethane (50 mL×3). The combined organic layer was concentrated in vacuum. The residue was purified by column chromatography (SiO₂, Petroleum ether/Ethyl acetate=100/1 to 10/1, TLC(petroleum ether:ethyl acetate=5:1), Rf(R=0.7, P=0.3)). Tert-butyl (5-((4-methoxybenzyl)thio)thiazol-2-yl)carbamate (590 mg, 1.67 mmol, 46% yield) was obtained as a yellow solid. ¹H NMR (400 MHz, CDCl₃) δ=7.12-7.03 (m, 3H), 6.86-6.74 (m, 2H), 3.85 (s, 2H), 3.79 (s, 3H), 3.71 (s, 1H), 1.54 (s, 9H).

### Example 18.3. Preparation of tert-butyl (5-(chlorosulfonyl)thiazol-2-yl)carbamate

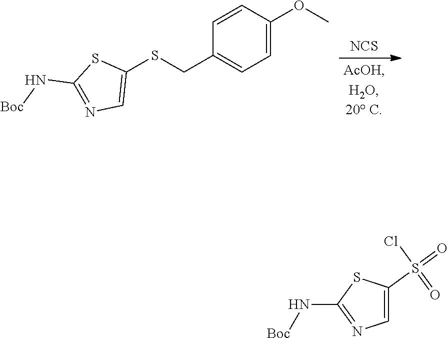

To a solution of tert-butyl (5-((4-methoxybenzyl)thio)thiazol-2-yl)carbamate (100 mg, 283 μmol, 1.00 eq) in acetic acid (4.00 mL), water(1.00 mL) and dichloromethane (1.00 mL) was added N-chlorosuccinimide (113.65 mg, 851.13 mol, 3 eq) at 0° C. The mixture was stirred 16° C. for 2 h. The mixture was poured into water (10 mL) and extracted with ethyl acetate (20 mL×3). The combined organic layer was washed with brine (10 mL), dried over with sodium sulfate and concentrated in vacuum. tert-butyl (5-(chlorosulfonyl)thiazol-2-yl)carbamate (80.0 mg, crude) was obtained as a yellow solid.

### Example 18.4. Preparation of tert-butyl (5-(N-(3-cyano-4-methyl-1H-indol-7-yl)sulfamoyl)thiazol-2-yl)carbamate (Compound 18)

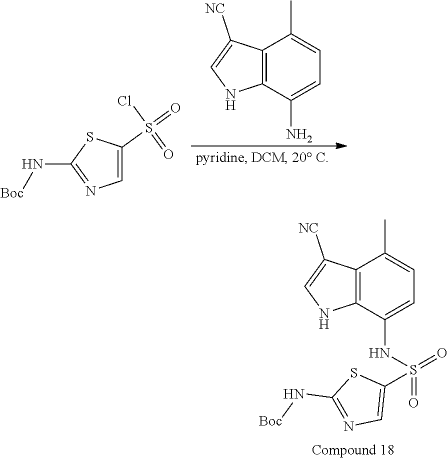 [SMILES: CC(C)(C)OC(=O)Nc1ncc(S(=O)(=O)Cl)s1 ; Cc1ccc(N)c2[nH]cc(C#N)c12 ; Cc1ccc(NS(=O)(=O)c2cnc(NC(=O)OC(C)(C)C)s2)c2[nH]cc(C#N)c12]

To a solution of 7-amino-4-methyl-1H-indole-3-carbonitrile (41.2 mg, 240 μmol, 0.900 eq) in dichloromethane (1.00 mL) was added pyridine (211 mg, 2.68 mmol, 216 L, 10.0 eq) and tert-butyl (5-(chlorosulfonyl)thiazol-2-yl)carbamate (80.0 mg, 267 μmol, 1.00 eq) at 0° C. The mixture was stirred at 16° C. for 10 min. The mixture was poured into water (5.00 mL) and extracted with dichloromethane (15.0 mL×2). The combined organic layer was concentrated in vacuum. The crude product was purified by prep-HPLC(column: Phenomenex luna C18 150*25 mm*10 μm; mobile phase: [water(FA)-ACN]; gradient: 35%-65% B over 8 min). tert-butyl (5-(N-(3-cyano-4-methyl-1H-indol-7-yl)sulfamoyl)thiazol-2-yl)carbamate (40 mg, 90.43 μmol, 33.77% yield, 98% purity) was obtained as a yellow solid, 8.06 mg of product was used for delivery. MS (ESI) m/z 434.0/334.0 [M+H]⁺. ¹H NMR (400 MHz, CDCl₃)=9.63 (s, 1H), 9.33-9.01 (m, 1H), 7.80 (d, J=2.8 Hz, 1H), 7.50 (s, 1H), 6.86 (d, J=8.0 Hz, 1H), 6.67-6.56 (m, 2H), 2.76 (s, 3H), 1.52 (s, 9H).

### Example 19.1. Preparation of 2-amino-N-(3-cyano-4-methyl-1H-indol-7-yl)thiazole-5-sulfonamide (Compound 19

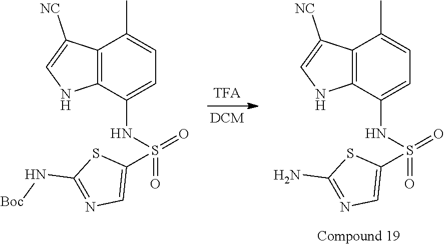 [SMILES: Cc1ccc(NS(=O)(=O)c2cnc(NC(=O)OC(C)(C)C)s2)c2[nH]cc(C#N)c12 ; Cc1ccc(NS(=O)(=O)c2cnc(N)s2)c2[nH]cc(C#N)c12]

To a solution of tert-butyl (5-(N-(3-cyano-4-methyl-1H-indol-7-yl)sulfamoyl)thiazol-2-yl)carbamate (25.0 mg, 57.6 μmol, 1.00 eq) in dichloromethane (0.5 mL) was added hydrochloric acid/dioxane (4 M, 1 mL, 69.3 eq) at 16° C. The mixture was stirred at 16° C. for 0.5 h. LCMS showed reactant 1 remained, trifluoroacetic acid (153 mg, 1.35 mmol, 0.1 mL, 23.3 eq) was added to the mixture. The resulting mixture was stirred at 16° C. for 2.5 h. The mixture was concentrated in vacuum. The mixture was purification by prep-HPLC (column: Phenomenex Luna C18 150*30 mm*5 μm; mobile phase: [water(HCl)-ACN]; gradient: 18%-48% B over 10 min). 2-amino-N-(3-cyano-4-methyl-1H-indol-7-yl)thiazole-5-sulfonamide (8.48 mg, 24.67 μmol, 42.78% yield, 97% purity) was obtained as off-white solid. MS (ESI) m/z 334.2 [M+H]⁺. ¹H NMR (400 MHz, DMSO-d₆) δ=11.95 (d, J=2.4 Hz, 1H), 9.89 (s, 1H), 8.18 (d, J=3.2 Hz, 1H), 8.04-7.68 (m, 2H), 7.26 (s, 1H), 6.91-6.81 (m, 2H), 2.60 (s, 3H).

### Example 20. Preparation of N-(3-chloro-4-methyl-1H-indol-7-yl)-2-methyl-thiazole-5-sulfonamide (Compound 20)

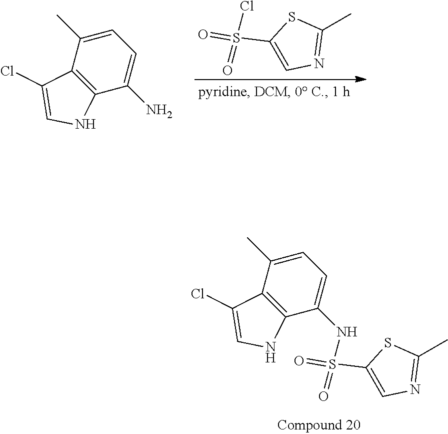

To a solution of 3-chloro-4-methyl-1H-indol-7-amine (50.0 mg, 276 mol, 1.00 eq) in dichloromethane (1.00 mL) was added Py (65.7 mg, 830 μmol, 67.0 μL, 3.00 eq) and 2-methylthiazole-5-sulfonyl chloride (60.2 mg, 304 μmol, 1.10 eq) at 0° C. The mixture was stirred at 0° C. for 1h. The mixture was concentrated under reduced pressure to give a residue. The crude product was purified by prep-HPLC(column: Phenomenex luna C18 150*25 mm*10 μm; mobile phase: [water(FA)-ACN]; gradient: 40%-70% B over 8 min). N-(3-chloro-4-methyl-1H-indol-7-yl)-2-methyl-thiazole-5-sulfonamide was obtained as solid. MS (ESI) m/z 342.1 [M+H]⁺. ¹H NMR (400 MHz, DMSO-d₆) δ=11.05 (s, 1H), 10.13 (s, 1H), 7.91 (s, 1H), 7.41 (d, J=2.8 Hz, 1H), 6.74-6.65 (m, 2H), 2.67 (s, 3H), 2.63 (s, 3H).

### Example 21. Synthetic Scheme of Compound 21

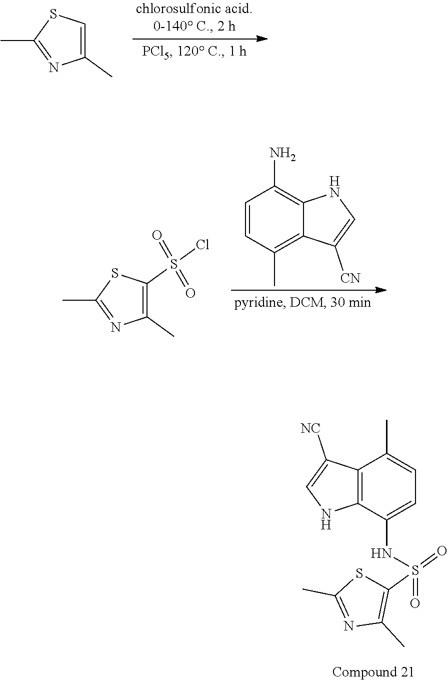

### Example 21.1. Preparation of 2,4-dimethylthiazole-5-sulfonyl chloride

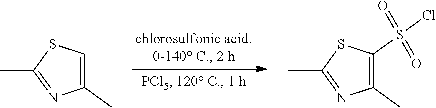

To a solution of 2,4-dimethylthiazole (200 mg, 1.77 mmol, 1.00 eq) added to sulfurochloridic acid (877 mg, 7.52 mmol, 500 μL, 4.26 eq) at 0° C. under N₂, and then the mixture was stirred at 140° C. for 16 h under N₂. pentachloro-phosphane (735 mg, 3.53 mmol, 2.00 eq) added to the mixture at 120° C., and then the mixture was stirred at 120° C. for 1h. The reaction mixture was poured into water and extracted with dichloromethane 50.0 mL×3. The organic layer was concentrated under reduced pressure to give a residue. The residue was purified by flash silica gel chromatography (ISCO®; 4 g SepaFlash® Silica Flash Column, Eluent of 0-25% Ethyl acetate/Petroleum ether gradient @40 mL/min). 2,4-Dimethylthiazole-5-sulfonyl chloride (370 mg, 1.73 mmol, 97% yield, 99% purity) was obtained as a yellow oil. MS (ESI) m/z 212.2/214.2 [M+H]⁺.

### Example 21.2. Preparation of N-(3-cyano-4-methyl-1H-indol-7-yl)-2,4-dimethylthiazole-5-sulfonamide (Compound 21)

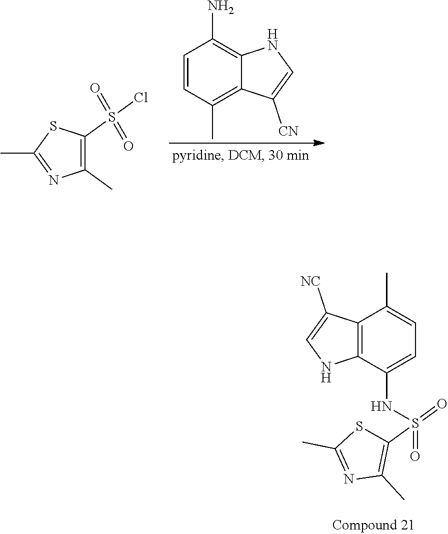

To a solution of 2,4-dimethylthiazole-5-sulfonyl chloride (49.5 mg, 234 mol, 1.00 eq) in dichloromethane (1.00 mL) was added 7-amino-4-methyl-1H-indole-3-carbonitrile (40.0 mg, 234 mol, 1.00 eq) and pyridine (55.4 mg, 701 mol, 56.6 L, 3.00 eq) at 16° C. The mixture was stirred at 25° C. for 1 h. The mixture was poured into water (20 mL), and then extracted with dichloromethane (20 mL×2). The combined organic layers were dried over sodium sulphate, filtered and concentrated in vacuum to give a residue. The residue was purified by prep-HPLC (column: Waters xbridge 150*25 mm 10 μm; mobile phase: [water(NH₄HCO₃)-ACN]; gradient: 15%-45% B over 10 min). N-(3-cyano-4-methyl-1H-indol-7-yl)-2,4-dimethyl-thiazole-5-sulfonamide (22.87 mg, 66.02 μmol, 28.25% yield, 100% purity) was obtained as a yellow solid. MS (ESI) m/z 347.2 [M+H]⁺. ¹H NMR (400 MHz, DMSO-d₆) δ=11.95 (d, J=1.2 Hz, 1H), 10.20 (s, 1H), 8.18 (s, 1H), 6.86 (d, J=7.6 Hz, 1H), 6.65 (d, J=7.6 Hz, 1H), 2.61 (s, 3H), 2.59 (s, 3H), 2.18 (s, 3H).

### Example 22. Synthetic Scheme of Compound 22

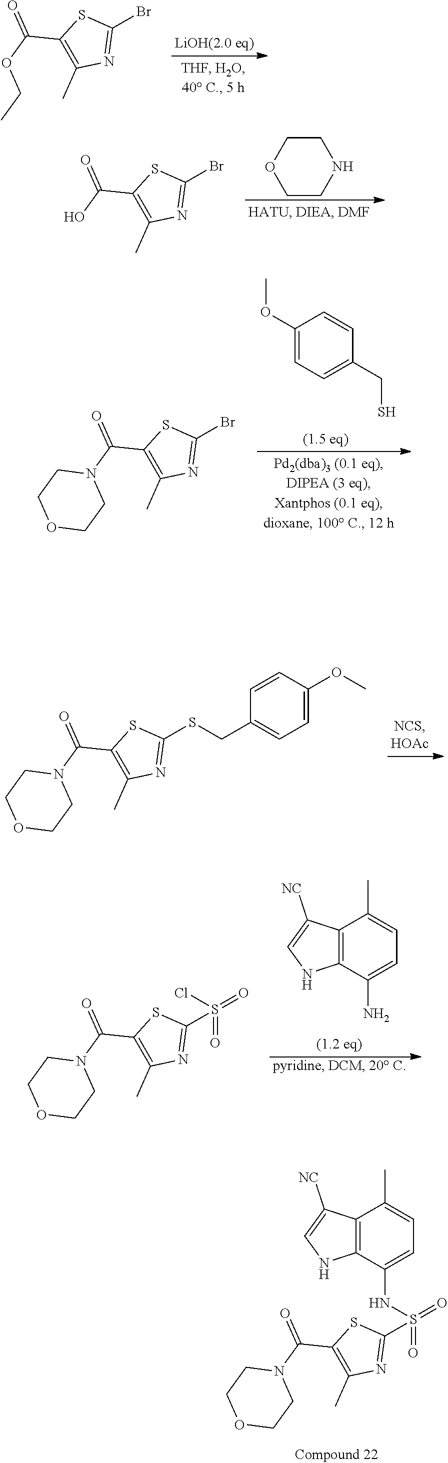

### Example 22.1. Preparation of 2-bromo-4-methylthiazole-5-carboxylic acid

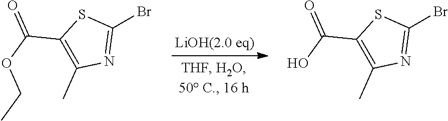

To a solution of ethyl 2-bromo-4-methylthiazole-5-carboxylate (2.00 g, 8.00 mmol, 1.00 eq) in tetrahydrofuran (20.0 mL) was added lithium hydroxide (671 mg, 16.0 mmol, 2.00 eq), water (2 mL) and ethanol (1.00 mL) at 20° C., the mixture was stirred at 50° C. for 16 h. The mixture was acidified by concentrated hydrochloric acid (1 M) adjust to pH=2, and then extracted with ethyl acetate (2×100 mL). The combined organic layers were dried over sodium sulfate, filtered and concentrated in vacuum. 2-bromo-4-methylthiazole-5-carboxylic acid (1.6 g, crude) was obtained as a yellow solid. MS (ESI) m/z 221.9/223.9. [M+H]⁺. ¹H NMR (400 MHz, DMSO-d₆) δ ppm 13.27-14.14 (m, 1H) 2.61 (s, 3H).

### Example 22.2. Preparation of (2-bromo-4-methylthiazol-5-yl)(morpholino)methanone

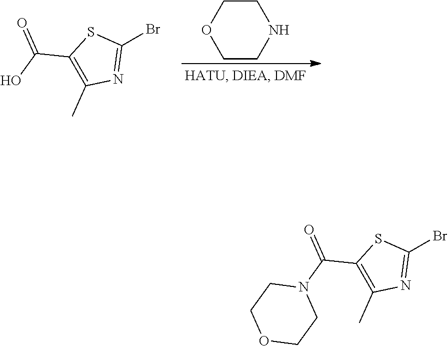

To a solution of 2-bromo-4-methylthiazole-5-carboxylic acid (500 mg, 2.25 mmol, 1.00 eq) in dimethylformamide (5.00 mL) was added O-(7-azabenzotriazol-1-yl)-N,N,N,N-tetramethyluroniumhexafluorophosphate (1.28 g, 3.38 mmol, 1.50 eq) and N,N-diisopropylethylamine (582 mg, 4.50 mmol, 784 μL, 2.00 eq) at 20° C., the mixture was stirred at 20° C. for 30 min, and then added morpholine (235 mg, 2.70 mmol, 237 μL, 1.20 eq) at 20° C., the mixture was stirred at 20° C. for 2 h. The mixture was diluted with water (100 mL). And then extracted with ethyl acetate (2×100 mL). The combined organic layers were dried over sodium sulfate, and concentrated in vacuum to give a residue. The crude product was purified by column chromatography on silica gel eluted with petroleum ether/ethyl acetate=100:1 to 1:1. (2-bromo-4-methylthiazol-5-yl)(morpholino)methanone (500 mg, 1.72 mmol, 76% yield) was obtained as a yellow solid. MS (ESI) m/z 291.0/292.9. [M+H]⁺. ¹H NMR (400 MHz, DMSO-d₆) δ ppm 3.56-3.63 (m, 4H) 3.50 (s, 4H) 2.33 (s, 3H).

### Example 22.3 Preparation of (2-((4-methoxybenzyl)thio)-4-methylthiazol-5-yl)(morpholino)methanone

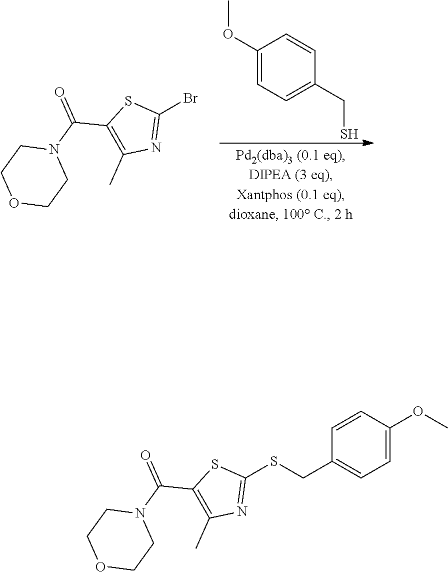

To a solution of (2-bromo-4-methylthiazol-5-yl)(morpholino)methanone (500 mg, 1.72 mmol, 1.00 eq) in dioxane (8.00 mL) was added N,N-diisopropylethylamine (443 mg, 3.43 mmol, 598 μL, 2.00 eq), (4-methoxyphenyl)methanethiol (529 mg, 3.43 mmol, 478 μL, 2.00 eq), 4,5-bis(diphenylphosphino)-9,9-dimethylxanthene (99.3 mg, 171 μmol, 0.100 eq) and tris(dibenzylideneacetone)dipalladium(0) (314.50 mg, 343 μmol, 0.200 eq) at 20° C., the mixture was stirred at 100° C. for 2 h under nitrogen. The mixture was diluted with water (100 mL). And then extracted with ethyl acetate (2×100 mL). The combined organic layers were dried over sodium sulfate, and concentrated in vacuum to give a residue. The crude product was purified by column chromatography on silica gel eluted with petroleum ether/ethyl acetate=100:1 to 1:1. (2-((4-methoxybenzyl)thio)-4-methylthiazol-5-yl)(morpholino)methanone (500 mg, 1.37 mmol, 79.88% yield) was obtained as a yellow solid. MS (ESI) m/z 365.3 [M+H]⁺. ¹H NMR (400 MHz, DMSO-d₆) δ ppm 7.36 (d, J=8.4 Hz, 2H) 6.90 (d, J=8.4 Hz, 2H) 4.43 (s, 2H) 3.74 (s, 3H) 3.59 (d, J=4.4 Hz, 4H) 3.48 (s, 4H) 2.31 (s, 3H).

### Example 22.4. Preparation of 4-methyl-5-(morpholine-4-carbonyl)thiazole-2-sulfonyl chloride

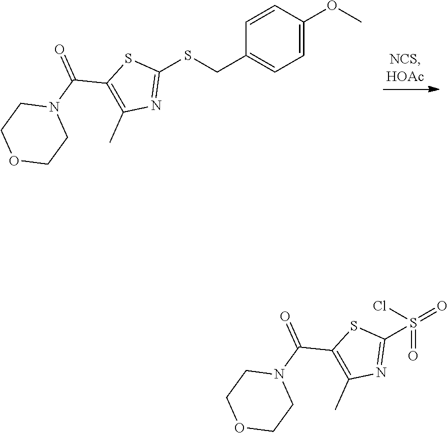 [SMILES: COc1ccc(CSc2nc(C)c(C(=O)N3CCOCC3)s2)cc1 ; Cc1nc(S(=O)(=O)Cl)sc1C(=O)N1CCOCC1]

To a solution of (2-((4-methoxybenzyl)thio)-4-methylthiazol-5-yl)(morpholino)methanone (100 mg, 274 μmol, 1.00 eq) in acetic acid (3 mL) and water (1.00 mL) was added 1-chloropyrrolidine-2,5-dione (146 mg, 1.10 mmol, 4.00 eq) at 0° C., the mixture was stirred at 20° C. for 1 h. The mixture was diluted with ice water (10.0 mL). And then extracted with dichloromethane (2×20.0 mL). The combined organic layers were dried over sodium sulfate, and concentrated in vacuum to give a residue. It was not purified and used for the next step. 4-methyl-5-(morpholine-4-carbonyl)thiazole-2-sulfonyl chloride (80.0 mg, crude) was obtained as a yellow oil. MS (ESI) m/z 311.0/313.0. [M+H]⁺.

### Example 22.5. Preparation of N-(3-cyano-4-methyl-1H-indol-7-yl)-4-methyl-5-(morpholine-4-carbonyl)thiazole-2-sulfonamide (Compound 22)

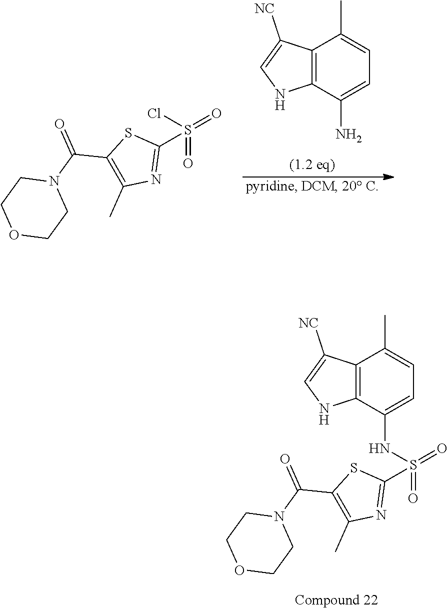 [SMILES: Cc1nc(S(=O)(=O)Cl)sc1C(=O)N1CCOCC1 ; Cc1ccc(N)c2[nH]cc(C#N)c12 ; "pyridine, DCM, 20° C." ; Cc1nc(S(=O)(=O)Nc2ccc(C)c3c(C#N)c[nH]c23)sc1C(=O)N1CCOCC1]

To a solution of 7-amino-4-methyl-1H-indole-3-carbonitrile (48.4 mg, 283 μmol, 1.10 eq) in dichloromethane (1.00 mL) was added pyridine (20.3 mg, 257 μmol, 20.7 μL, 1.00 eq) and 4-methyl-5-(morpholine-4-carbonyl)thiazole-2-sulfonyl chloride (80.0 mg, 257 mol, 1.00 eq) at 0° C., the mixture was stirred at 0° C. for 10 min. The mixture was diluted with water (20.0 mL). And then extracted with ethyl acetate (2×20.0 mL). The combined organic layers were dried over sodium sulfate, and concentrated in vacuum to give a residue. The crude product was purified by prep-HPLC (column: Phenomenex luna C18 150*25 mm*10 μm; mobile phase: [water(FA)-ACN]; gradient: 27%-57% B over 8 min). The desired fraction was lyophilized. N-(3-cyano-4-methyl-1H-indol-7-yl)-4-methyl-5-(morpholine-4-carbonyl)thiazole-2-sulfonamide (15.54 mg, 34.88 μmol, 13.55% yield, 100% purity) was obtained as a yellow solid. MS (ESI) m/z 446.0. [M+H]⁺. ¹H NMR (400 MHz, DMSO-d₆) δ ppm 12.06 (s, 1H) 10.79 (s, 1H) 8.20 (s, 1H) 6.85 (d, J=7.2 Hz, 1H) 6.73 (d, J=7.2 Hz, 1H) 3.59 (s, 4H) 3.33-3.52 (m, 4H) 2.60 (s, 3H) 2.40 (s, 3H).

### Example 23. Synthetic Scheme of Compound 23

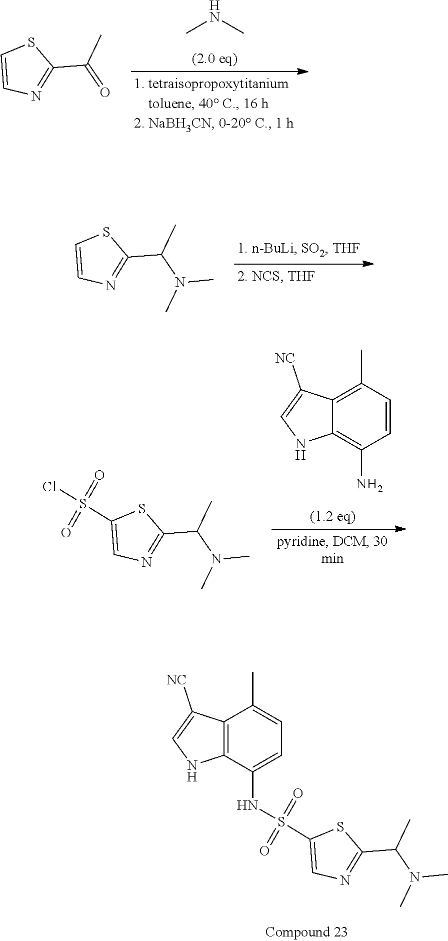

### Example 23.1. Preparation of N,N-dimethyl-1-(thiazol-2-yl)ethan-1-amine

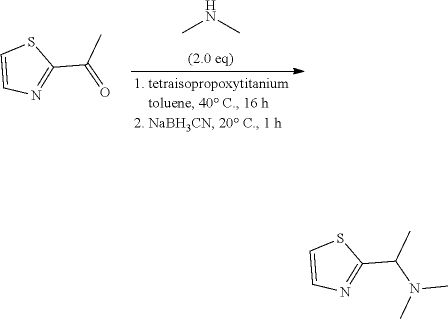

To a solution of 1-thiazol-2-ylethanone (1.00 g, 7.86 mmol, 815 μL, 1.00 eq), N-methylmethanamine (2 M, 7.86 mL, 2.00 eq) and tetraisopropoxytitanium (4.82 g, 16.9 mmol, 5.00 mL, 2.15 eq) in toluene (10.0 mL) the mixture was stirred at 40° C. for 16 h, And then added sodium cyanoborohydride (1.98 g, 31.4 mmol, 4.00 eq) at 20° C., the mixture was stirred at 20° C. for 1 h. The mixture was diluted with water (50 mL). And then extracted with ethyl acetate (2×50.0 mL). The combined organic layers were dried over sodium sulfate, and concentrated in vacuum to give a residue. The crude product was purified by prep-HPLC (column: Waters xbridge 150*25 mm 10 μm; mobile phase: [water(NH₄HCO₃)-ACN]; gradient: 6%-36% B over 10 min). The desired fraction was lyophilized. N,N-dimethyl-1-thiazol-2-yl-ethanamine (120 mg, 768.01 μmol, 9% yield) was obtained as a yellow liquid.

MS (ESI) m/z 179.1. [M+Na]⁺. ¹H NMR (400 MHz, CHLOROFORM) 6 ppm 7.71 (d, J=3.2 Hz, 1H) 7.28 (d, J=3.2 Hz, 1H) 3.96 (q, J=6.8 Hz, 1H) 2.32 (s, 6H) 1.48 (d, J=6.8 Hz, 3H).

### Example 23.2. Preparation of 2-(1-(dimethylamino)ethyl)thiazole-5-sulfonyl chloride

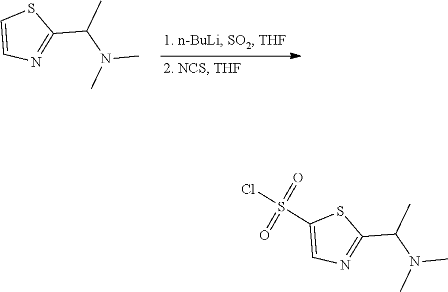

To a solution of N,N-dimethyl-1-(thiazol-2-yl)ethan-1-amine (100 mg, 640 mol, 1.00 eq) in tetrahydrofuran (3.00 mL) was added n-butyllithium (2.5 M, 384 μL, 1.50 eq) at −78° C. The mixture was stirred at −78° C. for 0.5 h. sulfur dioxide (41.0 mg, 640 μmol, 1.00 eq) was bubbled into at −65° C. for 30 min. The reaction mixture was warmed to 20° C. slowly and stirred for 2 h. The mixture was stirred at 20° C. for 1 h. The mixture was diluted with ice water (20.0 mL). And then extracted with ethyl acetate (2×20.0 mL). The combined organic layers were dried over sodium sulfate, and concentrated in vacuum to give a residue. It was not purified and used for the next step. 2-[1-(dimethylamino)ethyl]thiazole-5-sulfinic acid (141 mg, crude) was obtained as a yellow oil. MS (ESI) m/z 221.1. [M+H]⁺.

To a solution of 2-[1-(dimethylamino)ethyl]thiazole-5-sulfinic acid (141 mg, 640 μmol, 1.00 eq) in tetrahydrofuran (3.00 mL) was added 1-chloropyrrolidine-2,5-dione (256 mg, 1.92 mmol, 3.00 eq) at 0° C., the mixture was stirred at 20° C. for 16 h. The mixture was diluted with ice water (20.0 mL). And then extracted with dichloromethane (2×20.0 mL). The combined organic layers were dried over sodium sulfate, and concentrated in vacuum to give a residue. It was not purified and used for the next step. 2-(1-(dimethylamino)ethyl)thiazole-5-sulfonyl chloride (80.0 mg, 314 mol, 49% yield) was obtained as a yellow oil. MS (ESI) m/z 255.1/257.1. [M+H]⁺.

### Example 23.3. Preparation of N-(3-cyano-4-methyl-1H-indol-7-yl)-2-(1-(dimethylamino)ethyl)thiazole-5-sulfonamide (Compound 23)

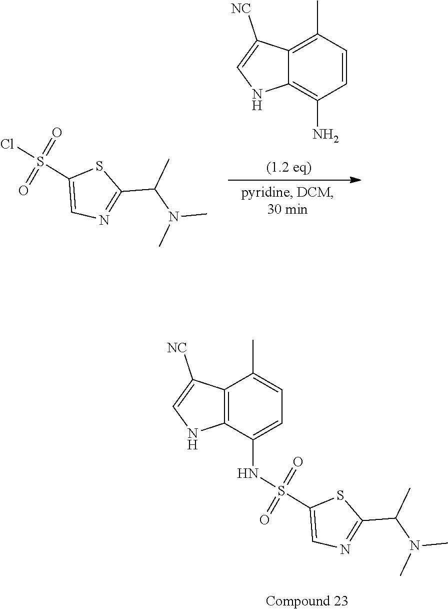 [SMILES: CC(c1ncc(S(=O)(=O)Cl)s1)N(C)C ; Cc1ccc(N)c2[nH]cc(C#N)c12 ; Cc1ccc(NS(=O)(=O)c2cnc(C(C)N(C)C)s2)c2[nH]cc(C#N)c12]

To a solution of 7-amino-4-methyl-1H-indole-3-carbonitrile (26.8 mg, 157 mol, 1.00 eq) in dichloromethane (1.00 mL) was added pyridine (24.8 mg, 314 mol, 25.3 μL, 2.00 eq) and 2-(1-(dimethylamino)ethyl)thiazole-5-sulfonyl chloride (80.0 mg, 157 μmol, 1.00 eq) at 0° C., the mixture was stirred at 0° C. for 20 min. The mixture was diluted with water (20.0 mL). And then extracted with dichloromethane (2×20.0 mL). The combined organic layers were dried over sodium sulfate, and concentrated in vacuum to give a residue. The crude product was purified by prep-HPLC (column: Waters Xbridge C18 150*50 mm*10 μm; mobile phase: [water(NH3H2O)-ACN]; gradient: 3%-33% B over 10 min). The desired fraction was lyophilized. N-(3-cyano-4-methyl-1H-indol-7-yl)-2-(1-(dimethylamino)ethyl)thiazole-5-sulfonamide (7.99 mg, 20.31 μmol, 12.93% yield, 99% purity) was obtained as a yellow solid. MS (ESI) m/z 390.0. [M+H]⁺. ¹H NMR (400 MHz, DMSO-d₆) δ ppm 11.84-11.96 (m, 1H) 10.10-10.29 (m, 1H) 8.16 (s, 1H) 7.86-8.01 (m, 1H) 6.81-6.95 (m, 1H) 6.67-6.78 (m, 1H) 3.88-4.05 (m, 1H) 2.60 (s, 3H) 2.17 (s, 6H) 1.28 (d, J=6.0 Hz, 3H).

### Example 24. Synthetic Scheme of Compound 24

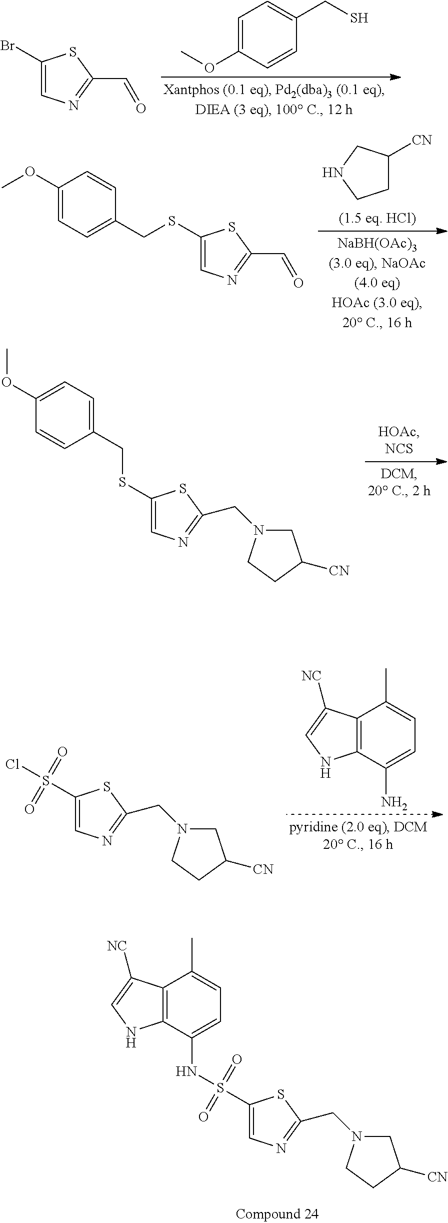

### Example 24.1. Preparation of 5-((4-methoxybenzyl)thio)thiazole-2-carbaldehyde

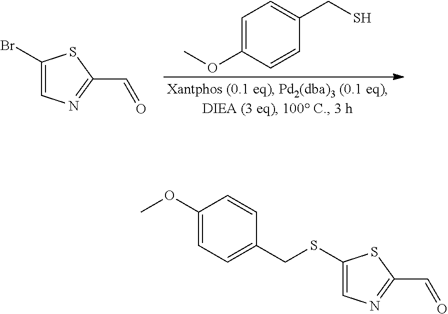

A mixture of (4-methoxyphenyl)methanethiol (386 mg, 2.50 mmol, 348 μL, 1.20 eq), 5-bromothiazole-2-carbaldehyde (400 mg, 2.08 mmol, 1.00 eq), 4,5-bis(diphenylphosphino)-9,9-dimethylxanthene (120 mg, 208 mol, 0.100 eq), tris(dibenzylideneacetone)dipalladium(0) (191 mg, 208 μmol, 0.100 eq) and N,N-diisopropylethylamine (808 mg, 6.25 mmol, 1.09 mL, 3.00 eq) in dioxane (4.00 mL) was degassed and purged with nitrogen for 3 times, and then the mixture was stirred at 100° C. for 3 hr under nitrogen atmosphere. The mixture was diluted with water (50.0 mL) and extracted with ethyl acetate (50.0 mL×2), dried over sodium sulfate, filtered and concentrated under reduced pressure. The residue was purified by silica gel column chromatography (petroleum ether/ethyl acetate=10/1-1/1). Compound 5-[(4-methoxyphenyl)methylsulfanyl]thiazole-2-carbaldehyde (380 mg, 1.32 mmol, 63% yield, 92% purity) was obtained as a white solid. ¹H NMR (400 MHz, chloroform) 6=9.84 (s, 1H), 7.79 (S, 1H), 7.19-7.16 (m, 2H), 6.86-6.83 (m, 2H), 4.09 (s, 2H), 3.78 (s, 3H).

### Example 24.2. Preparation of 1-((5-((4-methoxybenzyl)thio)thiazol-2-yl)methyl)pyrolidine-3-carbonitrile

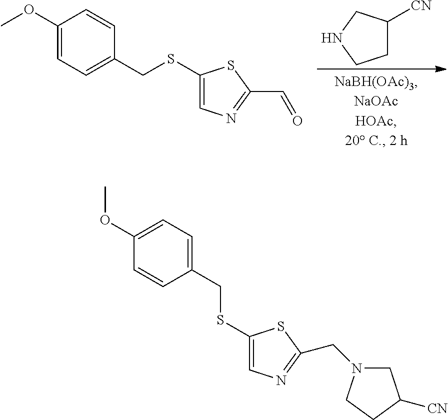 [SMILES: COc1ccc(CSc2cnc(C=O)s2)cc1 ; N#CC1CCNC1 ; COc1ccc(CSc2cnc(CN3CCC(C#N)C3)s2)cc1]

To a solution of pyrrolidine-3-carbonitrile (130 mg, 980 μmol, 1.30 eq, HCl) in tetrahydrofuran (5.00 mL) was added potassium acetate (222 mg, 2.26 mmol, 3.00 eq) at 20° C. The mixture was stirred at 20° C. for 20 min. 5-((4-methoxybenzyl)thio)thiazole-2-carbaldehyde (200 mg, 754 mol, 1.00 eq) and acetic acid (90.5 mg, 1.51 mmol, 86.3 μL, 2.00 eq), sodium triacetoxyhydroborate (320 mg, 1.51 mmol, 2.00 eq) was added to the mixture at 20° C. The resulting mixture was stirred at 20° C. for 2 h. The mixture was poured into water (30.0 mL), and then extracted with ethyl acetate (50.0 mL×2). The combined organic layers were dried over sodium sulfate, filtered and concentrated in vacuum to give a residue. The residue was purified by silica gel chromatography (petroleum ether/ethyl acetate=10:1-4:1, petroleum ether/ethyl acetate=3:1, R1(Rf=0.6), P1 (Rf=0.2)) to give a residue, which was determined by HNMR (EC1850-1164-P1A). 1-((5-((4-methoxybenzyl)thio)thiazol-2-yl)methyl)pyrrolidine-3-carbonitrile (160 mg, 416.81 mol, 55.30% yield, 90% purity) was obtained as a yellow solid. ¹H NMR (400 MHz, chloroform) 6=7.37 (s, 1H), 7.03 (s, 2H), 6.75-6.73 (m, 2H), 3.84 (s, 3H), 3.72 (s, 4H), 3.03-2.92 (m, 2H), 2.78-2.70 (m, 3H), 2.27-2.16 (m, 1H), 2.13-2.04 (m, 1H).

### Example 24.3. Preparation of 2-((3-cyanopyrrolidin-1-yl)methyl)thiazole-5-sulfonyl chloride

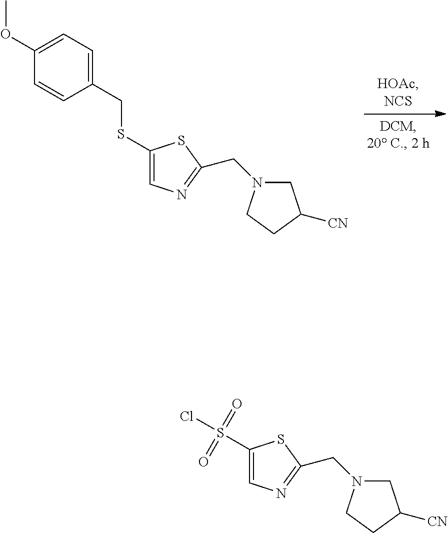 [SMILES: COc1ccc(CSc2cnc(CN3CCC(C#N)C3)s2)cc1 ; N#CC1CCN(Cc2ncc(S(=O)(=O)Cl)s2)C1]

To a solution of 1-[[5-[(4-methoxyphenyl)methylsulfanyl]thiazol-2-yl]methyl]pyrrolidine-3-carbonitrile (100 mg, 289 μmol, 1.00 eq) in dichloromethane (1.00 mL) and Water (1.00 mL), acetic acid (3.15 g, 52.4 mmol, 3.00 mL, 181 eq) was added 1-chloropyrrolidine-2,5-dione (193 mg, 1.45 mmol, 5.00 eq) at 0° C. The mixture was stirred at 20° C. for 2 h. The mixture was poured into water (20.0 mL), and then extracted with dichloromethane (10.0 mL×2). The combined organic layers were dried over sodium sulfate, filtered and concentrated in vacuum. It was not purified and used for the next step. 2-((3-cyanopyrrolidin-1-yl)methyl)thiazole-5-sulfonyl chloride (80.0 mg, 274 μmol, 94% yield) was obtained as a white solid.

### Example 24.4. Preparation of N-(3-cyano-4-methyl-1H-indol-7-yl)-2-((3-cyanopyrrolidin-1-yl)methyl)thiazole-5-sulfonamide (Compound 24)

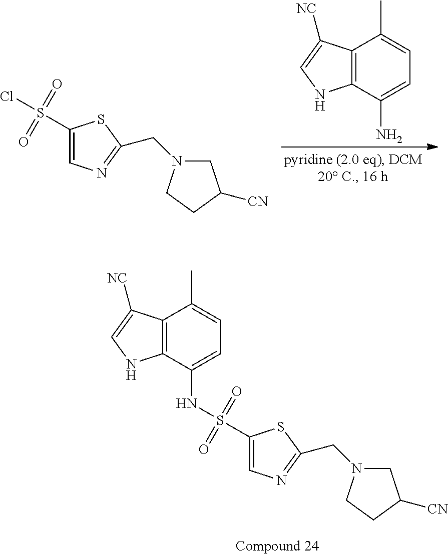 [SMILES: N#CC1CCN(Cc2ncc(S(=O)(=O)Cl)s2)C1 ; Cc1ccc(N)c2[nH]cc(C#N)c12 ; Cc1ccc(NS(=O)(=O)c2cnc(CN3CCC(C#N)C3)s2)c2[nH]cc(C#N)c12]

To a solution of 7-amino-4-methyl-1H-indole-3-carbonitrile (46.9 mg, 274 μmol, 1.00 eq) and pyridine (65.1 mg, 822 μmol, 66.4 μL, 3.00 eq) in dichloromethane (3.00 mL) was added 2-((3-cyanopyrrolidin-1-yl)methyl)thiazole-5-sulfonyl chloride (80.0 mg, 274 mol, 1.00 eq) at 0° C. The mixture was stirred at 20° C. for 2 h. The mixture was poured into water (30.0 mL), and then extracted with dichloromethane (30.0 mL×3). The combined organic layers were washed with 1 N hydrochloric acid (10.0 mL), brine (20.0 mL), dried over sodium sulfate, filtered and concentrated in vacuum to give a residue, which was determined by LCMS. The residue was purified by prep-HPLC (column: Waters Xbridge C18 150*50 mm*10 μm; mobile phase: [water(NH3H2O)-ACN]; gradient: 0%-30% B over 10 min). N-(3-cyano-4-methyl-1H-indol-7-yl)-2-((3-cyanopyrrolidin-1-yl)methyl)thiazole-5-sulfonamide (3.61 mg, 8.46 μmol, 3.09% yield, 100% purity) was obtained as a white solid. MS (ESI) m/z 425.0 [M−H]⁺¹H NMR (400 MHz, chloroform) 6=9.70-9.60 (m, 1H), 7.88 (s, 1H), 7.82 (d, J=3.2 Hz, 1H), 6.86 (d, J=8.0 Hz, 1H), 6.82-6.71 (m, 1H), 6.60 (d, J=8.0 Hz, 1H), 4.08-3.95 (m, 2H), 3.16-2.86 (m, 4H), 2.77 (s, 4H), 2.37-2.15 (m, 2H).

### Example 25 and Example 26. Synthetic Scheme of Compound 25 and Compound 26

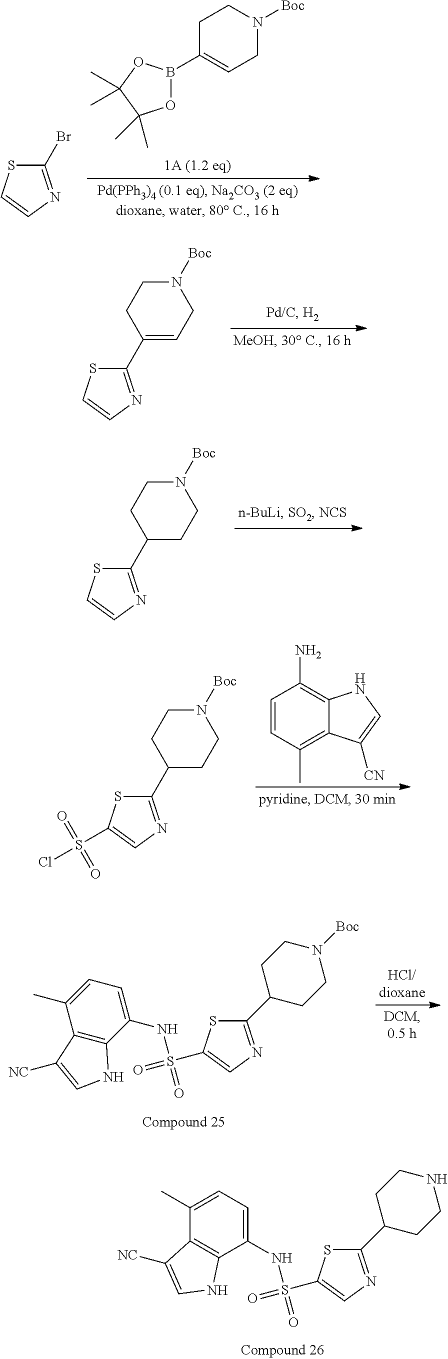

### Example 25.1. Preparation of tert-butyl 4-(thiazol-2-yl)-3,6-dihydropyridine-1(2H)-carboxylate

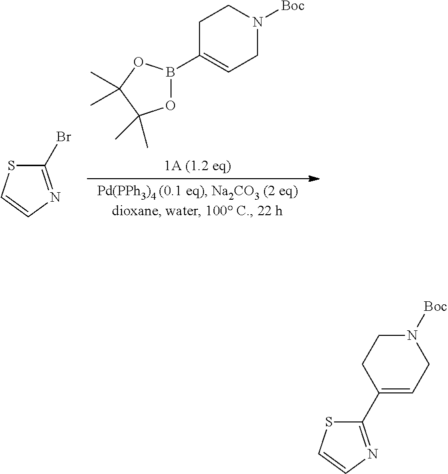

To a solution of 2-bromothiazole (9.00 g, 54.8 mmol, 4.95 mL, 1.00 eq) in dioxane (100 mL) and water (10.0 mL) was added tert-butyl 4-(4,4,5,5-tetramethyl-1,3,2-dioxaborolan-2-yl)-3,6-dihydropyridine-1(2H)-carboxylate (20.3 g, 65.8 mmol, 1.20 eq), potassium carbonate (15.1 g, 109 mmol, 2.00 eq) and tetrakis[triphenylphosphine]palladium(0) (1.00 g, 865 mol, 1.58e⁻² eq) at 16° C. The mixture was stirred at 100° C. for 22 h. The reaction mixture was quenched by addition water (50.0 mL), and then extracted with ethyl acetate (150 mL×3). The combined organic layers were washed with brine (50 mL×2), dried over [sodium sulfate], filtered and concentrated under reduced pressure to give a residue. The residue was purified by column chromatography (SiO₂, Petroleum ether/Ethyl acetate=100/1 to 10/1(TLC(petroleum ether: ethyl acetate=5:1), Rf(R=0.5, P=0.3)). Tert-butyl 4-(thiazol-2-yl)-3,6-dihydropyridine-1(2H)-carboxylate (5.20 g, 19.5 mmol, 35% yield) was obtained as a yellow oil. ¹H NMR (400 MHz, CDCl₃) δ=7.76 (d, J=3.2 Hz, 1H), 7.23 (d, J=3.2 Hz, 1H), 6.58 (s, 1H), 4.17-4.07 (m, 2H), 3.64 (t, J=5.2 Hz, 2H), 2.71 (br d, J=1.2 Hz, 2H), 1.49 (s, 9H).

### Example 25.2. Preparation of tert-butyl 4-(thiazol-2-yl)piperidine-1-carboxylate

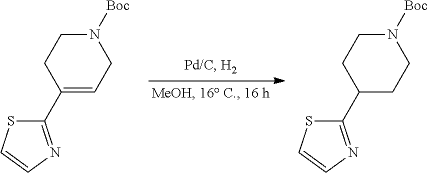 [SMILES: CC(C)(C)OC(=O)N1CC=C(c2nccs2)CC1 ; CC(C)(C)OC(=O)N1CCC(c2nccs2)CC1]

To a solution of tert-butyl 4-(thiazol-2-yl)-3,6-dihydropyridine-1(2H)-carboxylate (5.00 g, 18.7 mmol, 1.00 eq) in methanol (50.0 mL) was added palladium/carbon (1.50 g, 1.41 mmol, 10% purity, 7.51e⁻² eq) at 16° C. The mixture was stirred at 16° C. for 16 h under hydrogen (37.9 mg, 18.7 mmol, 1.00 eq) (15 Psi). The mixture was filtered and washed with methanol (500 mL). The filtrate was concentrated in vacuum. The residual was purified by column chromatography (SiO₂, Petroleum ether/Ethyl acetate=100/1 to 0/1(TLC(petroleum ether:ethyl acetate=1:1), Rf(R=0.6, P=0.3)). Tert-butyl 4-(thiazol-2-yl)piperidine-1-carboxylate (2.40 g, 8.94 mmol, 47% yield) was obtained as colorless oil. ¹H NMR (400 MHz, DMSO-d₆) δ=7.72 (d, J=3.2 Hz, 1H), 7.61 (d, J=3.6 Hz, 1H), 4.03-3.94 (m, 2H), 3.21 (tt, J=3.6, 11.6 Hz, 1H), 2.90 (d, J=0.8 Hz, 2H), 2.06-2.00 (m, 2H), 1.55 (dq, J=4.0, 12.4 Hz, 2H), 1.41 (s, 9H).

### Example 25.3. Preparation of tert-butyl 4-(5-(chlorosulfonyl)thiazol-2-yl)piperidine-1-carboxylate

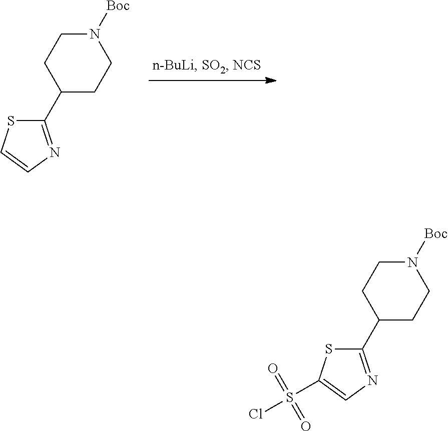 [SMILES: CC(C)(C)OC(=O)N1CCC(c2nccs2)CC1 ; CC(C)(C)OC(=O)N1CCC(c2ncc(S(=O)(=O)Cl)s2)CC1]

To a solution of tert-butyl 4-(thiazol-2-yl)piperidine-1-carboxylate (200 mg, 745 μmol, 1.00 eq) in tetrahydrofuran (5.00 mL) was added n-butyllithium (2 M, 447 μL, 1.20 eq) at −78° C. The mixture was stirred at −78° C. for 0.5 h. Sulfur dioxide (47.7 mg, 745 mol, 1.00 eq) was bubbled into at −65° C. for 30 min (15 Psi). The reaction mixture was warmed to 20° C. slowly and stirred for 2 h. N-chlorosuccinimide (199 mg, 1.49 mmol, 2.00 eq) was added to the mixture at 20° C. The mixture was stirred at 20° C. for 15 h. The reaction mixture was poured into water and extracted with dichloromethane (50 mL×3). The organic layer was concentrated under reduced pressure to give a residue. tert-butyl 4-(5-(chlorosulfonyl)thiazol-2-yl)piperidine-1-carboxylate (210 mg, crude) was obtained as colorless oil. ¹H NMR (400 MHz, DMSO-d₆) δ=7.61 (s, 1H), 4.03-3.90 (m, 2H), 3.14 (tt, J=3.6, 11.4 Hz, 1H), 2.95-2.81 (m, 2H), 2.07-1.92 (m, 2H), 1.57-1.47 (m, 2H), 1.41-1.40 (m, 2H), 1.40 (s, 9H).

### Example 25.4. Preparation of tert-butyl 4-[5-[(3-cyano-4-methyl-1H-indol-7-yl)sulfamoyl]thiazol-2-yl]piperidine-1-carboxylate (Compound 25)

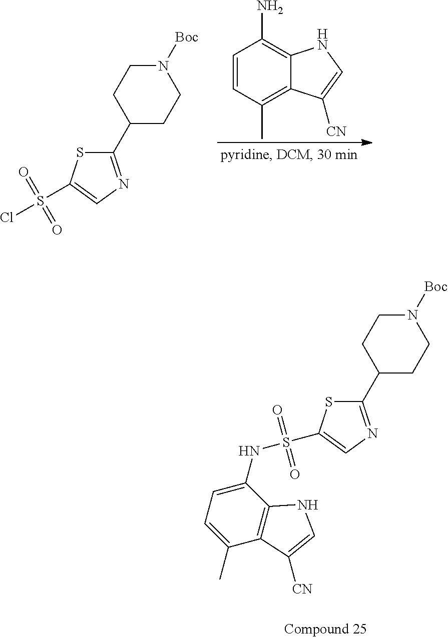 [SMILES: CC(C)(C)OC(=O)N1CCC(c2ncc(S(=O)(=O)Cl)s2)CC1 ; Cc1ccc(N)c2[nH]cc(C#N)c12 ; Cc1ccc(NS(=O)(=O)c2cnc(C3CCN(C(=O)OC(C)(C)C)CC3)s2)c2[nH]cc(C#N)c12]

To a solution of tert-butyl 4-(5-(chlorosulfonyl)thiazol-2-yl)piperidine-1-carboxylate (100 mg, 272 mol, 1.00 eq) in dichloromethane (1.00 mL) was added 7-amino-4-methyl-1H-indole-3-carbonitrile (51.3 mg, 299 μmol, 1.10 eq), pyridine (43.1 mg, 545 μmol, 44.0 μL, 2.00 eq) at 20° C. The mixture was stirred at 20° C. for 5 min. The mixture was concentrated under reduced pressure to give a residue. Tert-butyl 4-[5-[(3-cyano-4-methyl-1H-indol-7-yl)sulfamoyl]thiazol-2-yl]piperidine-1-carboxylate (85.0 mg, crude) was obtained as a white solid. The crude product was purified by prep-HPLC (column: Phenomenex luna C18 150*25 mm*10 μm; mobile phase: [water(FA)-ACN]; gradient: 47%-77% B over 11 min). Tert-butyl 4-[5-[(3-cyano-4-methyl-1H-indol-7-yl)sulfamoyl]thiazol-2-yl]piperidine-1-carboxylate (2.15 mg, 4.23 mol, 7.07% yield, 98.72% purity) was obtained as a yellow gum. MS (ESI) m/z 402.2 [M+H]⁺. ¹H NMR (400 MHz, DMSO-d₆) δ=8.10 (s, 1H), 7.91 (s, 1H), 6.86 (d, J=7.2 Hz, 1H), 6.74-6.65 (m, 1H), 3.92 (d, J=12.4 Hz, 2H), 3.26-3.14 (m, 1H), 2.94-2.76 (m, 2H), 2.57 (s, 3H), 1.99-1.88 (m, 2H), 1.51-1.40 (m, 2H), 1.40-1.32 (m, 9H).

### Example 26. Preparation of N-(3-cyano-4-methyl-1H-indol-7-yl)-2-(4-piperidyl)thiazole-5-sulfonamide (Compound 26)

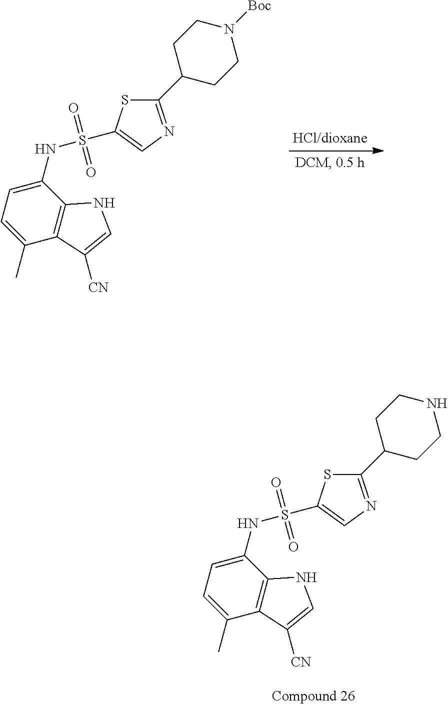 [SMILES: Cc1ccc(NS(=O)(=O)c2cnc(C3CCN(C(=O)OC(C)(C)C)CC3)s2)c2[nH]cc(C#N)c12 ; Cc1ccc(NS(=O)(=O)c2cnc(C3CCNCC3)s2)c2[nH]cc(C#N)c12]

To a solution of tert-butyl 4-[5-[(3-cyano-4-methyl-1H-indol-7-yl)sulfamoyl]thiazol-2-yl]piperidine-1-carboxylate (80.0 mg, 159 μmol, 1.00 eq) in dichloromethane (1.00 mL) was added hydrochloric acid/dioxane (6 M, 2.00 mL, 75.2 eq) at 16° C. The mixture was stirred at 20° C. for 0.5 h. The mixture was concentrated under reduced pressure to give a residue. The crude product was purified by prep-HPLC (column: Phenomenex luna C18 150*25 mm*10 μm; mobile phase: [water(FA)-ACN]; gradient: 6%-36% B over 10 min). N-(3-cyano-4-methyl-1H-indol-7-yl)-2-(4-piperidyl)thiazole-5-sulfonamide (35.77 mg, 89.09 mol, 55.86% yield, 100% purity) was obtained as a white solid. 5.77 mg product for delivery. MS (ESI) m/z 402.2 [M+H]⁺. ¹H NMR (400 MHz, DMSO-d₆) d=12.02-11.72 (m, 1H), 8.20 (s, 1H), 7.86 (s, 1H), 7.78 (s, 1H), 6.84 (d, J=7.6 Hz, 1H), 6.63 (br d, J=7.6 Hz, 1H), 3.30-3.17 (m, 3H), 2.95 (t, J=12.4 Hz, 2H), 2.49 (s, 3H), 2.09 (d, J=13.2 Hz, 2H), 1.83-1.71 (m, 2H).

### Example 27. Preparation of N-(3-cyano-4-methyl-1H-indol-7-yl)-2-(piperidin-3-yl)thiazole-5-sulfonamide (Compound 27)

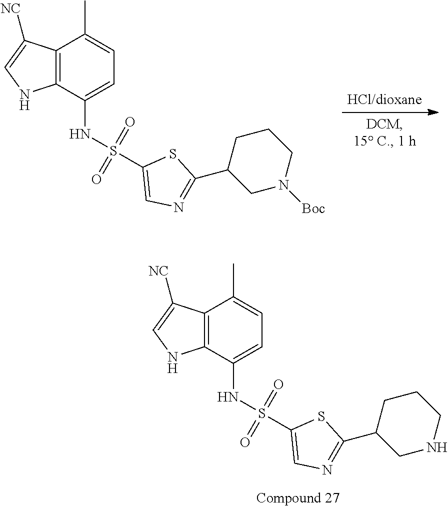 [SMILES: Cc1ccc(NS(=O)(=O)c2cnc(C3CCCN(C(=O)OC(C)(C)C)C3)s2)c2[nH]cc(C#N)c12 ; Cc1ccc(NS(=O)(=O)c2cnc(C3CCCNC3)s2)c2[nH]cc(C#N)c12]

To a solution of tert-butyl 3-(5-(N-(3-cyano-4-methyl-1H-indol-7-yl)sulfamoyl)thiazol-2-yl)piperidine-1-carboxylate (108 mg, 215 mol, 1.00 eq) in dichloromethane (1.00 mL) was added hydrochloric acid/dioxane (4.00 M, 1.00 mL, 18.6 eq). The mixture was stirred at 15° C. for 1 h. The mixture was concentrated in vacuum. The residue was dissolved with acetonitrile (1.00 mL) and diluted with water (10.0 mL). The solution was lyophilized in vacuo. Compound N-(3-cyano-4-methyl-1H-indol-7-yl)-2-(piperidin-3-yl)thiazole-5-sulfonamide (90 mg, 203 mol, 94.5% yield, 99% purity, hydrochloric acid salt) was obtained as a yellow solid. MS (ESI) m/z 402.1 [M+H]⁺. ¹H NMR (400 MHz, DMSO-d₆) δ=12.10 (d, J=2.4 Hz, 1H), 10.48 (s, 1H), 9.07-8.75 (m, 2H), 8.20 (d, J=3.2 Hz, 1H), 8.04 (s, 1H), 6.88 (d, J=8.0 Hz, 1H), 6.75 (d, J=8.0 Hz, 1H), 3.59-3.46 (m, 2H), 3.25 (d, J=13.2 Hz, 1H), 3.17-3.06 (m, 1H), 2.89 (d, J=9.2 Hz, 1H), 2.61 (s, 3H), 2.11 (d, J=12.0 Hz, 1H), 1.93-1.58 (m, 3H).

### Example 28. Preparation of N-(3-cyano-4-methyl-1H-indol-7-yl)-2-(1-methylpiperidin-3-yl)thiazole-5-sulfonamide (Compound 28)

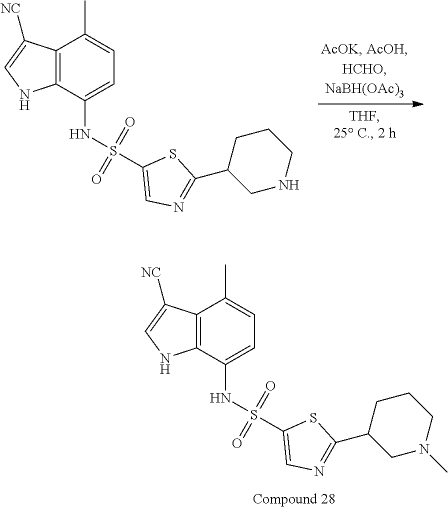 [SMILES: Cc1ccc(NS(=O)(=O)c2cnc(C3CCCNC3)s2)c2[nH]cc(C#N)c12 ; Cc1ccc(NS(=O)(=O)c2cnc(C3CCCN(C)C3)s2)c2[nH]cc(C#N)c12]

To a solution of N-(3-cyano-4-methyl-1H-indol-7-yl)-2-(piperidin-3-yl)thiazole-5-sulfonamide (50.0 mg, 124 mol, 1.00 eq) in tetrahydrofuran (1.00 mL) was added potassium acetate (36.6 mg, 374 μmol, 3.00 eq), formaldehyde (20.2 mg, 249 μmol, 18.5 μL, 2.00 eq), acetic acid (22.4 mg, 374 μmol, 21.4 μL, 3.00 eq) and sodium triacetoxyhydroborate (26.4 mg, 124 μmol, 1.00 eq). The mixture was stirred at 25° C. for 2 h. The mixture was blown dry with nitrogen. The residue was purified by prep-HPLC (column: Waters xbridge 150*25 mm 10 μm; mobile phase: [water(NH₄HCO₃)-ACN]; gradient: 13%-43% B over 14 min). Compound N-(3-cyano-4-methyl-1H-indol-7-yl)-2-(1-methylpiperidin-3-yl)thiazole-5-sulfonamide (22.79 mg, 54.8 μmol, 44.0% yield, 100% purity) was obtained as a white solid. MS (ESI) m/z 416.1 [M+H]⁺. ¹H NMR (400 MHz, DMSO-d₆+D₂O) δ=8.06 (s, 1H), 7.88 (s, 1H), 6.77 (q, J=8.0 Hz, 2H), 3.29 (s, 2H), 2.94 (d, J=4.8 Hz, 1H), 2.67 (s, 1H), 2.56 (s, 3H), 2.38 (s, 1H), 2.33 (s, 3H), 1.90 (d, J=2.8 Hz, 1H), 1.71-1.38 (m, 3H).

### Example 29. Preparation of 2-(1-acetyl-4-piperidyl)-N-(3-cyano-4-methyl-1H-indol-7-yl)thiazole-5-sulfonamide (Compound 29)

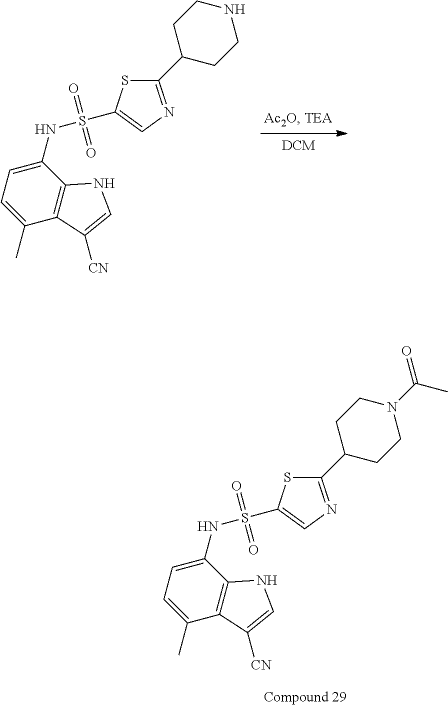 [SMILES: Cc1ccc(NS(=O)(=O)c2cnc(C3CCNCC3)s2)c2[nH]cc(C#N)c12 ; CC(=O)N1CCC(c2ncc(S(=O)(=O)Nc3ccc(C)c4c(C#N)c[nH]c34)s2)CC1]

To a solution of Ac₂O (8.39 mg, 82.2 mol, 7.72 μL, 1.20 eq) in dichloromethane (0.50 mL) was added triethylamine (34.7 mg, 342 mol, 47.6 μL, 5.00 eq) and N-(3-cyano-4-methyl-1H-indol-7-yl)-2-(4-piperidyl)thiazole-5-sulfonamide (30.0 mg, 68.5 μmol, 1.00 eq, hydrochloric acid) at 0° C. The mixture was stirred at 15° C. for 1 h. The reaction mixture was poured into water(10.0 mL) and extracted with dichloromethane (30 mL×3), dried over[sodium sulfate], filtered and concentrated under reduced pressure to give a residue. The crude product was purified by Prep-HPLC(column: Waters Xbridge C18 150*50 mm*10 μm; mobile phase: [water (NH₄HCO₃)-ACN]; gradient: 11%-41% B over 10 min). 2-(1-acetyl-4-piperidyl)-N-(3-cyano-4-methyl-1H-indol-7-yl)thiazole-5-sulfonamide was obtained as solid. MS (ESI) m/z 444.1 [M+H]⁺. ¹H NMR (400 MHz, DMSO-d₆) δ=8.10 (s, 1H), 7.96-7.83 (m, 1H), 6.84 (d, J=7.6 Hz, 1H), 6.71 (d, J=7.6 Hz, 1H), 4.33 (d, J=13.2 Hz, 1H), 3.82 (d, J=13.2 Hz, 1H), 3.28 (t, J=10.8 Hz, 1H), 3.14 (t, J=12.4 Hz, 1H), 2.68 (t, J=12.4 Hz, 1H), 2.57 (s, 3H), 2.04-1.92 (m, 5H), 1.65-1.51 (m, 1H), 1.49-1.35 (m, 1H).

### Example 30. Synthetic Scheme of Compound 30

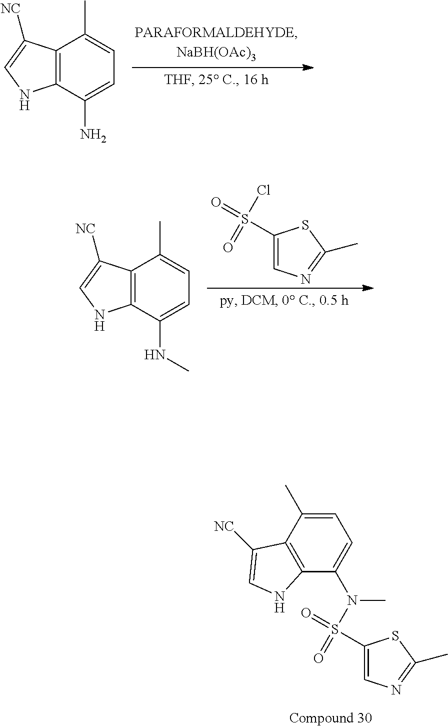

### Example 30.1. Preparation of 4-methyl-7-(methylamino)-1H-indole-3-carbonitrile

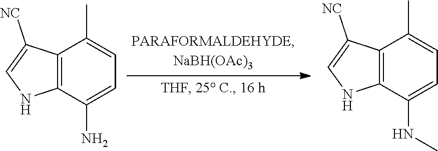

To a solution of 7-amino-4-methyl-1H-indole-3-carbonitrile (100 mg, 584 μmol, 1.00 eq) in tetrahydrofuran (20.0 mL) was added paraformaldehyde (80.0 mg, 584 μmol, 1.00 eq) and sodium triacetoxyhydroborate (371 mg, 1.75 mmol, 3.00 eq) at 16° C. The mixture was stirred at 25° C. for 16 h. The reaction mixture was poured into water (40 mL) and extracted with ethyl acetate (60 mL×3). dried over sodium sulfate, filtered and concentrated under reduced pressure to give a residue. The crude product was purified by prep-HPLC(column: Waters xbridge 150*25 mm 10 μm; mobile phase: [water(NH₄HCO₃)-ACN]; gradient: 24%-54% B over 10 min). 4-methyl-7-(methylamino)-1H-indole-3-carbonitrile (25 mg, 134.97 mol, 23.11% yield) was obtained as a yellow solid. ¹H NMR (400 MHz, DMSO-d₆) δ=11.65 (s, 1H), 8.07 (s, 1H), 6.75 (d, J=7.6 Hz, 1H), 6.22 (d, J=7.6 Hz, 1H), 5.34 (d, J=3.6 Hz, 1H), 3.30 (s, 3H), 2.81 (d, J=4.0 Hz, 3H).

### Example 30.2. Preparation of N-(3-cyano-4-methyl-1H-indol-7-yl)-N,2-dimethyl-thiazole-5-sulfonamide (Compound 30)

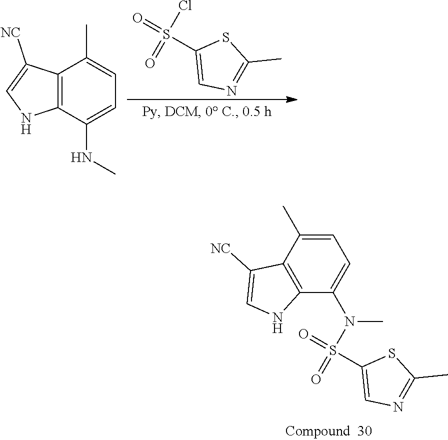

To a solution of 4-methyl-7-(methylamino)-1H-indole-3-carbonitrile (20.0 mg, 107 mol, 1.00 eq) in dichloromethane (1.00 mL) was added Py (25.6 mg, 323 μmol, 26.2 μL, 3.00 eq) and 2-methylthiazole-5-sulfonyl chloride (23.5 mg, 118 μmol, 1.10 eq) at 0° C. The mixture was stirred at 0° C. for 0.5 h. The reaction mixture was poured into water (10 mL) and extracted with dichloromethane (30 mL×3), dried over sodium sulfate, filtered and concentrated under reduced pressure to give a residue. The crude product was purified by prep-HPLC (column: Phenomenex luna C18 150*25 mm*10 μm; mobile phase: [water(FA)-ACN]; gradient: 31%-61% B over 11 min). N-(3-cyano-4-methyl-1H-indol-7-yl)-N,2-dimethyl-thiazole-5-sulfonamide (10.72 mg, 30.57 μmol, 28.31% yield, 98.78% purity) was obtained as gray solid. MS (ESI) m/z 347.2 [M+H]⁺. ¹H NMR (400 MHz, DMSO-d₆) δ=12.54-12.31 (m, 1H), 8.24 (s, 1H), 8.04 (s, 1H), 6.92 (d, J=7.6 Hz, 1H), 6.70 (d, J=7.6 Hz, 1H), 3.25 (s, 3H), 2.76 (s, 3H), 2.65 (s, 3H).

### Example 31. Synthetic Scheme of Compound 31

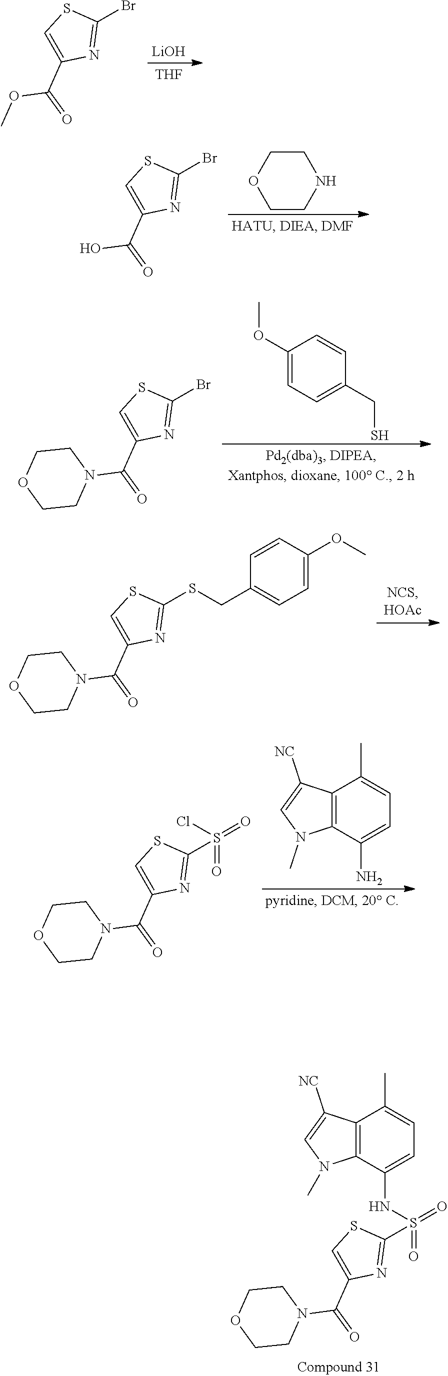

### Example 31.1. Preparation of 2-bromothiazole-4-carboxylic acid

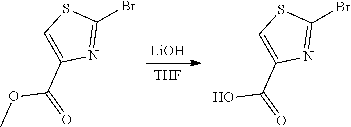

A mixture of methyl 2-bromothiazole-4-carboxylate (2.50 g, 11.3 mmol, 1.00 eq) in tetrahydrofuran (71.0 mL) and Lithium hydroxide monohydrate (1.00 M, 29.8 mL, 2.65 eq) was heated at 70° C. for 1 h. The organic solvent was removed in vacuo. The residual aqueous solution was cooled to 0° C. and acidified to pH=1 with 1N HCl solution. The mixture was filtered. The filter cake was washed with water and concentrated in vacuum. 2-Bromothiazole-4-carboxylic acid (2.20 g, 10.6 mmol, 94% yield) was obtained as a white solid.

¹H NMR (400 MHz, DMSO-d₆) δ=13.53-13.07 (m, 1H), 8.46 (s, 1H).

### Example 31.2. Preparation of (2-bromothiazol-4-yl)(morpholino)methanone

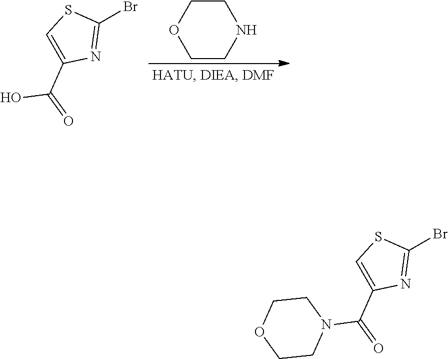

To a mixture of 2-bromothiazole-4-carboxylic acid (1.00 g, 4.81 mmol, 1.00 eq) and N,N-diisopropylethylamine (1.86 g, 14.4 mmol, 2.51 mL, 3.00 eq) in dimethylformamide (15.0 mL) was added O-(7-azabenzotriazol-1-yl)-N,N,N,N-tetramethyluroniumhexafluorophosphate (2.19 g, 5.77 mmol, 1.20 eq). The mixture was stirred at 25° C. for 30 min, then morpholine (461 mg, 5.29 mmol, 465 L, 1.10 eq) was added. The mixture was stirred at 25° C. for 1.5 h. The mixture was poured into water (40.0 mL) and extracted with ethyl acetate (20.0 ml×3). The combined organic phase was washed with brine (20.0 mL), dried with anhydrous sodium sulfate, filtered and concentrated in vacuum. The residue was purified by silica gel chromatography (petroleum ether/ethyl acetate=20:80). (2-Bromothiazol-4-yl)-morpholino-methanone (1.30 g, 4.69 mmol, 98% yield) was obtained as a white solid. ¹H NMR (400 MHz, DMSO-d₆) δ=8.16 (s, 1H), 3.63 (s, 8H).

### Example 31.3. Preparation of (2-((4-methoxybenzyl)thio)thiazol-4-yl)(morpholino)methanone

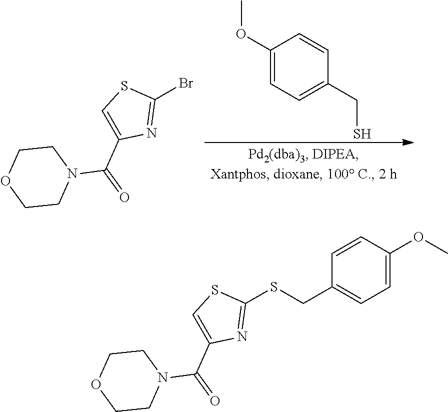

To a mixture of (2-bromothiazol-4-yl)-morpholino-methanone (500 mg, 1.80 mmol, 1.00 eq) and (4-methoxyphenyl)methanethiol (417 mg, 2.71 mmol, 377 μL, 1.50 eq) in dioxane (10.0 mL) was added tris(dibenzylideneacetone)dipalladium(0) (165 mg, 180 μmol, 0.100 eq), N,N-diisopropylethylamine (700 mg, 5.41 mmol, 943 μL, 3.00 eq) and Xantphos (104 mg, 180 mol, 0.100 eq) under nitrogen. The mixture was stirred at 100° C. for 2 h. The mixture was concentrated in vacuum. The residue was purified by silica gel chromatography (petroleum ether/ethyl acetate=60:40). [2-[(4-Methoxyphenyl)methylsulfanyl]thiazol-4-yl]-morpholino-methanone (600 mg, 1.71 mmol, 95% yield) was obtained as a white solid. ¹H NMR (400 MHz, DMSO-d₆) δ=8.02 (s, 1H), 7.33 (br d, J=8.0 Hz, 2H), 6.89 (d, J=8.0 Hz, 2H), 4.44 (s, 2H), 3.73 (s, 3H), 3.36-3.25 (m, 8H).

### Example 31.4. Preparation of 4-(morpholine-4-carbonyl)thiazole-2-sulfonyl chloride

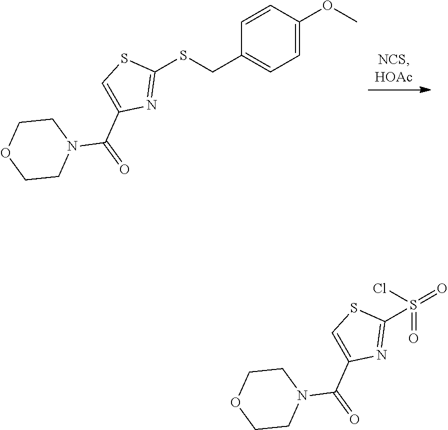 [SMILES: COc1ccc(CSc2nc(C(=O)N3CCOCC3)cs2)cc1 ; O=C(c1csc(S(=O)(=O)Cl)n1)N1CCOCC1]

Acetic acid (1.00 mL) was added to water (0.300 mL). [2-[(4-methoxyphenyl)methylsulfanyl]thiazol-4-yl]-morpholino-methanone (100 mg, 285 μmol, 1.00 eq) and 1-chloropyrrolidine-2,5-dione (152 mg, 1.14 mmol, 4.00 eq) were added to the mixture. The mixture was stirred at 0° C. for 1h. The mixture was poured into sat. sodium bicarbonate (20.0 mL), and adjusted to pH=8, separated the organic phase and the aqueous phase was extracted with ethyl acetate (20.0 ml×3). The organic layers were combined and washed with brine (10.0 mL), dried over anhydrous sodium sulfate and concentrated to give a residue. The crude can used to next step without any purification. 4-(morpholine-4-carbonyl)thiazole-2-sulfonyl chloride (84.0 mg, crude) was obtained as a yellow solid.

### Example 31.5. Preparation of N-(3-cyano-4-methyl-1H-indol-7-yl)-4-(morpholine-4-carbonyl)thiazole-2-sulfonamide (Compound 31)

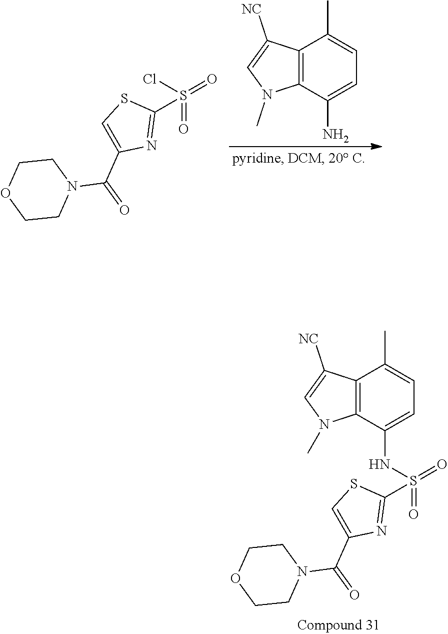

To a solution of 7-amino-4-methyl-1H-indole-3-carbonitrile (20.2 mg, 118 mol, 1.00 eq) and pyridine (18.7 mg, 236 μmol, 19.0 L, 2.00 eq) in dichloromethane (0.200 mL) was added dropwise a solution of 4-(morpholine-4-carbonyl)thiazole-2-sulfonyl chloride (35.0 mg, 118 μmol, 1.00 eq) in dichloromethane (0.200 mL) at 0° C. The mixture was stirred at 0° C. for 1 h. The mixture was concentrated in vacuum. The residue was purified by prep-HPLC (column: Phenomenex luna C18 150*25 mm*10 μm; mobile phase: [water(FA)-ACN]; gradient: 25%-55% B over 8 min). N-(3-cyano-4-methyl-1H-indol-7-yl)-4-(morpholine-4-carbonyl)thiazole-2-sulfonamide (8.98 mg, 20.40 mol, 17.29% yield, 98% purity) was obtained as a pink solid. MS (ESI) m/z 432.2 [M+H]⁺. ¹H NMR (400 MHz, DMSO-d₆) δ=12.04 (s, 1H), 10.89-10.71 (m, 1H), 8.43 (s, 1H), 8.20 (br d, J=2.4 Hz, 1H), 6.85 (d, J=7.6 Hz, 1H), 6.72 (d, J=7.6 Hz, 1H), 3.75-3.52 (m, 4H), 3.43 (br d, J=11.6 Hz, 4H), 2.60 (s, 3H).

### Example 32. Synthetic Scheme of Compound 32

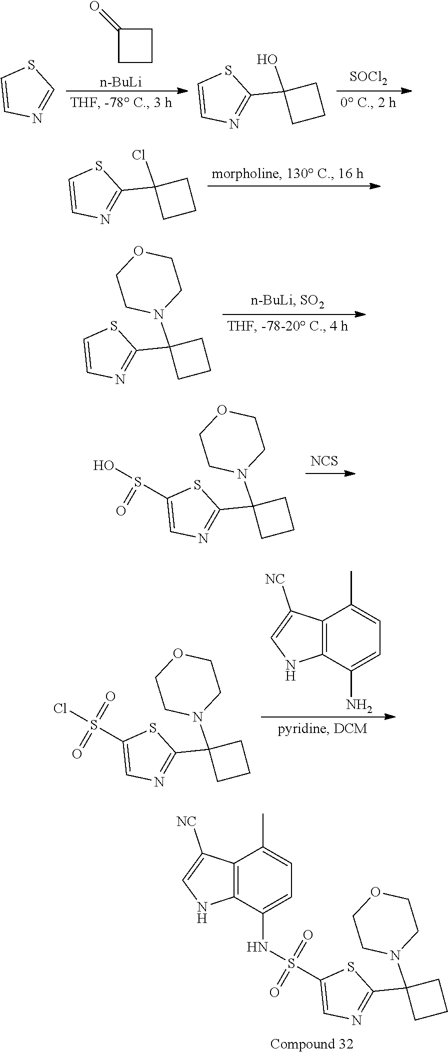

### Example 32.1. Preparation of 1-(thiazol-2-yl)cyclobutan-1-ol

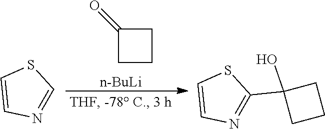 [SMILES: "n-BuLi" ; c1cscn1 ; O=C1CCC1 ; OC1(c2nccs2)CCC1]

To a solution of n-BuLi (2.50 M, 11.3 mL, 1.20 eq) in tetrahydrofuran (30.0 mL) was slowly added a solution of thiazole (2.00 g, 23.5 mmol, 1.00 eq) in tetrahydrofuran (20.0 mL) at −78° C. The resulting mixture was stirred for 1 h and then cyclobutanone (3.29 g, 47.0 mmol, 3.51 mL, 2.00 eq) in tetrahydrofuran (7.00 mL) was added. The mixture was stirred for 2 h at −78° C. Then sat. ammonium chloride (200 ml) was added and the phases were separated. The aqueous phase was extracted with ethyl acetate (200 ml×3) and the combined organics were washed with water, brine (30.0 ml), dried (sodium sulfate) and concentrated. The crude was combined with EC2166-867 to purification. The residue was purified by silica gel chromatography (Petroleum ether/Ethyl acetate=3/1). 1-thiazol-2-ylcyclobutanol (3.90 g, crude) was obtained as a yellow oil. ¹H NMR (400 MHz, DMSO-d₆) δ=7.73 (d, J=3.2 Hz, 1H), 7.59 (d, J=3.2 Hz, 1H), 6.44 (s, 1H), 2.49-2.45 (m, 2H), 2.37-2.26 (m, 2H), 1.94-1.82 (m, 2H).

### Example 32.2. Preparation of 2-(1-chlorocyclobutyl)thiazole

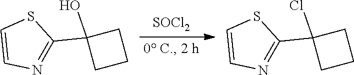

A mixture of 1-thiazol-2-ylcyclobutanol (2.47 g, 15.9 mmol, 1.00 eq) in thionyl chloride (9.90 mL) was stirred at 0° C. for 2 h. The mixture was concentrated in reduced pressure. The residue was poured into sat. sodium bicarbonate (100 mL) and extracted with ethyl acetate (30.0 ml×3). The combined organic phase was washed with brine (30.0 mL), dried with anhydrous sodium sulfate, filtered and concentrated in vacuum. The residue was purified by silica gel chromatography (Petroleum ether/Ethyl acetate=90/10). 2-(1-Chlorocyclobutyl)thiazole (2.30 g, 12.8 mmol, 80% yield, 96% purity) was obtained as a colorless oil. ¹H NMR (400 MHz, DMSO-d₆) δ=7.85-7.81 (m, 2H), 3.04-2.98 (m, 2H), 2.82-2.75 (m, 2H), 2.22 (ttd, J=5.6, 9.6, 10.8 Hz, 1H), 1.98-1.86 (m, 1H).

### Example 32.3. Preparation of 4-(1-(thiazol-2-yl)cyclobutyl)morpholine

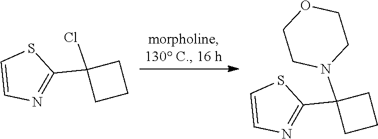 [SMILES: ClC1(c2nccs2)CCC1 ; c1csc(C2(N3CCOCC3)CCC2)n1]

A mixture of 2-(1-chlorocyclobutyl)thiazole (1.96 g, 11.3 mmol, 1.00 eq) in morpholine (30.0 mL) was stirred at 130° C. for 16 h. The mixture was poured into water (20.0 mL) and acidified with hydrochloric acid (1 M) to pH about 8. The resulting mixture was extracted with ethyl acetate (20.0 ml×3). The combined organic phase was washed with brine (20.0 mL), dried with anhydrous sodium sulfate, filtered and concentrated in vacuum. The residue was purified by silica gel chromatography (petroleum ether/ethyl acetate=85:15). 4-(1-thiazol-2-ylcyclobutyl)morpholine (1.00 g, 4.46 mmol, 39% yield) was obtained as a yellow solid. ¹H NMR (400 MHz, DMSO-d₆) δ=7.79 (d, J=3.3 Hz, 1H), 7.69 (d, J=3.2 Hz, 1H), 3.60-3.51 (m, 4H), 2.41-2.28 (m, 8H), 1.85-1.66 (m, 2H).

### Example 32.4. Preparation of 2-(1-morpholinocyclobutyl)thiazole-5-sulfinic acid

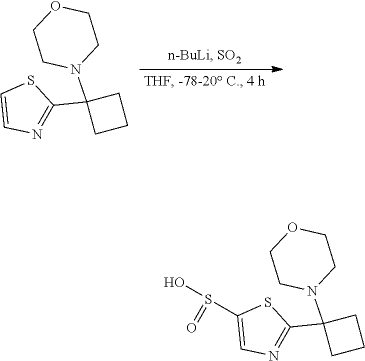

To a solution of 4-(1-thiazol-2-ylcyclobutyl)morpholine (300 mg, 1.34 mmol, 1.00 eq) in tetrahydrofuran (6.00 mL) was added n-butyllithium (2.50 M, 802 μL, 1.50 eq) dropwise at −78° C. and stirred 30 min under nitrogen, and then sulfur dioxide (85.7 mg, 1.34 mmol, 1.00 eq) was bubbled into at −65° C. for 30 min. The reaction mixture was warmed to 20° C. slowly and stirred for 3 h. The mixture can used to next step without any work-up. The mixture can used to next step without any purification. A solution of 2-(1-morpholinocyclobutyl)thiazole-5-sulfinic acid (386 mg, crude) in THF (6.00 mL) was obtained as a yellow liquid. The solution was used to next step directly.

### Example 32.5. Preparation of 2-(1-morpholinocyclobutyl)thiazole-5-sulfonyl chloride

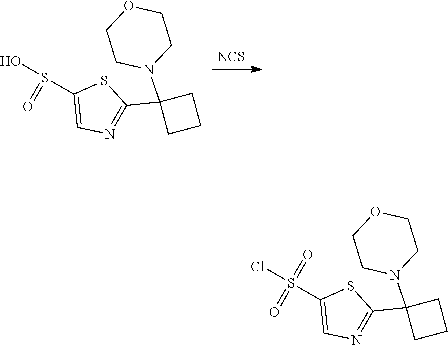 [SMILES: O=S(O)c1cnc(C2(N3CCOCC3)CCC2)s1 ; "NCS" ; O=S(=O)(Cl)c1cnc(C2(N3CCOCC3)CCC2)s1]

To a solution of 2-(1-morpholinocyclobutyl)thiazole-5-sulfinic acid (386 mg, 1.34 mmol, 1.00 eq) in tetrahydrofuran (the reaction from EC2166-897) was added 1-chloropyrrolidine-2,5-dione (536 mg, 4.02 mmol, 3.00 eq) at 0° C. and stirred another 16 h at 20° C. The mixture was poured into water (10.0 mL) and extracted with dichloromethane (10.0 ml×3). The combined organic phase was washed with brine (10.0 mL), dried with anhydrous sodium sulfate, filtered and concentrated in vacuum. The residue was purified by silica gel chromatography (petroleum ether/ethyl acetate=5:1). 2-(1-morpholinocyclobutyl)thiazole-5-sulfonyl chloride (350 mg, 1.08 mmol, 81% yield) was obtained as a yellow oil. ¹H NMR (400 MHz, DMSO-d₆) δ=7.96 (s, 1H), 3.89 (s, 4H), 3.08-2.96 (m, 4H), 2.59-2.55 (m, 2H), 1.99-1.92 (m, 2H), 1.83-1.69 (m, 2H).

### Example 32.6. Preparation of N-(3-cyano-4-methyl-1H-indol-7-yl)-2-(1-morpholinocyclobutyl)thiazole-5-sulfonamide (Compound 32)

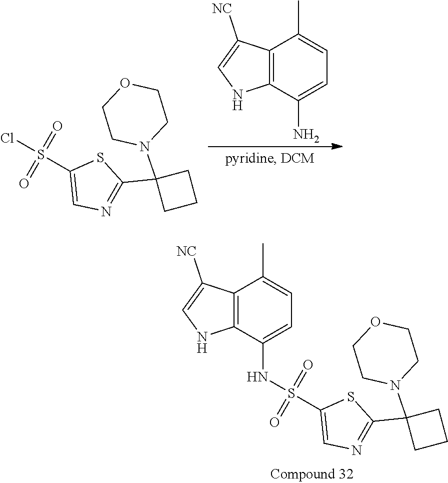 [SMILES: O=S(=O)(Cl)c1cnc(C2(N3CCOCC3)CCC2)s1 ; Cc1ccc(N)c2[nH]cc(C#N)c12 ; Cc1ccc(NS(=O)(=O)c2cnc(C3(N4CCOCC4)CCC3)s2)c2[nH]cc(C#N)c12]

To a solution of 7-amino-4-methyl-1H-indole-3-carbonitrile (26.5 mg, 155 μmol, 1.00 eq) and pyridine (24.5 mg, 310 μmol, 25.0 μL, 2.00 eq) in dichloromethane (0.500 mL) was added dropwise a solution of 2-(1-morpholinocyclobutyl)thiazole-5-sulfonyl chloride (50.0 mg, 155 μmol, 1.00 eq) in dichloromethane (0.500 mL) at 0° C. The mixture was stirred at 0° C. for 1 h. The mixture was concentrated in vacuum. The residue was purified by prep-HPLC (column: Phenomenex luna C18 150*25 mm*10 μm; mobile phase: [water(FA)-ACN]; gradient: 30%-60% B over 8 min). N-(3-cyano-4-methyl-1H-indol-7-yl)-2-(1-morpholinocyclobutyl)thiazole-5-sulfonamide (30.52 mg, 66.70 μmol, 43.07% yield, 100% purity) was obtained as a pink solid. MS (ESI) m/z 458.2 [M+H]⁺. ¹H NMR (400 MHz, DMSO-d₆) δ=11.89 (d, J=2.4 Hz, 1H), 10.17 (s, 1H), 8.16 (d, J=3.2 Hz, 1H), 8.03 (s, 1H), 6.87 (d, J=8.0 Hz, 1H), 6.71 (d, J=7.6 Hz, 1H), 3.54 (s, 4H), 2.61 (s, 3H), 2.44 (d, J=10.8 Hz, 2H), 2.28 (s, 4H), 2.16 (t, J=8.8 Hz, 2H), 1.88-1.69 (m, 2H).

### Example 33. Synthetic Scheme of Compound 33

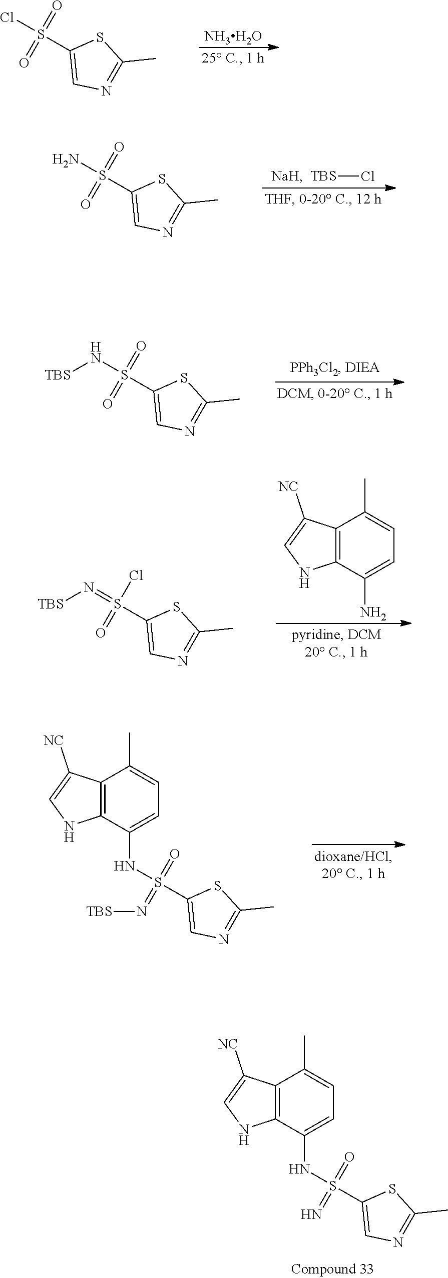

### Example 33.1. Preparation of 2-methylthiazole-5-sulfonamide

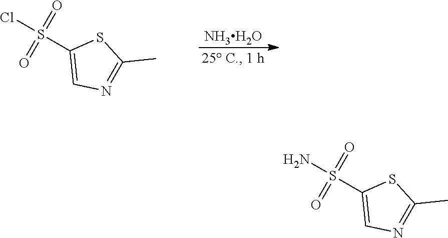

To a solution of 2-methylthiazole-5-sulfonyl chloride (450 mg, 2.28 mmol, 1.00 eq) in dichloromethane (0.500 mL) was added ammonium hydroxide (4.10 g, 29.2 mmol, 4.50 mL, 25% purity, 12.8 eq). The mixture was stirred at 20° C. for 2 h. The reaction mixture was concentrated under reduced pressure to remove solvent. The residue was purified by column chromatography (SiO₂, Petroleum ether/Ethyl acetate=1/0 to 1/1) and concentrated under reduced pressure to remove solvent and give the 2-methylthiazole-5-sulfonamide (360 mg, 1.92 mmol, 84.3% yield, 95% purity) as a white solid. ¹H NMR (400 MHz, DMSO-d₆) δ=7.97 (s, 1H), 7.85 (s, 2H), 2.71 (s, 3H).

### Example 33.2. Preparation of N-[tert-butyl(dimethyl)silyl]-2-methyl-thiazole-5-sulfonamide

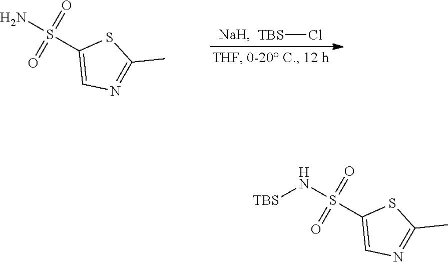

To a solution of 2-methylthiazole-5-sulfonamide (300 mg, 1.68 mmol, 1.00 eq) in tetrahydrofuran (7.00 mL) was added dropwise sodium hydride (135 mg, 3.37 mmol, 60% purity, 2.00 eq) at 0° C. After addition, the mixture was stirred at this temperature for 30 min, and then tert-butylchlorodimethylsilane (1.27 g, 8.42 mmol, 1.04 mL, 5.00 eq) was added dropwise at 0° C. The resulting mixture was stirred at 20° C. for 12 h. The reaction mixture was quenched by addition water (10.0 mL) at 0° C., and then extracted with ethyl acetate (10.0 mL). The combined organic layers were washed with brine (10.0 mL), dried over sodium sulfate, filtered and concentrated under reduced pressure to give a residue. The residue was purified by column chromatography (SiO₂, Petroleum ether/Ethyl acetate=1/0 to 3/1) and concentrated under reduced pressure to give the N-[tert-butyl(dimethyl)silyl]-2-methyl-thiazole-5-sulfonamide (400 mg, 1.30 mmol, 77.2% yield, 95% purity) as a white solid. ¹H NMR (400 MHz, DMSO-d₆) δ=8.10 (s, 1H), 7.94 (s, 1H), 2.71 (s, 3H), 0.88 (s, 9H), 0.17-0.14 (m, 6H).

### Example 33.3. Preparation of tert-butyl-[[chloro-(2-methylthiazol-5-yl)-oxo-sulfanylidene]amino]-dimethyl-silane

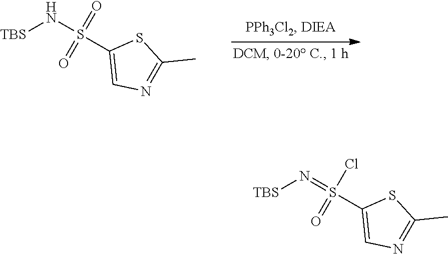

To a solution of dichloro(triphenyl)-phosphane (17.1 mg, 51.3 μmol, 1.50 eq) in trichloromethane (0.100 mL) was added dropwise triethylamine (5.54 mg, 54.7 mol, 7.61 μL, 1.60 eq) at 0° C. After addition, the mixture was stirred at 20° C. for 15 min, and then N-[tert-butyl(dimethyl)silyl]-2-methyl-thiazole-5-sulfonamide (10.0 mg, 34.2 μmol, 1.00 eq) in trichloromethane (0.100 mL) was added dropwise at 0° C. The resulting mixture was stirred at 0° C. for 30 min. The crude product tert-butyl-[[chloro-(2-methylthiazol-5-yl)-oxo-sulfanylidene]amino]-dimethyl-silane (100 mg, crude) in trichloromethane (1.00 mL) was obtained as a yellow liquid. The mixture was used into the next step without further purification.

### Example 33.4. Preparation of N-(3-cyano-4-methyl-1H-indol-7-yl)-2-morpholino-thiazole-5-sulfonamide

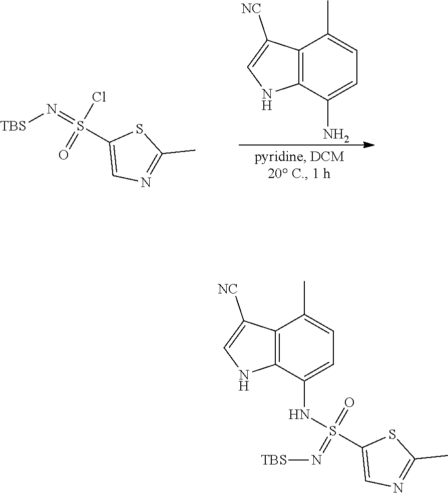

To a solution of tert-butyl-[[chloro-(2-methylthiazol-5-yl)-oxo-sulfanylidene]amino]-dimethyl-silane (10.0 mg, 32.2 μmol, 1.00 eq) and 7-amino-4-methyl-1H-indole-3-carbonitrile (5.51 mg, 32.2 mol, 1.00 eq) in dichloromethane (1.00 mL) was added pyridine (5.09 mg, 64.3 μmol, 5.19 μL, 2.00 eq). The mixture was stirred at 20° C. for 1 h. The reaction mixture was diluted with water (5.00 mL) and extracted with ethyl acetate (5.00 mL). The combined organic layers were washed with brine (5.00 mL), dried over sodium sulfate, filtered and concentrated under reduced pressure to give a residue. The residue was purified by column chromatography (SiO₂, Petroleum ether/Ethyl acetate=1/0 to 3/1) and concentrated under reduced pressure to remove solvent to give the 7-[[N-[tert-butyl(dimethyl)silyl]-S-(2-methylthiazol-5-yl)sulfonimidoyl]amino]-4-methyl-1H-indole-3-carbonitrile (25.0 mg, 48.8 mol, 15% yield, 87% purity) as a white solid. MS (ESI) m/z 446.1 [M+H]⁺.

### Preparation of 33.5. Preparation of 4-methyl-7-[[(2-methylthiazol-5-yl)sulfonimidoyl]amino]-1H-indole-3-carbonitrile (Compound 33)

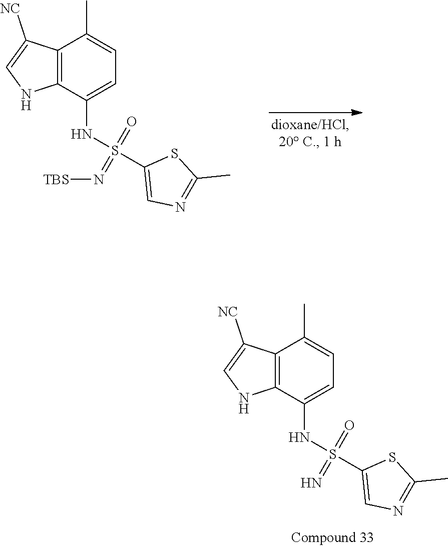

To a solution of 7-[[N-[tert-butyl(dimethyl)silyl]-S-(2-methylthiazol-5-yl)sulfonimidoyl]amino]-4-methyl-1H-indole-3-carbonitrile (20.0 mg, 44.9 μmol, 1.00 eq) in dichloromethane (0.100 mL) was added dioxane (4 M, 11.2 μL, 1.00 eq). The mixture was stirred at 20° C. for 3 h. The reaction mixture was concentrated under reduced pressure to remove solvent. The crude product was purified by prep-HPLC (column: Phenomenex luna C18 150*25 mm*10 μm; mobile phase: [water(FA)-ACN]; gradient: 23%-53% B over 8 min) and lyophilized to give the 4-methyl-7-[[(2-methylthiazol-5-yl)sulfonimidoyl]amino]-1H-indole-3-carbonitrile (5.08 mg, 15.0 mol, 33% yield, 98% purity) as a yellow gum. MS (ESI) m/z 354.0 [M+H]⁺. ¹H NMR (400 MHz, DMSO-d₆) δ=11.83-11.57 (m, 1H), 8.19 (s, 1H), 8.08 (s, 1H), 7.66 (br s, 2H), 6.86-6.80 (m, 1H), 6.79-6.72 (m, 1H), 2.68 (s, 3H), 2.54 (s, 3H)

### Example 34. Preparation of N-(5-(N-(3-cyano-4-methyl-1H-indol-7-yl)sulfamoyl)thiazol-2-yl)acetamide (Compound 34)

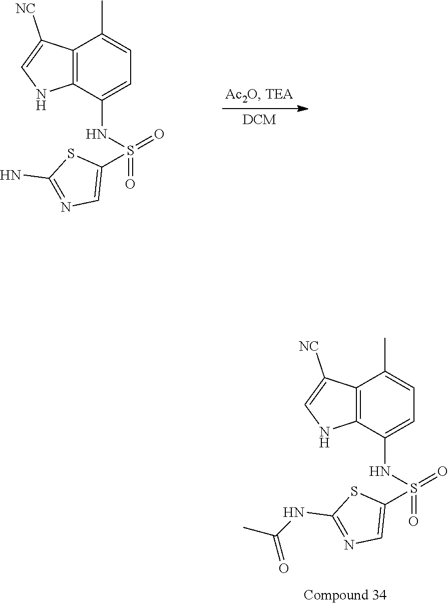 [SMILES: Cc1ccc(NS(=O)(=O)c2cnc(N)s2)c2[nH]cc(C#N)c12 ; CC(=O)Nc1ncc(S(=O)(=O)Nc2ccc(C)c3c(C#N)c[nH]c23)s1]

To a solution of 2-amino-N-(3-cyano-4-methyl-1H-indol-7-yl)thiazole-5-sulfonamide (50.0 mg, 111 mol, 1.00 eq, trifluoroacetic acid) in dichloromethane (1 mL) was added triethylamine (113 mg, 1.12 mmol, 155 μL, 10 eq) and acetic anhydride (11.4 mg, 111 μmol, 10.5 μL, 1.00 eq) at 0° C. The mixture was stirred at 16° C. for 3.5 h. The mixture was quenched with water (10 mL) and extracted with dichloromethane (20 mL×3). The combined organic layer was concentrated in vacuum. The crude product was purified by prep-HPLC (column: Phenomenex luna C18 150*25 mm*10 μm; mobile phase: [water(FA)-ACN]; gradient: 23%-53% B over 10 min). N-(5-(N-(3-cyano-4-methyl-1H-indol-7-yl)sulfamoyl)thiazol-2-yl)acetamide (10.86 mg, 28.64 mol, 25.63% yield, 99% purity) was obtained as a white solid. MS (ESI) m/z 376.0 [M+H]⁺. ¹H NMR (400 MHz, DMSO-d6) δ=12.91-12.30 (m, 1H), 8.40 (s, 1H), 8.29 (s, 1H), 8.14 (s, 2H), 7.59 (s, 1H), 7.09-6.92 (m, 2H), 2.72-2.67 (m, 3H), 1.86 (s, 3H).

### Example 35. Synthetic Scheme of Compound 35

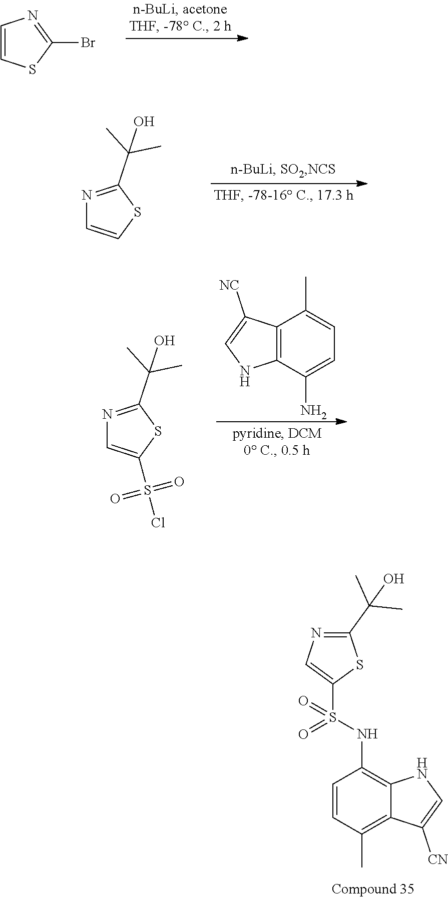

### Example 35.1. Preparation of 2-thiazol-2-ylpropan-2-ol

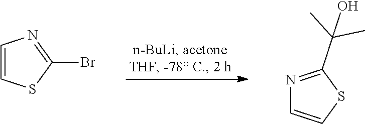

To a solution of 2-bromothiazole (2.00 g, 12.2 mmol, 1.10 mL, 1.00 eq) in tetrahydrofuran (25.0 mL) was added n-butyllithium (2.5 M, 5.37 mL, 1.10 eq) at −78° C.. The mixture was stirred at −78° C. for 0.5h, acetone (779 mg, 13.4 mmol, 986 μL, 1.10 eq) was added to the mixture, The mixture was stirred at −78° C. for 1.5 h. The mixture was poured into water (50.0 mL), separated the organic phase and the aqueous phase was extracted with ethyl acetate (3×60 mL). The organic layers were combined and washed with brine (50.0 mL), dried over sodium sulfate and concentrated to give a residue. The residue was purified by column chromatography (SiO₂, Petroleum ether/Ethyl acetate=100/1 to 4/1), TLC (Petroleum ether/Ethyl acetate=1:1, Rf(R=0.2, P=0.3)). 2-thiazol-2-ylpropan-2-ol (750 mg, 5.24 mmol, 42.95% yield) was obtained as brown oil. ¹H NMR (400 MHz, DMSO-d₆) δ=7.74 (d, J=3.2 Hz, 1H), 7.60 (d, J=3.2 Hz, 1H), 1.57 (s, 6H).

### Example 35.2. Preparation of 2-(1-hydroxy-1-methyl-ethyl)thiazole-5-sulfonyl chloride

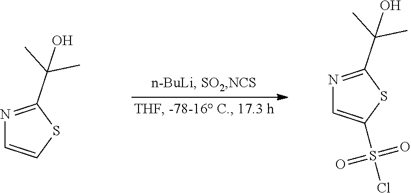

To a solution of 2-thiazol-2-ylpropan-2-ol (300 mg, 2.09 mmol, 1.00 eq) in tetrahydrofuran (7.00 mL) was added n-butyllithium (2.5 M, 2.51 mL, 3.00 eq) dropwise at −78° C.; the mixture was stirred for 30 min at −78° C. Then sulfur dioxide (134 mg, 2.09 mmol, 1.00 eq) was introduced in this solution for 20 minutes below −30° C. under 15 Psi. The solution was stirred for 0.5 hr at 16° C. NCS (419 mg, 3.14 mmol, 1.50 eq) was added to the mixture at 16° C. The solution was stirred for 16 hr at 16° C. The mixture was poured into water (30.0 mL), separated the organic phase and the aqueous phase was extracted with ethyl acetate (3×60 mL). The organic layers were combined and washed with brine (50.0 mL), dried over anhydrous sodium sulfate and concentrated to give a residue. The residue was purified by column chromatography (SiO₂. Petroleum ether/Ethyl acetate=100/1 to 3/1), TLC (Petroleum ether/Ethyl acetate=1:1, Rf (R=0.3, P=0.5)). 2-(1-hydroxy-1-methyl-ethyl)thiazole-5-sulfonyl chloride (100 mg, 413.71 μmol, 19.75% yield, 100% purity) was obtained as a white solid. MS (ESI) m/z 241.9 [M+H]⁺.

### Example 35.5. Preparation of N-(3-cyano-4-methyl-1H-indol-7-yl)-2-(1-hydroxy-1-methyl-ethyl)thiazole-5-sulfonamide (Compound 35)

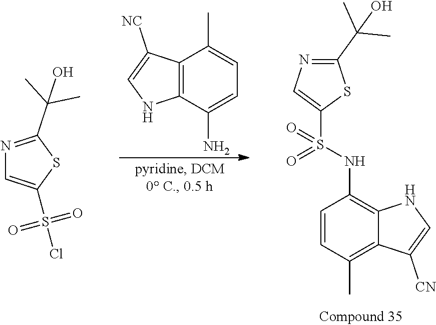 [SMILES: CC(C)(O)c1ncc(S(=O)(=O)Cl)s1 ; Cc1ccc(N)c2[nH]cc(C#N)c12 ; Cc1ccc(NS(=O)(=O)c2cnc(C(C)(C)O)s2)c2[nH]cc(C#N)c12]

To a solution of 2-(1-hydroxy-1-methyl-ethyl)thiazole-5-sulfonyl chloride (50.0 mg, 206 μmol, 1.10 eq) in dichloromethane (1.00 mL) was added pyridine (44.6 mg, 564 mol, 45.5 μL, 3.00 eq) and 7-amino-4-methyl-1H-indole-3-carbonitrile (32.1 mg, 188 mol, 1.00 eq) at 0° C. The mixture was stirred at 0° C. for 0.5 h. The mixture was concentrated under reduced pressure to give a residue. The crude product was purified by prep-HPLC(column: Phenomenex luna C18 150*25 mm*10 μm; mobile phase: [water(FA)-ACN]; gradient: 23%-53% B over 10 min). N-(3-cyano-4-methyl-1H-indol-7-yl)-2-(1-hydroxy-1-methyl-ethyl)thiazole-5-sulfonamide (8.81 mg, 23.36 mol, 12.42% yield, 99.81% purity) was obtained as a white solid. MS (ESI) m/z 377.2 [M+H]⁺. ¹H NMR (400 MHz, DMSO-d₆) δ=11.93 (s, 1H), 10.27 (d, J=1.5 Hz, 1H), 8.18 (s, 1H), 7.94 (s, 1H), 6.89 (d, J=7.6 Hz, 1H), 6.74 (d, J=8.0 Hz, 1H), 6.30 (s, 1H), 2.62 (s, 3H), 1.47 (s, 6H).

### Example 36. Synthetic Scheme of Compound 143

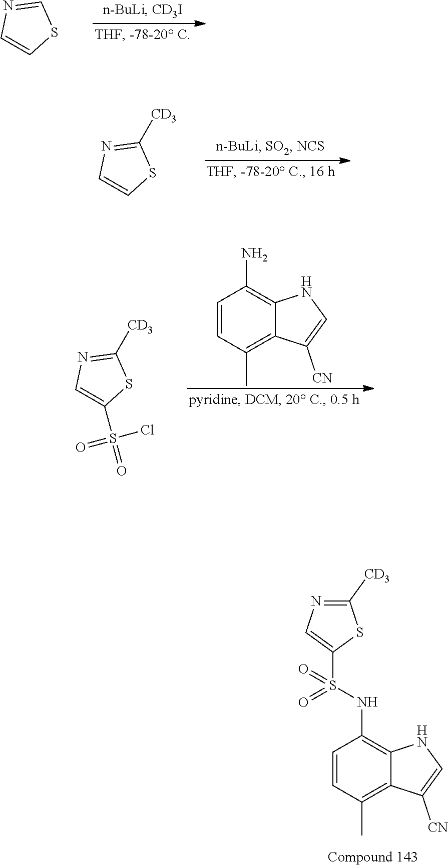 [SMILES: c1cscn1 ; [2H]C([2H])([2H])c1nccs1 ; [2H]C([2H])([2H])c1ncc(S(=O)(=O)Cl)s1 ; Cc1ccc(N)c2[nH]cc(C#N)c12 ; [2H]C([2H])([2H])c1ncc(S(=O)(=O)Nc2ccc(C)c3c(C#N)c[nH]c23)s1]

### Example 36.1. Preparation of 2-(methyl-d3)thiazole

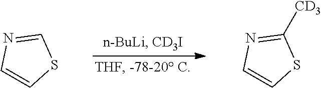 [SMILES: c1cscn1 ; [2H]C([2H])([2H])c1nccs1]

To a solution of thiazole (1.00 g, 11.8 mmol, 1.00 eq) in tetrahydrofuran (15.0 mL) was added dropwise n-butyllithium (2.5 M, 5.17 mL, 1.10 eq) at −78° C. trideuterio(iodo)methane (2.21 g, 15.3 mmol, 950 μL, 1.30 eq) was added at −60° C. and stirred at 20° C. for another 2 h. The mixture was quenched by adding saturated ammonium chloride (50.0 mL) and extracted with ethyl acetate (50 mL×2). The residue was purified by column chromatography (silicon dioxide, petroleum ether/ethyl acetate=3/1) to give 2-(methyl-d₃)thiazole (1.30 g, 12.7 mmol, 54% yield) as a yellow oil. ¹H NMR (400 MHz, CHLOROFORM-d) δ=7.65 (d, J=3.6 Hz, 1H), 7.17 (d, J=3.6 Hz, 1H) Example 36.2. Preparation of 3-2-(methyl-d3)thiazole-5-sulfonyl chloride

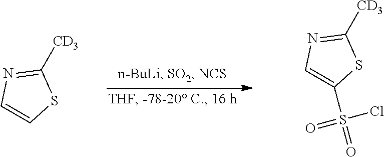 [SMILES: [2H]C([2H])([2H])c1nccs1 ; [2H]C([2H])([2H])c1ncc(S(=O)(=O)Cl)s1]

To a solution of 2-(methyl-d₃)thiazole (1.20 g, 11.7 mmol, 1.00 eq) in tetrahydrofuran (15.0 mL) was added n-butyllithium (2.5 M, 7.99 mL, 1.70 eq) dropwise at −78° C. and stirred 30 min. under nitrogen, and then sulfur dioxide (752 mg, 11.7 mmol, 1.00 eq) was bubbled into at −65° C. for 30 min. The reaction mixture was warmed to 20° C. slowly and stirred for 3 h. N-chlorosuccinimide (2.89 g, 21.7 mmol, 3.00 eq) was added at 0° C. The mixture was stirred at 20° C. for 12 h. The mixture was poured into water (100 mL) and extracted with ethyl acetate (50 mL×3). The combined organic layer was dried over anhydrous sodium sulfate, filtered and concentrated under vacuum to give a residue. The residue was purified by column chromatography (silicon dioxide, petroleum ether/ethyl acetate=3/1) to give 2-(methyl-d3)thiazole-5-sulfonyl chloride (470 mg, 2.32 mmol, 32% yield, 99% purity) as a yellow oil.

### Example 36.3. Preparation of N-(3-cyano-4-methyl-1H-indol-7-yl)-2-(methyl-d3)thiazole-5-sulfonamide (Compound 143)

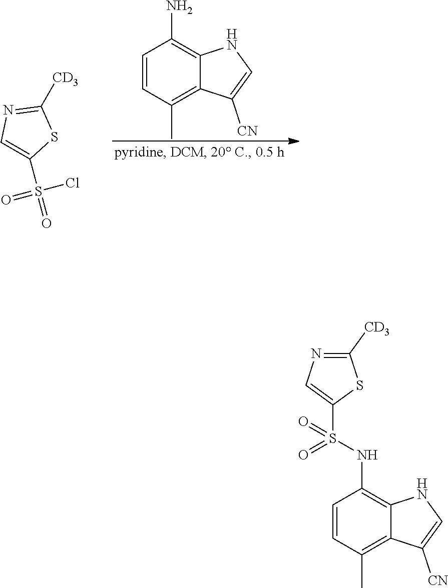 [SMILES: [2H]C([2H])([2H])c1ncc(S(=O)(=O)Cl)s1 ; Cc1ccc(N)c2[nH]cc(C#N)c12 ; [2H]C([2H])([2H])c1ncc(S(=O)(=O)Nc2ccc(C)c3c(C#N)c[nH]c23)s1]

To a solution of 2-(methyl-d₃)thiazole-5-sulfonyl chloride (50.0 mg, 249 μmol, 1.00 eq) in dichloromethane (1.00 mL) was added pyridine (39.4 mg, 498 μmol, 40.2 L, 2.00 eq) and 7-amino-4-methyl-1H-indole-3-carbonitrile (42.7 mg, 249 μmol, 1.00 eq). The mixture was stirred at 20° C. for 0.5 h. The mixture was concentrated under vacuum to give a residue. The residue was purified by prep-HPLC(column: Phenomenex luna C18 150*25 mm*10 um; mobile phase:[water(FA)-ACN]; gradient: 27%-57% B over 10 min) and lyophilized to give N-(3-cyano-4-methyl-1H-indol-7-yl)-2-(methyl-d3)thiazole-5-sulfonamide (58.52 mg, 174.47 μmol, 70.03% yield). MS (ESI) m/z 336.1 [M+H]⁺. ¹H NMR (400 MHz, DMSO-d₆) δ=11.97 (br s, 1H), 10.44-10.12 (m, 1H), 8.17 (d, J=2.0 Hz, 1H), 7.89 (s, 1H), 6.86 (d, J=7.6 Hz, 1H), 6.72 (d, J=7.6 Hz, 1H), 2.60 (s, 3H)

Compounds 36-151 were prepared or can be prepared by similar methods to those described herein and are shown in Table 1.

TABLE 1

| Cmpd. | |
|---|---|
| No. | Structure |
| 36 | 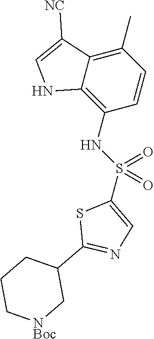 [SMILES: Cc1ccc(NS(=O)(=O)c2cnc(C3CCCN(C(=O)OC(C)(C)C)C3)s2)c2[nH]cc(C#N)c12] |
| 37 | 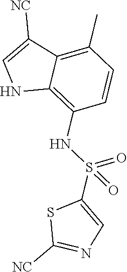 [SMILES: Cc1ccc(NS(=O)(=O)c2cnc(C#N)s2)c2[nH]cc(C#N)c12] |
| 38 | 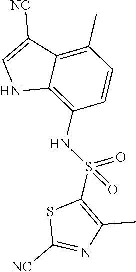 [SMILES: Cc1nc(C#N)sc1S(=O)(=O)Nc1ccc(C)c2c(C#N)c[nH]c12] |
| 39 | 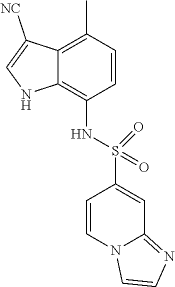 [SMILES: Cc1ccc(NS(=O)(=O)c2ccn3ccnc3c2)c2[nH]cc(C#N)c12] |
| 40 | 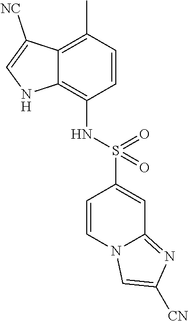 [SMILES: Cc1ccc(NS(=O)(=O)c2ccn3cc(C#N)nc3c2)c2[nH]cc(C#N)c12] |
| 41 | 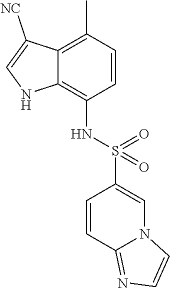 [SMILES: Cc1ccc(NS(=O)(=O)c2ccc3nccn3c2)c2[nH]cc(C#N)c12] |
| 42 | 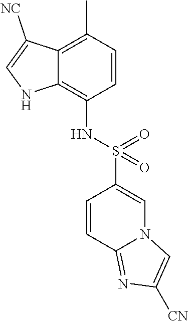 [SMILES: Cc1ccc(NS(=O)(=O)c2ccc3nc(C#N)cn3c2)c2[nH]cc(C#N)c12] |
| 43 | 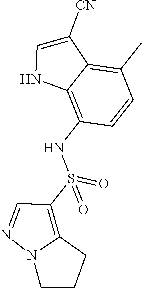 [SMILES: Cc1ccc(NS(=O)(=O)c2cnn3c2CCC3)c2[nH]cc(C#N)c12] |
| 44 | 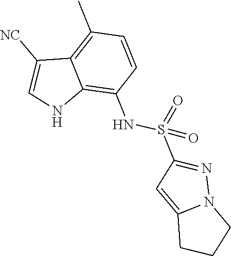 [SMILES: Cc1ccc(NS(=O)(=O)c2cc3n(n2)CCC3)c2[nH]cc(C#N)c12] |
| 45 | 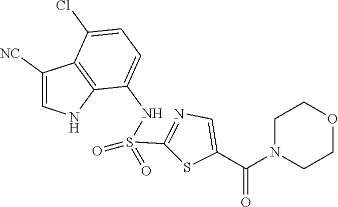 [SMILES: N#Cc1c[nH]c2c(NS(=O)(=O)c3ncc(C(=O)N4CCOCC4)s3)ccc(Cl)c12] |
| 46 | 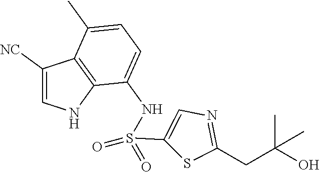 [SMILES: Cc1ccc(NS(=O)(=O)c2cnc(CC(C)(C)O)s2)c2[nH]cc(C#N)c12] |
| 47 | 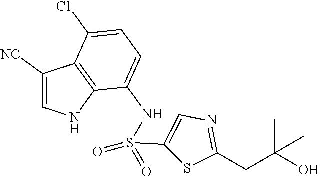 [SMILES: CC(C)(O)Cc1ncc(S(=O)(=O)Nc2ccc(Cl)c3c(C#N)c[nH]c23)s1] |
| 48 | 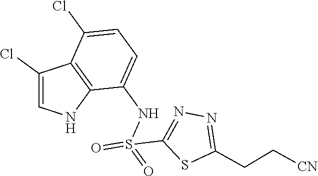 [SMILES: N#CCCc1nnc(S(=O)(=O)Nc2ccc(Cl)c3c(Cl)c[nH]c23)s1] |
| 49 | 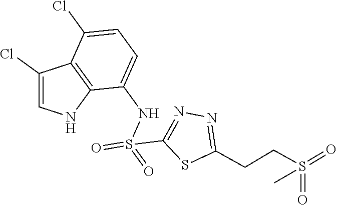 [SMILES: CS(=O)(=O)CCc1nnc(S(=O)(=O)Nc2ccc(Cl)c3c(Cl)c[nH]c23)s1] |
| 50 | 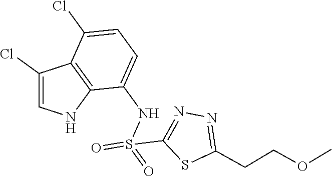 [SMILES: COCCc1nnc(S(=O)(=O)Nc2ccc(Cl)c3c(Cl)c[nH]c23)s1] |
| 51 | 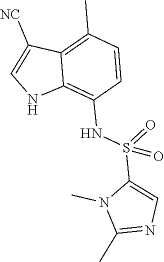 [SMILES: Cc1ccc(NS(=O)(=O)c2cnc(C)n2C)c2[nH]cc(C#N)c12] |
| 52 | 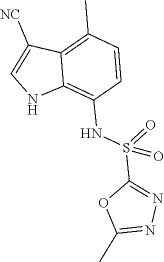 [SMILES: Cc1nnc(S(=O)(=O)Nc2ccc(C)c3c(C#N)c[nH]c23)o1] |
| 53 | 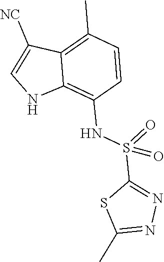 [SMILES: Cc1nnc(S(=O)(=O)Nc2ccc(C)c3c(C#N)c[nH]c23)s1] |
| 54 | 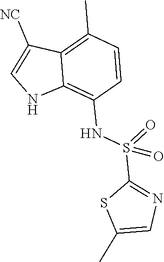 [SMILES: Cc1cnc(S(=O)(=O)Nc2ccc(C)c3c(C#N)c[nH]c23)s1] |
| 55 | 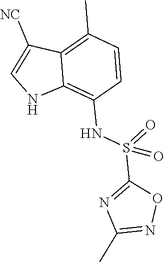 [SMILES: Cc1noc(S(=O)(=O)Nc2ccc(C)c3c(C#N)c[nH]c23)n1] |
| 56 | 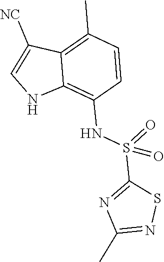 [SMILES: Cc1nsc(S(=O)(=O)Nc2ccc(C)c3c(C#N)c[nH]c23)n1] |
| 57 | 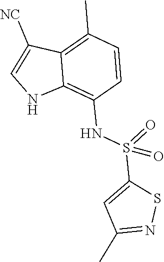 [SMILES: Cc1cc(S(=O)(=O)Nc2ccc(C)c3c(C#N)c[nH]c23)sn1] |
| 58 | 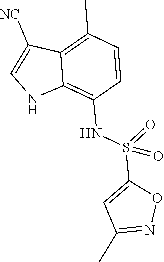 [SMILES: Cc1cc(S(=O)(=O)Nc2ccc(C)c3c(C#N)c[nH]c23)on1] |
| 59 | 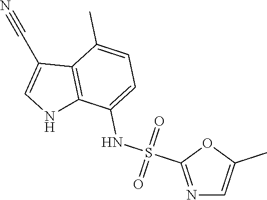 [SMILES: Cc1cnc(S(=O)(=O)Nc2ccc(C)c3c(C#N)c[nH]c23)o1] |
| 60 | 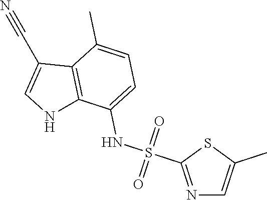 [SMILES: Cc1cnc(S(=O)(=O)Nc2ccc(C)c3c(C#N)c[nH]c23)s1] |
| 61 | 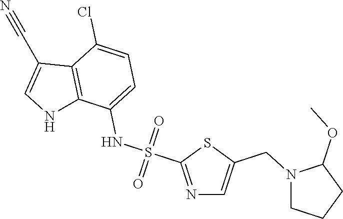 [SMILES: COC1CCCN1Cc1cnc(S(=O)(=O)Nc2ccc(Cl)c3c(C#N)c[nH]c23)s1] |
| 62 | 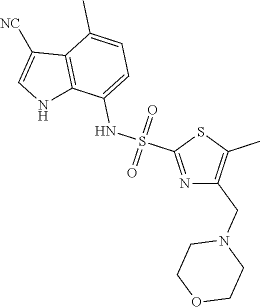 [SMILES: Cc1sc(S(=O)(=O)Nc2ccc(C)c3c(C#N)c[nH]c23)nc1CN1CCOCC1] |
| 63 | 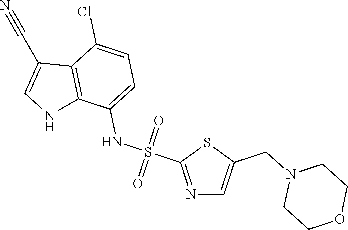 [SMILES: N#Cc1c[nH]c2c(NS(=O)(=O)c3ncc(CN4CCOCC4)s3)ccc(Cl)c12] |
| 64 | 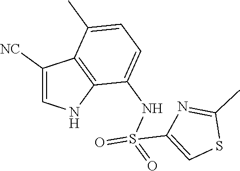 [SMILES: Cc1nc(S(=O)(=O)Nc2ccc(C)c3c(C#N)c[nH]c23)cs1] |
| 65 | 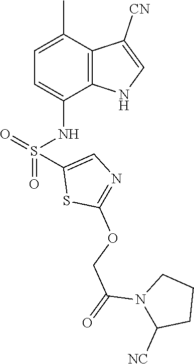 [SMILES: Cc1ccc(NS(=O)(=O)c2cnc(OCC(=O)N3CCCC3C#N)s2)c2[nH]cc(C#N)c12] |
| 66 | 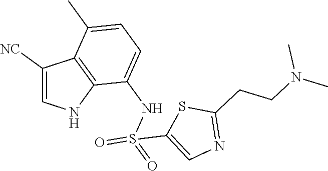 [SMILES: Cc1ccc(NS(=O)(=O)c2cnc(CCN(C)C)s2)c2[nH]cc(C#N)c12] |
| 67 | 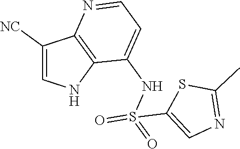 [SMILES: Cc1ncc(S(=O)(=O)Nc2ccnc3c(C#N)c[nH]c23)s1] |
| 68 | 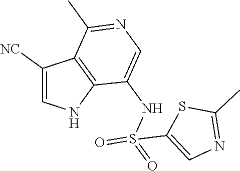 [SMILES: Cc1ncc(S(=O)(=O)Nc2cnc(C)c3c(C#N)c[nH]c23)s1] |
| 69 | 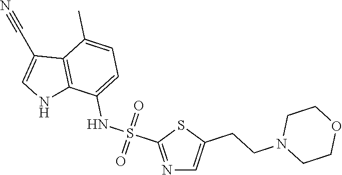 [SMILES: Cc1ccc(NS(=O)(=O)c2ncc(CCN3CCOCC3)s2)c2[nH]cc(C#N)c12] |
| 70 | 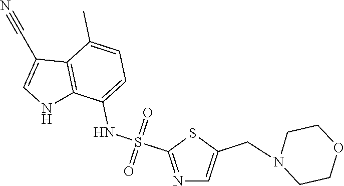 [SMILES: Cc1ccc(NS(=O)(=O)c2ncc(CN3CCOCC3)s2)c2[nH]cc(C#N)c12] |
| 71 | 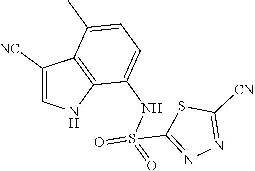 [SMILES: Cc1ccc(NS(=O)(=O)c2nnc(C#N)s2)c2[nH]cc(C#N)c12] |
| 72 | 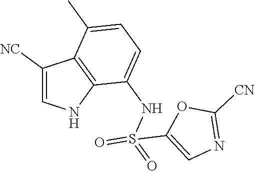 [SMILES: Cc1ccc(NS(=O)(=O)c2cnc(C#N)o2)c2[nH]cc(C#N)c12] |
| 73 | 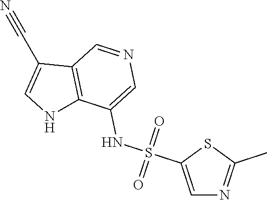 [SMILES: Cc1ncc(S(=O)(=O)Nc2cncc3c(C#N)c[nH]c23)s1] |
| 74 | 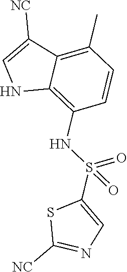 [SMILES: Cc1ccc(NS(=O)(=O)c2cnc(C#N)s2)c2[nH]cc(C#N)c12] |
| 75 | 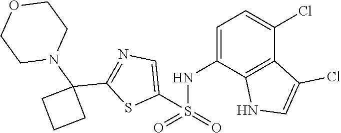 [SMILES: O=S(=O)(Nc1ccc(Cl)c2c(Cl)c[nH]c12)c1cnc(C2(N3CCOCC3)CCC2)s1] |
| 76 | 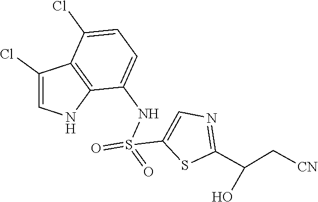 [SMILES: N#CCC(O)c1ncc(S(=O)(=O)Nc2ccc(Cl)c3c(Cl)c[nH]c23)s1] |
| 77 | 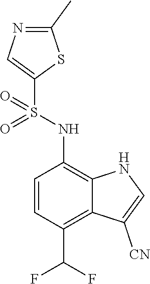 [SMILES: Cc1ncc(S(=O)(=O)Nc2ccc(C(F)F)c3c(C#N)c[nH]c23)s1] |
| 78 | 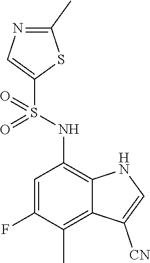 [SMILES: Cc1ncc(S(=O)(=O)Nc2cc(F)c(C)c3c(C#N)c[nH]c23)s1] |
| 79 | 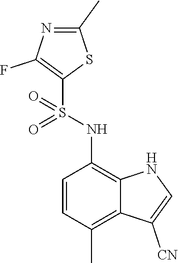 [SMILES: Cc1nc(F)c(S(=O)(=O)Nc2ccc(C)c3c(C#N)c[nH]c23)s1] |
| 80 | 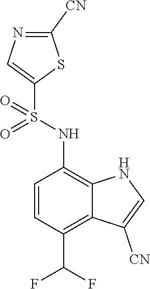 [SMILES: N#Cc1ncc(S(=O)(=O)Nc2ccc(C(F)F)c3c(C#N)c[nH]c23)s1] |
| 81 | 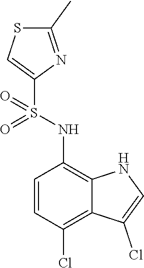 [SMILES: Cc1nc(S(=O)(=O)Nc2ccc(Cl)c3c(Cl)c[nH]c23)cs1] |
| 82 | 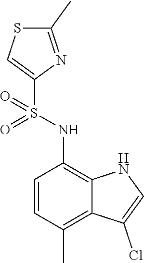 [SMILES: Cc1nc(S(=O)(=O)Nc2ccc(C)c3c(Cl)c[nH]c23)cs1] |
| 83 | 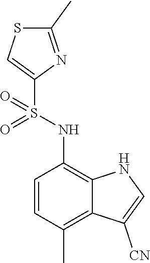 [SMILES: Cc1nc(S(=O)(=O)Nc2ccc(C)c3c(C#N)c[nH]c23)cs1] |
| 84 | 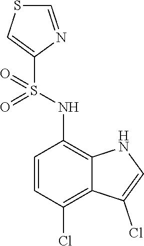 [SMILES: O=S(=O)(Nc1ccc(Cl)c2c(Cl)c[nH]c12)c1cscn1] |
| 85 | 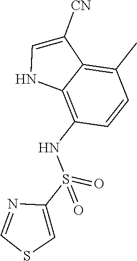 [SMILES: Cc1ccc(NS(=O)(=O)c2cscn2)c2[nH]cc(C#N)c12] |
| 86 | 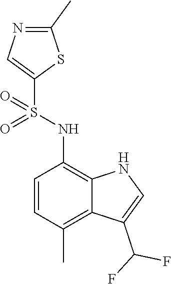 [SMILES: Cc1ncc(S(=O)(=O)Nc2ccc(C)c3c(C(F)F)c[nH]c23)s1] |
| 87 | 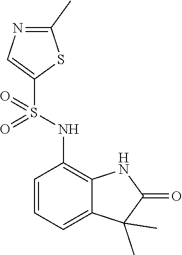 [SMILES: Cc1ncc(S(=O)(=O)Nc2cccc3c2NC(=O)C3(C)C)s1] |
| 88 | 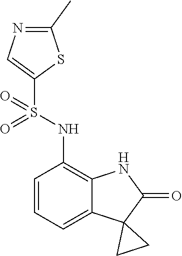 [SMILES: Cc1ncc(S(=O)(=O)Nc2cccc3c2NC(=O)C32CC2)s1] |
| 89 | 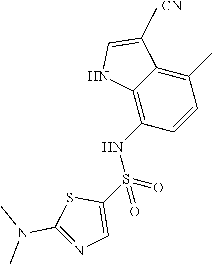 |
| 90 | 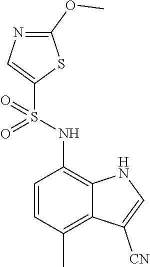 [SMILES: COc1ncc(S(=O)(=O)Nc2ccc(C)c3c(C#N)c[nH]c23)s1] |
| 91 | 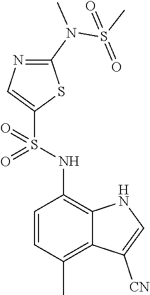 [SMILES: Cc1ccc(NS(=O)(=O)c2cnc(N(C)S(C)(=O)=O)s2)c2[nH]cc(C#N)c12] |
| 92 | 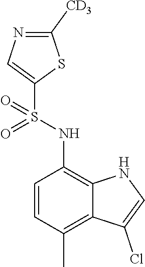 [SMILES: [2H]C([2H])([2H])c1ncc(S(=O)(=O)Nc2ccc(C)c3c(Cl)c[nH]c23)s1] |
| 93 | 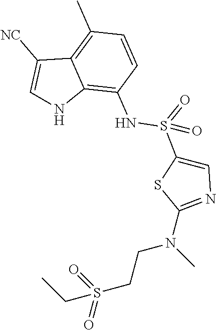 [SMILES: CCS(=O)(=O)CCN(C)c1ncc(S(=O)(=O)Nc2ccc(C)c3c(C#N)c[nH]c23)s1] |
| 94 | 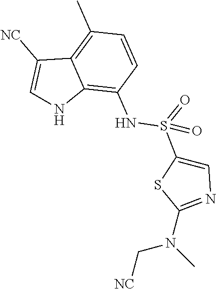 [SMILES: Cc1ccc(NS(=O)(=O)c2cnc(N(C)CC#N)s2)c2[nH]cc(C#N)c12] |
| 95 | 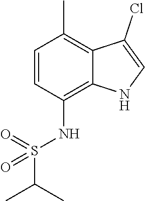 [SMILES: Cc1ccc(NS(=O)(=O)C(C)C)c2[nH]cc(Cl)c12] |
| 96 | 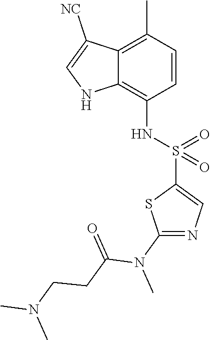 [SMILES: Cc1ccc(NS(=O)(=O)c2cnc(N(C)C(=O)CCN(C)C)s2)c2[nH]cc(C#N)c12] |
| 97 | 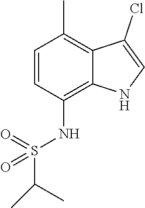 [SMILES: Cc1ccc(NS(=O)(=O)C(C)C)c2[nH]cc(Cl)c12] |
| 98 | 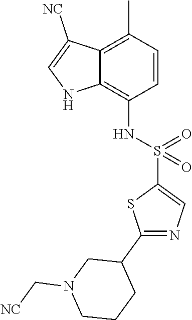 [SMILES: Cc1ccc(NS(=O)(=O)c2cnc(C3CCCN(CC#N)C3)s2)c2[nH]cc(C#N)c12] |
| 99 | 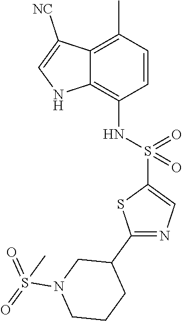 [SMILES: Cc1ccc(NS(=O)(=O)c2cnc(C3CCCN(S(C)(=O)=O)C3)s2)c2[nH]cc(C#N)c12] |
| 100 | 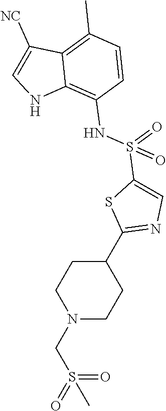 [SMILES: Cc1ccc(NS(=O)(=O)c2cnc(C3CCN(CS(C)(=O)=O)CC3)s2)c2[nH]cc(C#N)c12] |
| 101 | 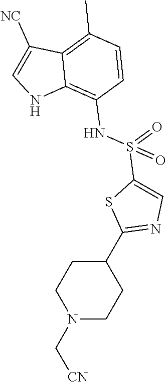 [SMILES: Cc1ccc(NS(=O)(=O)c2cnc(C3CCN(CC#N)CC3)s2)c2[nH]cc(C#N)c12] |
| 102 | 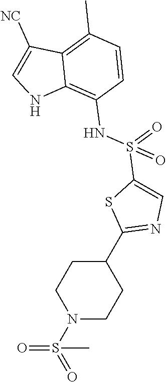 [SMILES: Cc1ccc(NS(=O)(=O)c2cnc(C3CCN(S(C)(=O)=O)CC3)s2)c2[nH]cc(C#N)c12] |
| 103 | 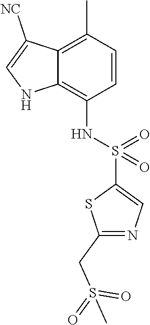 [SMILES: Cc1ccc(NS(=O)(=O)c2cnc(CS(C)(=O)=O)s2)c2[nH]cc(C#N)c12] |
| 104 | 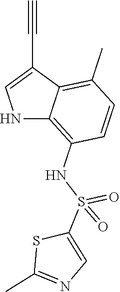 [SMILES: C#Cc1c[nH]c2c(NS(=O)(=O)c3cnc(C)s3)ccc(C)c12] |
| 105 | 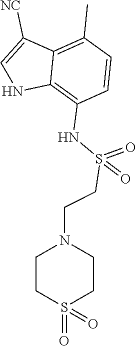 [SMILES: Cc1ccc(NS(=O)(=O)CCN2CCS(=O)(=O)CC2)c2[nH]cc(C#N)c12] |
| 106 | 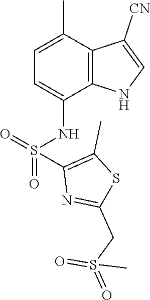 [SMILES: Cc1sc(CS(C)(=O)=O)nc1S(=O)(=O)Nc1ccc(C)c2c(C#N)c[nH]c12] |
| 109 | 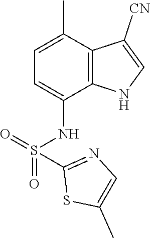 [SMILES: Cc1cnc(S(=O)(=O)Nc2ccc(C)c3c(C#N)c[nH]c23)s1] |
| 110 | 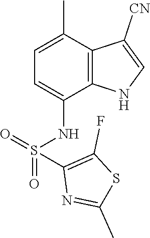 [SMILES: Cc1nc(S(=O)(=O)Nc2ccc(C)c3c(C#N)c[nH]c23)c(F)s1] |
| 111 | 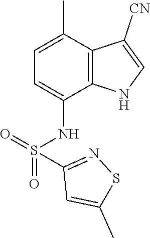 [SMILES: Cc1cc(S(=O)(=O)Nc2ccc(C)c3c(C#N)c[nH]c23)ns1] |
| 113 | 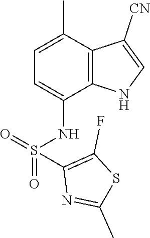 [SMILES: Cc1nc(S(=O)(=O)Nc2ccc(C)c3c(C#N)c[nH]c23)c(F)s1] |
| 114 | 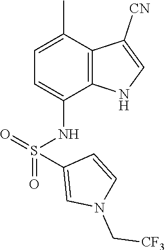 [SMILES: Cc1ccc(NS(=O)(=O)c2ccn(CC(F)(F)F)c2)c2[nH]cc(C#N)c12] |
| 115 | 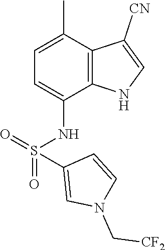 |
| 116 | 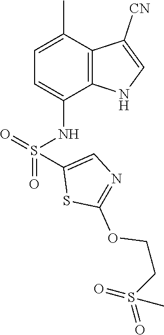 [SMILES: Cc1ccc(NS(=O)(=O)c2cnc(OCCS(C)(=O)=O)s2)c2[nH]cc(C#N)c12] |
| 117 | 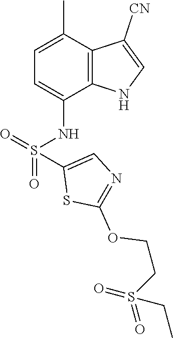 [SMILES: CCS(=O)(=O)CCOc1ncc(S(=O)(=O)Nc2ccc(C)c3c(C#N)c[nH]c23)s1] |
| 118 | 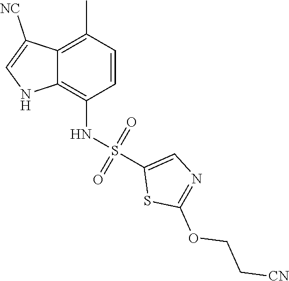 [SMILES: Cc1ccc(NS(=O)(=O)c2cnc(OCCC#N)s2)c2[nH]cc(C#N)c12] |
| 119 | 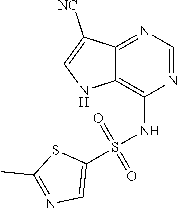 [SMILES: Cc1ncc(S(=O)(=O)Nc2ncnc3c(C#N)c[nH]c23)s1] |
| 120 | 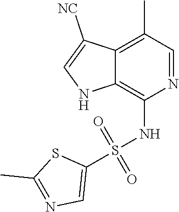 [SMILES: Cc1ncc(S(=O)(=O)Nc2ncc(C)c3c(C#N)c[nH]c23)s1] |
| 121 | 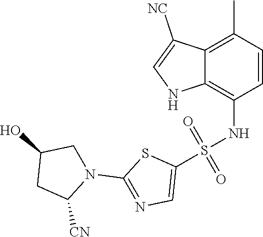 [SMILES: Cc1ccc(NS(=O)(=O)c2cnc(N3C[C@H](O)C[C@H]3C#N)s2)c2[nH]cc(C#N)c12] |
| 122 | 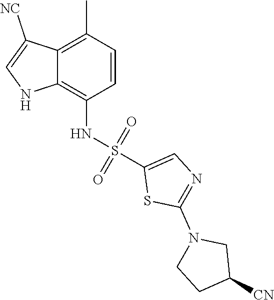 [SMILES: Cc1ccc(NS(=O)(=O)c2cnc(N3CC[C@H](C#N)C3)s2)c2[nH]cc(C#N)c12] |
| 122 | 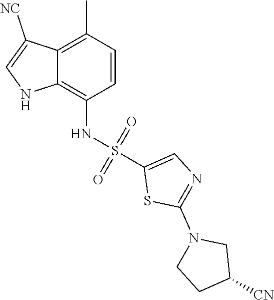 [SMILES: Cc1ccc(NS(=O)(=O)c2cnc(N3CC[C@@H](C#N)C3)s2)c2[nH]cc(C#N)c12] |
| 124 | 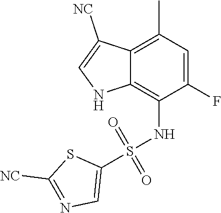 [SMILES: Cc1cc(F)c(NS(=O)(=O)c2cnc(C#N)s2)c2[nH]cc(C#N)c12] |
| 125 | 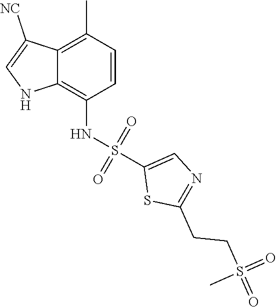 [SMILES: Cc1ccc(NS(=O)(=O)c2cnc(CCS(C)(=O)=O)s2)c2[nH]cc(C#N)c12] |
| 126 | 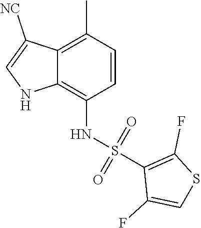 [SMILES: Cc1ccc(NS(=O)(=O)c2c(F)csc2F)c2[nH]cc(C#N)c12] |
| 127 | 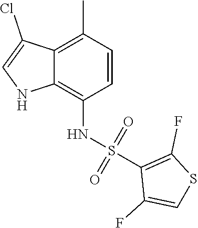 [SMILES: Cc1ccc(NS(=O)(=O)c2c(F)csc2F)c2[nH]cc(Cl)c12] |
| 128 | 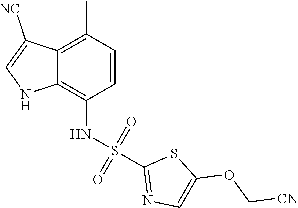 [SMILES: Cc1ccc(NS(=O)(=O)c2ncc(OCC#N)s2)c2[nH]cc(C#N)c12] |
| 129 | 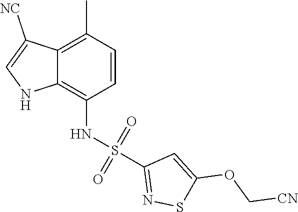 [SMILES: Cc1ccc(NS(=O)(=O)c2cc(OCC#N)sn2)c2[nH]cc(C#N)c12] |
| 130 | 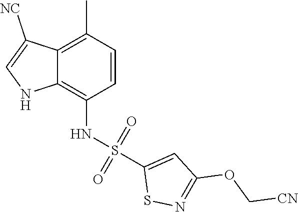 [SMILES: Cc1ccc(NS(=O)(=O)c2cc(OCC#N)ns2)c2[nH]cc(C#N)c12] |
| 131 | 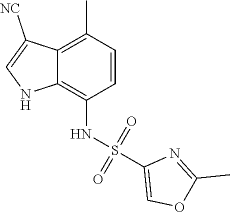 [SMILES: Cc1nc(S(=O)(=O)Nc2ccc(C)c3c(C#N)c[nH]c23)co1] |
| 132 | 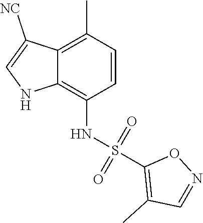 [SMILES: Cc1cnoc1S(=O)(=O)Nc1ccc(C)c2c(C#N)c[nH]c12] |
| 133 | 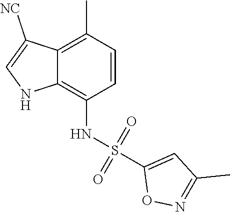 [SMILES: Cc1cc(S(=O)(=O)Nc2ccc(C)c3c(C#N)c[nH]c23)on1] |
| 134 | 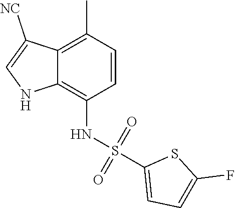 [SMILES: Cc1ccc(NS(=O)(=O)c2ccc(F)s2)c2[nH]cc(C#N)c12] |
| 135 | 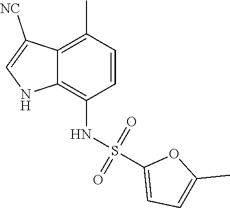 [SMILES: Cc1ccc(S(=O)(=O)Nc2ccc(C)c3c(C#N)c[nH]c23)o1] |
| 136 | 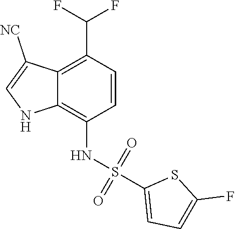 [SMILES: N#Cc1c[nH]c2c(NS(=O)(=O)c3ccc(F)s3)ccc(C(F)F)c12] |
| 137 | 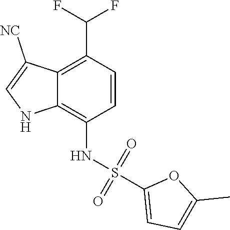 [SMILES: Cc1ccc(S(=O)(=O)Nc2ccc(C(F)F)c3c(C#N)c[nH]c23)o1] |
| 138 | 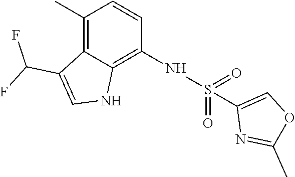 [SMILES: Cc1nc(S(=O)(=O)Nc2ccc(C)c3c(C(F)F)c[nH]c23)co1] |
| 139 | 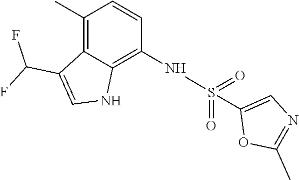 [SMILES: Cc1ncc(S(=O)(=O)Nc2ccc(C)c3c(C(F)F)c[nH]c23)o1] |
| 140 | 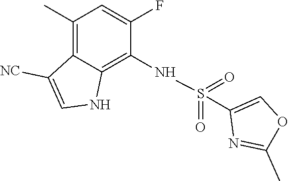 [SMILES: Cc1nc(S(=O)(=O)Nc2c(F)cc(C)c3c(C#N)c[nH]c23)co1] |
| 141 | 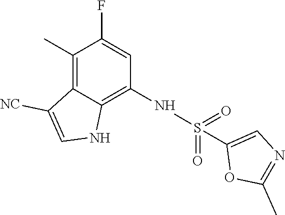 [SMILES: Cc1ncc(S(=O)(=O)Nc2cc(F)c(C)c3c(C#N)c[nH]c23)o1] |
| 142 | 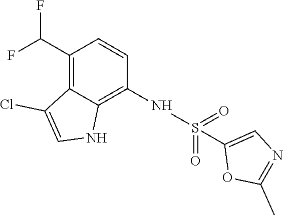 [SMILES: Cc1ncc(S(=O)(=O)Nc2ccc(C(F)F)c3c(Cl)c[nH]c23)o1] |
| 143 | 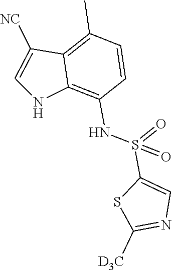 [SMILES: [2H]C([2H])([2H])c1ncc(S(=O)(=O)Nc2ccc(C)c3c(C#N)c[nH]c23)s1] |
| 144 | 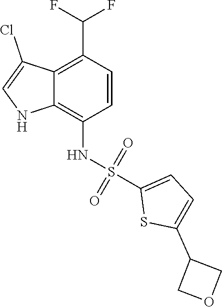 [SMILES: O=S(=O)(Nc1ccc(C(F)F)c2c(Cl)c[nH]c12)c1ccc(C2COC2)s1] |
| 145 | 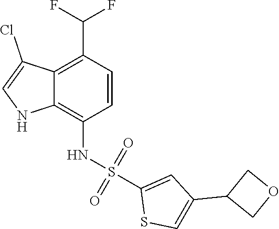 [SMILES: O=S(=O)(Nc1ccc(C(F)F)c2c(Cl)c[nH]c12)c1cc(C2COC2)cs1] |
| 146 | 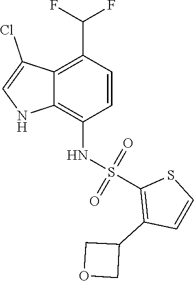 [SMILES: O=S(=O)(Nc1ccc(C(F)F)c2c(Cl)c[nH]c12)c1sccc1C1COC1] |
| 147 | 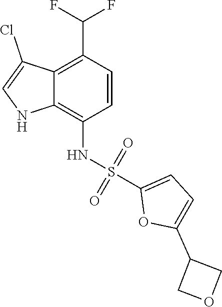 [SMILES: O=S(=O)(Nc1ccc(C(F)F)c2c(Cl)c[nH]c12)c1ccc(C2COC2)o1] |
| 148 | 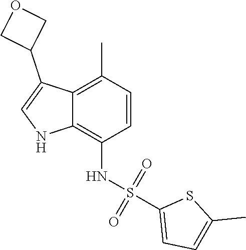 [SMILES: Cc1ccc(S(=O)(=O)Nc2ccc(C)c3c(C4COC4)c[nH]c23)s1] |
| 149 | 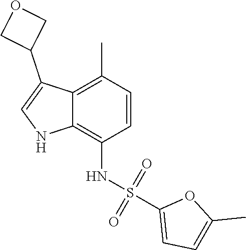 [SMILES: Cc1ccc(S(=O)(=O)Nc2ccc(C)c3c(C4COC4)c[nH]c23)o1] |
| 150 | 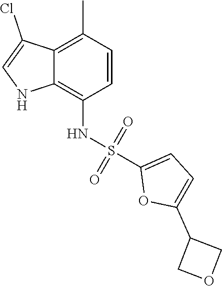 [SMILES: Cc1ccc(NS(=O)(=O)c2ccc(C3COC3)o2)c2[nH]cc(Cl)c12] |
| 151 | 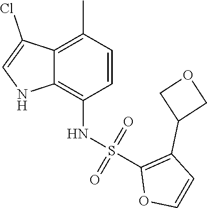 [SMILES: Cc1ccc(NS(=O)(=O)c2occc2C2COC2)c2[nH]cc(Cl)c12] |

### Example 37. Cell Viability Assays

HCT116 colorectal cells were used in these experiments. HCT116 cells were obtained from ATCC. The CellTiter-Glo® Luminescent Cell Viability Assay Reagent was obtained from Promega.

Into a 96-well plate, 3000 HCT116 cells were seeded with 100 μL of media (McCoy's 5A Medium supplemented with 10% FBS, 100 units penicillin, and 100 μg streptomycin per mL) 24h before the experiment. For compound treatment, the liquid handling system-Pico machine was used to prepare all the compounds. Each master plate contained serial dilution of 2 compounds, including the control compound E7820. 10 mM compound stock was added to each well of the assay plate to give final concentrations of 10, 3, 1, 0.3, 0.1, 0.001, 0.003, 0.002 and 0 μM. Each concentration was tested in triplicate. After 72h, cell viability was determined using CellTiter-Glo Luminescent Cell Viability Assay Reagent following the manufacturer's recommended protocol. Briefly, equilibrate the assay plate and its contents at room temperature for approximately 30 minutes. 100 μL of CellTiter-Glo reagent was added to each well of the assay plate and the contents were mixed for 2 minutes at 500 rpm on an orbital shaker to induce cell lysis (Fisherbrand). The plate was incubated at room temperature for 10 minutes to stabilize luminescent signal. Then the plate was immediately placed in a plate reader (PerkinElmer Multimode Plate Reader Envision2105) and the luminescence signal (0.5 second per well integration time) was determined. IC50 values were calculated using Graphpad Prism 9 software.

Compounds described herein as exemplified in the Examples, showed IC₅₀ values in the following ranges: A: IC₅₀<500 nM; B: 500 nM≤IC₅₀≤1000 nM; C: IC₅₀>1000 nM.

TABLE 2

| Cmpd. No. | HCT116 Assay (IC₅₀) |
|---|---|
| 1 | A |
| 2 | A |
| 4 | B |
| 5 | B |
| 6 | A |
| 7 | A |
| 8 | C |
| 9 | B |
| 10 | C |
| 11 | C |
| 12 | C |
| 13 | B |
| 15 | B |
| 16 | C |
| 17 | B |
| 18 | B |
| 19 | B |
| 20 | A |
| 21 | B |
| 23 | B |
| 24 | B |
| 25 | C |
| 26 | C |
| 27 | C |
| 28 | C |
| 29 | C |
| 30 | C |
| 31 | C |
| 32 | B |
| 33 | C |
| 34 | B |
| 35 | B |
| 40 | B |
| 41 | B |
| 42 | C |
| 43 | B |
| 44 | B |
| 57 | A |
| 65 | C |
| 75 | B |
| 84 | B |
| 85 | B |
| 90 | A |
| 91 | A |
| 92 | B |
| 104 | C |
| 143 | A |

### Example 38: ADME Studies

### General Solubility Protocol

Into a 96-well rack, 15 μL of stock solution (10 mM) of each sample was placed. Into each vial of a cap-less Solubility Sample plate 485 μL of buffer was added. The assay was performed in duplicate. To each vial one stir stick was added and each vial was sealed using a molded PTFE/Silicone plug. The Solubility Sample plate was then transferred to an Eppendorf Thermomixer Comfort plate shaker and shake at 25° C. at 1100 RPM for 2 hours. After 2 hours, the stir sticks were removed using a big magnet and the samples were transferred from the solubility sample plate into the filter plate. Using a vacuum manifold, all the samples were filtered. An aliquot of 5 μL was taken from the filtrate and 5 μL blank DMSO followed by addition of 490 μL of a mixture of H₂O and acetonitrile containing an internal standard (1:1). The dilution factor may be changed according to the solubility value and the LC/MS signal response.

### General Liver Microsomes Stability Protocol:

A 100 μM test compound solution and PC solution (verapamil) was prepared by adding 2 μL of 10 mM stock solution in DMSO to 198 μL of 50% acetonitrile/50% water.

### Step 1: Incubation

Two separate experiments were performed as follows:

a) With Cofactors (NADPH): 25 μL of 10 mM NADPH was added to the incubations. The final concentrations of microsomes and NADPH were 0.5 mg/mL and 1 mM, respectively.

b) Without Cofactors (NADPH): 25 μL of 100 mM Phosphate buffer was added to the incubations. The final concentration of microsomes was 0.5 mg/mL. The mixture was pre-warmed at 37° C. for 10 minutes.

The reaction was started with the addition of 2.5 μL of 100 M control compound or test compound solutions. Verapamil was used as positive control in this study. The final concentration of test compound or control compound was 1 μM. The incubation solution was incubated in a water bath at 37° C.

### Step 2. Reaction Quenching

Aliquots of 30 μL were taken from the reaction solution at 0.5, 15, 30, 45 and 60 minutes. The reaction was stopped by the addition of 5 volumes of cold acetonitrile with IS (100 nM alprazolam, 200 nM caffeine and 100 nM tolbutamide).

Samples were then centrifuged at 3,220 g for 40 minutes. An aliquot of 100 μL of the supernatant was mixed with 100 μL of ultra-pure H₂O and then used for LC-MS/MS analysis.

### General MDCK-MDR1 Assay Protocol:

To prepare transport buffer (HBSS with 10 mM HEPES, pH 7.4), 2.383 g of HEPES and 0.35 g sodium hydrogen carbonate was accurately weighed and added into 900 mL of pure water, then sonicated to dissolve the content. Into the solution was transferred 100 mL of 10×HBSS, and the solution was placed on a stirrer. The pH of the solution was slowly adjusted with sodium hydroxide to 7.4, followed with filtering.

MDCK-MDR1 plate(s) were removed from the incubator. The monolayer was washed the monolayer twice with pre-warmed HBSS (10 mM HEPES, pH 7.4). Then the plate(s) were incubated at 37° C. for 30 minutes.

Propranolol was used as the high permeability marker. Digoxin was used as the substrate of breast cancer resistant protein (MDR1). Stock solutions of test compound(s) and digoxin in DMSO at 0.2 mM were prepared and diluted with HBSS (10 mM HEPES, pH 7.4) to get 1 μM working solutions. Stock solutions of propranolol in DMSO at 1 mM was prepared and diluted with HBSS (10 mM HEPES, pH 7.4) to get 5 μM working solutions.

To determine the rate of drug transport in the apical to basolateral direction, 125 μL of the working solution was added to the Transwell insert (apical compartment), and a 50 μL sample was immediately transferred from the apical compartment to 200 μL quenching solvents (acetonitrile with 100 nM alprazolam, 200 nM caffeine, 200 nM labetalol and 100 nM tolbutamide) in a new 96-well plate as the initial donor sample (A-B). The plate was shaken at 1000 rpm for 10 minutes. The wells in the receiver plate (basolateral compartment) were filled with 235 μL of transport buffer. All incubations were performed in duplicate.

To determine the rate of drug transport in the basolateral to apical direction, 285 μL of the 1 μM working solution was added to the receiver plate wells (basolateral compartment), and a 50 μL sample was immediately transferred from the basolateral compartment to 200 μL quenching solvents (acetonitrile with 100 nM alprazolam, 200 nM caffeine, 200 nM labetalol and 100 nM tolbutamide) in a new 96-well plate as the initial donor sample (B-A). Shake the plate at 1000 rpm 10 minutes. The Transwell insert (apical compartment) was filled with 75 μL of transport buffer. The apical to basolateral direction and the basolateral to apical direction were done at the same time.

The Transwell insert plate was inserted into the basolateral plate, transferred into the incubator and incubated at 37° C. for 2 hours.

At the end of the incubation, 50 μL samples from donor sides (apical compartment for Ap→Bl flux, and basolateral compartment for Bl→Ap flux) and receiver sides (basolateral compartment for Ap→Bl flux, and apical compartment for Bl→Ap flux) were transferred to wells of a new 96-well plate, followed by the addition of 4 volume of quenching solvents (acetonitrile with 100 nM alprazolam, 200 nM caffeine, 200 nM labetalol and 100 nM tolbutamide). Samples were Vortexed for 10 minutes and then centrifuged at 3,220 g for 40 minutes. An aliquot of 100 μL of the supernatant was mixed with an appropriate volume of ultra-pure water before LC-MS/MS analysis.

To determine the Lucifer yellow leakage after 2-hour transport period, Lucifer yellow working solutions were prepared by diluting the stock solution with HBSS (10 mM HEPES, pH 7.4) to reach the final concentration of 100 μM. To the apical compartment, 100 μL of the Lucifer yellow solution were added. The plate(s) were incubated at 37° C. for 30 minutes and 80 μL was directly removed from the apical and basolateral wells and transferred to new 96 wells plates. Lucifer yellow fluorescence (to monitor monolayer integrity) was measured in a fluorescence plate reader at 485 nM excitation and 530 nM emission.

TABLE 3
ADME Properties

| | Solubility | Metabolic Stability | MDCK-MDR1 |
|---|---|---|---|
| | (PBS, pH 7.4) | (human, mouse) | Efflux |
| Compound | (μM) | Half-life (min) | ratio |
| E7820 | 80.73 | 50.73; 28.68 | 29.90 |
| 1 | 51.55 | 66.17; 47.13 | 25.46 |
| 2 | 292 | >231, 161 | 6.74 |
| 20 | 1.91 | 23.74, NV | 1.11 |
| 39 | 1.24 | 69.43, 33.98 | 50.06 |
| 57 | 293.64 | 33.42, 61.45 | 4.84 |

NV = no value

### Example 39. Pharmacokinetics Studies

Single dose PK parameters in plasma were determined following intravenous administration of compound 1 and comparative compound E7820 at 1 mg/kg by using the formulation of 10% DMSO/30% PEG400/60% Saline. The plasma samples were collected at the following time points: 0.083, 0.25, 0.5, 1, 2, 4, 8 and 24 hrs post-dose. The concentrations in plasma were determined.

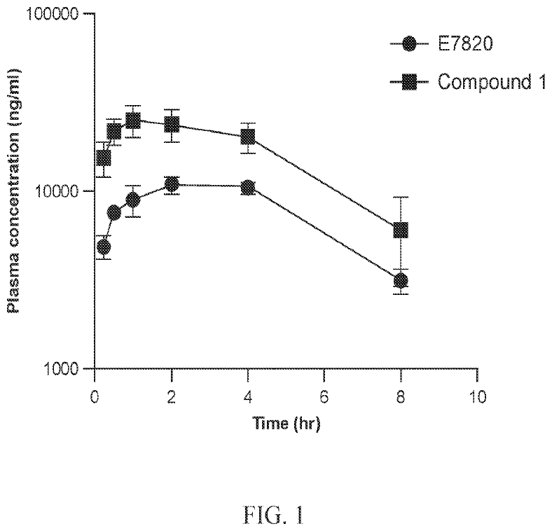

Single dose PK parameters in plasma were determined following oral gavage of Compound 1 and comparative compound E7820 at 20 mg/kg by using the formulation of 0.5% MC (400 cps) and 0.1% Tween80 in water. The plasma samples were collected at the following time points: 0.25, 0.5, 1, 2, 4, 8 and 24 hrs post dose. The concentrations in plasma were determined as shown in FIG. 1.

TABLE 4
Mean Plasma Pharmacokinetic Parameters of Compound 1 Following
IV and PO Administration in Female BALB/C Mice

| | CL | Vₛₛ | C₀ | Cₘₐₓ | Tₘₐₓ | AUC₍₀₋ₜ₎ | AUC_{(0-∞)} | t_{1/2} | F |
|---|---|---|---|---|---|---|---|---|---|
| Group | (mL/min/kg) | (L/kg) | (ng/mL) | (ng/mL) | (h) | (h*ng/mL) | (h*ng/mL) | (h) | (%) |
| IV | 9.4 | 0.201 | 6055 | NA | NA | 1862 | 1863 | 0.67 | NA |
| PO | NA | NA | NA | 25867 | 2.33 | 151455 | 189912 | 2.52 | 407 |

TABLE 5
Mean Plasma and Telencephalon Pharmacokinetic Parameters of E7820
Following IV and PO Administration in Female BALB/C Mice

| | CL | Vₛₛ | C₀ | Cₘₐₓ | Tₘₐₓ | AUC₍₀₋ₜ₎ | AUC_{(0-∞)} | t_{1/2} | F |
|---|---|---|---|---|---|---|---|---|---|
| Group | (mL/min/kg) | (L/kg) | (ng/mL) | (ng/mL) | (h) | (h*ng/mL) | (h*ng/mL) | (h) | (%) |
| IV | 10.2 | 0.393 | 3154 | NA | NA | 1641 | 1648 | 0.541 | NA |
| PO | NA | NA | NA | 11533 | 2.67 | 66163 | NA | NA | 202 |

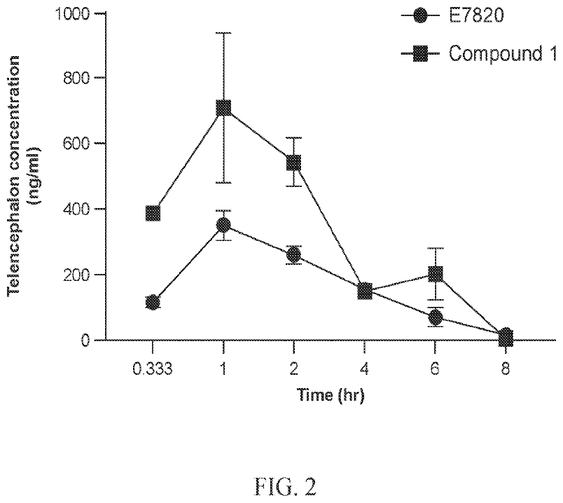

The data in FIG. 1 and Tables 4 and 5 show that Compound 1 demonstrates a greater than 2-fold increase in plasma AUC after oral administration compared to the control compound E7820. As shown in FIG. 2, Compound 1 also exhibits a 2-fold increase in telencephalon AUC after oral administration as compared to E7820.

### Example 40. Deuterated Derivative

Single dose PK parameters were also determined for Compound 143, a deuterated derivative of Compound 1, according to the methods described herein. The data in Table 6 show that Compound 143 demonstrates improved metabolic stability and permeability compared to Compound 1 and comparative compound E7820. Metabolic stability was assessed by measuring the half-life of the test compounds in human and mouse liver microsomes. Compound 143 also demonstrated an improved efflux ratio in MDCK cells.

TABLE 6

| | | | | Metabolic | | MDCK | |
|---|---|---|---|---|---|---|---|
| | | | | Stability | | Permeability | |
| | Physical Property | | | Half-life (min) | | | Efflux |
| Compound | MW | tPSA | clogP | Human | Mouse | P_{app(A-B)} | Ratio |
| E7820 | 336.4 | 109.5 | 3.1 | 51 | 29 | 1.1 | 30 |
| Compound 1 | 332.4 | 98.6 | 2.5 | 66 | 47 | 1.9 | 26 |
| Compound 143 | 335.4 | 98.6 | 2.5 | 93 | 67 | 2.9 | 12 |

TABLE 7

| Compound | Cₘₐₓ (ng/mL) | AUC_{(0-8 hr)} (h*ng/mL) |
|---|---|---|
| Compound 1 | 9847 | 62335 |
| Compound 143 | 19333 | 107406 |

TABLE 8

| Compound | Cₘₐₓ (ng/mL) | AUC_{(0-8 hr)} (h*ng/mL) |
|---|---|---|
| Compound 1 | 193 | 774 |
| Compound 143 | 373 | 2060 |

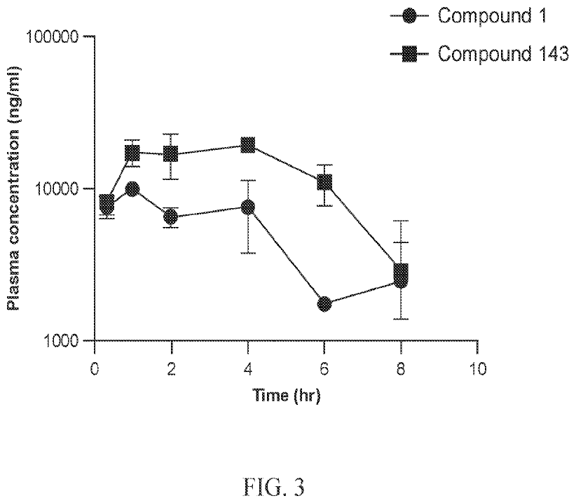

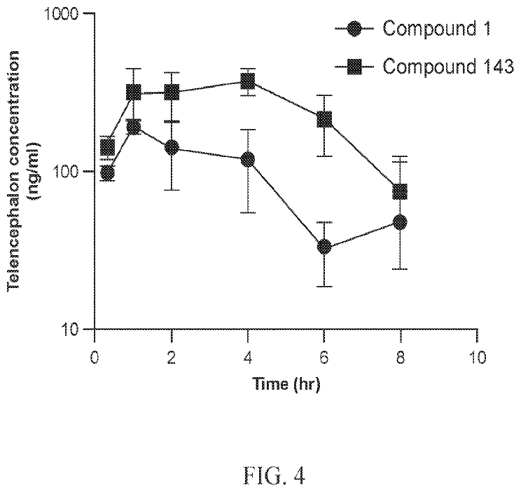

The data in FIG. 3 and Table 7 show that Compound 143 demonstrates an increase in plasma AUC after oral administration compared to Compound 1. As shown in FIG. 4 and Table 8, Compound 143 also exhibits a 2.5-fold increase in telencephalon AUC after oral administration compared to Compound 1.

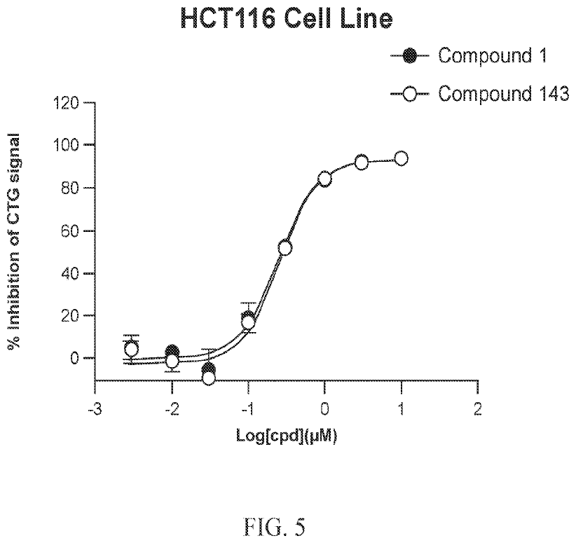

Cell viability assays were performed to compare the potency of Compound 1 to Compound 143. As shown in FIG. 5, no significant difference was observed between Compound 1 and Compound 143 for inhibiting cell growth in HCT116 colorectal cells. Compound 1 exhibits an IC₅₀ of 0.2587 μM, whereas Compound 143 exhibits an IC₅₀ of 0.2554 μM.

### Example 41. hERG Inhibition Assay

Inhibition of compounds on human ether-a-go-go related gene (hERG) channel was evaluated using a SyncroPatch 3848/384 automated patch clamp system. The SynchroPatch system is an independent method of measuring ion flux through ion channel proteins by measuring currents induced by ion flux into and out of the cell.

CHO herg-DUO cells stably expressing hERG channel were cultured in a medium containing F12 (HAM) medium, 10% FBS, 100 μg/mL penicillin-streptomycin, 100 100 g/mL hygromycin and 100 g/mL G418. Cells were split using TrypLE™ Express about three times a week, and maintained at about 80% confluence.

Compounds were prepared as a 50 mM stock solution in DMSO. The stock solution of each compound was serially diluted with DMSO to prepare 30 mM, 10 mM, 3.3 mM and 1.1 mM solutions. Working solutions 60 μM, 20 μM, 6.6 μM and 2.22 μM were prepared by 500-fold dilution of the 50 mM, 30 mM, 10 mM, 3.3 mM and 1.1 mM solutions, respectively using extracellular NMDG60 solution (80 mM NaCl, 60 mM N-methyl-d-glucamine, 4 mM KCl, 2 mM CaCl₂, 1 mM MgCl₂, 5 mM D-glucose and 10 mM HEPES).

Cells were harvested and added to SyncroPatch 384 chip (Nanion Technologies) and washed four times. Following establishment of the whole-cell voltage clamp configuration, cells were set to a holding potential of −90 mV for measured for 500 ms and currents were at 500 Hz and filtered at 3 kHz. The hERG current was elicited by depolarizing the membrane to +30 mV for 4.8 sec and the voltage was then taken back to −50 mV for 5.2 sec to remove the inactivation and measure the deactivating tail current for a sample interval of 15 s. The maximum amount of tail current size was used to determine hERG current amplitude. hERG current in the presence of test compounds was recorded for at least 5 min to reach a steady state and then 5 sweeps were captured. If a steady state was not reached within 10 minutes, the average peak current of the last 5 sweeps was substituted for the steady state value. Cisapride was used as a positive control.

TABLE 9

| Compound | hERG IC₅₀ (μM) |
|---|---|
| Cisapride | 0.015 |
| E7820 | 48.37 |
| Compound 1 | >50 |
| Compound 143 | >50 |

As shown in Table 9, Compound 1 and Compound 143 demonstrate less inhibition compared to E7820.

While some embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the present technology. This includes the generic description of the present technology with a proviso or negative limitation removing any subject matter from the genus, regardless of whether or not the excised material is specifically recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

Although the invention has been described with reference to embodiments and examples, it should be understood that numerous and various modifications can be made without departing from the spirit of the invention. Accordingly, the invention is limited only by the following claims.

1-72. (canceled)

73. A compound selected from the group consisting of: 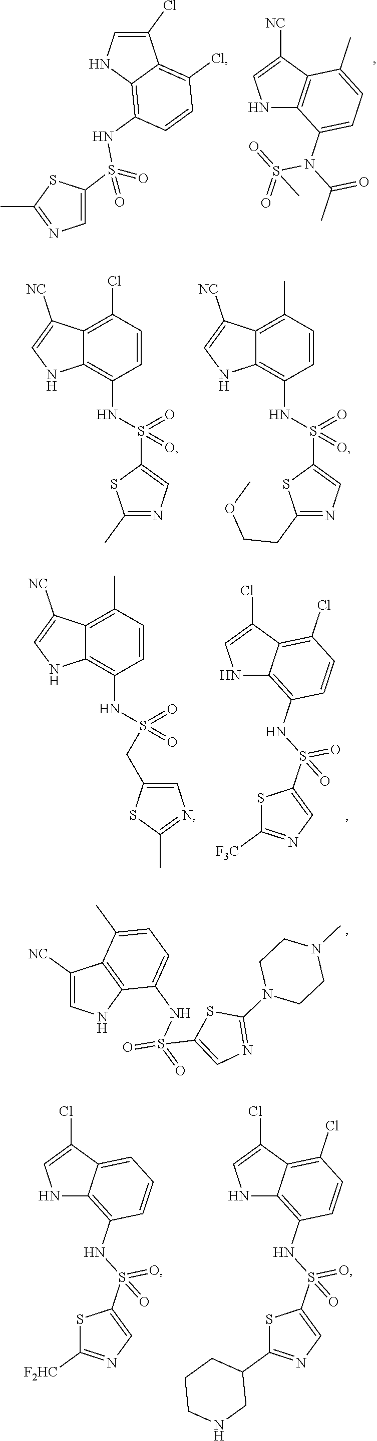 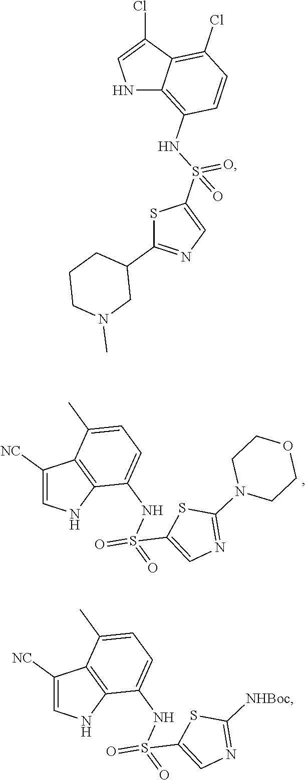 [SMILES: CN1CCCC(c2ncc(S(=O)(=O)Nc3ccc(Cl)c4c(Cl)c[nH]c34)s2)C1 ; Cc1ccc(NS(=O)(=O)c2cnc(N3CCOCC3)s2)c2[nH]cc(C#N)c12 ; Cc1ccc(NS(=O)(=O)c2cnc(NC(=O)OC(C)(C)C)s2)c2[nH]cc(C#N)c12] 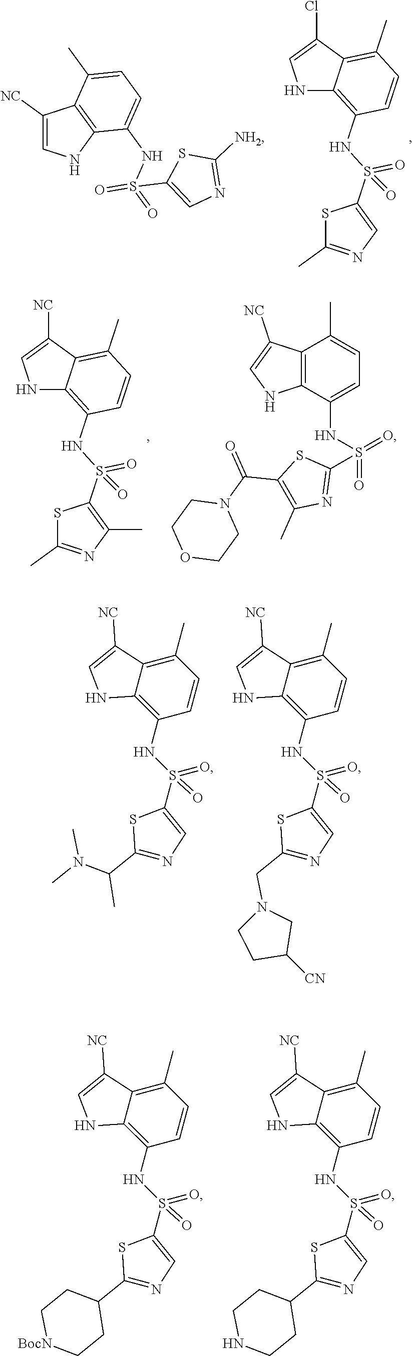 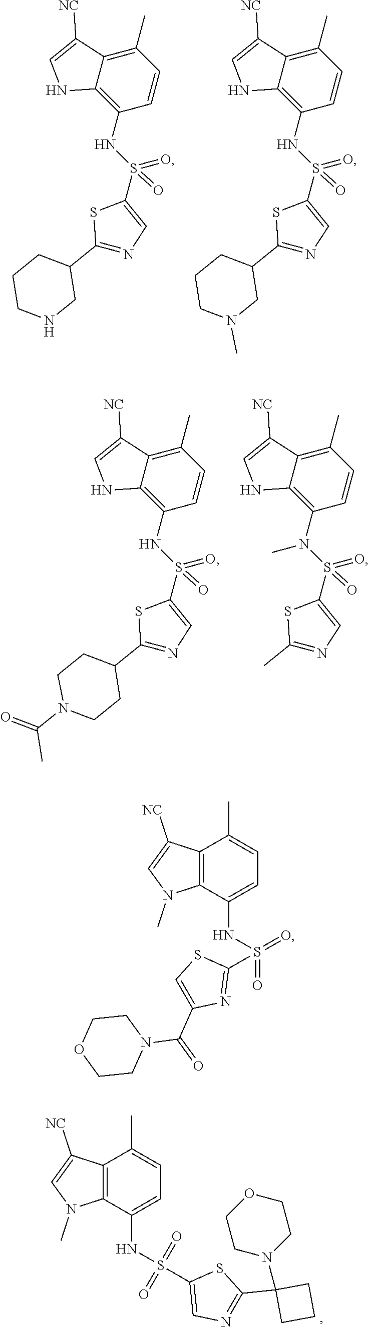 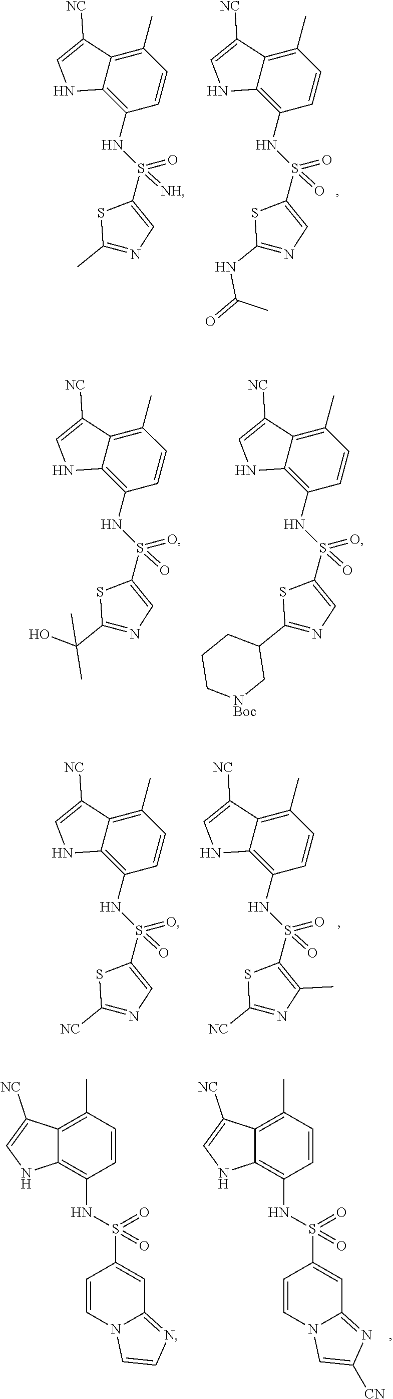 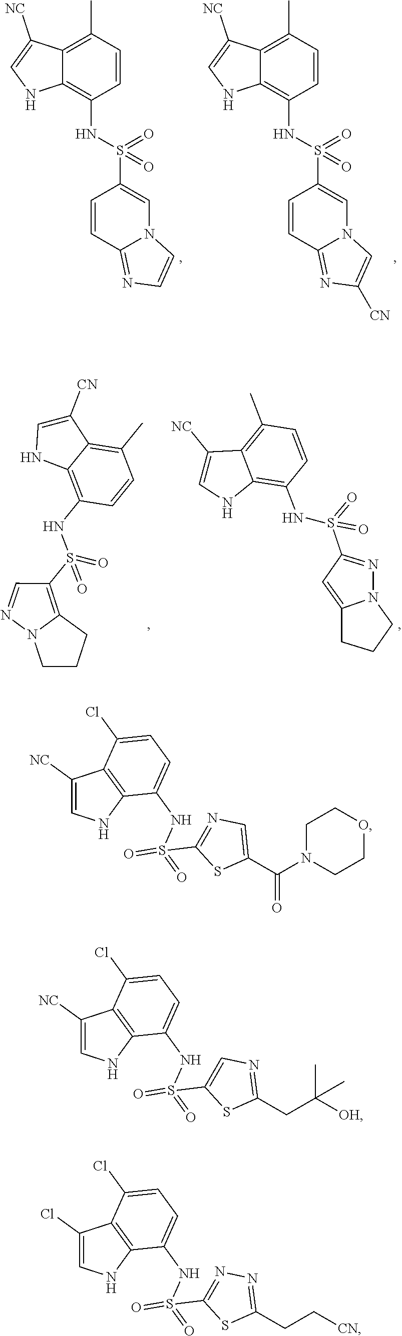 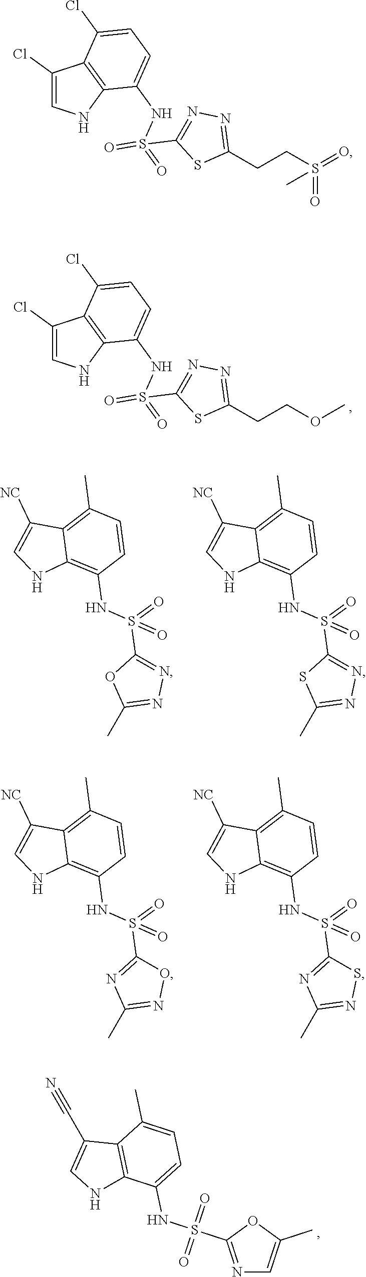 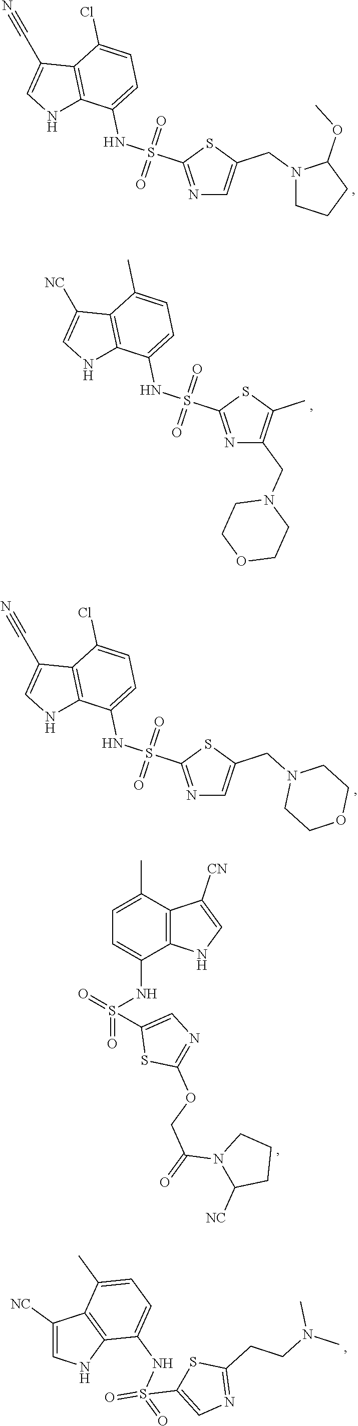 [SMILES: COC1CCCN1Cc1cnc(S(=O)(=O)Nc2ccc(Cl)c3c(C#N)c[nH]c23)s1 ; Cc1sc(S(=O)(=O)Nc2ccc(C)c3c(C#N)c[nH]c23)nc1CN1CCOCC1 ; N#Cc1c[nH]c2c(NS(=O)(=O)c3ncc(CN4CCOCC4)s3)ccc(Cl)c12 ; Cc1ccc(NS(=O)(=O)c2cnc(OCC(=O)N3CCCC3C#N)s2)c2[nH]cc(C#N)c12 ; Cc1ccc(NS(=O)(=O)c2cnc(CCN(C)C)s2)c2[nH]cc(C#N)c12] 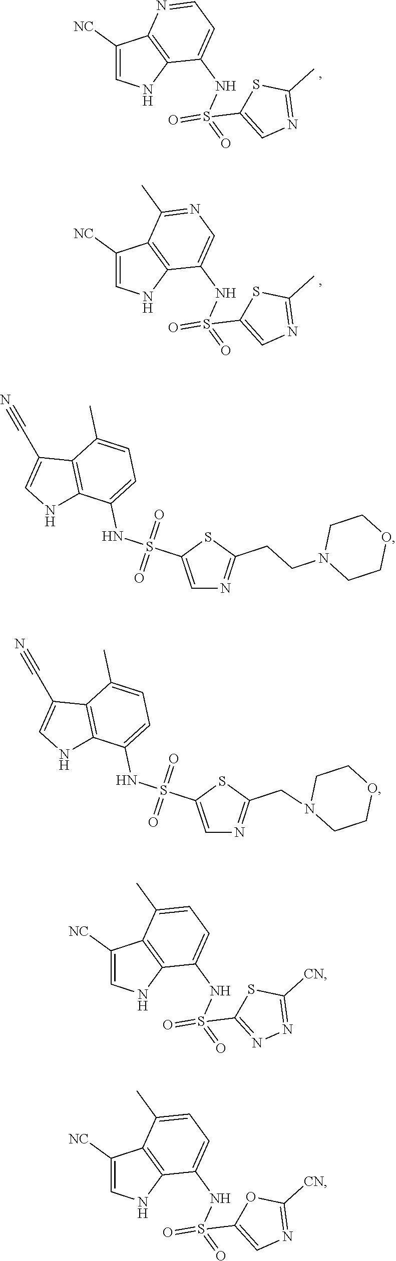 [SMILES: Cc1ncc(S(=O)(=O)Nc2ccnc3c(C#N)c[nH]c23)s1 ; Cc1ncc(S(=O)(=O)Nc2cnc(C)c3c(C#N)c[nH]c23)s1 ; Cc1ccc(NS(=O)(=O)c2cnc(CCN3CCOCC3)s2)c2[nH]cc(C#N)c12 ; Cc1ccc(NS(=O)(=O)c2cnc(CN3CCOCC3)s2)c2[nH]cc(C#N)c12 ; Cc1ccc(NS(=O)(=O)c2nnc(C#N)s2)c2[nH]cc(C#N)c12 ; Cc1ccc(NS(=O)(=O)c2cnc(C#N)o2)c2[nH]cc(C#N)c12] 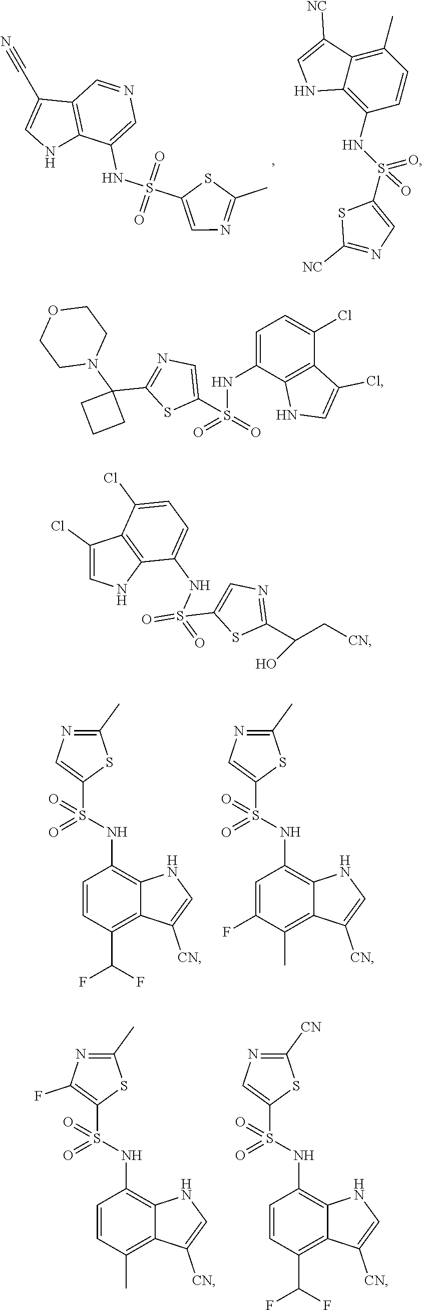 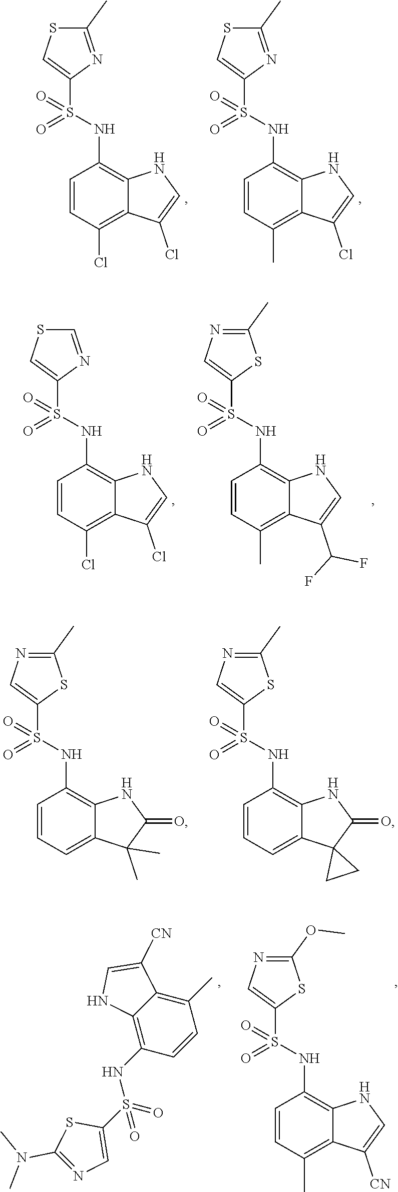 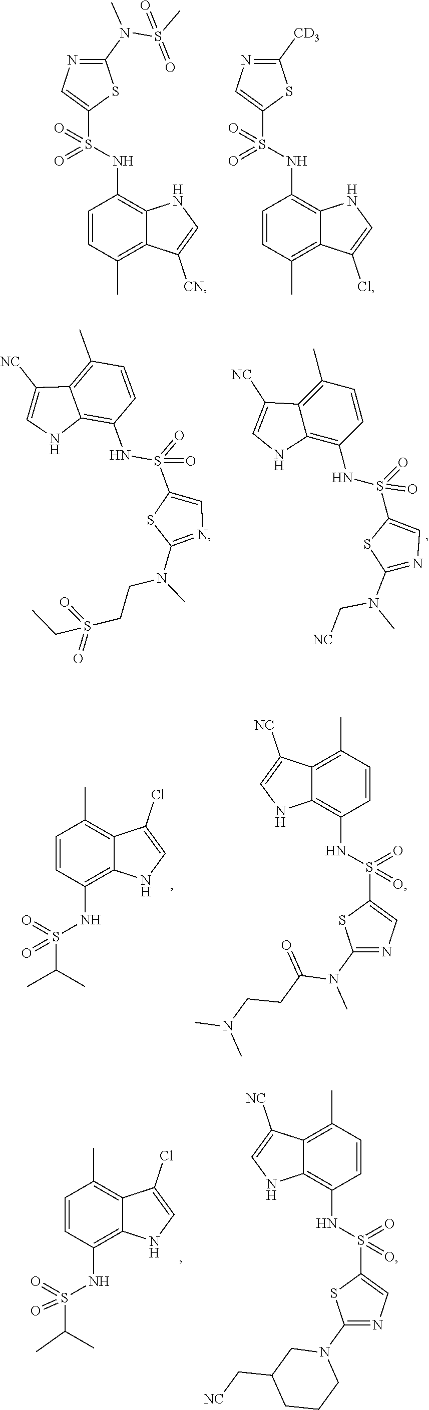 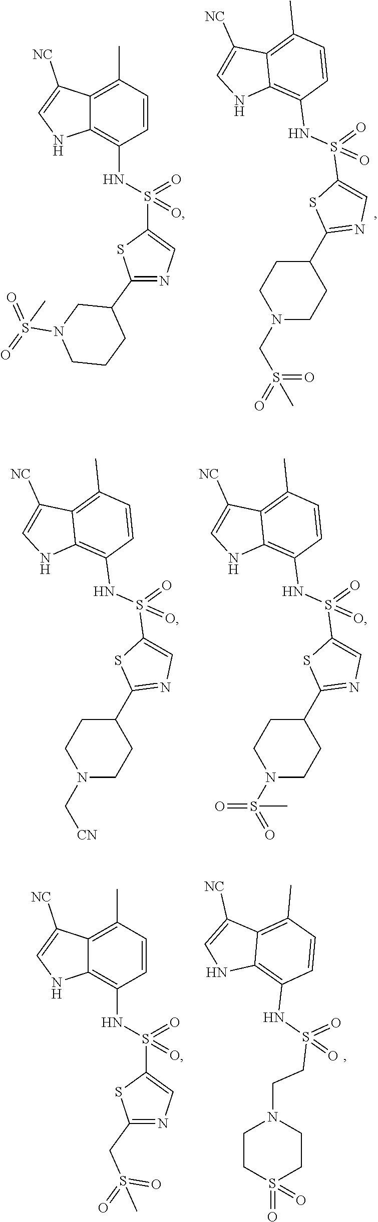 [SMILES: Cc1ccc(NS(=O)(=O)c2cnc(C3CCCN(S(C)(=O)=O)C3)s2)c2[nH]cc(C#N)c12 ; Cc1ccc(NS(=O)(=O)c2cnc(C3CCN(CS(C)(=O)=O)CC3)s2)c2[nH]cc(C#N)c12 ; Cc1ccc(NS(=O)(=O)c2cnc(C3CCN(CC#N)CC3)s2)c2[nH]cc(C#N)c12 ; Cc1ccc(NS(=O)(=O)c2cnc(C3CCN(S(C)(=O)=O)CC3)s2)c2[nH]cc(C#N)c12 ; Cc1ccc(NS(=O)(=O)c2cnc(CS(C)(=O)=O)s2)c2[nH]cc(C#N)c12 ; Cc1ccc(NS(=O)(=O)CCN2CCS(=O)(=O)CC2)c2[nH]cc(C#N)c12] 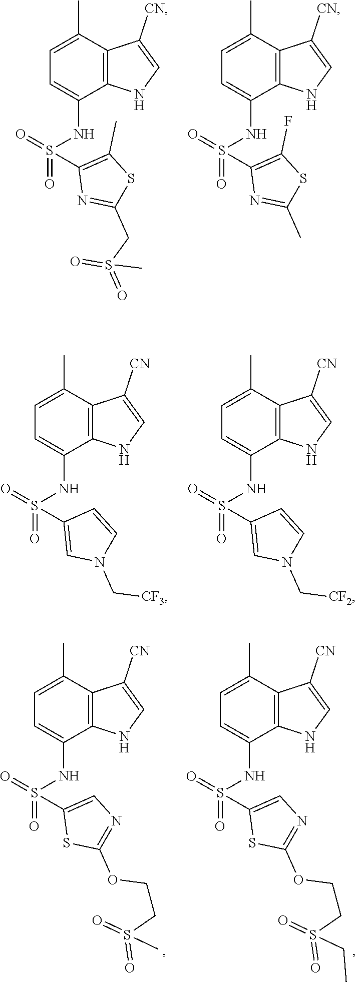 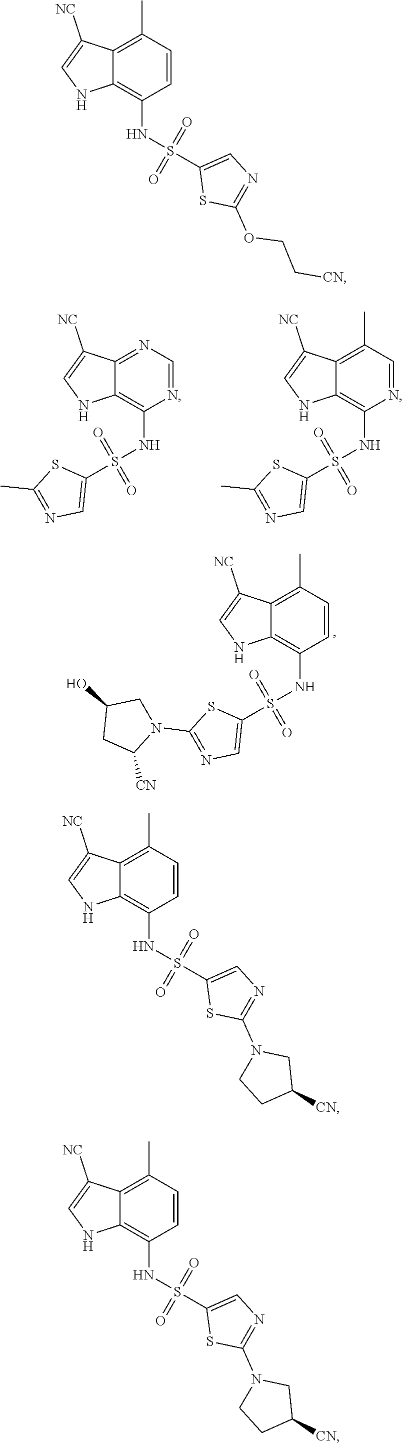 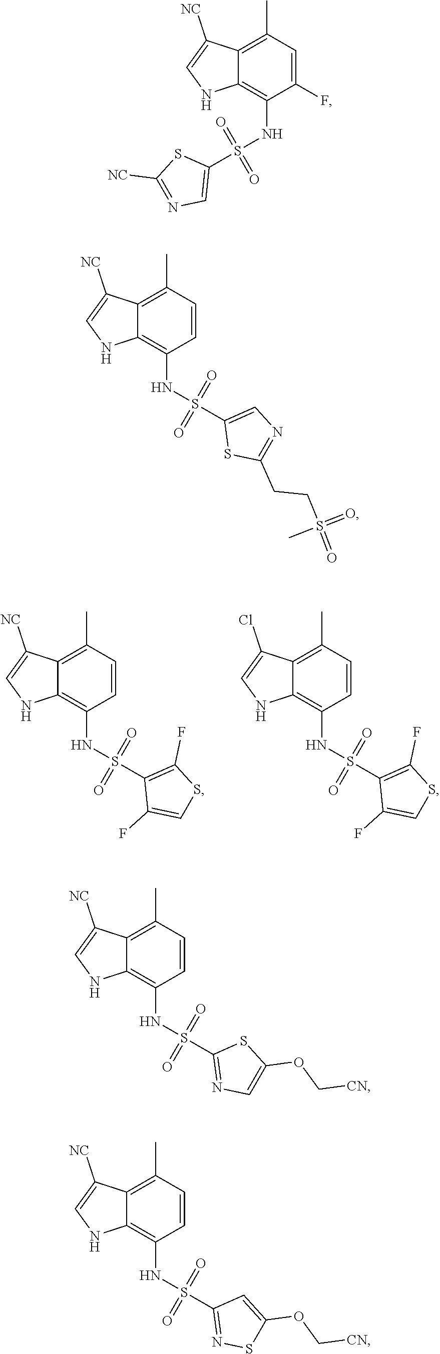 [SMILES: Cc1cc(F)c(NS(=O)(=O)c2cnc(C#N)s2)c2[nH]cc(C#N)c12 ; Cc1ccc(NS(=O)(=O)c2cnc(CCS(C)(=O)=O)s2)c2[nH]cc(C#N)c12 ; Cc1ccc(NS(=O)(=O)c2c(F)csc2F)c2[nH]cc(C#N)c12 ; Cc1ccc(NS(=O)(=O)c2c(F)csc2F)c2[nH]cc(Cl)c12 ; Cc1ccc(NS(=O)(=O)c2ncc(OCC#N)s2)c2[nH]cc(C#N)c12 ; Cc1ccc(NS(=O)(=O)c2cc(OCC#N)sn2)c2[nH]cc(C#N)c12] 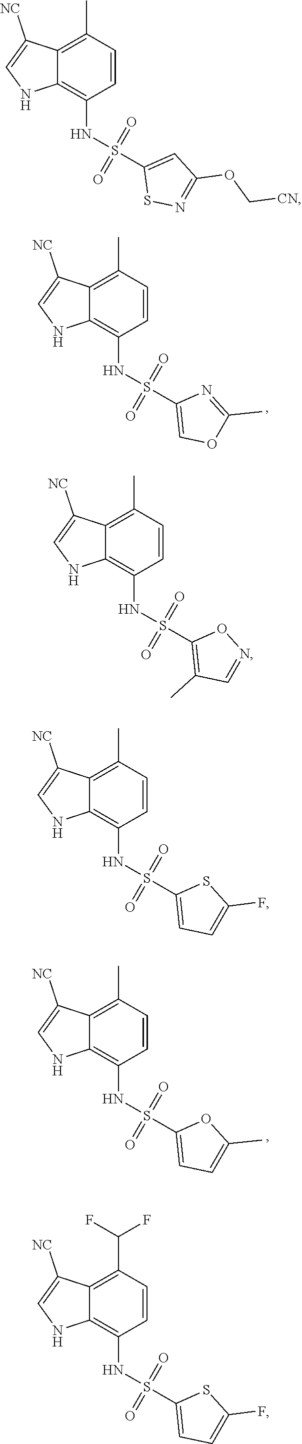 [SMILES: Cc1ccc(NS(=O)(=O)c2cc(OCC#N)ns2)c2[nH]cc(C#N)c12 ; Cc1nc(S(=O)(=O)Nc2ccc(C)c3c(C#N)c[nH]c23)co1 ; Cc1cnoc1S(=O)(=O)Nc1ccc(C)c2c(C#N)c[nH]c12 ; Cc1ccc(NS(=O)(=O)c2ccc(F)s2)c2[nH]cc(C#N)c12 ; Cc1ccc(S(=O)(=O)Nc2ccc(C)c3c(C#N)c[nH]c23)o1 ; N#Cc1c[nH]c2c(NS(=O)(=O)c3ccc(F)s3)ccc(C(F)F)c12] 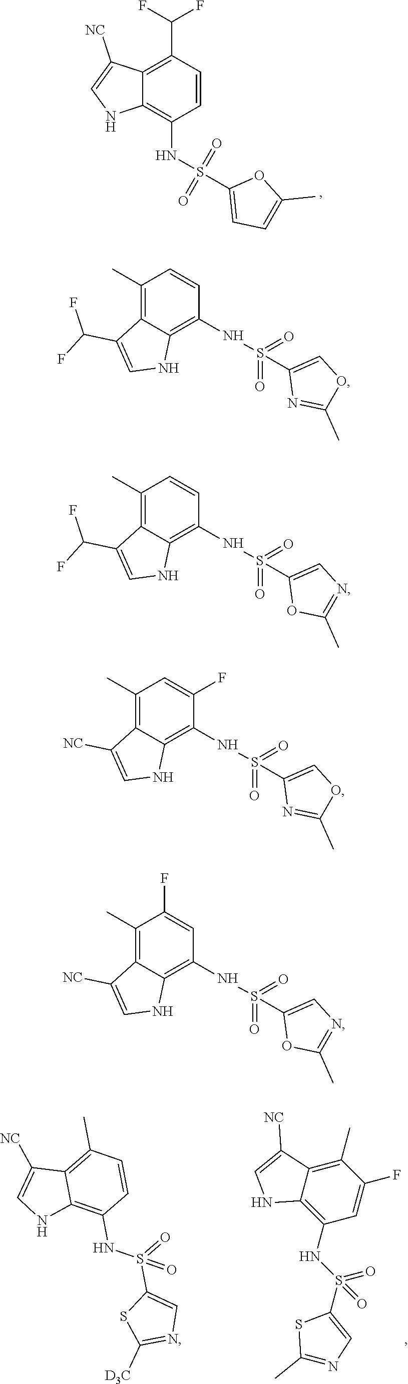 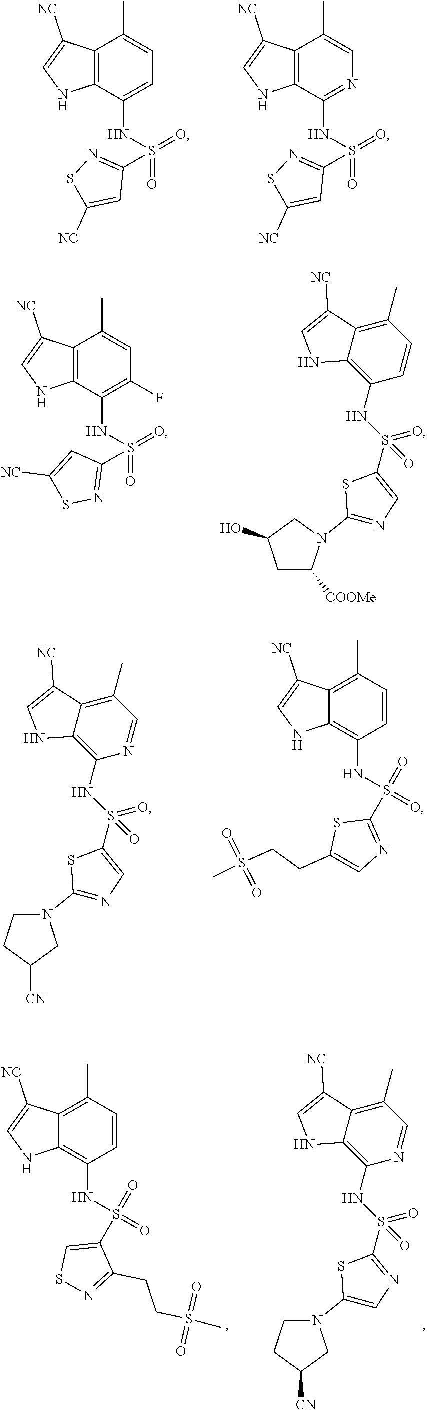 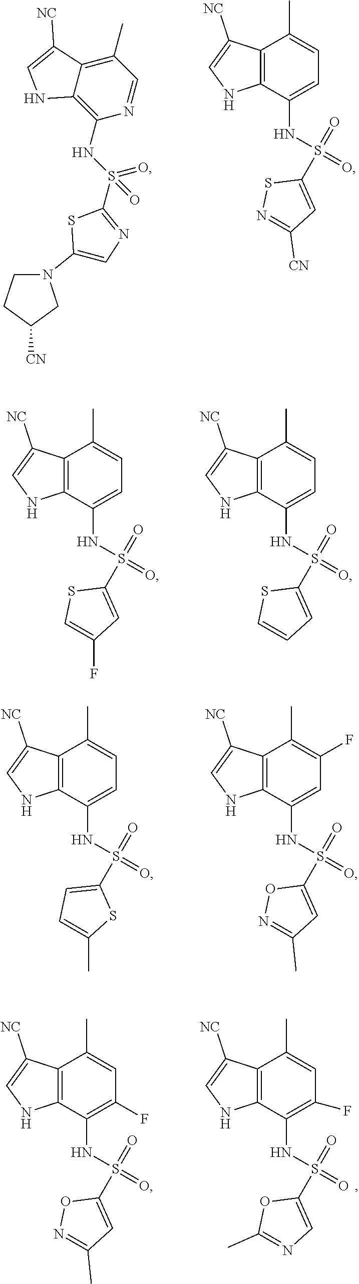 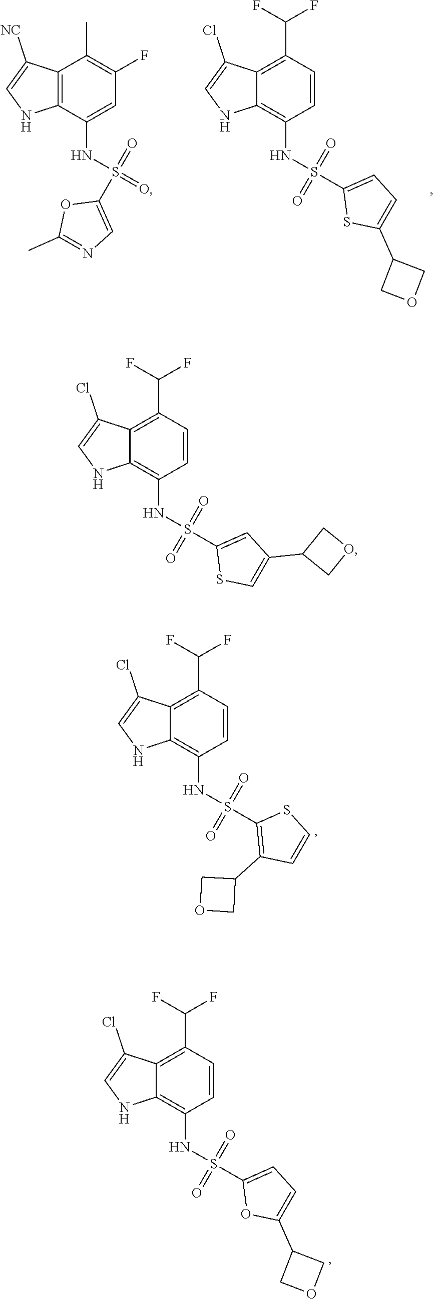 [SMILES: Cc1ncc(S(=O)(=O)Nc2cc(F)c(C)c3c(C#N)c[nH]c23)o1 ; O=S(=O)(Nc1ccc(C(F)F)c2c(Cl)c[nH]c12)c1ccc(C2COC2)s1 ; O=S(=O)(Nc1ccc(C(F)F)c2c(Cl)c[nH]c12)c1cc(C2COC2)cs1 ; O=S(=O)(Nc1ccc(C(F)F)c2c(Cl)c[nH]c12)c1sccc1C1COC1 ; O=S(=O)(Nc1ccc(C(F)F)c2c(Cl)c[nH]c12)c1ccc(C2COC2)o1] 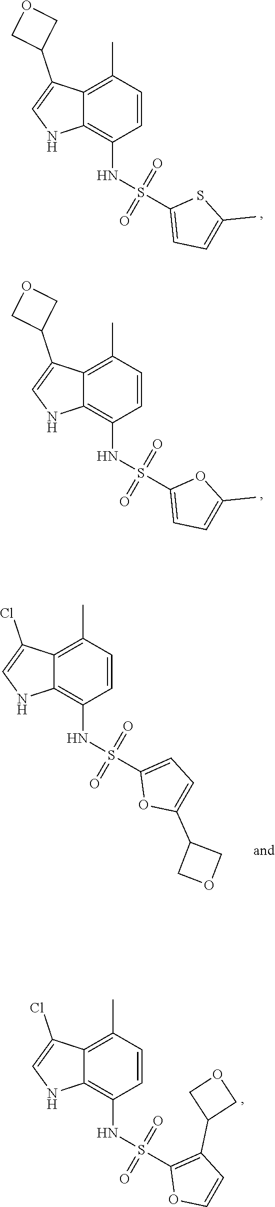 [SMILES: Cc1ccc(S(=O)(=O)Nc2ccc(C)c3c(C4COC4)c[nH]c23)s1 ; Cc1ccc(S(=O)(=O)Nc2ccc(C)c3c(C4COC4)c[nH]c23)o1 ; Cc1ccc(NS(=O)(=O)c2ccc(C3COC3)o2)c2[nH]cc(Cl)c12 ; Cc1ccc(NS(=O)(=O)c2occc2C2COC2)c2[nH]cc(Cl)c12]
or a pharmaceutically acceptable salt of any of the foregoing.

74. A pharmaceutical composition comprising an effective amount of a compound of claim 73, or a pharmaceutically acceptable salt thereof, and an excipient.

75. A method of treating cancer in a subject comprising administering to a subject in need thereof an effective amount of a compound of claim 73, or a pharmaceutically acceptable salt thereof.

76. The method of claim 75, further comprising administering surgery, radiation therapy, chemotherapy, targeted therapy, immunotherapy, immune checkpoint therapy, hormonal therapy, or antiviral therapy.

77-82. (canceled)

83. The compound of claim 73, wherein the compound is selected from the group consisting of:

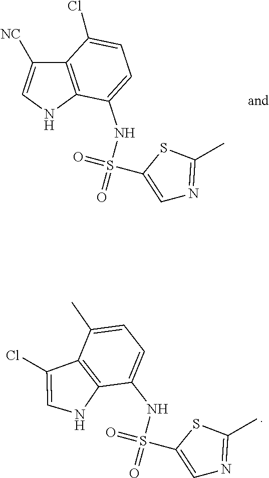 [SMILES: Cc1ncc(S(=O)(=O)Nc2ccc(Cl)c3c(C#N)c[nH]c23)s1 ; Cc1ncc(S(=O)(=O)Nc2ccc(C)c3c(Cl)c[nH]c23)s1]

84. The compound of claim 73, wherein the compound is selected from the group consisting of:

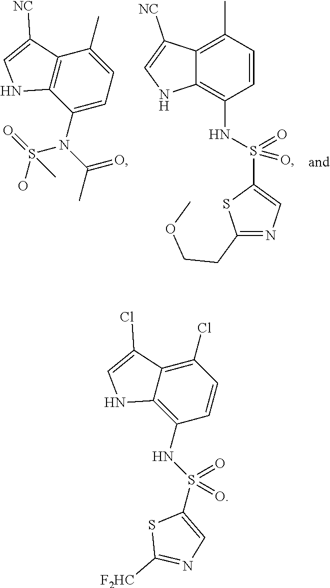

85. The compound of claim 73, wherein the compound is selected from the group consisting of:

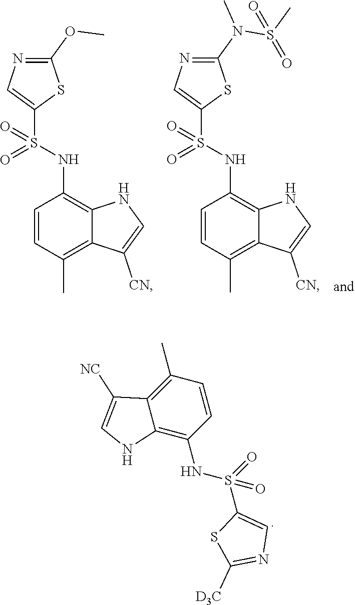 [SMILES: COc1ncc(S(=O)(=O)Nc2ccc(C)c3c(C#N)c[nH]c23)s1 ; Cc1ccc(NS(=O)(=O)c2cnc(N(C)S(C)(=O)=O)s2)c2[nH]cc(C#N)c12 ; [2H]C([2H])([2H])c1ncc(S(=O)(=O)Nc2ccc(C)c3c(C#N)c[nH]c23)s1]

86. The compound of claim 73, wherein the compound is selected from the group consisting of:

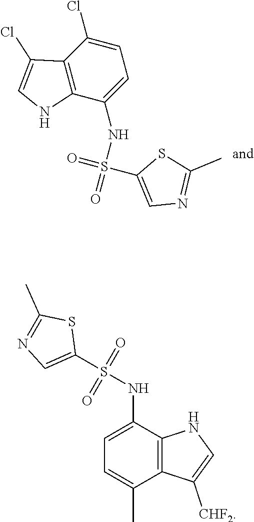 [SMILES: Cc1ncc(S(=O)(=O)Nc2ccc(Cl)c3c(Cl)c[nH]c23)s1 ; Cc1ncc(S(=O)(=O)Nc2ccc(C)c3c(C(F)F)c[nH]c23)s1]

87. The compound of claim 73, wherein the compound is selected from the group consisting of:

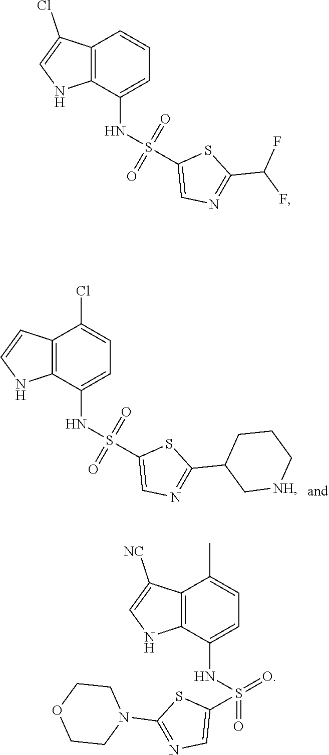 [SMILES: O=S(=O)(Nc1cccc2c(Cl)c[nH]c12)c1cnc(C(F)F)s1 ; O=S(=O)(Nc1ccc(Cl)c2cc[nH]c12)c1cnc(C2CCCNC2)s1 ; Cc1ccc(NS(=O)(=O)c2cnc(N3CCOCC3)s2)c2[nH]cc(C#N)c12]

88. The compound of claim 73, wherein the compound is selected from the group consisting of:

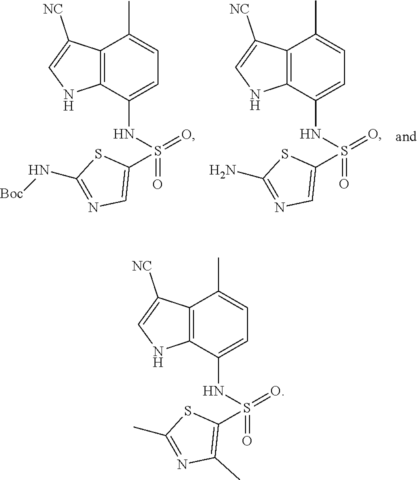 [SMILES: Cc1ccc(NS(=O)(=O)c2cnc(NC(=O)OC(C)(C)C)s2)c2[nH]cc(C#N)c12 ; Cc1ccc(NS(=O)(=O)c2cnc(N)s2)c2[nH]cc(C#N)c12 ; Cc1nc(C)c(S(=O)(=O)Nc2ccc(C)c3c(C#N)c[nH]c23)s1]

89. The compound of claim 73, wherein the compound is selected from the group consisting of:

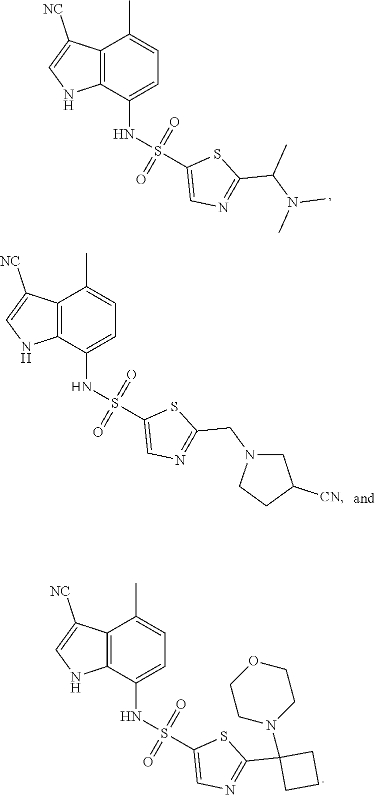 [SMILES: Cc1ccc(NS(=O)(=O)c2cnc(C(C)N(C)C)s2)c2[nH]cc(C#N)c12 ; Cc1ccc(NS(=O)(=O)c2cnc(CN3CCC(C#N)C3)s2)c2[nH]cc(C#N)c12 ; Cc1ccc(NS(=O)(=O)c2cnc(C3(N4CCOCC4)CCC3)s2)c2[nH]cc(C#N)c12]

90. The compound of claim 73, wherein the compound is selected from the group consisting of:

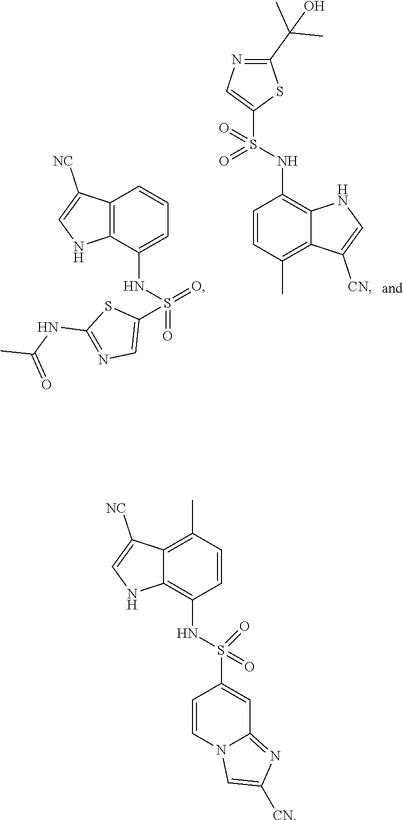 [SMILES: CC(=O)Nc1ncc(S(=O)(=O)Nc2cccc3c(C#N)c[nH]c23)s1 ; Cc1ccc(NS(=O)(=O)c2cnc(C(C)(C)O)s2)c2[nH]cc(C#N)c12 ; Cc1ccc(NS(=O)(=O)c2ccn3cc(C#N)nc3c2)c2[nH]cc(C#N)c12]

91. The compound of claim 73, wherein the compound is selected from the group consisting of:

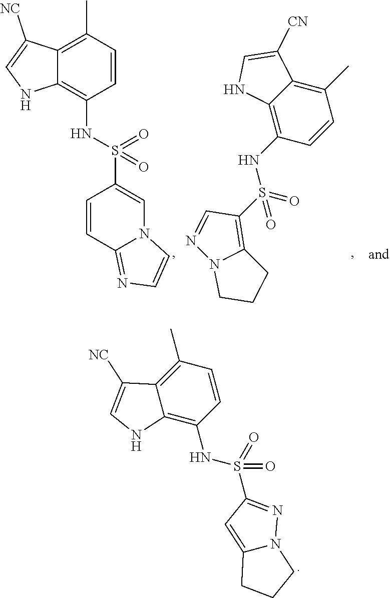

92. The compound of claim 73, wherein the compound is selected from the group consisting of:

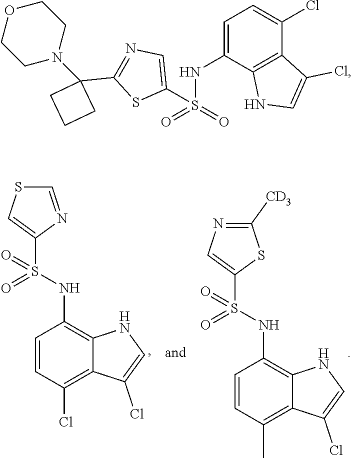 [SMILES: O=S(=O)(Nc1ccc(Cl)c2c(Cl)c[nH]c12)c1cnc(C2(N3CCOCC3)CCC2)s1 ; O=S(=O)(Nc1ccc(Cl)c2c(Cl)c[nH]c12)c1cscn1 ; [2H]C([2H])([2H])c1ncc(S(=O)(=O)Nc2ccc(C)c3c(Cl)c[nH]c23)s1]

93. The compound of claim 73, wherein the compound is selected from the group consisting of:

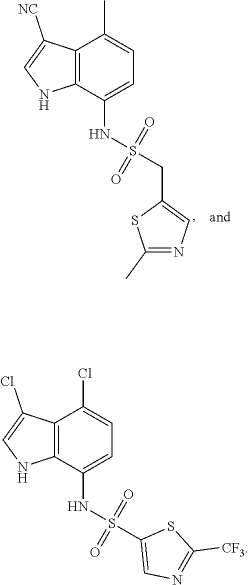 [SMILES: Cc1ncc(CS(=O)(=O)Nc2ccc(C)c3c(C#N)c[nH]c23)s1 ; O=S(=O)(Nc1ccc(Cl)c2c(Cl)c[nH]c12)c1cnc(C(F)(F)F)s1]

94. The compound of claim 73, wherein the compound is selected from the group consisting of:

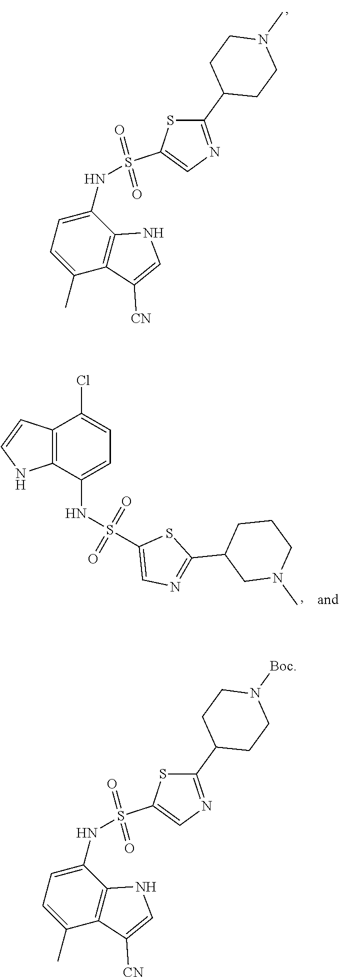 [SMILES: Cc1ccc(NS(=O)(=O)c2cnc(C3CCN(C)CC3)s2)c2[nH]cc(C#N)c12 ; CN1CCCC(c2ncc(S(=O)(=O)Nc3ccc(Cl)c4cc[nH]c34)s2)C1 ; Cc1ccc(NS(=O)(=O)c2cnc(C3CCN(C(=O)OC(C)(C)C)CC3)s2)c2[nH]cc(C#N)c12]

95. The compound of claim 73, wherein the compound is selected from the group consisting of:

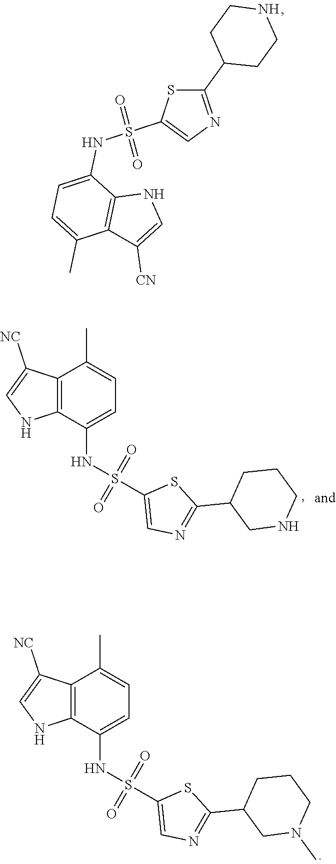 [SMILES: Cc1ccc(NS(=O)(=O)c2cnc(C3CCNCC3)s2)c2[nH]cc(C#N)c12 ; Cc1ccc(NS(=O)(=O)c2cnc(C3CCCNC3)s2)c2[nH]cc(C#N)c12 ; Cc1ccc(NS(=O)(=O)c2cnc(C3CCCN(C)C3)s2)c2[nH]cc(C#N)c12]

96. The compound of claim 73, wherein the compound is selected from the group consisting of:

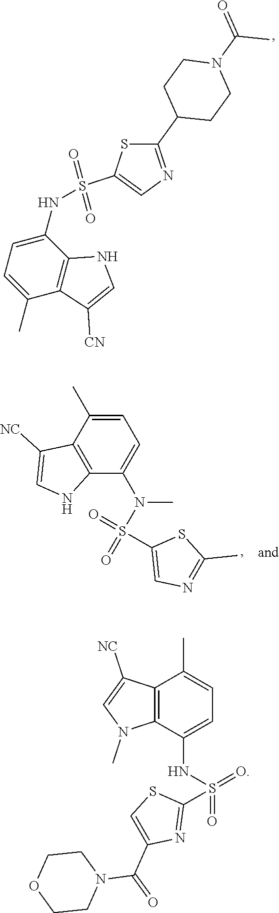 [SMILES: CC(=O)N1CCC(c2ncc(S(=O)(=O)Nc3ccc(C)c4c(C#N)c[nH]c34)s2)CC1 ; Cc1ncc(S(=O)(=O)N(C)c2ccc(C)c3c(C#N)c[nH]c23)s1 ; Cc1ccc(NS(=O)(=O)c2nc(C(=O)N3CCOCC3)cs2)c2c1c(C#N)cn2C]

97. The compound of claim 73, wherein the compound is selected from the group consisting of:

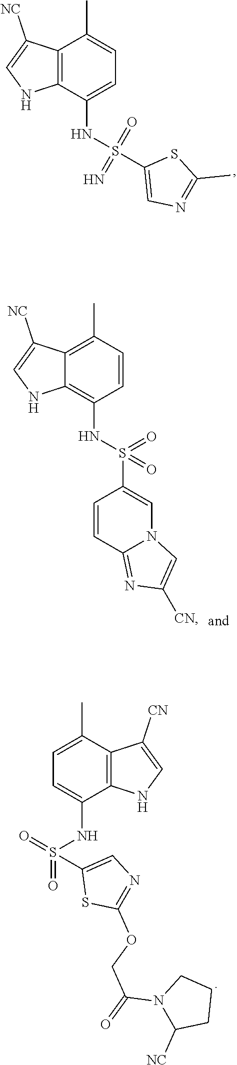 [SMILES: Cc1ncc(S(=N)(=O)Nc2ccc(C)c3c(C#N)c[nH]c23)s1 ; Cc1ccc(NS(=O)(=O)c2ccc3nc(C#N)cn3c2)c2[nH]cc(C#N)c12 ; Cc1ccc(NS(=O)(=O)c2cnc(OCC(=O)N3CCCC3C#N)s2)c2[nH]cc(C#N)c12]

98. The compound of claim 73, wherein the compound is selected from the group consisting of: 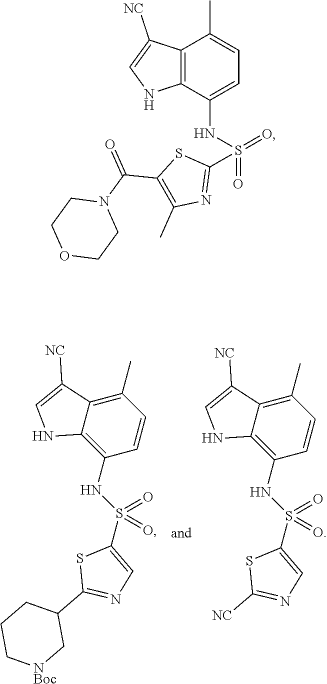 [SMILES: Cc1nc(S(=O)(=O)Nc2ccc(C)c3c(C#N)c[nH]c23)sc1C(=O)N1CCOCC1 ; Cc1ccc(NS(=O)(=O)c2cnc(C3CCCN(C(=O)OC(C)(C)C)C3)s2)c2[nH]cc(C#N)c12 ; Cc1ccc(NS(=O)(=O)c2cnc(C#N)s2)c2[nH]cc(C#N)c12]
